(19) United States
(12) Patent Application Publication
(10) Pub. No.: US 2019/0004065 A1
(43) Pub. Date: Jan. 3, 2019

(54) BIOMARKERS FOR STROKE

(71) Applicant: MOREHOUSE SCHOOL OF MEDICINE, Atlanta, GA (US)

(72) Inventors: Byron D. FORD, Riverside, CA (US); Gregory FORD, Cartersville, GA (US)

(21) Appl. No.: 15/901,523

(22) Filed: Feb. 21, 2018

Related U.S. Application Data
(63) Continuation of application No. 13/204,358, filed on Aug. 5, 2011, now abandoned.

(51) Int. Cl. G01N 33/68; C12Q 1/6883

(52) U.S. Cl. CPC G01N 33/6893; G01N 2800/60; G01N 2800/56; G01N 2800/52; G01N 2800/2871; C12Q 2600/158; C12Q 1/6883

(57) ABSTRACT

Biomarkers for stroke and methods for their detection are disclosed. In one aspect, the present application discloses biomarkers for the diagnosis of stroke in a subject. In another aspect, the application discloses a method for the diagnosis of stroke in a subject. The method comprises detection of stroke biomarkers in cerebrospinal fluid, blood, serum or PMBCs of a subject. Also disclosed is a kit for the detection of biomarkers for the diagnosis of stroke in a subject.

This application is a Continuation of U.S. application Ser. No. 13/204,358, filed Aug. 5, 2011, which claims priority from U.S. Provisional Patent Application No. 61/344,517, filed on Aug. 13, 2010. The entirety of the aforementioned application is incorporated herein by reference.

### FIELD

This application generally relates to the diagnosis, management and therapy of stroke. In particular, the invention relates to methods and kits for the diagnosis and monitoring the progression and treatment of stroke in a subject.

### BACKGROUND

Stroke is a debilitating condition with limited treatment options. Currently, thrombolysis is the only approved therapy for stroke. However, thrombolysis is effective in only 3-5% of stroke patients, primarily due to its very short therapeutic window. Stabilization of stroke patients inside and outside the therapeutic window for thrombolysis is also essential to prevent the progression of the initial ischemic lesion into expanding regions of brain damage. Imaging techniques, including computed tomography (CT) scanning and magnetic resonance imaging (MRI) are very useful in detecting the occurrence, type and severity of strokes. However, these technologies are expensive and require considerable amounts of time to develop a complete and correct diagnosis. This is particularly true in rural areas and cities with large, underserved populations.

In addition to the failure to produce effective therapies for stroke treatment, a dramatic disparity exists in the rate of mortality from stroke between African-Americans and white counterparts. Reducing health disparities associated with stroke remains a major public health challenge in the United States. Despite dramatic declines in stroke mortality between 1970 and 1990 for both white and African-Americans, African-American men and women still have almost twice the rate of death due to stroke as their white counterparts.

Prompt diagnosis of a stroke is important, as delays in diagnosis and medical intervention may contribute to clinical deterioration and disability. Early diagnosis enables clinicians to more effectively choose the emergency intervention such as anti-platelet and/or neuroprotective therapy, and also to make better prognoses of disease outcome. Successful treatment of stroke requires rapid state diagnosis. Delays in diagnosis reduce the amount of time available in which the brain can respond to reperfusion, and significantly increase the risk of hemorrhage after most of the permanent injury has occurred.

There has been a long-felt need to develop rapid, accessible and easy to use diagnostic tools to identify and treat stroke symptoms. The use of blood biomarkers for stroke has been long considered an excellent method to determine the occurrence, timing, subtype and severity of stroke. Blood biomarkers can also be used to determine the efficacy of existing and novel treatment strategies for stroke.

The search for specific, reliable and clinically useful biomarkers has been largely unsuccessful. Many biomarkers that were believed to have promise are only detectable many hours or days after a stroke, by which time they would no longer be helpful (Anand, N et al. (2005) Cerebrovasc Dis 20:213-219). Additionally, some assays for markers of interest require labor-intensive laboratory work, requiring long turn-around time for results and limited availability.

An ideal biomarker should be specific to the type of stroke (such as ischemic stroke), sensitive (early and immediate release), predictive (proportionate to the extent of injury), robust (accurate and inexpensive), non-invasive and bridge pre-clinical results and clinical validation. Advanced technologies including genomic analysis and proteomics have facilitated the discovery of effective cancer biomarkers and could lead to discovery of stroke markers. One advantage of high throughput microarray-based genomic and proteomic analyses is the capacity to identify a group or cluster of genes and proteins with altered expression patterns in tissue or body fluids that encode putative secreted or cell-surface proteins. An ischemic stroke genomic biomarker would be a measurable RNA characteristic that is an indicator of normal biological processes, ischemic injury and/or response to therapeutic interventions. A proteomic biomarker would detect peptides or proteins in plasma or serum. It is also important that a stroke biomarker correlate well with findings from neuroimaging and clinical diagnostic examinations.

### SUMMARY

One aspect of the present invention relates to a method for diagnosing stroke in a subject. The method comprises the steps of (a) measuring the level of one or more biomarkers in a sample from the subject; (b) comparing the level of the one or more biomarkers to a reference level of the one or more biomarkers; and (c) making a diagnosis based on the result of the comparing step. The one or more biomarkers comprise gene products expressed from genes selected from the group consisting of IL-1α, IL-1β, IL-1ra, IL-3, IL-2, IL-4, IL-5, IL-6, IL-7, IL-8, IL-9, IL-10, IL-12 (p40), IL-12(p70), IL-13, IL-15, IL-17, EGF, Eotaxin, FGF-2, FTL-3 ligand, Fractalkine, G-CSF, GM-CSF, GRO, IFN-α2, IFN-γ, IP-10, MCP-1, MCP-3, MCD, MIP-1α, MIP-1β, PDGF-aa, PGDF-aa bb, RANTES, sCD40L, sIL2-rα, TNF-α, TNF-β, VEGF and genes listed in Tables 4, 5, 6 and 8.

In one embodiment, the one or more biomarkers are polynucleotides.

In another embodiment, the one or more biomarkers are proteins or peptides.

In another embodiment, step (a) includes measuring a panel of three or more biomarkers in the sample from the subject.

In another embodiment, step (a) includes measuring a panel of three or more biomarkers in the sample from the subject. In certain embodiments, the panel comprises at least one immediate early stroke biomarker (i.e., markers diferentially expressed at 1 hour post-stroke), at least one early stroke biomarker (i.e., markers diferentially expressed at 2 hour post-stroke) and at least one late stroke biomarker (i.e., diferentially expressed at 24 hour post-stroke). In certain other embodiments, the panel comprises at least two immediate early stroke biomarker (diferentially expressed at 1 hour post-stroke), at least two early stroke biomarker (diferentially expressed at 2 hour post-stroke) and at least two late stroke biomarker (diferentially expressed at 24 hour post-stroke). In certain other embodiments, the panel comprises at least three immediate early stroke biomarkers, at least three early stroke biomarkers and at least three late stroke biomarkers.

Another aspect of the present invention relates to a method for determining disease progression in a subject after a stroke. The method comprises the steps of (a) measuring the level of one or more biomarkers in a first sample obtained from the subject at a first time point; (b) measuring the level of the one or more biomarkers in a second sample obtained from the subject at a second time point; (c) comparing the level of the one or more biomarkers at the first time point to the level of the one or more biomarkers at the second time point; and (d) determining the disease progression between the first and the second time point based on the result of step (c). The one or more biomarkers comprise gene products expressed from genes selected from the group consisting of IL-1α, IL-1β, IL-1ra, IL-3, IL-2, IL-4, IL-5, IL-6, IL-7, IL-8, IL-9, IL-10, IL-12 (p40), IL-12(p70), IL-13, IL-15, IL-17, EGF, Eotaxin, FGF-2, FTL-3 ligand, Fractalkine, G-CSF, GM-CSF, GRO, IFN-α2, IFN-γ, IP-10, MCP-1, MCP-3, MCD, MIP-1α, MIP-1β, PDGF-aa, PGDF-aa bb, RANTES, sCD40L, sIL2-rα, TNF-α, TNF-β, VEGF and genes listed in Tables 4, 5, 6 and 8.

Another aspect of the present invention relates to a method for determining the efficacy of a treatment for stroke in a subject. The method comprises the steps of (a) measuring the level of one or more biomarkers in a first sample obtained from the subject at a first time point; (b) measuring the level of the one or more biomarkers in a second sample obtained from the subject at a second time point, wherein the subject is under treatment at the second time point; (c) comparing the level of the one or more biomarkers at the first time point to the level of the one or more biomarkers at the second time point; and (d) determining the efficacy of the treatment based on the result of step (c). The one or more biomarkers comprise gene products expressed from genes selected from the group consisting of IL-1α, IL-1β, IL-1ra, IL-3, IL-2, IL-4, IL-5, IL-6, IL-7, IL-8, IL-9, IL-10, IL-12 (p40), IL-12(p70), IL-13, IL-15, IL-17, EGF, Eotaxin, FGF-2, FTL-3 ligand, Fractalkine, G-CSF, GM-CSF, GRO, IFN-α2, IFN-γ, IP-10, MCP-1, MCP-3, MCD, MIP-1α, MIP-1β, PDGF-aa, PGDF-aa bb, RANTES, sCD40L, sIL2-rα, TNF-α, TNF-β, VEGF and genes listed in Tables 4, 5, 6 and 8.

Another aspect of the present invention relates to a kit for detecting biomarkers for stroke in a biological sample. The kit comprises (a) reagents for detecting a panel of biomarkers for stroke, and (b) a instruction listing the reference range for each of the biomarkers. The panel of biomarkers comprise two or more biomarkers, and wherein the two or more biomarkers comprise gene products expressed from genes selected from the group consisting of IL-1α, IL-1β, IL-1ra, IL-3, IL-2, IL-4, IL-5, IL-6, IL-7, IL-8, IL-9, IL-10, IL-12 (p40), IL-12(p70), IL-13, IL-15, IL-17, EGF, Eotaxin, FGF-2, FTL-3 ligand, Fractalkine, G-CSF, GM-CSF, GRO, IFN-α2, IFN-γ, IP-10, MCP-1, MCP-3, MCD, MIP-1α, MIP-1β, PDGF-aa, PGDF-aa bb, RANTES, sCD40L, sIL2-rα, TNF-α, TNF-β, VEGF, NMDA receptor, neuronal specific enolase, GFAP, Apo C-III, MMP-9, D-dimer, CRP, brain natriuretic peptide, S100B and genes listed in Tables 4, 5, 6 and 8.

### BRIEF DESCRIPTION OF THE DRAWINGS

FIG. 1 shows diffusion weighted (DWI; FIGS. 1A, 1C) and T2-weighted (FIGS. 1B, 1D) MRI images collected immediately after and 24 hours following middle cerebral artery occlusion (MCAO) in a monkey subject (Monkey A035), increase in infarct size as measured by DWI 24 hours following MCAO (FIG. 1E), and infarct size as measured by DWI correlated with the motor impairment observed in the task-oriented neurological assessment (FIG. 1F).

FIG. 2 shows microarray analysis of RNA from PBMCs of non-human primates following MCAO. Blood was collected before MCAO (Pre-O) and after ischemia (1, 2 and 24 hours). Green indicates genes with low level or no expression. Red indicates genes induced following ischemia.

FIG. 3 shows levels of the cytokine monocyte chemotactic protein-1 (MCP-1) in serum increase significantly 1-2 hours after MCAO then return to baseline at 24 hours following stroke using a Luminex assay (FIG. 1A) and a Western blot analysis of serum confirming the Luminex results (FIG. 1b).

FIG. 4 shows peptidomic analysis of proteins from serum of non-human primates following MCAO. Blood was collected before MCAO (Pre-O) and after ischemia (1, 2 and 24 hours). Green indicates proteins with low level expression. Red indicates proteins increased following ischemia.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

As used herein, the following terms shall have the following meanings:

A “biomarker,” as used herein, refers to a small molecule, protein, or nucleic acid that can be detected and measured in parts of the body like the blood or tissue whose presence or concentration reflects the presence, severity, type or progression of stroke in a subject. More generally a biomarker is anything that can be used as an indicator of a particular disease state or some other biological state of an organism. In molecular terms biomarker is the subset of markers that might be detected in a subject using genomics, proteomics technologies or imaging technologies. A biomarker may include any of, but is not limited to, a cytokine, chemokine, growth factor, enzyme, or a protein associated with thrombosis. A biomarker can also include a nucleic acid that encodes any of the above proteins or an mRNA or microRNA that is differentially expressed during or following a stroke. A biomarker may be a gene product whose presence is increased following a stroke or whose presence is decreased following a stroke.

As used herein, the term “gene product” or “expression product of a gene” refers to the transcriptional products of a gene, such as mRNAs and cDNAs encoded by the gene, and/or the translational products of a gene, such as peptides encoded by the gene, and fragments thereof.

As used herein, the term “antibody” refers to immunoglobulin molecules and immunologically active portions of immunoglobulin (Ig) molecules, i.e., “antigen binding fragments thereof” or molecules that contain an antigen binding site that specifically binds (immunoreacts with) an antigen. The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. By “specifically bind” or “immunoreacts with” is meant that the antibody reacts with one or more antigenic determinants of the desired antigen and does not react (i.e., bind) with other polypeptides or binds at much lower affinity with other polypeptides. The term “antibody” also includes antibody fragments that comprise a portion of a full length antibody, generally the antigen binding or variable region thereof. Examples of antibody fragments include Fab, Fab′, F(ab′)2, and Fv fragments; diabodies; linear antibodies; single-chain antibody (scFv) molecules; and multispecific antibodies formed from antibody fragments. In certain embodiments of the invention, it may be desirable to use an antibody fragment, rather than an intact antibody, to increase tumor penetration, for example. In this case, it may be desirable to use an antibody fragment that has been modified by any means known in the art in order to increase its serum half life.

The term “monoclonal antibody” as used herein refers to an antibody obtained from a population of substantially homogeneous antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

The term “biological sample” refers to a sample of biological material obtained from a mammal subject, preferably a human subject, including a tissue, a tissue sample, a cell sample, peripheral blood mononuclear cells, and a biological fluid, e.g., blood, plasma, serum, saliva, urine, cerebral or spinal fluid, and lymph liquid. A biological sample may be obtained in the form of, e.g., a tissue biopsy, such as, an aspiration biopsy, a brush biopsy, a surface biopsy, a needle biopsy, a punch biopsy, an excision biopsy, an open biopsy, an incision biopsy and an endoscopic biopsy. In one embodiment, the biological sample is a blood, serum or plasma sample. In another embodiment, the biological sample is peripheral blood mononuclear cells.

An “isolate” of a biological sample (e.g., an isolate of a tissue or tumor sample) refers to a material or composition (e.g., a biological material or composition) which has been separated, derived, extracted, purified or isolated from the sample and preferably is substantially free of undesirable compositions and/or impurities or contaminants associated with the biological sample.

The term “increased level” refers to a level that is higher than a normal or control level customarily defined or used in the relevant art. For example, an increased level of immunostaining in a tissue is a level of immunostaining that would be considered higher than the level of immunostaining in a control tissue by a person of ordinary skill in the art.

The term “decreased level” refers to a level that is lower than a normal or control level customarily defined or used in the relevant art. For example, a decreased level of immunostaining in a tissue is a level of immunostaining that would be considered lower than the level of immunostaining in a control tissue by a person of ordinary skill in the art.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

The term “expression level of a stroke biomarker” may be measured at the transcription level, in which case the presence and/or the amount of a polynucleotide is determined, or at the translation level, in which case the presence and/or the amount of a polypeptide is determined. Stroke biomarker expression may be characterized using any suitable method.

One aspect of the present invention relates to a method for diagnosing stroke in a subject. One aspect of the present invention relates to a method for diagnosing stroke in a subject. The method comprises the steps of (a) measuring the level of one or more biomarkers in a sample from the subject; (b) comparing the level of the one or more biomarkers to a reference level of the one or more biomarkers; and (c) making a diagnosis based on the result of the comparing step.

In one embodiment, the stroke biomarker level in the biological sample is compared with a stroke marker level associated with a reference sample, such as a normal control sample. The phrase “normal control level” refers to the level of a stroke biomarker typically found in a biological sample of a population not suffering from stroke. The reference sample is preferably of a similar nature to that of the test sample. For example, if the test sample comprises patient serum, the reference sample should also be serum. The stroke biomarker level in the biological samples from control and test subjects may be determined at the same time or, alternatively, the normal control level may be determined by a statistical method based on the results obtained by analyzing the level of the stroke biomarker in samples previously collected from a control group.

In one embodiment, biomarkers are gene products expressed from genes selected from the group consisting of IL-1α, IL-1β, IL-1ra, IL-3, IL-2, IL-4, IL-5, IL-6, IL-7, IL-8, IL-9, IL-10, IL-12 (p40), IL-12(p70), IL-13, IL-15, IL-17, EGF, Eotaxin, FGF-2, FTL-3 ligand, Fractalkine, G-CSF, GM-CSF, GRO, IFN-α2, IFN-γ, IP-10, MCP-1, MCP-3, MCD, MIP-1α, MIP-1β, PDGF-aa, PGDF-aa bb, RANTES, sCD40L, sIL2-rα, TNF-α, TNF-β, VEGF, NMDA receptor, neuronal specific enolase, GFAP, Apo C-III, MMP-9, D-dimer, CRP, brain natriuretic peptide, S100B and genes listed in Tables 4, 5, 6 and 8.

In a related embodiment, the biomarkers are expression products of cytokine genes, chemokine genes or growth factor genes. In a further related embodiment, the cytokine, chemokine or growth factor genes are selected from genes encoding IL-1α, IL-1β, IL-1ra, IL-2, IL-3, IL-4, IL-5, IL-6, IL-7, IL-8, IL-9, IL-10, IL-12 (p40), IL-12 (p70), IL-13, IL-15, IL-17, epidermal growth factor (EGF), Eotaxin, FGF-2, FTL-3 ligand, Fractalkine, G-CSF, GM-CSF, GRO, IFN-α2, IFN-γ, IP-10, monocyte chemotactic protein-1 (MCP-1), MCP-3, MCD, MIP-1α, MIP-1β, PDGF-aa, PDGF-aa bb, RANTES, sCD40L, sIL2-rα, TNF-α, TNF-β, and VEGF.

In certain embodiments, the biomarkers are protein or peptide markers.

In certain other embodiments, the biomarkers are nucleic acid biomarkers. In certain related embodiments, the nucleic acid biomarkers are DNA or RNA biomarkers. In certain embodiments, expression of the stroke biomarkers is determined at the mRNA level by quantitative RT-PCR, Northern blot or other methods known to a person of ordinary skill in the art.

In some embodiments, a panel of multiple biomarkers is measured in step (a). Multimarker panels improve the diagnostic sensitivity and specificity of individual biomarkers. For example, by simultaneously targeting different components of the ischemic cascade, a panel of biomarkers can distinguish patients with acute stroke from age and gender-matched control subjects and establish the timing and severity of the stroke. In some embodiments, multimarker panels include biomarkers that indicate stroke incidence, timing after ischemia and severity of neuronal damage. In other embodiments, the panels would also include markers to confirm or rule out hemorrhage and transient ischemic attack (e.g. irreversible neuronal damage).

In other embodiments, step (a) includes measuring a panel of three or more biomarkers in the sample from the subject. In certain embodiments, the panel comprises at least one immediate early stroke biomarker (diferentially expressed at 1 hour post-stroke), at least one early stroke biomarker (diferentially expressed at 2 hour post-stroke) and at least one late stroke biomarker (diferentially expressed at 24 hour post-stroke). In certain other embodiments, the panel comprises at least two immediate early stroke biomarker (diferentially expressed at 1 hour post-stroke), at least two early stroke biomarker (diferentially expressed at 2 hour post-stroke) and at least two late stroke biomarker (diferentially expressed at 24 hour post-stroke). In certain other embodiments, the panel comprises at least three immediate early stroke biomarker (diferentially expressed at 1 hour post-stroke), at least three early stroke biomarker (diferentially expressed at 2 hour post-stroke) and at least three late stroke biomarker (diferentially expressed at 24 hour post-stroke).

Examples of immediate early stroke biomarkers include, but are not limited to, gene products of CD163, PKC delta, pyruvate kinase, muscle (PKM2), thyroid hormone receptor associated protein 2, thyroid hormone receptor associated protein 5, nod-like receptor (NLR) family, pyrin domain containing 1, pancreatic ribonuclease (Rnase 1), cytochrome b-245, beta polypeptide, MCP-1, as well as the genes listed in Table 4.

Examples of early stroke biomarkers include, but are not limited to, gene products of CD163, PKC delta, AKT1 substrate 1 (proline-rich) isoform 1, Cmtm3, WD repeat 19 (WDR19), alpha-2 type IX collagen, thyroid hormone receptor associated protein 2, thyroid hormone receptor associated protein 5, nod-like receptor (NLR) family, pyrin domain containing 1, pancreatic ribonuclease (Rnase 1), complement factor H isoform a precursor, MCP-1, as well as the genes listed in Table 5.

Examples of late stroke biomarkers include, but are not limited to, gene products of NODS (NLRX1), aspartyl-tRNAsynthetase, lymphocyte cytosolic protein 1 (L-plastin), chitinase 1 (chitotriosidase), proteasome subunit alpha type 1(PSMA4), PKC delta, Cmtm3, WDR19, alpha-2 type IX collagen, PKM2, thyroid hormone receptor associated protein 2, thyroid hormone receptor associated protein 5, nod-like receptor family, pyrin domain containing 1, pancreatic ribonuclease (Rnase 1), cytochrome b-245, beta polypeptide, complement factor H isoform a precursor, as well as the genes listed in Table 6.

In certain embodiments, the biomarker panel further comprises comprises one or more stroke incidence biomarkers that can only be detected in blood after stroke. Examples of such biomarkers include, but are not limited to, alpha-2-plasmin inhibitor, WDR19, alpha-2 type IX collagen, PKM2, thyroid hormone receptor associated protein 2, thyroid hormone receptor associated protein 5, nod-like receptor family, pyrin domain containing 1 pancreatic ribonuclease (Rnase 1), and cytochrome b-245, beta polypeptide S100-B.

In certain other embodiments, the biomarker panel further comprises one or more stroke severity biomarkers whose levels increase in blood over time after stroke. Examples of such biomarkers include, but are not limited to, PKC delta, chemokine-like superfamily 3 (Cmtm3), and NLR family, pyrin domain containing 1.

In other embodiments, the above-described biomarker panel may further comprise one or more neuronal injury markers, such as gene products of NMDA receptor and neuronal specific enolase.

In other embodiments, the above-described biomarker panel may further comprise one or more emorrhagic stroke markers, such as GFAP, Apo C-III and MMP-9.

In other embodiments, the above-described biomarker panel may further comprise one or more biomarkes selected from the group consisting of D-dimer, CRP, brain natriuretic peptide (BNP) and S1000B.

In other embodiments, the biomarker panel comprises at least one expression product from the genes listed in Table 4, at least one expression product from the genes listed in Table 5 and at least one expression product from the genes listed in Table 6. In other embodiments, the biomarker panel comprises at least three expression product from the genes listed in Table 4, at least three expression product from the genes listed in Table 5 and at least three expression product from the genes listed in Table 6. In other embodiments, the biomarker panel comprises at least five expression product from the genes listed in Table 4, at least five expression product from the genes listed in Table 5 and at least five expression product from the genes listed in Table 6. These biomarker panels may further include expression products from the stroke severity biomarkers, neuronal injury markers, and emorrhagic stroke markers.

In some embodiments, the biomarkers and biomarker panels are specific for ischemic stroke. In related embodiments, the ischemic stroke is large vessel ischemic stroke.

In another embodiment, the biomarkers are detected in a tissue sample.

In certain other embodiments, the biomarkers are detected in a body fluid. As used hereinafter, the term “body fluid” refers to blood, blood plasma or serum, urine, saliva and cerebrospinal fluid. In a related embodiment, the biomarkers are detected in cells obtained from a body fluid, for example, peripheral blood mononuclear cells isolated from a blood sample.

In another embodiment, the measuring step includes measuring two or more biomarkers in the sample from the subject.

In another embodiment, the measuring step includes measuring three or more biomarkers in the sample from the subject.

In another embodiment, the measuring step includes measuring four or more biomarkers in the sample from the subject.

In another embodiment, the measuring step includes measuring five, 10, 15, 20, 25 or more biomarkers in the sample from the subject.

Another aspect of the present invention is a method for determining disease progression in a subject after a stroke. The method comprises the steps of (a) measuring the level of one or more biomarkers in a sample from the subject receiving the treatment and (b) determining the disease progression based on the level of the one or more biomarkers in the sample.

Another aspect of the present invention relates to a method for determining disease progression in a subject after a stroke. The method comprises the steps of (a) measuring the level of one or more biomarkers in a first sample obtained from the subject at a first time point; (b) measuring the level of the one or more biomarkers in a second sample obtained from the subject at a second time point; (c) comparing the level of the one or more biomarkers at the first time point to the level of the one or more biomarkers at the second time point; and (d) determining the disease progression between the first and the second time point based on the result of step (c).

Another aspect of the present invention relates to a method for determining the efficacy of a treatment for stroke in a subject. The method comprises the steps of (a) measuring the level of one or more biomarkers in a first sample obtained from the subject at a first time point; (b) measuring the level of the one or more biomarkers in a second sample obtained from the subject at a second time point, wherein the subject is under treatment at the second time point; (c) comparing the level of the one or more biomarkers at the first time point to the level of the one or more biomarkers at the second time point; and (d) determining the efficacy of the treatment based on the result of step (c).

### Detection of Biomarkers for Stroke in a Subject

Methods for detecting stroke biomarkers in a tissue or body fluid sample have many applications. For example, one or more biomarkers can be measured to aid in the diagnosis of stroke. In another example, the methods for detection of the biomarkers can be used to determine the severity of a stroke. In another example, the methods for detection of the biomarkers can be used to determine the time elapsed following the occurrence of a stroke. In another example, the methods for detection of the biomarkers can be used to determine what type of stroke a subject experienced. For example, differences in the types of stroke biomarkers, and/or the quantity of a particular biomarker in the subject may elucidate whether the subject experienced a hemorrhagic stroke or an ischemic stroke, or whether an ischemic stroke was a large vessel ischemic stroke. In yet another example, the methods for detecting markers can be used to monitor the progression of stroke in a subject. In still another example, the methods for detection of the biomarkers can be used to monitor a subject's response to treatment or to determine the type of treatment that should be used for that subject. In still another example, biomarkers can be used to detect whether a subject has experienced transient ischemia, such as that associated with a reperfusion injury. In a further example, biomarkers can elucidate whether a subject has experienced a permanent stroke or a transient stroke. In another example, biomarkers can be used to assess the effectiveness of a neuroprotective drug or compound.

In order to detect stroke biomarkers in a subject, a cerebrospinal fluid, blood or serum sample is contacted with at least one antibody specific for a biomarker for stroke. Alternatively, a sample can be obtained by lysing the PBMCs by methods known in the art and contacting the lysate with at least one antibody specific for a biomarker for stroke. In addition, biomarkers may also be detected in saliva, urine, tears, and specific subsets of leukocytes, such as B lymphocytes, T lymphocytes, granulocytes (such as neutrophils), and monocytes.

Exemplary biomarkers for stroke include gene products expressed from genes selected from the group consisting of IL-1α, IL-1β, IL-1ra, IL-3, IL-2, IL-4, IL-5, IL-6, IL-7, IL-8, IL-9, IL-10, IL-12 (p40), IL-12(p70), IL-13, IL-15, IL-17, EGF, Eotaxin, FGF-2, FTL-3 ligand, Fractalkine, G-CSF, GM-CSF, GRO, IFN-α2, IFN-γ, IP-10, MCP-1, MCP-3, MCD, MIP-1α, MIP-1β, PDGF-aa, PGDF-aa bb, RANTES, sCD40L, sIL2-rα, TNF-α, TNF-β, VEGF and genes listed in Tables 4, 5, 6 and 8.

Stroke biomarkers can be detected and/or quantified using any of suitable immunological binding assays known in the art. Useful assays include, for example, an enzyme immune assay (EIA) such as enzyme-linked immunosorbent assay (ELISA), a radioimmunoassay (MA), a Western blot assay, a slot blot assay or a dipstick assay.

Generally, a sample from a subject can be contacted with an antibody that specifically binds a stroke biomarker. Optionally, the antibody can be fixed to a solid support to facilitate washing and subsequent isolation of the complex, prior to contacting the antibody with a sample. Examples of solid supports include glass or plastic in the form of a microtiter plate, a stick, a bead, or a microbead. Examples of solid supports encompassed herein include those formed partially or entirely of glass (e.g., controlled pore glass), polysaccharides (e.g., agarose), polyacrylamides, silicones, and plastics such as polystyrene, polypropylene and polyvinyl alcohol. The sample can be diluted with a suitable diluant or eluant before contacting the sample to the antibody.

After incubating the sample with antibodies, the mixture is washed and the antibody-biomarker complex formed can be detected. This can be accomplished by incubating the washed mixture with a detection reagent. This detection reagent may be a second antibody which is labeled with a detectable label, for example. Exemplary detectable labels include magnetic beads (e.g., DYNABEADS™), fluorescent dyes, radiolabels, enzymes (for example, horse radish peroxidase, alkaline phosphatase and others commonly used in an ELISA), and colorimetric labels such as colloidal gold or colored glass or plastic beads. Alternatively, the biomarker in the sample can be detected using an indirect assay, wherein, for example, a second, labeled antibody is used to detect bound biomarker-specific antibody, and/or in a competition or inhibition assay wherein, for example, a monoclonal antibody which binds to a distinct epitope of the marker is incubated simultaneously with the mixture.

Immunoassays can be used to determine presence or absence of a stroke biomarker in a sample as well as the quantity of a biomarker in a sample. First, a test amount of a biomarker in a sample can be detected using the immunoassay methods described above. If a marker is present in the sample, it will form an antibody-marker complex with an antibody that specifically binds the marker under suitable incubation conditions described above. The amount of an antibody-marker complex can be determined by comparing to a standard. A standard can be a known compound or another protein known to be present in a sample, for example. As noted above, the test amount of marker need not be measured in absolute units, as long as the unit of measurement can be compared to a control.

### Enzyme-Linked Immunosorbent Assay (ELISA)

In certain embodiments, the stroke biomarkers are detected using enzyme-linked immunosorbent assay (ELISA) which is typically carried out using antibody coated assay plate or wells. Commonly used ELISA assay employs either a sandwich immunoassay or a competitive binding immunoassay.

Briefly, a sandwich immunoassay is a method using two antibodies, which bind to different sites on the antigen or ligand. The primary antibody, which is highly specific for the antigen, is attached to a solid surface. The antigen is then added followed by addition of a second antibody referred to as the detection antibody. The detection antibody binds the antigen to a different epitope than the primary antibody. As a result the antigen is ‘sandwiched’ between the two antibodies. The antibody binding affinity for the antigen is usually the main determinant of immunoassay sensitivity. As the antigen concentration increases the amount of detection antibody increases leading to a higher measured response. The standard curve of a sandwich-binding assay has a positive slope. To quantify the extent of binding different reporters can be used. Typically an enzyme is attached to the secondary antibody which must be generated in a different species than primary antibodies (i.e. if the primary antibody is a rabbit antibody than the secondary antibody would be an anti-rabbit from goat, chicken, etc., but not rabbit). The substrate for the enzyme is added to the reaction that forms a colorimetric readout as the detection signal. The signal generated is proportional to the amount of target antigen present in the sample.

The antibody linked reporter used to measure the binding event determines the detection mode. A spectrophotometric plate reader may be used for colorimetric detection. Several types of reporters have been developed in order to increase sensitivity in an immunoassay. For example, chemiluminescent substrates have been developed which further amplify the signal and can be read on a luminescent plate reader. Also, a fluorescent readout where the enzyme step of the assay is replaced with a fluorophor tagged antibody is becoming quite popular. This readout is then measured using a fluorescent plate reader.

A competitive binding assay is based upon the competition of labeled and unlabeled ligand for a limited number of antibody binding sites. Competitive inhibition assays are often used to measure small analytes. These assays are also used when a matched pair of antibodies to the analyte does not exist. Only one antibody is used in a competitive binding ELISA. This is due to the steric hindrance that occurs if two antibodies would attempt to bind to a very small molecule. A fixed amount of labeled ligand (tracer) and a variable amount of unlabeled ligand are incubated with the antibody. According to law of mass action the amount of labeled ligand is a function of the total concentration of labeled and unlabeled ligand. As the concentration of unlabeled ligand is increased, less labeled ligand can bind to the antibody and the measured response decreases. Thus the lower the signal, the more unlabeled analyte there is in the sample. The standard curve of a competitive binding assay has a negative slope.

### Microbeads

In certain other embodiments, the stroke biomarkers are detected using antibody coated microbeads. In some embodiments, the microbeads are magnetic beads. In other embodiments, the beads are internally color-coded with fluorescent dyes and the surface of the bead is tagged with an anti-stroke biomarker antibody that can bind a stroke biomarker in a test sample. The stroke biomarker, in turn, is either directly labeled with a fluorescent tag or indirectly labeled with an anti-marker antibody conjugated to a fluorescent tag. Hence, there are two sources of color, one from the bead and the other from the fluorescent tag. Alternatively, the beads can be internally coded by different sizes.

By using a blend of different fluorescent intensities from the two dyes, as well as beads of different sizes, the assay can measure up to hundreds of different stroke biomarkers. During the assay, a mixture containing the color/size-coded beads, fluorescence labeled anti-marker antibodies, and the sample are combined and injected into an instrument that uses precision fluidics to align the beads. The beads then pass through a laser and, on the basis of their color or size, either get sorted or measured for color intensity, which is processed into quantitative data for each reaction.

When samples are directly labeled with fluorophores, the system can read and quantitate only fluorescence on beads without removing unbound fluorophores in solution. The assays can be multiplexed by differentiating various colored or sized beads. Real time measurement is achievable when a sample is directly required for unlabeled samples. Standard assay steps include incubation of a sample with anti-marker antibody coated beads, incubation with biotin or fluorophore-labeled secondary antibody, and detection of fluorescence signals. Fluorescent signals can be developed on bead (by adding streptavidin-fluorophore conjugates for biotinylated secondary antibody) and read out by a bead analyzer. Depending on the anti-marker immobilized on the bead surface, a bead-based immunoassay can be a sandwich type or a competitive type immunoassay.

### Test Stick

In some other embodiments, the stroke biomarkers in a liquid biosample are detected using a test stick or dip stick. The test stick typically contains a fluid impermeable housing and a fluid permeable “stick” having one or more detection zones. In one embodiment, each detection zone contains a dried binding reagent that binds to a stroke biomarker in a biosample. In another embodiment, the dried binding reagent is a labeled binding reagent. In another embodiment, the test stick may further comprise a control zone to indicate that the assay test has been carried out satisfactorily, namely the reagents were present in the test stick and that they become mobilized during running the test and have been transported along the flow path. The control zone can also indicate that the reagents within the device are capable of immunochemical interactions, confirming the chemical integrity of the device. This is important when considering the storage and shipment of the device under desiccated conditions within a certain temperature range. The control zone is typically positioned downstream from the detection zone(s) and may, for example, comprise an immobilized binding reagent for a labeled binding reagent. The labeled binding reagent may be present in a mobilizable form upstream from the control zone and detection zone. The labeled binding reagent may be the same or different to the labeled binding reagent for the stroke biomarker.

In one embodiment, the test stick comprise a porous sample receiver in fluid connection with and upstream from one or more flow-paths. The porous sample receiver may be common to all assays. Thus a fluid sample applied to the common sample application region of the device is able to travel along the one or more flow-paths to the respective detection zones. The porous sample receiver may be provided within a housing or may at least partially extend out of said housing and may serve for example to collect a body fluid. The porous sample receiver may also act as a fluid reservoir. The porous sample receiving member can be made from any bibulous, porous or fibrous material capable of absorbing liquid rapidly. The porosity of the material can be unidirectional (i.e. with pores or fibers running wholly or predominantly parallel to an axis of the member) or multidirectional (omnidirectional, so that the member has an amorphous sponge-like structure). Porous plastics material, such as polypropylene, polyethylene (preferably of very high molecular weight), polyvinylidene fluoride, ethylene vinylacetate, acrylonitrile and polytetrafluoro-ethylene can be used. Other suitable materials include glass-fiber.

If desired, an absorbent “sink” can be provided at the distal end of the carrier material. The absorbent sink may comprise, for example, Whatman 3MM chromatography paper, and should provide sufficient absorptive capacity to allow any unbound labeled binding reagent to wash out of the detection zone(s). As an alternative to such a sink it can be sufficient to have a length of porous solid phase material which extends beyond the detection zone(s).

Following the application of a binding reagent to a detection zone, the remainder of the porous solid phase material may be treated to block any remaining binding sites. Blocking can be achieved by treatment for example with protein (e.g. bovine serum albumin or milk protein), or with polyvinyl alcohol or ethanolamine, or combinations thereof. To assist the free mobility of the labeled binding reagent when the porous carrier is moistened with the sample, the porous carrier may further comprise a sugar such as sucrose or lactose and/or other substances, such as polyvinyl alcohol (PVA) or polyvinyl pyrrolidone (PVP). Such material may be deposited, for example, as an aqueous solution in the region to which the labeled binding reagent is to be applied. Such materials could be applied to the porous carrier as a first application followed by the application of the label; alternatively, such materials could be mixed with the label and applied to the porous carrier or combinations of both. Such material may be deposited upstream from or at the labeled binding reagent.

Alternatively, the porous carrier may not be blocked at the point of manufacture; instead the means for blocking the porous carrier are included in a material upstream from the porous carrier. On wetting the test strip, the means for blocking the porous carrier are mobilized and the blocking means flow into and through the porous carrier, blocking as the flow progresses. The blocking means include proteins such as BSA and casein as well as polymers such as PVP, PVA as well as sugars and detergents such as Triton-X100. The blocking means could be present in the macroporous carrier material.

The dried binding reagents may be provided on a porous carrier material provided upstream from a porous carrier material comprising the detection zone. The upstream porous carrier material may be macroporous. The macroporous carrier material should be low or non-protein-binding, or should be easily blockable by means of reagents such as BSA or PVA, to minimize non-specific binding and to facilitate free movement of the labeled reagent after the macroporous body has become moistened with the liquid sample. The macroporous carrier material can be pre-treated with a surface active agent or solvent, if necessary, to render it more hydrophilic and to promote rapid uptake of the liquid sample. Suitable materials for a macroporous carrier include plastic materials such as polyethylene and polypropylene, or other materials such as paper or glass-fiber. In the case that the labeled binding reagent is labeled with a detectable particle, the macroporous body may have a pore size at least ten times greater than the maximum particle size of the particle label. Larger pore sizes give better release of the labeled reagent. As an alternative to a macroporous carrier, the labeled binding reagent may be provided on a non-porous substrate provided upstream from the detection zone, said non-porous substrate forming part of the flow-path.

In another embodiment, the test stick may further comprise a sample receiving member for receiving the fluid sample. The sample receiving member may extend from the housing.

The housing may be constructed of a fluid impermeable material. The housing will also desirably exclude ambient light. The housing will be considered to substantially exclude ambient light if less than 10%, preferably less than 5%, and most preferably less than 1%, of the visible light incident upon the exterior of the device penetrates to the interior of the device. A light-impermeable synthetic plastics material such as polycarbonate, ABS, polystyrene, polystyrol, high density polyethylene, or polypropylene containing an appropriate light-blocking pigment is a suitable choice for use in fabrication of the housing. An aperture may be provided on the exterior of the housing which communicates with the assay provided within the interior space within the housing. Alternatively, the aperture may serve to allow a porous sample receiver to extend from the housing to a position external from the housing.

### Microarray

In other embodiments, the stroke biomarkers are detected by a protein microarray containing immobilized stroke biomarker-specific antibodies on its surface. The microarray can be used in a “sandwich” assay in which the antibody on the microarray captures a stroke biomarker in the test sample and the captured marker is detected by a labeled secondary antibody that specifically binds to the captured marker. In a preferred embodiment, the secondary antibody is biotinylated or enzyme-labeled. The detection is achieved by subsequent incubation with a streptavidin-fluorophore conjugate (for fluorescence detection) or an enzyme substrate (for colorimetric detection).

Typically, a microarray assay contains multiple incubation steps, including incubation with the samples and incubation with various reagents (e.g., primary antibodies, secondary antibodies, reporting reagents, etc.). Repeated washes are also needed between the incubation steps. In one embodiment, the microarray assays is performed in a fast assay mode that requires only one or two incubations. It is also conceivable that the formation of a detectable immune complex (e.g., a captured stroke biomarker/anti-marker antibody/label complex) may be achieved in a single incubation step by exposing the protein microarray to a mixture of the sample and all the necessary reagents. In one embodiment, the primary and secondary antibodies are the same antibody.

In another embodiment, the protein microarray provides a competitive immunoassay. Briefly, a microarray comprising immobilized anti-marker antibodies is incubated with a test sample in the presence of a labeled stroke biomarker standard. The labeled stroke biomarker competes with the unlabeled stroke biomarker in the test sample for the binding to the immobilized antigen-specific antibody. In such a competitive setting, an increased concentration of the specific stroke biomarker in the test sample would lead to a decreased binding of the labeled stroke biomarker standard to the immobilized antibody and hence a reduced signal intensity from the label.

The microarray can be processed in manual, semi-automatic or automatic modes. Manual mode refers to manual operations for all assay steps including reagent and sample delivery onto microarrays, sample incubation and microarray washing. Semi-automatic modes refer to manual operation for sample and reagent delivery onto microarray, while incubation and washing steps operate automatically. In an automatic mode, three steps (sample/reagent delivery, incubation and washing) can be controlled by a computer or an integrated breadboard unit with a keypad. For example, the microarray can be processed with a ProteinArray Workstation (PerkinElmer Life Sciences, Boston, Mass.) or Assay 1200™. Workstation (Zyomyx, Hayward, Calif.). Scanners by fluorescence, colorimetric and chemiluminescence, can be used to detect microarray signals and capture microarray images. Quantitation of microarray-based assays can also be achieved by other means, such as mass spectrometry and surface plasma resonance. Captured microarray images can be analyzed by stand-alone image analysis software or with image acquisition and analysis software package. For example, quantification of an antigen microarray can be achieved with a fluorescent PMT-based scanner—ScanArray 3000 (General Scanning, Watertown, Mass.) or colorimetric CCD-based scanner—VisionSpot (Allied Biotech, Ijamsville, Md.). Typically, the image analysis would include data acquisition and preparation of assay report with separate software packages. To speed up the whole assay process from capturing an image to generating an assay report, all the analytical steps including image capture, image analysis, and report generation, can be confined in and/or controlled by one software package. Such an unified control system would provide the image analysis and the generation of assay report in a user-friendly manner.

### Implantable Biosensors

In at-risk subjects, the stroke biomarkers may be detected using implantable biosensors. Biosensors are electronic devices that produce electronic signals as the result of biological interactions. In one embodiment, the biosensors use antibodies, receptors, nucleic acids, or other members of a binding pair to bind with a stroke biomarker, which is typically the other member of the binding pair. Biosensors may be used with a blood sample to determine the presence of a stroke biomarker without the need for sample preparation and/or separation steps typically required for the automated immunoassay systems.

In one embodiment, the sensor is a nanoscale device. The sensor system includes a biological recognition element attached to a nanowire and a detector that is capable of determining a property associated with the nanowire. The biological recognition element is one member of a binding pair (e.g., a receptor of the stroke biomarker or an anti-stroke biomarker antibody) where the stroke biomarker being measured is the other member of the binding pair. Preferably, the nanowire sensor includes a semiconductor nanowire with an exterior surface formed thereon to form a gate electrode and a first end in electrical contact with a conductor to form a source electrode and a second end in contact with a conductor to form a drain electrode. In one embodiment the sensor is a field effect transistor comprising a substrate formed of an insulating material, a source electrode, a drain electrode and a semiconductor nanowire disposed there between with a biological recognition element attached on a surface of the nanowire. When a binding event occurs between the biological recognition element and its specific binding partner, a detectable change is caused in a current-voltage characteristic of the field effect transistor.

In another embodiment, the sensor system includes an array of sensors. One or more of the sensors in the array is associated with a protective member that prevents the associated sensor from interacting with the surrounding environment. At a selected time, the protective member may be disabled, thereby allowing the sensor to begin operating to interact with the surrounding fluid or tissue so that the biological recognition element can interact with the other member of its binding pair if that pair member is present.

In another embodiment, the protective member is formed of a conductive material that can oxidize, is biocompatible, bio-absorbable, and that may be dissolved in solution such as blood upon application of an electric potential. For example, a sensor may be formed within a well of a substrate that is capped by a conductive material such as a biocompatible metal or an electrically-erodible polymer. In another embodiment, the protective member is formed using a material that dissolves over a predetermined period of time. Implantable biosensors are described in, for example, US Patent Application Publication No. 20050049472, which is incorporated herein by reference.

### Determination of Standard Value, Specificity and Sensitivity

In the present invention, the standard expression level of a stroke biomarker, such as the concentration of a stroke biomarker in a biological sample, can be determined statistically. For example, the concentration of a stroke biomarker in a biological sample in healthy individuals can be measured to determine the standard concentration of said stroke biomarker statistically. When a statistically sufficient population can be gathered, a value in the range of twice or three times the standard deviation (S.D.) from the mean value is often used as the standard value. Therefore, values corresponding to the mean value+2×S.D. or mean value+3×S.D. may be used as standard values. The standard values set as described theoretically comprise 90% and 99.7% of healthy individuals, respectively.

Alternatively, standard values can also be set based on the actual expression level (e.g., blood concentration of a stroke biomarker) in stroke patients. Generally, standard values set this way minimize the percentage of false positives, and are selected from a range of values satisfying conditions that can maximize detection sensitivity. Herein, the percentage of false positives refers to a percentage, among healthy individuals, of patients whose concentration of a stroke biomarker in a biological sample is judged to be higher or lower than a standard value. On the contrary, the percentage, among healthy individuals, of patients whose concentration of said stroke biomarker in a biological sample is judged to be lower or higher, respectively, than a standard value indicates specificity. That is, the sum of the false positive percentage and the specificity is always 1. The detection sensitivity refers to the percentage of patients whose concentration of a stroke biomarker in a biological sample is judged to deviate from a standard value, among all stroke patients within a population of individuals for whom the presence of stroke has been determined.

As used herein, the term “test sensitivity” is the ability of a screening test to identify stroke, also characterized by being a test with high sensitivity has few false negatives, additionally a test independent of stroke prevalence. The test sensitivity is calculated as true positive tests per total affected patients tested, expressed as a percentage. Test specificity” refers to a screening test which is correctly negative in the absence of stroke, has high specificity and few false positives, is independent of stroke prevalence. The test specificity is calculated as true negative tests per unaffected individuals tested, expressed as a percentage.

The term “PPV” (Positive Predictive Value) is the percent of patients with a positive test having had a stroke, and thus assesses reliability of positive test. Calculation:

PPV=(True positive)/(True+False positives). 1.

The term “NPV” (Negative Predictive Value) refers to patients with negative test that have not had a stroke, and assesses reliability of negative test. Calculation:

NPV=(True negative)/(true and false negatives). 2.

As the relationship shown above indicates, each of the values for sensitivity, specificity, positive predictive value, and negative predictive value, which are indexes for evaluating the diagnostic accuracy, varies depending on the standard value for judging the level of the concentration of a stroke biomarker in a biological sample.

A standard value is usually set such that the false positive ratio is low and the sensitivity is high. However, as also apparent from the relationship shown above, there is a trade-off between the false positive ratio and sensitivity. That is, if the standard value is decreased, the detection sensitivity increases. However, since the false positive ratio also increases, it is difficult to satisfy the conditions to have a low false positive ratio. Considering this situation, for example, values that give the following predicted results may be selected as the preferable standard values in the present invention: (1) standard values for which the false positive ratio is 50% or less (that is, standard values for which the specificity is not less than 50%) and (2) standard values for which the sensitivity is not less than 20%.

The standard values can be set using receiver operating characteristic (ROC) curve. An ROC curve is a graph that shows the detection sensitivity on the vertical axis and the false positive ratio on the horizontal axis. A ROC curve can be obtained by plotting the changes in the sensitivity and the false positive ratio, which were obtained after continuously varying the standard value for determining the high/low degree of the concentration of a stroke biomarker in a biological sample.

The “standard value” for obtaining the ROC curve is a value temporarily used for the statistical analyses. The standard value for obtaining the ROC curve can generally be continuously varied within a range that allows to cover all selectable standard values. For example, the standard value can be varied between the smallest and largest measured stroke biomarker values in biological samples from an analyzed population.

Based on the obtained ROC curve, a preferable standard value to be used in the present invention can be selected from a range that satisfies the above-mentioned conditions. Alternatively, a standard value can be selected based on a ROC curve produced by varying the standard values from a range that comprises most of the measured stroke biomarker level in a biological sample.

### Kits

In yet another aspect, the invention provides kits for detecting the stroke biomarkers of the present invention. For example, the kits may contain reagents for the detection of one or more biomarkers and a list of reference levels of the one or more biomarkers.

In certain embodiments, the biomarker are gene products expressed from genes selected from the group consisting of IL-1α, IL-1β, IL-1ra, IL-3, IL-2, IL-4, IL-5, IL-6, IL-7, IL-8, IL-9, IL-10, IL-12 (p40), IL-12(p70), IL-13, IL-15, IL-17, EGF, Eotaxin, FGF-2, FTL-3 ligand, Fractalkine, G-CSF, GM-CSF, GRO, IFN-α2, IFN-γ, IP-10, MCP-1, MCP-3, MCD, MIP-1α, MIP-1β, PDGF-aa, PGDF-aa bb, RANTES, sCD40L, sIL2-rα, TNF-α, VEGF and genes listed in Tables 4, 5, 6 and 8.

In other embodiments, the kit comprises reagents for the detection of a biomarker panel. In certain embodiments, the biomarker panel comprises two or more biomarkers. In certain other embodiments, the biomarker panel comprises five or more biomarkers. In certain other embodiments, the biomarker panel comprises ten or more biomarkers. In certain other embodiments, the biomarker panel comprises twenty or more biomarkers.

The kits of the invention have many applications. For example, the kits can be used to diagnose whether a subject has had a stroke, how severe that stroke was, and how long ago the subject had the stroke. In another example, the kits can be used to determine what type of stroke a subject experienced. For example, differences in the types of stroke biomarkers, and/or the quantity of a particular biomarker in the subject may elucidate whether the subject experienced a hemorrhagic stroke or an ischemic stroke, or whether an ischemic stroke was a large vessel ischemic stroke. In still another example, the kit can be used to monitor the progression of stroke in a subject or a subject's response to treatment or to determine the type of treatment that should be used for that subject.

In one embodiment, a kit comprises (a) an antibody that specifically binds to a stroke biomarker; and (b) a detection reagent. Such kits can be prepared from the materials described above, and the previous discussion regarding the materials (e.g., antibodies, detection reagents, immobilized supports, etc.) is fully applicable to this. In a specific embodiment, the antibody is a monoclonal antibody. The detecting means of the kit may include a second, labeled monoclonal antibody. Alternatively, or in addition, the detecting means may include a labeled, competing antigen. In some embodiments, the kit may further comprise instructions for suitable operation parameters in the form of a label or a separate insert.

Optionally, the kit may further comprise a standard or control information so that the test sample can be compared with the control information standard to determine if the test amount of a marker detected in a sample is a diagnostic amount consistent with a diagnosis of stroke in general, the type of stroke, degree of severity of the stroke, time elapsed since the occurrence of the stroke, progression of the stroke, and/or effect of treatment on the subject.

Optionally, the kit can further comprise instructions for suitable operational parameters in the form of a label or a separate insert. For example, the kit may have standard instructions informing a consumer how to wash the probe after a sample is contacted on the probe. In another example, the kit may have instructions for pre-fractionating a sample to reduce complexity of proteins in the sample. In another example, the kit may have instructions for automating the fractionation or other processes.

The present invention is further illustrated by the following examples which should not be construed as limiting. The contents of all references, patents and published patent applications cited throughout this application, as well as the Figures and Tables, are incorporated herein by reference.

### Example 1: Determination of Biomarkers in a Non-Human Primate Model of Stroke

Three juvenile healthy Specific Pathogen Free (SPF) rhesus monkeys (Macaca mulata), weighing 4.5-6.5 kg were screened for metabolic diseases by complete blood count and serum chemistry analysis. Coagulation evaluation tests were also performed. Animals were fasted 12 hours prior to surgery. Anesthesia was induced by ketamine (10-20 mg/kg IM). Atropine (O.Q4 mg/kg) was administered IM as part of the anesthesia pre-medications. Anesthesia was maintained with propofol (0.3-0.4 mg/kg/min) in a constant rate into the saphenous vein. A 3.0-3.5 mmID cuffed endotracheal tube was placed. Animals were connected to monitoring equipment (BM5 VET, Bionet America, CA) which included EKG, pulse oxymeter, capnographer, HR, RR, rectal temperature, and placed on an Air Warmer (Bair Hugger; Arizant, Eden Prairie, Minn.). Physiologic variables were kept within the normal range. Following sterile preparation with betadine and alcohol scrubbing of the monkey's inguinal area, the area was infiltrated with lidocaine 2% to prevent vasospasm of the femoral artery as well as local anesthesia. The Seldinger technique was used, which consists of an incision in the groin near the pulsation wave of the femoral artery. Using a Micropuncture Introducer Set 21G angiography puncture needle (Cook Medical, Bloomington, Ind.), a guide wire was introduced and a 4 fr. femoral sheath placed, while irrigating with a normal saline pressure bag. A 4 fr. diagnostic catheter (Terumo, Somerset, N.J.) was introduced over a 0.035 fr. guide wire (Terumo, Somerset, N.J.) and navigated the abdominal thoracic aorta until the aortic arch was reached, and from there the brachiocephalic trunk, common carotid artery and internal carotid artery were catheterized. Confirmation of catheter placement was done by injection of contrast material (Conray; Covidien, Hazelwood, Mo.) under digital angiographic control by using a C-Arm Fluoroscopy system. A Rapid Transit Microcatheter (Cordis, Bridgewater, N.J.) with a Traxcess 0.014 guiding wire (Terumo, Somerset, N.J.) was introduced and navigated into the M1 segment of the right middle cerebral artery (MCA). Occlusion of the M1 segment of the MCA was accomplished by injecting 6 to 8 (2 mm length) thrombogenic silk sutures (3-0 silk, Ethicon, San Lorenzo, PR) into the MCA through the catheter in saline using a 3 cc syringe. The incisions were closed with non-absorbent 4-0 sterile suture, and the monkeys were transported to the MRI facility for imaging. After the MRI, animals were returned to the recovery room and closely monitored by veterinary staff. The endotracheal tubes were removed when the swallowing reflex was restored. Once recovered enough to be able to sit, they were returned to their cages.

MRI was performed immediately after (1-3 hours following MRI while the animal was under anesthesia from the surgery) and 24 hours following MCAO. Images were acquired using a Philips Achieva 1.5-T scanner (Philips Medical Systems, Best, Netherlands) using an 8-channel SENSE head coil. Coronal slices were obtained perpendicular to a line joining the inferior surface of the genu and the splenium of the corpus callosum, which facilitated the re-acquisition of similar slices on the second imaging session. T2-weighted images were acquired with a Turbo Spin Echo sequence with 0.76×0.76×3 mm voxel resolution (TE=110 ms, TR=2316-2322 ms, Turbo factor=10, NSA=2, FOV=17 cm, matrix=224×222, 30 slices, no gap). Diffusion-weighted images were acquired with a single-shot Spin Echo EPI sequence with 1.72×1.72×2 mm resolution (b-values=0 and 1,000 s/mm², TE=58 ms, TR=9,977-10,047 ms, SENSE=2, NSA=6, FOV=11 cm, matrix=64×64, 41 slices, no gap). The diffusion-weighted images were registered to account for motion prior to generating Apparent Diffusion Coefficient (ADC) maps using the scanner's diffusion analysis software. Infarct volumes were approximated from both the ADC map and the T2-weighted images. Regions of interest (ROI) were hand-drawn by a radiologist on a slice-by-slice basis using the OsiriX software package (Rosset et al., 2004). The ROIs were drawn along the outer margin of the lesions. The total lesion volume was approximated by multiplying the total ROI area over all slices by the corresponding slice thickness.

Spontaneous behavior was recorded simultaneously on two different neurological scales: Standard Neurological Scale (Spetzler et al. 1980; Mack et al. 2003; and D'Arceuil et al. 2006) and Task-Oriented Neurological Scale (Mack et al. 2003). Each subject was observed twice: one or two days before MCAO to establish the baseline behavior and 20 to 23 hours after MCAO to test for the effect of the occlusion. In each session, behavior was recorded for 30 minutes by one observer who was not informed on which side of the brain the MCAO was performed. In addition to manual scoring, the animals were video recorded during each session. Since the scores obtained with the Standard Neurological Scale are measured in an ordinal scale, they were analyzed with the nonparametric Wilcoxon signed rank test. On the other hand, the scores obtained with the task-oriented scales are measured on a numeric scale and therefore, these data were analyzed with the paired t-test. After having the last MRI, animals were perfused with PBS under deep anesthesia. Brains were rapidly removed, cut continuously into 2 cm coronal slices, and biopsy punches were taken from peri-infarct regions based on the MRI data. To measure infarction, sections were stained with 2,3,5-triphenyltetrazolium chloride (TTC) then placed directly in 10% formalin. Coronal 20 mm fixed, frozen sections were used for histochemistry. Fluoro jadeB was carried out according to the manufacturer's protocols. Sections were mounted with an antifade medium.

Venous blood samples were collected at the pre-occlusion and 1-hour, 2-hours and 24-hours post-occlusion time points. The serum and peripheral blood mononuclear cells (PBMCs) were separated and stored at −80° C. for protein and RNA analysis. Cerebrospinal fluid (CSF) was collected according to well-established protocols.

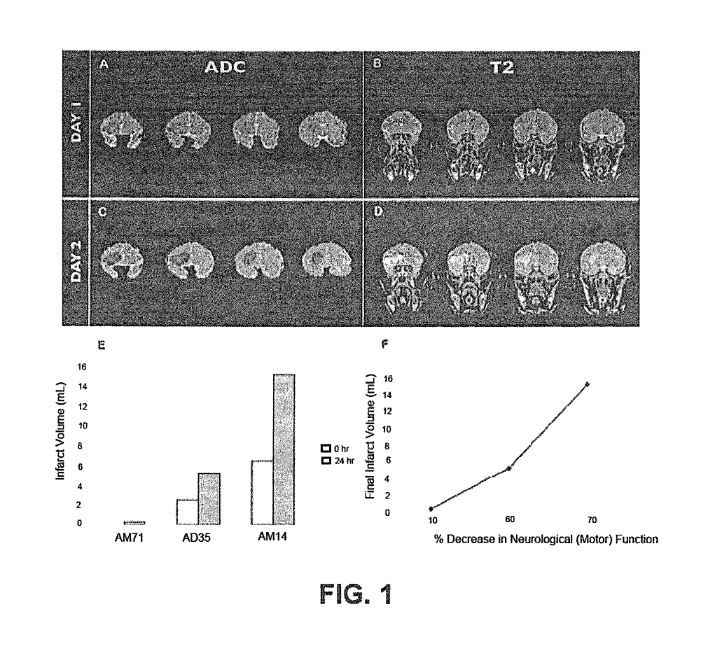

FIGS. 1A-D show representative MRI images of the evolution of cerebral infarction from one of the monkeys using diffusion weighted (DWI) and T2-weighted imaging. Immediately after MCAO, restricted diffusion in the infarcted tissues was observed in two of the three monkeys observed (FIGS. 1A and 1E). There were minimal or no significant abnormalities in the T2-weighted images of any of the monkeys (FIG. 1B) as seen in human stroke patients. The infarcts, as defined by DWI, expanded 24 hours following MCAO in all animals (FIG. 1C). There was a corresponding increased T2 signal as well after 24 hours (FIG. 1D). While there was inter-animal variability in the size of the infarcts, the calculated infarct volumes increased in size compared to measurements obtained the previous day (FIG. 1E). Using a Task-Oriented Neurological Scale, the comparison of scores obtained before and after MCAO indicated a selective and significant (p<0.05) impairment in the category Distal Strength and Coordination, while no differences were observed in the categories measuring awareness and self care (Behavior) or Tone and Posture. The Task-Oriented Neurological Scale reflected a robust impairment in the left arm motor function with little or no impairment of the general neurobehavioral status of the animal. We also observed a correlation between the infarct size measured by DWI and the Distal Strength and Coordination measure (FIG. 1F).

### Example 2: RNA/Protein Isolation and Microarray Analysis in Non-Human Primate Model of Stroke

RNA and protein from PBMCs and brain biopsy punches were isolated using TRIZOL (InVitrogen, Inc., Carlsbad, Calif.) according to the manufacturers instructions. For microarray analysis, total RNA from PBMCs was converted to double-stranded cDNA. cRNA was synthesized using a RNA transcript labeling kit (Enzo Diagnostics, Farmingdale, N.Y., USA). Biotin-labeled cRNA was cleaned up using a GENECHIP Sample Cleanup Module (Affymetrix Inc, Santa Clara, Calif., USA). Twenty μg of the in vitro transcription product was fragmented in Fragmentation Buffer, by placing at 94° C. for 35 minutes. Following fragmentation, 15 μg of the biotinylated cRNA was hybridized to an GENECHIP® Rhesus Macaque Genome Array (Affymetrix, Inc. Santa Clara, Calif.). The chips were hybridized at 45° C. for 16 h, and then washed, stained with streptavidin-phycoerythrin, and scanned (GENECHIP® 3000 7G Scanner) according to manufacturing guidelines.

Data analysis was performed using Affymetrix Expression Console™ software that supports probe set summarization and CHP file generation of 3′ expression using the MASS Statistical algorithm. Resulting CHP data was normalized and further analyzed using Genespring GX10 (Agilent Technologies, Santa Clara, Calif.) and Genesis (Institute for Biomedical Engineering, Graz University of Technology (IBMT-TUG), Austria) Software. Gene expression values between time-points that increased or decreased by 2-fold or more were statistically significant (p<0.005) using 2-way ANOVA. Data were analyzed using Expression Console (Affymetrix Inc, Santa Clara, Calif. USA) and Genespring GX-10 (Agilent Technologies, Santa Clara, Calif.) software for initial analysis, normalization and replicate analysis. Ingenuity Pathway Analysis tools (Ingenuity Systems, www.ingenuity.com) was used to analyze gene networks, biological functions and canonical pathways. A data set containing gene identifiers and corresponding expression values was uploaded into in the application. Each gene identifier was mapped to its corresponding gene object in the Ingenuity Pathways Knowledge Base. Single array analysis was used to build the databases of gene expression profiles. Expression Console normalized data for each chip. Detection p-value (set at p<0.5) was used to statistically determine whether a transcript is expressed on a chip. The software generated a present (P), marginal (M) or absent (A) call for each transcript based on the p-value. Transcripts were considered differentially expressed if statistical logic generated calls of present and were then grouped as ‘known genes’ or ‘unknown genes’. Genes that were absent in all controls (pre-occlusion) and present at either time point are idenitifed in Tables 1-3. Self organizing map clustering and hierarchical clustering were used to identity candidates that were not present in blood prior to stroke and were induced in particular patterns following MCAO. Biomarkers that appeared in both the genomic and proteomic analyses were identified. IPA was used to predict whether these proteins are normally expressed in blood or perhaps released from the brain in response to ischemia and neuronal injury.

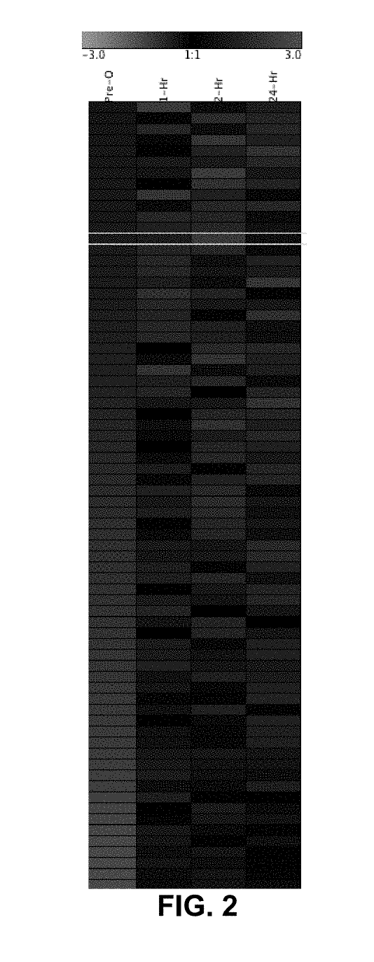

FIG. 2 is a representative image showing that a number of genes are induced in PBMCs (red bars) following stroke in the NHP stroke model compared to no or low expression at baseline (green bars). Genes that are differentially regulated in the NHP stroke model are listed in Tables 1-3. The corresponding human genes are listed in Tables 4-6. These gene products can be used as immediate early biomarkers (1 hr post stroke), early biomarkers (2 hr post stroke) or late biomarkers (24 hr post stroke) for stroke.

### Example 3: High Throughput Proteomic Analysis of Blood, Cerebrospinal Fluid and Brain Tissues from Monkeys Following Focal Ischemia

Stroke biomarker candidates identified from the genomic analysis as well as novel proteins were examined using LUMINEX technology, Western blot, peptidomics and mass spectroscopy.

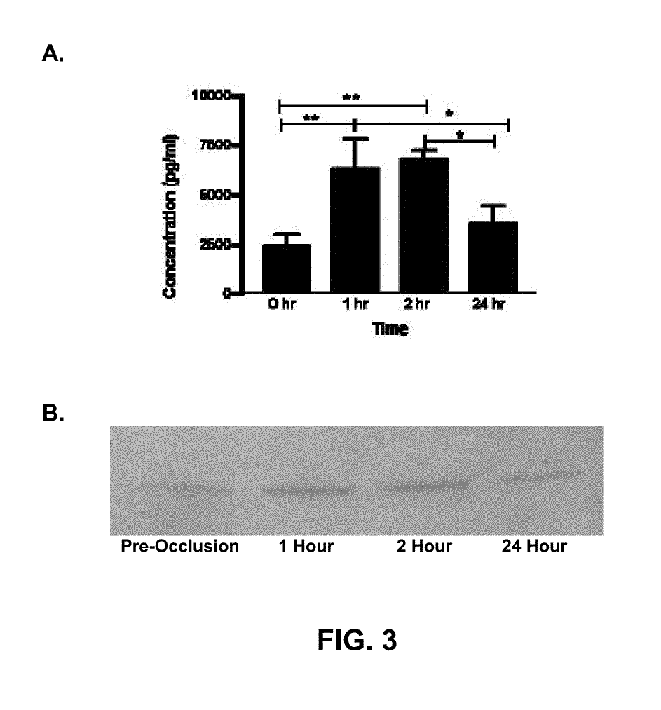

Blood was collected from animals before MCAO and 1, 2 and 24 hours following stroke. Cytokines, chemokines and growth factors in blood serum, CSF and brain extracts were analyzed by multiplex bead immunoassay (LUMINEX 100, Luminex Corporation, Austin, Tex., USA) according to the manufacturer's instructions and using kits from Biosource (Cat. No.; LHC 0001, LHC 0151, LHC 9121, LHC 0171, Invitrogen, Carlsbad, Calif., USA). Pre-occlusion and 1, 2 and 24-hour serum samples from three Rhesus monkeys were evaluated simultaneously for circulating cytokines, chemokines and growth factors (IL-1α, IL-1β, IL-1ra, IL-3, IL-2, IL-4, IL-5, IL-6, IL-7, IL-8, IL-9, IL-10, IL-12 (p40), IL-12(p70), IL-13, IL-15, IL-17, EGF, Eotaxin, FGF-2, FTL-3 ligand, Fractalkine, G-CSF, GM-CSF, GRO, IFN-α2, IFN-γ, IP-10, MCP-1, MCP-3, MCD, MIP-1β, PDGF-aa, PGDF-aa bb, RANTES, sCD40L, sIL2-rα, TNF-α, TNF-β and VEGF) using a commercially available multiplex colorimetric bead-based cytokine immunoassay coupled with the LUMINEX system and human-specific bead sets (BioRad, San Diego, Calif.), according to the manufacturer's instructions. The results were interpolated from 5-parameter-fit standard curves generated using the relevant recombinant human proteins (BioRad). Proteins were also validated using antibodies in Western and dot blot analyses. In additional to the cytokines, chemokines and growth factors mentioned, expression of proteins identified by the microarray screen and compare to other stroke biomarkers from the published literature was examined (Foerch et al., 2009). FIG. 3A is a graph showing the protein levels of the cytokine MCP-1 in serum following MCAO in the NHP. MCP-1 increases significantly 1-2 hours after MCAO then returns to baseline at 24 hours following stroke using a LUMINEX assay. Western blot analysis of proteins from PBMCs indicates that the increased MCP-1 found in serum was induced in cells flowing stroke in the NHP (FIG. 3B).

Plasma or serum samples (˜0.5 μl) were first processed to remove larger proteins by passing them through a Microcon (Millipore cat #42406) YM-10 filter at 20,000×g for 20 min as described (Faith Maria, et al. MCP 2006; 5(6):998-1005). The filtrate was acidified to 0.25% final concentration of acetic acid before separation. The resulting peptides (10 μl) will be then separated on a 0.1×50 mm C18 column (Michrom Bioresources) using a linear gradient of acetonitrile (Sigma) in water (Burdick and Jackson) from 5% to 35% over 30 minutes. The column was then washed with 80% acetonitrile for 10 minutes followed by re-equilibration at 2% acetonitrile for an additional 10 minutes. Spectra were collected on an LTQ mass spectrometer (Thermo Scientific) using Excalibur 2.2 software. The minimum signal for DTA generation was set at 200.0 with an isolation width of 2.00, normalized collision energy of 35.0. Precursor scans were followed by MS/MS scans of the three most intense ions. Raw files were searched against a human database using BioWorks 3.3 software. Each sample was run in duplicate and all SRF files produced by BioWorks were compared using ProteoIQ (Bioinquire, Athens Ga.).

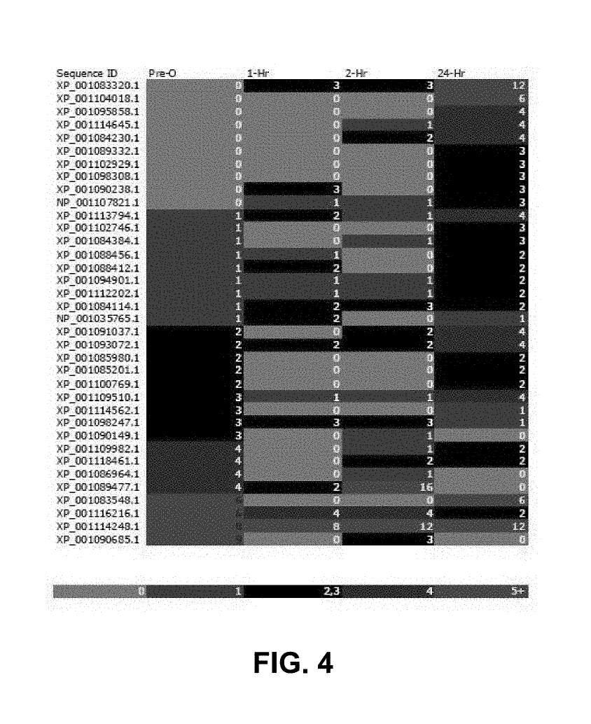

Confidence levels for each sample were set from false discovery rates of 0.05 determined from decoy database searches. FIG. 4 is a representative image showing that several proteins are increased (red bars) and decreased (green) in serum following stroke in the NHP stroke model. The numbers indicate the number of individual peptides in the serum that correspond to the presence of the specific protein. The result of the proteomic analysis is summarized in Tables 7 and 8.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

TABLE 1
Differentially expressed genes in PBMCs of NHPs 1-hr following MCAO

| Probe Set ID | Gene Symbol | Gene Title | UniGene ID | RefSeq Protein ID | Pre-O Signal | Pre-O detection | 1 hr Signal | 1-hr detection |
|---|---|---|---|---|---|---|---|---|
| MmugDNA.18724.1.S1_s_at | B4GALT6 | UDP-Gal:betaGlcNAc beta 1,4-galactosyltransferase, | Mmu.604 | XP_001099215 /// | 58.75183 | A | 55.18526 | P |
| | | polypeptide 6 | | XP_001099324 /// | | | | |
| | | | | XP_001099421 | | | | |
| MmugDNA.26392.1.S1_at | CCNF | cyclin F | Mmu.11675 | XP_001085466 | 114.6939 | A | 104.4854 | P |
| MmugDNA.25206.1.S1_at | CDC2 | cell division cycle 2, G1 to S and G2 to M | Mmu.16170 | XP_001095697 /// | 83.30299 | A | 49.54517 | P |
| | | | | XP_001095798 /// | | | | |
| | | | | XP_001095903 | | | | |
| Mmu.10362.1.S1_at | CLIC4 | chloride intracellular channel 4 | Mmu.4332 | XP_001106291 /// | 131.4646 | A | 89.32994 | P |
| | | | | XP_001106424 /// | | | | |
| | | | | XP_001106485 | | | | |
| MmunewRS.528.1.S1_at | CSF2RB | colony stimulating factor 2 | Mmu.1176 | XP_001086084 | 146.8577 | A | 325.4129 | P |
| | | receptor, beta, low-affinity | | | | | | |
| | | (granulocyte-macrophage) | | | | | | |
| Mmu.15973.2.S1_x_at | CSH-3 /// | chorionic somatommamotropin | Mmu.10849 | NP_001036203 /// | 122.2181 | A | 114.4969 | P |
| | CSH-4 /// | hormone 3 /// chorionic | | NP_001036207 /// | | | | |
| | GH1 /// | somatommamotropin hormone | | NP_001040618 /// | | | | |
| | LOC718474 | 4 /// growth hormone 1 /// | | NP_001040620 /// | | | | |
| | /// | similar to growth hormone 1 | | XP_001111200 /// | | | | |
| | LOC721256 | isoform 1 /// similar to growth | | XP_001116436 /// | | | | |
| | /// | hormone 1 isoform 1 /// | | XP_001117351 | | | | |
| | LOC735307 | chorionic somatomammotropin-1 | | | | | | |
| MmugDNA.5060.1.S1_at | CTNND1 | catenin (cadherin-associated protein), delta 1 | Mmu.1419 | XP_001094621 /// | 79.00558 | A | 296.0597 | P |
| | | | | XP_001095087 /// | | | | |
| | | | | XP_001095314 /// | | | | |
| | | | | XP_001095429 /// | | | | |
| | | | | XP_001095545 /// | | | | |
| | | | | XP_001095646 /// | | | | |
| | | | | XP_001095745 /// | | | | |
| | | | | XP_001095859 /// | | | | |
| | | | | XP_001095967 /// | | | | |
| | | | | XP_001096070 /// | | | | |
| | | | | XP_001096183 /// | | | | |
| | | | | XP_001096303 /// | | | | |
| | | | | XP_001096427 /// | | | | |
| | | | | XP_001096539 /// | | | | |
| | | | | XP_001096644 /// | | | | |
| | | | | XP_001096747 /// | | | | |
| | | | | XP_001096864 /// | | | | |
| | | | | XP_001096974 /// | | | | |
| | | | | XP_001097086 /// | | | | |
| | | | | XP_001097202 /// | | | | |
| | | | | XP_001097299 /// | | | | |
| | | | | XP_001097408 /// | | | | |
| | | | | XP_001097505 /// | | | | |
| | | | | XP_001097599 /// | | | | |
| | | | | XP_001097712 /// | | | | |
| | | | | XP_001097810 /// | | | | |
| | | | | XP_001097908 /// | | | | |
| | | | | XP_001097998 /// | | | | |
| | | | | XP_001098090 /// | | | | |
| | | | | XP_001098194 /// | | | | |
| | | | | XP_001098301 /// | | | | |
| | | | | XP_001098398 /// | | | | |
| | | | | XP_001098491 /// | | | | |
| | | | | XP_001098588 /// | | | | |
| | | | | XP_001098689 /// | | | | |
| | | | | XP_001098792 /// | | | | |
| | | | | XP_001098897 /// | | | | |
| | | | | XP_001098993 /// | | | | |
| | | | | XP_001099102 /// | | | | |
| | | | | XP_001099203 /// | | | | |
| | | | | XP_001099314 /// | | | | |
| | | | | XP_001099411 /// | | | | |
| | | | | XP_001099522 /// | | | | |
| | | | | XP_001099613 /// | | | | |
| | | | | XP_001099715 /// | | | | |
| | | | | XP_001099819 /// | | | | |
| | | | | XP_001099920 | | | | |
| MmugDNA.1267.1.S1_s_at | CYP3A64 /// | cytochrome P450 3A64 /// | Mmu.9706 | NP_001035504 /// | 89.83363 | A | 288.6809 | P |
| | LOC718917 | similar to Cytochrome P450 3A7 (CYPIIIA7) | | XP_001097930 /// | | | | |
| | | (P450-HFLA) | | XP_001112281 | | | | |
| MmugDNA.36232.1.S1_at | DAB2 | disabled homolog 2 | Mmu.12958 | XP_001084792 | 72.17534 | A | 60.9826 | P |
| MmugDNA.13802.1.S1_at | DCN | decorin | Mmu.1687 | XP_001103412 /// | 52.87949 | A | 78.89815 | P |
| | | | | XP_001103495 /// | | | | |
| | | | | XP_001103577 /// | | | | |
| | | | | XP_001103663 /// | | | | |
| | | | | XP_001103749 /// | | | | |
| | | | | XP_001103834 /// | | | | |
| | | | | XP_001103918 /// | | | | |
| | | | | XP_001104009 /// | | | | |
| | | | | XP_001104095 /// | | | | |
| | | | | XP_001104178 /// | | | | |
| | | | | XP_001104261 /// | | | | |
| | | | | XP_001104340 | | | | |
| MmugDNA.22700.1.S1_at | DCN | decorin | Mmu.1687 | XP_001103412 /// | 120.1081 | A | 180.4038 | P |
| | | | | XP_001103495 /// | | | | |
| | | | | XP_001103577 /// | | | | |
| | | | | XP_001103663 /// | | | | |
| | | | | XP_001103749 /// | | | | |
| | | | | XP_001103834 /// | | | | |
| | | | | XP_001103918 /// | | | | |
| | | | | XP_001104009 /// | | | | |
| | | | | XP_001104095 /// | | | | |
| | | | | XP_001104178 /// | | | | |
| | | | | XP_001104261 /// | | | | |
| | | | | XP_001104340 | | | | |
| MmugDNA.41798.1.S1_at | DHRS7 | dehydrogenase/reductase (SDR family) member 7 | Mmu.4703 | XP_001095590 /// | 122.1582 | A | 142.4385 | P |
| | | | | XP_001095688 /// | | | | |
| | | | | XP_001095790 | | | | |
| MmugDNA.26154.1.S1_at | DRD2 | dopamine receptor D2 | Mmu.3401 | XP_001085336 /// | 94.5069 | A | 172.3374 | P |
| | | | | XP_001085449 /// | | | | |
| | | | | XP_001085571 | | | | |
| MmugDNA.26188.1.S1_at | ECEL1 | endothelin converting enzyme-like 1 | Mmu.11796 | XP_001117070 | 178.3571 | A | 271.1654 | P |
| MmugDNA.38477.1.S1_at | ELK4 | ELK4, ETS-domain protein (SRF accessory protein 1) | — | XP_001087748 | 144.6712 | A | 136.5781 | P |
| MmugDNA.27688.1.S1_at | EPOR | erythropoietin receptor | — | XP_001105833 | 188.3416 | A | 249.6902 | P |
| MmuSTS.4736.1.S1_at | ERBB3 | v-erb-b2 erythroblastic leukemia viral oncogene | Mmu.758 | XP_001113794 /// | 161.6664 | A | 180.3977 | P |
| | | homolog 3 (avian) | | XP_001113900 /// | | | | |
| | | | | XP_001113928 /// | | | | |
| | | | | XP_001113953 | | | | |
| MmugDNA.18128.1.S1_at | F7 | coagulation factor VII | Mmu.11080 | NP_001073605 /// | 95.21188 | A | 159.5128 | P |
| | | | | XP_001118128 | | | | |
| MmuSTS.4781.1.S1_at | FKBP10 | FK506 binding protein 10, 65 kDa | Mmu.14407 | XP_001107581 /// | 144.692 | A | 199.3497 | P |
| | | | | XP_001107638 /// | | | | |
| | | | | XP_001107696 /// | | | | |
| | | | | XP_001107756 /// | | | | |
| | | | | XP_001107821 | | | | |
| MmuSTS.4804.1.S1_at | FPR2 | formyl peptide receptor 2 | Mmu.15383 | XP_001116437 /// | 314.0186 | A | 651.3593 | P |
| | | | | XP_001116445 /// | | | | |
| | | | | XP_001116452 | | | | |
| MmugDNA.22053.1.S1_at | GGCX | gamma-glutamyl carboxylase | Mmu.13911 | XP_001086016 /// | 309.7049 | A | 350.4313 | P |
| | | | | XP_001086474 | | | | |
| MmugDNA.13746.1.S1_s_at | GK | glycerol kinase | Mmu.16427 | XP_001100778 /// | 21.59358 | A | 53.15634 | P |
| | | | | XP_001100861 /// | | | | |
| | | | | XP_001100968 | | | | |
| MmugDNA.37125.1.S1_at | GLRB | glycine receptor, beta | Mmu.1658 | XP_001093718 /// | 140.4933 | A | 195.3956 | P |
| | | | | XP_001093832 | | | | |
| Mmu.8905.1.S1_at | GPD1 | glycerol-3-phosphate | Mmu.3010 | XP_001102726 | 70.93366 | A | 112.7022 | P |
| | | dehydrogenase 1 (soluble) | | | | | | |
| Mmu.11695.1.S1_at | GPR34 | G protein-coupled receptor 34 | Mmu.3224 | XP_001087742 /// | 146.0326 | A | 99.20039 | P |
| | | | | XP_001087858 /// | | | | |
| | | | | XP_001087981 /// | | | | |
| | | | | XP_001088105 | | | | |
| MmuSTS.2489.1.S1_at | GPR77 | G protein-coupled receptor 77 | Mmu.15399 | XP_001112895 /// | 85.30246 | A | 155.9195 | P |
| | | | | XP_001112923 /// | | | | |
| | | | | XP_001112956 | | | | |
| MmugDNA.7761.1.S1_at | HIPK2 | homeodomain interacting protein kinase 2 | Mmu.4514 | XP_001106719 | 112.2257 | A | 149.4128 | P |
| MmuSTS.1348.1.S1_at | HOXB7 | homeobox B7 | Mmu.16913 | XP_001088822 | 134.1311 | A | 174.3028 | P |
| MmugDNA.18776.1.S1_at | HPD | 4-hydroxyphenylpyruvate dioxygenase | Mmu.14575 | XP_001096061 /// | 81.20009 | A | 180.3834 | P |
| | | | | XP_001096175 | | | | |
| MmugDNA.28970.1.S1_at | IL1RN | interleukin 1 receptor antagonist | Mmu.15827 | XP_001091493 /// | 126.3157 | A | 288.0709 | P |
| | | | | XP_001091717 /// | | | | |
| | | | | XP_001091833 | | | | |
| MmuSTS.681.1.S1_at | IL1RN | interleukin 1 receptor antagonist | Mmu.15827 | XP_001091493 /// | 108.0968 | A | 374.1611 | P |
| | | | | XP_001091717 /// | | | | |
| | | | | XP_001091833 | | | | |
| MmugDNA.36020.1.S1_at | ITGB5 | integrin, beta 5 | Mmu.1239 | XP_001113831 /// | 118.9835 | A | 125.353 | P |
| | | | | XP_001113881 /// | | | | |
| | | | | XP_001113909 | | | | |
| Mmu.7460.6.S1_x_at | KIR3DH5 | killer-cell Ig-like receptor KIR3DH5 | Mmu.17415 | NP_001098643 | 251.4261 | A | 276.8067 | P |
| Mmu.9316.1.S1_at | KLF11 | Kruppel-like factor 11 | Mmu.11355 | XP_001090608 | 501.1391 | A | 764.6097 | P |
| MmuSTS.1757.1.S1_at | LOC693556 | similar to ras responsive | Mmu.14828 | XP_001082265 /// | 109.3152 | A | 140.1759 | P |
| | | element binding protein 1 isoform 1 | | XP_001082516 /// | | | | |
| | | | | XP_001082652 /// | | | | |
| | | | | XP_001082784 /// | | | | |
| | | | | XP_001082911 | | | | |
| MmugDNA.20287.1.S1_at | LOC693696 | similar to longevity assurance homolog 2 | Mmu.16301 | XP_001082530 /// | 9.598577 | A | 66.55032 | P |
| | | | | XP_001082667 /// | | | | |
| | | | | XP_001082802 | | | | |
| MmugDNA.17057.1.S1_at | LOC694043 | Similar to RECK protein precursor | Mmu.12983 | XP_001083242 /// | 36.28584 | A | 109.4269 | P |
| | | | | XP_001083599 | | | | |
| MmugDNA.14488.1.S1_at | LOC694192 | similar to Protein C18orf54 precursor | Mmu.15470 | XP_001082497 /// | 158.6407 | A | 85.90692 | P |
| | | | | XP_001082628 /// | | | | |
| | | | | XP_001082761 | | | | |
| MmugDNA.32899.1.S1_at | LOC694220 | similar to glutathione S- | Mmu.16453 | XP_001083156 /// | 151.0824 | A | 150.7997 | P |
| | | transferase, C-terminal domain | | XP_001083273 /// | | | | |
| | | containing isoform 1 | | XP_001083401 | | | | |
| MmugDNA.35637.1.S1_at | LOC694461 | similar to RIO kinase 1 isoform 1 | Mmu.13193 | XP_001084416 /// | 195.3009 | A | 278.8399 | P |
| | | | | XP_001084537 /// | | | | |
| | | | | XP_001084661 | | | | |
| MmugDNA.7498.1.S1_at | LOC695519 | hypothetical protein LOC695519 | Mmu.14586 | XP_001088156 | 103.4704 | A | 289.532 | P |
| MmugDNA.23700.1.S1_at | LOC695660 | similar to glutamate receptor 6 isoform 1 precursor | Mmu.14770 | XP_001086511 /// | 71.56487 | A | 134.0835 | P |
| | | | | XP_001086640 /// | | | | |
| | | | | XP_001086762 | | | | |
| MmugDNA.40736.1.S1_at | LOC695861 | similar to regulatory factor X3 isoform b | Mmu.15656 | XP_001086023 /// | 59.62864 | A | 159.93 | P |
| | | | | XP_001086140 /// | | | | |
| | | | | XP_001086369 /// | | | | |
| | | | | XP_001086480 /// | | | | |
| | | | | XP_001086606 /// | | | | |
| | | | | XP_001086729 | | | | |
| MmugDNA.3419.1.S1_at | LOC696314 | similar to tumor necrosis factor, | Mmu.9026 | — | 133.7824 | A | 178.2315 | P |
| | | alpha-induced protein 2 | | | | | | |
| MmugDNA.40590.1.S1_at | LOC696991 | similar to CG13322-PA, isoform A | Mmu.15342 | XP_001090213 /// | 151.6879 | A | 117.6347 | P |
| | | | | XP_001090328 /// | | | | |
| | | | | XP_001090443 /// | | | | |
| | | | | XP_001090556 | | | | |
| MmugDNA.23596.1.S1_at | LOC697028 | similar to dedicator of cytokinesis 7 | Mmu.4738 | XP_001085641 | 42.86666 | A | 83.91882 | P |
| MmugDNA.19935.1.S1_at | LOC697140 | similar to LIM domain binding 3 | Mmu.11452 | XP_001084921 /// | 127.76 | A | 82.97771 | P |
| | | | | XP_001085158 /// | | | | |
| | | | | XP_001085274 | | | | |
| MmugDNA.25784.1.S1_at | LOC697300 | Similar to DNA topoisomerase I | Mmu.12250 | XP_001088021 | 107.8373 | A | 168.2853 | P |
| MmugDNA.41587.1.S1_at | LOC697948 | similar to Apical Junction | Mmu.15719 | XP_001094873 /// | 231.1788 | A | 220.4972 | P |
| | | Molecule family member (ajm-1) | | XP_001094990 /// | | | | |
| | | | | XP_001095091 /// | | | | |
| | | | | XP_001095202 /// | | | | |
| | | | | XP_001095318 /// | | | | |
| | | | | XP_001095432 /// | | | | |
| | | | | XP_001095548 | | | | |
| MmugDNA.8688.1.S1_s_at | LOC698576 | similar to plakophilin 4 isoform a | Mmu.90 | XP_001090763 /// | 57.58762 | A | 168.3158 | P |
| | | | | XP_001091487 /// | | | | |
| | | | | XP_001091595 /// | | | | |
| | | | | XP_001091710 /// | | | | |
| | | | | XP_001091829 | | | | |
| MmugDNA.25652.1.S1_at | LOC698610 | similar to netrin-G1 ligand | Mmu.14852 | XP_001090221 /// | 159.8217 | A | 335.8282 | P |
| | | | | XP_001090333 /// | | | | |
| | | | | XP_001090447 | | | | |
| MmugDNA.7880.1.S1_at | LOC698922 | similar to transcription | Mmu.1273 | XP_001087401 | 67.81429 | A | 129.1399 | P |
| | | elongation factor B (SIII), polypeptide 1 | | | | | | |
| MmugDNA.26991.1.S1_x_at | LOC699049 | similar to SMT3 suppressor of | Mmu.10807 | XP_001082298 /// | 132.9624 | A | 98.17895 | P |
| | /// | mif two 3 homolog 2 /// similar | | XP_001093875 /// | | | | |
| | LOC705512 | to SMT3 suppressor of mif two | | XP_001101180 | | | | |
| | /// | 3 homolog 2 isoform b | | | | | | |
| | LOC712242 | precursor /// similar to SMT3 | | | | | | |
| | /// SUMO2 | suppressor of mif two 3 | | | | | | |
| | | homolog 2 /// SMT3 suppressor | | | | | | |
| | | of mif two 3 homolog 2 (S. cerevisiae) | | | | | | |
| MmugDNA.30038.1.S1_at | LOC699233 | similar to J domain protein C21orf55 | Mmu.14908 | XP_001091149 /// | 20.38795 | A | 40.94059 | P |
| | | | | XP_001091275 /// | | | | |
| | | | | XP_001091400 | | | | |
| MmugDNA.24540.1.S1_at | LOC699255 | similar to c-Mpl binding protein | Mmu.8566 | XP_001103235 | 97.93331 | A | 95.29922 | P |
| | /// | isoform c /// similar to c-Mpl | | | | | | |
| | LOC713773 | binding protein isoform a | | | | | | |
| MmugDNA.36570.1.S1_at | LOC699320 | similar to keratin 1 | Mmu.15995 | XP_001097706 /// | 82.466 | A | 52.41996 | P |
| | | | | XP_001097800 /// | | | | |
| | | | | XP_001097902 /// | | | | |
| | | | | XP_001097988 /// | | | | |
| | | | | XP_001098082 /// | | | | |
| | | | | XP_001098182 /// | | | | |
| | | | | XP_001098292 | | | | |
| MmugDNA.34762.1.S1_at | LOC699606 | similar to YKT6 v-SNARE protein | Mmu.14457 | XP_001092458 /// | 352.2617 | A | 340.2292 | P |
| | | | | XP_001092567 | | | | |
| MmugDNA.35909.1.S1_at | LOC700246 | hypothetical protein LOC700246 | Mmu.16339 | XP_001091537 /// | 141.387 | A | 109.4642 | P |
| | | | | XP_001091653 /// | | | | |
| | | | | XP_001091770 | | | | |
| MmugDNA.2141.1.S1_at | LOC700632 | similar to tumor protein p53 | Mmu.12749 | XP_001090474 /// | 103.4481 | A | 110.7 | P |
| | | inducible nuclear protein 1 | | XP_001090592 | | | | |
| MmugDNA.6969.1.S1_s_at | LOC700634 | similar to PTK2 protein tyrosine kinase 2 isoform a | Mmu.3254 | XP_001091188 /// | 318.6551 | A | 548.1872 | P |
| | | | | XP_001092257 /// | | | | |
| | | | | XP_001092479 /// | | | | |
| | | | | XP_001092599 /// | | | | |
| | | | | XP_001092714 /// | | | | |
| | | | | XP_001092835 /// | | | | |
| | | | | XP_001092947 /// | | | | |
| | | | | XP_001093060 /// | | | | |
| | | | | XP_001093166 /// | | | | |
| | | | | XP_001093282 | | | | |
| MmugDNA.11282.1.S1_at | LOC700766 | similar to CG14299-PA, isoform A | Mmu.13220 | XP_001089105 | 102.762 | A | 134.5001 | P |
| MmuSTS.1092.1.S1_at | LOC701162 | similar to solute carrier family 6, member 17 | Mmu.11269 | XP_001099500 | 62.05202 | A | 108.5072 | P |
| MmugDNA.9882.1.S1_at | LOC701376 | similar to NADPH oxidase 1 isoform long variant | Mmu.15254 | XP_001089791 /// | 59.80977 | A | 168.2042 | P |
| | | | | XP_001089905 /// | | | | |
| | | | | XP_001090025 /// | | | | |
| | | | | XP_001090252 /// | | | | |
| | | | | XP_001090373 | | | | |
| MmugDNA.24847.1.S1_at | LOC701547 | similar to glycine-N-acyltransferase-like 1 | Mmu.14222 | XP_001092072 /// | 104.8917 | A | 97.61919 | P |
| | | | | XP_001092197 /// | | | | |
| | | | | XP_001092316 /// | | | | |
| | | | | XP_001092427 | | | | |
| Mmu.11429.1.A1_at | LOC701690 | Similar to T-cell activation Rho | Mmu.12230 | XP_001095239 /// | 125.3303 | A | 148.4935 | P |
| | | GTPase-activating protein isoform b | | XP_001095349 /// | | | | |
| | | | | XP_001095467 /// | | | | |
| | | | | XP_001095579 /// | | | | |
| | | | | XP_001095675 /// | | | | |
| | | | | XP_001095775 | | | | |
| MmugDNA.14409.1.S1_at | LOC701722 | similar to nucleoporin 62 | Mmu.12987 | XP_001094695 | 17.99913 | A | 56.40857 | P |
| MmugDNA.12356.1.S1_at | LOC702392 | similar to peroxin1 | Mmu.13262 | XP_001100851 /// | 267.6477 | A | 482.0948 | P |
| | | | | XP_001101055 | | | | |
| MmugDNA.35938.1.S1_at | LOC702474 | similar to F46E10.1a /// similar | Mmu.11756 | XP_001093983 /// | 212.2583 | A | 148.9655 | P |
| | /// | to radical S-adenosyl | | XP_001097818 | | | | |
| | LOC705626 | methionine domain containing 1 | | | | | | |
| MmugDNA.30120.1.S1_at | LOC702596 | similar to nucleolar RNA- | Mmu.15669 | XP_001099108 /// | 517.206 | A | 779.2531 | P |
| | | associated protein alpha isoform | | XP_001099208 | | | | |
| MmugDNA.14645.1.S1_at | LOC702627 | similar to CNKSR family member 3 | Mmu.16800 | XP_001095122 | 112.3272 | A | 135.1359 | P |
| MmuSTS.4277.1.S1_at | LOC702809 | similar to eukaryotic translation | Mmu.7828 | XP_001096741 /// | 46.99945 | A | 76.99638 | P |
| | /// | elongation factor 1 alpha 2 /// | | XP_001107881 /// | | | | |
| | LOC703715 | similar to eukaryotic translation | | XP_001107938 /// | | | | |
| | /// | elongation factor 1 alpha 2 /// | | XP_001107997 /// | | | | |
| | LOC704199 | similar to eukaryotic translation | | XP_001108047 | | | | |
| | /// | elongation factor 1 alpha 2 /// | | | | | | |
| | LOC704438 | similar to eukaryotic translation | | | | | | |
| | /// | elongation factor 1 alpha 1 /// | | | | | | |
| | LOC709017 | similar to eukaryotic translation | | | | | | |
| | /// | elongation factor 1 alpha 1 /// | | | | | | |
| | LOC715351 | similar to eukaryotic translation | | | | | | |
| | | elongation factor 1 alpha 1 | | | | | | |
| MmugDNA.18234.1.S1_s_at | LOC703331 | similar to runt-related | Mmu.14787 | XP_001100592 /// | 58.99397 | A | 140.3681 | P |
| | | transcription factor 2 isoform b | | XP_001100682 /// | | | | |
| | | | | XP_001100772 /// | | | | |
| | | | | XP_001100855 /// | | | | |
| | | | | XP_001100961 /// | | | | |
| | | | | XP_001101058 | | | | |
| MmugDNA.42623.1.S1_at | LOC703350 | Similar to SLD5 | Mmu.12445 | XP_001094432 | 116.1626 | A | 122.4898 | P |
| MmugDNA.14145.1.S1_s_at | LOC703676 | similar to clusterin associated | Mmu.12076 | XP_001095007 | 9.514027 | A | 65.8841 | P |
| | | protein 1 isoform 1 | | | | | | |
| MmugDNA.6896.1.S1_at | LOC703747 | hypothetical protein LOC703747 | Mmu.11245 | XP_001097797 /// | 99.1186 | A | 203.7421 | P |
| | | | | XP_001097899 /// | | | | |
| | | | | XP_001097986 /// | | | | |
| | | | | XP_001098078 /// | | | | |
| | | | | XP_001098178 /// | | | | |
| | | | | XP_001098384 | | | | |
| MmugDNA.20879.1.S1_at | LOC703981 | similar to NOD9 protein isoform 1 | Mmu.15714 | XP_001104018 /// | 68.5675 | A | 220.5779 | P |
| | | | | XP_001104187 /// | | | | |
| | | | | XP_001104271 /// | | | | |
| | | | | XP_001104348 /// | | | | |
| | | | | XP_001104430 | | | | |
| MmugDNA.43058.1.S1_at | LOC704126 | similar to kinesin family member 13A | Mmu.14823 | XP_001096456 /// | 240.8623 | A | 350.718 | P |
| | | | | XP_001096670 /// | | | | |
| | | | | XP_001096780 /// | | | | |
| | | | | XP_001096896 | | | | |
| MmugDNA.32338.1.S1_at | LOC704620 | Hypothetical protein LOC704620 | Mmu.12860 | XP_001092983 | 484.9445 | A | 883.7935 | P |
| MmugDNA.26222.1.S1_at | LOC704779 | hypothetical protein LOC704779 | Mmu.1356 | XP_001088505 /// | 145.6491 | A | 301.6946 | P |
| | | | | XP_001088616 /// | | | | |
| | | | | XP_001088722 /// | | | | |
| | | | | XP_001088845 | | | | |
| MmugDNA.41256.1.S1_at | LOC705369 | similar to SET domain containing 3 | Mmu.12527 | XP_001103146 /// | 307.612 | A | 308.8239 | P |
| | | | | XP_001103224 /// | | | | |
| | | | | XP_001103370 | | | | |
| MmugDNA.32069.1.S1_at | LOC705781 | similar to CG10721-PA | Mmu.11312 | XP_001098386 | 139.6408 | A | 177.6773 | P |
| MmugDNA.7008.1.S1_at | LOC705781 | similar to CG10721-PA | Mmu.11312 | XP_001098386 | 257.4816 | A | 150.1654 | P |
| MmugDNA.11389.1.S1_at | LOC705913 | similar to cytidine deaminase | Mmu.14713 | XP_001096632 | 157.2027 | A | 225.252 | P |
| Mmu.8604.1.A1_at | LOC706093 | Similar to golgi-associated | Mmu.12976 | XP_001099454 /// | 105.2247 | A | 94.18667 | P |
| | | microtubule-binding protein HOOK3 | | XP_001099564 | | | | |
| MmugDNA.6257.1.S1_at | LOC706392 | similar to Temporarily Assigned | Mmu.15492 | XP_001100027 /// | 28.65171 | A | 176.2832 | P |
| | | Gene name family member (tag-241) | | XP_001100112 /// | | | | |
| | | | | XP_001100204 | | | | |
| MmugDNA.2760.1.S1_at | LOC706531 | similar to SRY (sex determining region Y)-box 4 | Mmu.2172 | XP_001098923 | 494.1494 | A | 361.4545 | P |
| MmugDNA.21203.1.S1_x_at | LOC707383 | similar to amyloid beta (A4) | Mmu.669 | XP_001101993 | 428.4578 | A | 214.6058 | P |
| | | precursor protein-binding, | | | | | | |
| | | family B, member 1 interacting protein | | | | | | |
| MmugDNA.35674.1.S1_at | LOC707661 | similar to additional sex combs like 1 | Mmu.15484 | XP_001101400 /// | 110.2575 | A | 137.5026 | P |
| | | | | XP_001101495 /// | | | | |
| | | | | XP_001101584 /// | | | | |
| | | | | XP_001101677 | | | | |
| MmugDNA.14411.1.S1_at | LOC707686 | similar to angiogenic factor VG5Q | Mmu.12022 | XP_001106588 /// | 312.2592 | A | 193.1674 | P |
| | | | | XP_001106645 /// | | | | |
| | | | | XP_001106704 /// | | | | |
| | | | | XP_001106767 | | | | |
| MmugDNA.6011.1.S1_at | LOC707982 | similar to rhomboid domain | Mmu.15898 | XP_001110015 /// | 253.2026 | A | 444.7268 | P |
| | /// | containing 1 /// hypothetical | | XP_001110070 /// | | | | |
| | LOC708189 | protein LOC708189 | | XP_001110119 /// | | | | |
| | | | | XP_001110165 /// | | | | |
| | | | | XP_001110210 | | | | |
| MmugDNA.37057.1.S1_at | LOC708094 | similar to mannosyl (alpha-1,3-)- | Mmu.15839 | XP_001102758 /// | 104.4101 | A | 124.6013 | P |
| | | glycoprotein beta-1,4-N- | | XP_001102852 | | | | |
| | | acetylglucosaminyltransferase, | | | | | | |
| | | isoenzyme A | | | | | | |
| MmugDNA.30697.1.S1_at | LOC708649 | similar to leucine rich repeat containing 28 | Mmu.13704 | XP_001101795 /// | 163.4347 | A | 185.626 | P |
| | | | | XP_001101886 /// | | | | |
| | | | | XP_001101987 /// | | | | |
| | | | | XP_001102074 | | | | |
| MmuSTS.4837.1.S1_at | LOC708940 | similar to Guanine nucleotide- | Mmu.14862 | XP_001108555 /// | 67.89697 | A | 77.73469 | P |
| | | binding protein G(i), alpha-1 | | XP_001108609 /// | | | | |
| | | subunit (Adenylate cyclase- | | XP_001108656 | | | | |
| | | inhibiting G alpha protein) | | | | | | |
| MmuSTS.508.1.S1_at | LOC709049 | similar to activating signal | Mmu.14734 | XP_001105587 /// | 145.0563 | A | 121.9355 | P |
| | | cointegrator 1 complex subunit 1 | | XP_001105655 /// | | | | |
| | | | | XP_001105727 /// | | | | |
| | | | | XP_001105789 /// | | | | |
| | | | | XP_001105856 /// | | | | |
| | | | | XP_001105929 /// | | | | |
| | | | | XP_001106003 | | | | |
| MmugDNA.32637.1.S1_at | LOC709253 | similar to nucleosomal binding protein 1 | Mmu.13802 | XP_001103630 | 80.94846 | A | 79.39787 | P |
| Mmu.10346.1.S1_at | LOC709544 | hypothetical protein LOC709544 | Mmu.16556 | XP_001096738 | 507.0977 | A | 328.4313 | P |
| MmugDNA.1217.1.S1_at | LOC709920 | similar to Helix-loop-helix | Mmu.15111 | XP_001112029 /// | 8.452376 | A | 74.10219 | P |
| | | protein 2 (HEN2) (Nescient | | XP_001112063 /// | | | | |
| | | helix loop helix 2) (NSCL-2) | | XP_001112098 | | | | |
| MmugDNA.26478.1.S1_at | LOC710518 | similar to mitochondrial solute | Mmu.11000 | XP_001105927 /// | 455.317 | A | 606.7848 | P |
| | | carrier-like protein | | XP_001106001 /// | | | | |
| | | | | XP_001106076 /// | | | | |
| | | | | XP_001106141 /// | | | | |
| | | | | XP_001106211 | | | | |
| MmugDNA.28666.1.S1_at | LOC710731 | similar to palmdelphin | Mmu.12773 | XP_001105606 /// | 37.84584 | A | 95.51393 | P |
| | | | | XP_001105677 /// | | | | |
| | | | | XP_001105748 /// | | | | |
| | | | | XP_001105804 /// | | | | |
| | | | | XP_001105954 | | | | |
| MmugDNA.17269.1.S1_at | LOC711164 | similar to sulfite oxidase | Mmu.15990 | XP_001113374 /// | 171.6959 | A | 246.8745 | P |
| | | | | XP_001113406 /// | | | | |
| | | | | XP_001113430 /// | | | | |
| | | | | XP_001113456 | | | | |
| MmugDNA.4283.1.S1_at | LOC711279 | similar to leucine zipper, | Mmu.16155 | XP_001109524 /// | 156.9818 | A | 240.0757 | P |
| | | putative tumor suppressor 2 | | XP_001109574 /// | | | | |
| | | | | XP_001109628 /// | | | | |
| | | | | XP_001109674 /// | | | | |
| | | | | XP_001109715 /// | | | | |
| | | | | XP_001109770 /// | | | | |
| | | | | XP_001109817 | | | | |
| MmugDNA.33876.1.S1_s_at | LOC711336 | similar to olfactomedin 2 | Mmu.15445 | XP_001099531 /// | 105.1338 | A | 116.0832 | P |
| | | | | XP_001099628 /// | | | | |
| | | | | XP_001099726 /// | | | | |
| | | | | XP_001099831 | | | | |
| MmugDNA.17217.1.S1_at | LOC711485 | similar to acyl-Coenzyme A | Mmu.12959 | XP_001109931 | 123.1174 | A | 238.599 | P |
| | | dehydrogenase, long chain precursor | | | | | | |
| MmugDNA.15166.1.S1_at | LOC711633 | similar to zinc finger CCCH-type containing 10 | Mmu.15988 | XP_001114089 /// | 185.2786 | A | 222.4264 | P |
| | | | | XP_001114107 /// | | | | |
| | | | | XP_001114132 /// | | | | |
| | | | | XP_001114150 | | | | |
| MmuSTS.4200.1.S1_at | LOC711691 | Similar to pre-B-cell leukemia | Mmu.4832 | XP_001100529 | 105.6631 | A | 385.9178 | P |
| | | transcription factor 3 | | | | | | |
| MmugDNA.30723.1.S1_at | LOC711984 | Similar to zinc finger CCCH- | Mmu.12594 | XP_001106124 /// | 175.1743 | A | 445.4023 | P |
| | | type domain containing 7 | | XP_001106316 /// | | | | |
| | | | | XP_001106384 /// | | | | |
| | | | | XP_001106446 | | | | |
| MmugDNA.20892.1.S1_at | LOC712033 | similar to PCTAIRE protein kinase 1 | Mmu.14735 | XP_001100911 | 120.9395 | A | 238.5381 | P |
| MmugDNA.10446.1.S1_at | LOC712115 | similar to ADP-ribosylation | Mmu.14062 | XP_001101005 | 75.08247 | A | 68.66737 | P |
| | | factor guanine nucleotide factor 6 isoform a | | | | | | |
| MmugDNA.12728.1.S1_at | LOC712272 | similar to fibronectin type 3 and | Mmu.13034 | XP_001109699 | 37.6952 | A | 102.0832 | P |
| | | SPRY domain-containing protein | | | | | | |
| MmugDNA.22601.1.S1_at | LOC713029 | hypothetical protein LOC713029 | Mmu.14564 | XP_001110409 | 11.16738 | A | 58.5388 | P |
| MmugDNA.12133.1.S1_at | LOC713035 | similar to BCL2-like 1 isoform 1 | Mmu.16103 | XP_001109879 /// | 159.8234 | A | 137.4199 | P |
| | | | | XP_001109926 /// | | | | |
| | | | | XP_001109964 /// | | | | |
| | | | | XP_001110009 /// | | | | |
| | | | | XP_001110062 /// | | | | |
| | | | | XP_001110113 /// | | | | |
| | | | | XP_001110162 /// | | | | |
| | | | | XP_001110205 /// | | | | |
| | | | | XP_001110245 /// | | | | |
| | | | | XP_001110285 /// | | | | |
| | | | | XP_001110326 | | | | |
| MmugDNA.17300.1.S1_s_at | LOC713324 | similar to erythrocyte | Mmu.14133 | XP_001102639 | 68.31221 | A | 110.1094 | P |
| | | membrane protein band 4.1 like 4B isoform 2 | | | | | | |
| MmuSTS.2766.1.S1_at | LOC713353 | Similar to unactive progesterone receptor, 23 Kd | Mmu.4022 | XP_001115328 /// | 77.942 | A | 160.7406 | P |
| | | | | XP_001115356 /// | | | | |
| | | | | XP_001115374 /// | | | | |
| | | | | XP_001115388 | | | | |
| MmugDNA.37205.1.S1_at | LOC713800 | similar to plexin A2 | Mmu.15066 | XP_001110877 /// | 40.86835 | A | 79.4382 | P |
| | | | | XP_001110951 /// | | | | |
| | | | | XP_001110980 | | | | |
| MmugDNA.23412.1.S1_x_at | LOC715553 | similar to Protein PP2447 | Mmu.16050 | XP_001111734 /// | 651.9257 | A | 657.9822 | P |
| | | | | XP_001111774 /// | | | | |
| | | | | XP_001111813 /// | | | | |
| | | | | XP_001111850 /// | | | | |
| | | | | XP_001111878 /// | | | | |
| | | | | XP_001111918 /// | | | | |
| | | | | XP_001111956 | | | | |
| MmuSTS.563.1.S1_at | LOC715706 | Similar to schlafen family member 13 | Mmu.11333 | XP_001114176 /// | 47.03052 | A | 79.03813 | P |
| | | | | XP_001114192 /// | | | | |
| | | | | XP_001114211 | | | | |
| MmugDNA.23724.1.S1_at | LOC715752 | similar to zinc finger protein 44 | Mmu.13185 | XP_001107149 /// | 177.6898 | A | 126.2318 | P |
| | | | | XP_001107204 /// | | | | |
| | | | | XP_001107269 | | | | |
| MmugDNA.30744.1.S1_s_at | LOC715853 | similar to interphase | Mmu.13940 | XP_001113603 | 221.8494 | A | 233.8704 | P |
| | | cyctoplasmic foci protein 45 | | | | | | |
| MmugDNA.11990.1.S1_at | LOC716153 | similar to myelin gene | Mmu.13074 | XP_001106720 | 32.29033 | A | 49.318 | P |
| | | expression factor 2 | | | | | | |
| MmugDNA.17648.1.S1_at | LOC716239 | similar to Pre-B-cell leukemia | Mmu.6054 | — | 117.4557 | A | 180.2829 | P |
| | | transcription factor 2 | | | | | | |
| | | (Homeobox protein PBX2) | | | | | | |
| | | (Protein G17) | | | | | | |
| MmugDNA.40257.1.S1_at | LOC716245 | similar to sphingosine-1- | Mmu.4100 | XP_001106861 | 122.3767 | A | 129.1462 | P |
| | | phosphate lyase 1 | | | | | | |
| MmugDNA.36455.1.S1_at | LOC716351 | similar to SEC22 vesicle | Mmu.14967 | XP_001115262 /// | 162.7943 | A | 137.2903 | P |
| | | trafficking protein homolog C isoform b | | XP_001115277 /// | | | | |
| | | | | XP_001115291 /// | | | | |
| | | | | XP_001115333 /// | | | | |
| | | | | XP_001115349 /// | | | | |
| | | | | XP_001115363 /// | | | | |
| | | | | XP_001115380 | | | | |
| Mmu.8945.1.S1_at | LOC717346 | similar to complement factor H isoform a precursor | Mmu.4677 | XP_001111499 /// | 90.61079 | A | 143.7754 | P |
| | | | | XP_001111535 /// | | | | |
| | | | | XP_001111875 | | | | |
| MmuSTS.2495.1.S1_at | LOC719814 | similar to zinc finger protein 577 | Mmu.15382 | XP_001116472 /// | 105.0435 | A | 110.711 | P |
| | | | | XP_001116485 /// | | | | |
| | | | | XP_001116492 | | | | |
| MmugDNA.11286.1.S1_at | LOC720186 | similar to immunoglobulin | Mmu.11138 | XP_001115307 | 385.697 | A | 568.326 | P |
| | | superfamily, member 8 | | | | | | |
| MmunewRS.138.1.S1_at | LOC721825 | similar to cAMP responsive | Mmu.11710 | XP_001118020 | 87.49419 | A | 151.4151 | P |
| | | element binding protein 3-like 3 | | | | | | |
| MmugDNA.16852.1.S1_at | LTB4R | leukotriene B4 receptor | — | XP_001114016 /// | 97.06477 | A | 171.7247 | P |
| | | | | XP_001114035 | | | | |
| MmugDNA.21143.1.S1_at | MAOA | monoamine oxidase A | Mmu.3283 | XP_001096840 | 127.6487 | A | 274.6833 | P |
| MmugDNA.17553.1.S1_at | MMP19 | matrix metallopeptidase 19 | — | XP_001111542 /// | 95.18174 | A | 216.4568 | P |
| | | | | XP_001111624 | | | | |
| MmugDNA.33389.1.S1_at | MPZL2 | myelin protein zero-like 2 | — | XP_001096977 /// | 93.9895 | A | 109.8271 | P |
| | | | | XP_001097090 | | | | |
| MmugDNA.15929.1.S1_at | MRAS | muscle RAS oncogene homolog | Mmu.14559 | XP_001114261 /// | 44.73249 | A | 105.5022 | P |
| | | | | XP_001114274 /// | | | | |
| | | | | XP_001114299 /// | | | | |
| | | | | XP_001114317 | | | | |
| MmuSTS.4170.1.S1_at | MRAS | muscle RAS oncogene homolog | Mmu.14559 | XP_001114261 /// | 150.9189 | A | 150.6777 | P |
| | | | | XP_001114274 /// | | | | |
| | | | | XP_001114299 /// | | | | |
| | | | | XP_001114317 | | | | |
| MmugDNA.3439.1.S1_at | NEB | nebulin | Mmu.10774 | XP_001084585 | 4.838272 | A | 79.8995 | P |
| MmugDNA.28543.1.S1_at | NR4A2 | Nuclear receptor subfamily 4, group A, member 2 | Mmu.11507 | XP_001085438 /// | 67.00845 | A | 127.2987 | P |
| | | | | XP_001085797 /// | | | | |
| | | | | XP_001085905 /// | | | | |
| | | | | XP_001086011 /// | | | | |
| | | | | XP_001086128 /// | | | | |
| | | | | XP_001086234 /// | | | | |
| | | | | XP_001086356 /// | | | | |
| | | | | XP_001086593 /// | | | | |
| | | | | XP_001086719 | | | | |
| MmugDNA.31823.1.S1_at | PRKACB | protein kinase, cAMP-dependent, catalytic, beta | Mmu.1940 | XP_001105459 /// | 145.2954 | A | 153.0346 | P |
| | | | | XP_001105534 /// | | | | |
| | | | | XP_001105605 /// | | | | |
| | | | | XP_001105676 /// | | | | |
| | | | | XP_001105747 /// | | | | |
| | | | | XP_001105803 /// | | | | |
| | | | | XP_001105875 /// | | | | |
| | | | | XP_001105953 /// | | | | |
| | | | | XP_001106026 /// | | | | |
| | | | | XP_001106097 /// | | | | |
| | | | | XP_001106164 | | | | |
| MmugDNA.7366.1.S1_at | PRKCA | protein kinase C, alpha | Mmu.5709 | — | 168.4086 | A | 185.0983 | P |
| MmugDNA.36754.1.S1_at | PRR3 | proline rich 3 | Mmu.12623 | XP_001100535 | 92.55525 | A | 96.15895 | P |
| MmugDNA.7677.1.S1_at | RBM34 | RNA binding motif protein 34 | Mmu.12653 | XP_001102020 /// | 139.6196 | A | 132.859 | P |
| | | | | XP_001102111 | | | | |
| MmuSTS.3315.1.S1_at | SH3GLB2 | SH3-domain GRB2-like | Mmu.17131 | XP_001109262 | 415.9608 | A | 394.2061 | P |
| | | endophilin B2 | | | | | | |
| MmugDNA.34347.1.S1_s_at | SLC25A13 | solute carrier family 25, | Mmu.11416 | XP_001088340 | 83.63106 | A | 107.3469 | P |
| | | member 13 (citrin) | | | | | | |
| MmugDNA.43541.1.S1_at | SLC25A17 | Solute carrier family 25 | Mmu.4154 | XP_001102293 | 135.3007 | A | 74.23647 | P |
| | | (mitochondrial carrier; | | | | | | |
| | | peroxisomal membrane protein, | | | | | | |
| | | 34 kDa), member 17 | | | | | | |
| MmugDNA.15062.1.S1_at | SSBP1 | Single-stranded DNA binding protein 1 | Mmu.4481 | XP_001084058 /// | 129.9467 | A | 215.4136 | P |
| | | | | XP_001084179 /// | | | | |
| | | | | XP_001084295 /// | | | | |
| | | | | XP_001084414 /// | | | | |
| | | | | XP_001084536 | | | | |
| MmugDNA.9561.1.S1_at | TIMP-2 | tissue inhibitor of matrix | Mmu.1702 | XP_001106657 | 369.0166 | A | 492.1667 | P |
| | | metalloproteinase-2 | | | | | | |
| MmugDNA.502.1.S1_at | TIRAP | toll-interleukin 1 receptor (TIR) | Mmu.17493 | NP_001123907 /// | 205.38 | A | 169.1459 | P |
| | | domain containing adaptor protein | | XP_001112918 /// | | | | |
| | | | | XP_001112950 | | | | |
| MmugDNA.31314.1.S1_at | TMLHE | trimethyllysine hydroxylase, epsilon | Mmu.15229 | XP_001099467 /// | 183.9673 | A | 334.9409 | P |
| | | | | XP_001099575 /// | | | | |
| | | | | XP_001099674 | | | | |
| MmugDNA.86.1.S1_at | TRHDE | thyrotropin-releasing hormone | Mmu.130 | XP_001109391 | 53.7547 | A | 134.5263 | P |
| | | degrading enzyme | | | | | | |
| Mmu.5321.1.S1_at | TTC12 | tetratricopeptide repeat domain 12 | Mmu.13011 | XP_001084384 /// | 108.8476 | A | 113.706 | P |
| | | | | XP_001084508 /// | | | | |
| | | | | XP_001084630 /// | | | | |
| | | | | XP_001084748 /// | | | | |
| | | | | XP_001084868 /// | | | | |
| | | | | XP_001084979 | | | | |
| MmugDNA.33980.1.S1_at | TTLL4 | tubulin tyrosine ligase-like family, member 4 | Mmu.15909 | XP_001094019 /// | 273.2361 | A | 211.3274 | P |
| | | | | XP_001094615 /// | | | | |
| | | | | XP_001094742 /// | | | | |
| | | | | XP_001094864 | | | | |
| MmugDNA.20532.1.S1_at | WDR77 | WD repeat domain 77 | Mmu.4390 | XP_001105103 | 179.0304 | A | 343.9792 | P |
| MmugDNA.33665.1.S1_at | WDR8 | WD repeat domain 8 | Mmu.15190 | XP_001083578 /// | 178.8616 | A | 259.0192 | P |
| | | | | XP_001083787 /// | | | | |
| | | | | XP_001083904 /// | | | | |
| | | | | XP_001084011 | | | | |
| MmugDNA.26867.1.S1_at | XG | Xg blood group | Mmu.11499 | XP_001086077 /// | 29.58194 | A | 40.78949 | P |
| | | | | XP_001086193 | | | | |
| MmugDNA.19387.1.S1_at | ZDHHC3 | zinc finger, DHHC-type containing 3 | Mmu.11811 | XP_001114690 /// | 387.6217 | A | 606.2077 | P |
| | | | | XP_001114737 /// | | | | |
| | | | | XP_001114753 | | | | |
| MmugDNA.42763.1.S1_at | ZFAND5 | zinc finger, AN1-type domain 5 | Mmu.1639 | XP_001096078 /// | 2.943856 | A | 79.52551 | P |
| | | | | XP_001096191 /// | | | | |
| | | | | XP_001096312 /// | | | | |
| | | | | XP_001096433 /// | | | | |
| | | | | XP_001096545 | | | | |
| MmugDNA.29066.1.S1_at | ZNRD1 | zinc ribbon domain containing 1 | Mmu.17470 | NP_001108417 /// | 87.50885 | A | 97.50604 | P |
| | | | | XP_001105355 /// | | | | |
| | | | | XP_001105431 | | | | |

TABLE 2
Differentially expressed genes in PBMCs of NHPs 2-hr following MCAO

| Probe Set ID | Gene Symbol | Gene Title | UniGene ID | First ID Parsed | Pre-O Signal | Pre-O detection | 2-hr Signal | 2-hr detection |
|---|---|---|---|---|---|---|---|---|
| MmugDNA.2736.1.S1_at | C1QA | Complement component 1, q | Mmu.4861 | XP_001101655 | 182.484 | A | 150.6514 | P |
| | | subcomponent, A chain | | | | | | |
| MmugDNA.26392.1.S1_at | CCNF | cyclin F | Mmu.11675 | XP_001085466 | 114.6939 | A | 244.6208 | P |
| MmugDNA.25206.1.S1_at | CDC2 | cell division cycle 2, G1 to S and G2 | Mmu.16170 | XP_001095697 | 83.30299 | A | 74.22997 | P |
| | | to M | | | | | | |
| MmunewRS.528.1.S1_at | CSF2RB | colony stimulating factor 2 receptor, | Mmu.1176 | XP_001086084 | 146.8577 | A | 477.5113 | P |
| | | beta, low-affinity (granulocyte- | | | | | | |
| | | macrophage) | | | | | | |
| Mmu.15973.2.S1_x_at | CSH-3 /// CSH-4 /// | chorionic somatommamotropin | Mmu.10849 | NP_001036203 | 122.2181 | A | 172.7128 | P |
| | GH1 /// | hormone 3 /// chorionic | | | | | | |
| | LOC718474 /// | somatommamotropin hormone 4 /// | | | | | | |
| | LOC721256 /// | growth hormone 1 /// similar to | | | | | | |
| | LOC735307 | growth hormone 1 isoform 1 /// | | | | | | |
| | | similar to growth hormone 1 isoform | | | | | | |
| | | 1 /// chorionic somatomammotropin-1 | | | | | | |
| MmugDNA.5060.1.S1_at | CTNND1 | catenin (cadherin-associated | Mmu.1419 | XP_001094621 | 79.00558 | A | 381.7483 | P |
| | | protein), delta 1 | | | | | | |
| MmugDNA.40586.1.S1_s_at | CYP3A5 /// | cytochrome P450, family 3, subfamily | Mmu.3065 | NP_001035309 | 176.8445 | A | 313.2471 | P |
| | CYP3A64 | A, polypeptide 5 /// cytochrome P450 | | | | | | |
| | | 3A64 | | | | | | |
| MmugDNA.1267.1.S1_s_at | CYP3A64 /// | cytochrome P450 3A64 /// similar to | Mmu.9706 | NP_001035504 | 89.83363 | A | 481.9885 | P |
| | LOC718917 | Cytochrome P450 3A7 (CYPIIIA7) (P450-HFLA) | | | | | | |
| MmugDNA.13802.1.S1_at | DCN | decorin | Mmu.1687 | XP_001103412 | 52.87949 | A | 124.1306 | P |
| MmugDNA.14528.1.S1_s_at | DCN | decorin | Mmu.1687 | XP_001103412 | 107.5135 | A | 183.8077 | P |
| MmugDNA.41798.1.S1_at | DHRS7 | dehydrogenase/reductase (SDR | Mmu.4703 | XP_001095590 | 122.1582 | A | 208.7733 | P |
| | | family) member 7 | | | | | | |
| MmugDNA.620.1.S1_at | ENPP1 | ectonucleotide pyrophosphatase/phosphodiesterase 1 | Mmu.14518 | XP_001103359 | 50.47231 | A | 96.11094 | P |
| MmuSTS.4736.1.S1_at | ERBB3 | v-erb-b2 erythroblastic leukemia viral | Mmu.758 | XP_001113794 | 161.6664 | A | 208.198 | P |
| | | oncogene homolog 3 (avian) | | | | | | |
| MmugDNA.11377.1.S1_s_at | FCAR | Fc fragment of IgA, receptor for | Mmu.3792 | NP_001035039 | 196.9096 | A | 388.9353 | P |
| MmugDNA.33925.1.S1_s_at | FOXP2 | forkhead box P2 | Mmu.4784 | NP_001028193 | 204.0107 | A | 255.1464 | P |
| MmuSTS.4804.1.S1_at | FPR2 | formyl peptide receptor 2 | Mmu.15383 | XP_001116437 | 314.0186 | A | 1280.783 | P |
| MmugDNA.13746.1.S1_s_at | GK | glycerol kinase | Mmu.16427 | XP_001100778 | 21.59358 | A | 114.4026 | P |
| MmugDNA.37125.1.S1_at | GLRB | glycine receptor, beta | Mmu.1658 | XP_001093718 | 140.4933 | A | 274.4669 | P |
| Mmu.11695.1.S1_at | GPR34 | G protein-coupled receptor 34 | Mmu.3224 | XP_001087742 | 146.0326 | A | 147.7894 | P |
| MmuSTS.2489.1.S1_at | GPR77 | G protein-coupled receptor 77 | Mmu.15399 | XP_001112895 | 85.30246 | A | 286.7114 | P |
| MmuSTS.1348.1.S1_at | HOXB7 | homeobox B7 | Mmu.16913 | XP_001088822 | 134.1311 | A | 125.7572 | P |
| MmugDNA.18776.1.S1_at | HPD | 4-hydroxyphenylpyruvate | Mmu.14575 | XP_001096061 | 81.20009 | A | 294.6741 | P |
| | | dioxygenase | | | | | | |
| Mmu.15730.2.S1_at | HSP90AB1 | heat shock 90 kDa protein 1, beta | Mmu.142 | XP_001098219 | 97.19216 | A | 321.4503 | P |
| MmuSTS.681.1.S1_at | IL1RN | interleukin 1 receptor antagonist | Mmu.15827 | XP_001091493 | 108.0968 | A | 635.2902 | P |
| MmugDNA.28970.1.S1_at | IL1RN | interleukin 1 receptor antagonist | Mmu.15827 | XP_001091493 | 126.3157 | A | 511.0923 | P |
| Mmu.9316.1.S1_at | KLF11 | Kruppel-like factor 11 | Mmu.11355 | XP_001090608 | 501.1391 | A | 666.5593 | P |
| Mmu.1906.5.S1_s_at | LOC574146 | NKG2-A | Mmu.3577 | NP_001028001 | 12.69617 | A | 117.3456 | P |
| MmuSTS.1757.1.S1_at | LOC693556 | similar to ras responsive element | Mmu.14828 | XP_001082265 | 109.3152 | A | 232.5773 | P |
| | | binding protein 1 isoform 1 | | | | | | |
| MmugDNA.35637.1.S1_at | LOC694461 | similar to RIO kinase 1 isoform 1 | Mmu.13193 | XP_001084416 | 195.3009 | A | 132.0237 | P |
| MmugDNA.32349.1.S1_at | LOC694620 | similar to regulator of G-protein | Mmu.16259 | XP_001084439 | 49.87419 | A | 160.4138 | P |
| | | signalling 6 | | | | | | |
| MmugDNA.8053.1.S1_at | LOC694961 | Similar to Scm-like with four mbt | Mmu.13251 | XP_001084282 | 111.6069 | A | 141.3901 | P |
| | | domains 1 | | | | | | |
| MmugDNA.7498.1.S1_at | LOC695519 | hypothetical protein LOC695519 | Mmu.14586 | XP_001088156 | 103.4704 | A | 197.977 | P |
| MmugDNA.9916.1.S1_at | LOC696275 | similar to neuregulin 1 isoform HRG- | Mmu.16225 | XP_001085266 | 109.3953 | A | 190.929 | P |
| | | beta1 | | | | | | |
| MmugDNA.20065.1.S1_at | LOC696749 | similar to runt-related transcription | Mmu.14911 | XP_001086266 | 216.7346 | A | 328.7002 | P |
| | | factor 1 isoform a | | | | | | |
| MmugDNA.40590.1.S1_at | LOC696991 | similar to CG13322-PA, isoform A | Mmu.15342 | XP_001090213 | 151.6879 | A | 172.5933 | P |
| MmugDNA.23596.1.S1_at | LOC697028 | similar to dedicator of cytokinesis 7 | Mmu.4738 | XP_001085641 | 42.86666 | A | 115.8433 | P |
| MmugDNA.19209.1.S1_s_at | LOC697208 | similar to Neutrophil gelatinase- | Mmu.11352 | XP_001083008 | 87.27077 | A | 98.78687 | P |
| | | associated lipocalin precursor (NGAL) | | | | | | |
| | | (p25) (25 kDa alpha-2-microglobulin-related | | | | | | |
| | | subunit of MMP-9) (Lipocalin-2) (Oncogene 24p3) | | | | | | |
| MmugDNA.34368.1.S1_at | LOC698257 | hypothetical protein LOC698257 | Mmu.14406 | XP_001091064 | 35.19047 | A | 47.13095 | P |
| MmugDNA.8688.1.S1_s_at | LOC698576 | similar to plakophilin 4 isoform a | Mmu.90 | XP_001090763 | 57.58762 | A | 168.3497 | P |
| MmugDNA.25652.1.S1_at | LOC698610 | similar to netrin-G1 ligand | Mmu.14852 | XP_001090221 | 159.8217 | A | 530.5047 | P |
| MmugDNA.7880.1.S1_at | LOC698922 | similar to transcription elongation | Mmu.1273 | XP_001087401 | 67.81429 | A | 124.0317 | P |
| | | factor B (SIII), polypeptide 1 | | | | | | |
| MmugDNA.30038.1.S1_at | LOC699233 | similar to J domain protein C21orf55 | Mmu.14908 | XP_001091149 | 20.38795 | A | 98.65044 | P |
| MmugDNA.6969.1.S1_s_at | LOC700634 | similar to PTK2 protein tyrosine | Mmu.3254 | XP_001091188 | 318.6551 | A | 400.0713 | P |
| | | kinase 2 isoform a | | | | | | |
| MmugDNA.11282.1.S1_at | LOC700766 | similar to CG14299-PA, isoform A | Mmu.13220 | XP_001089105 | 102.762 | A | 100.7975 | P |
| MmuSTS.1092.1.S1_at | LOC701162 | similar to solute carrier family 6, | Mmu.11269 | XP_001099500 | 62.05202 | A | 153.328 | P |
| | | member 17 | | | | | | |
| MmugDNA.24847.1.S1_at | LOC701547 | similar to glycine-N-acyltransferase- | Mmu.14222 | XP_001092072 | 104.8917 | A | 110.6059 | P |
| | | like 1 | | | | | | |
| MmugDNA.12356.1.S1_at | LOC702392 | similar to peroxin1 | Mmu.13262 | XP_001100851 | 267.6477 | A | 260.6007 | P |
| MmugDNA.30120.1.S1_at | LOC702596 | similar to nucleolar RNA-associated | Mmu.15669 | XP_001099108 | 517.206 | A | 746.5876 | P |
| | | protein alpha isoform | | | | | | |
| MmugDNA.18234.1.S1_s_at | LOC703331 | similar to runt-related transcription | Mmu.14787 | XP_001100592 | 58.99397 | A | 143.8091 | P |
| | | factor 2 isoform b | | | | | | |
| MmugDNA.42623.1.S1_at | LOC703350 | Similar to SLD5 | Mmu.12445 | XP_001094432 | 116.1626 | A | 145.9785 | P |
| MmugDNA.28192.1.S1_at | LOC703449 | similar to solute carrier family 22 | Mmu.14760 | XP_001098823 | 85.29906 | A | 81.56317 | P |
| | | member 1 isoform a | | | | | | |
| MmugDNA.18415.1.S1_at | LOC703912 | similar to zinc-finger protein NOLZ1 | Mmu.16177 | XP_001095490 | 66.7701 | A | 146.8729 | P |
| MmugDNA.20879.1.S1_at | LOC703981 | similar to NOD9 protein isoform 1 | Mmu.15714 | XP_001104018 | 68.5675 | A | 154.5716 | P |
| MmugDNA.43058.1.S1_at | LOC704126 | similar to kinesin family member 13A | Mmu.14823 | XP_001096456 | 240.8623 | A | 408.3128 | P |
| MmugDNA.36952.1.S1_at | LOC704226 | similar to metallophosphoesterase 1 | Mmu.1901 | XP_001095099 | 181.1201 | A | 250.6874 | P |
| | | precursor | | | | | | |
| MmugDNA.32338.1.S1_at | LOC704620 | Hypothetical protein LOC704620 | Mmu.12860 | XP_001092983 | 484.9445 | A | 1224.313 | P |
| MmugDNA.32288.1.S1_at | LOC705099 | similar to IBR domain containing 2 | Mmu.13564 | XP_001097834 | 169.2173 | A | 387.3177 | P |
| MmugDNA.13807.1.S1_at | LOC705221 | similar to CD2-associated protein | Mmu.14706 | XP_001103871 | 101.1595 | A | 127.5952 | P |
| MmugDNA.32676.1.S1_at | LOC705267 | similar to mitochondrial ribosomal | Mmu.4582 | XP_001093640 | 81.411 | A | 115.9029 | P |
| | | protein S5 | | | | | | |
| MmugDNA.3813.1.S1_at | LOC705358 | similar to PDZ and LIM domain 5 | Mmu.11535 | XP_001102789 | 196.3453 | A | 221.204 | P |
| | | isoform a | | | | | | |
| MmugDNA.41256.1.S1_at | LOC705369 | similar to SET domain containing 3 | Mmu.12527 | XP_001103146 | 307.612 | A | 241.3948 | P |
| MmugDNA.32069.1.S1_at | LOC705781 | similar to CG10721-PA | Mmu.11312 | XP_001098386 | 139.6408 | A | 183.3221 | P |
| MmugDNA.7008.1.S1_at | LOC705781 | similar to CG10721-PA | Mmu.11312 | XP_001098386 | 257.4816 | A | 322.0098 | P |
| MmugDNA.11389.1.S1_at | LOC705913 | similar to cytidine deaminase | Mmu.14713 | XP_001096632 | 157.2027 | A | 447.3467 | P |
| MmugDNA.31582.1.S1_at | LOC706263 | similar to potassium voltage-gated | Mmu.15899 | XP_001108426 | 64.51572 | A | 122.4288 | P |
| | | channel, Isk-related family, member 4 | | | | | | |
| MmugDNA.16996.1.S1_at | LOC706417 | similar to rap2 interacting protein x | Mmu.3060 | XP_001105219 | 77.67221 | A | 92.12048 | P |
| | | isoform 2 | | | | | | |
| MmugDNA.24929.1.S1_at | LOC707355 | similar to zinc finger protein 672 | Mmu.15300 | XP_001105495 | 160.5896 | A | 210.3326 | P |
| MmugDNA.21203.1.S1_x_at | LOC707383 | similar to amyloid beta (A4) | Mmu.669 | XP_001101993 | 428.4578 | A | 421.7363 | P |
| | | precursor protein-binding, family B, | | | | | | |
| | | member 1 interacting protein | | | | | | |
| MmugDNA.35674.1.S1_at | LOC707661 | similar to additional sex combs like 1 | Mmu.15484 | XP_001101400 | 110.2575 | A | 154.5192 | P |
| MmugDNA.14411.1.S1_at | LOC707686 | similar to angiogenic factor VG5Q | Mmu.12022 | XP_001106588 | 312.2592 | A | 323.3734 | P |
| MmuSTS.1472.1.S1_at | LOC707739 | Similar to serine/threonine protein | Mmu.12485 | XP_001096161 | 31.29481 | A | 62.03861 | P |
| | | kinase MASK | | | | | | |
| MmugDNA.6011.1.S1_at | LOC707982 /// | similar to rhomboid domain | Mmu.15898 | XP_001110015 | 253.2026 | A | 501.7335 | P |
| | LOC708189 | containing 1 /// hypothetical protein | | | | | | |
| | | LOC708189 | | | | | | |
| MmuSTS.4837.1.S1_at | LOC708940 | similar to Guanine nucleotide-binding | Mmu.14862 | XP_001108555 | 67.89697 | A | 97.95885 | P |
| | | protein G(i), alpha-1 subunit | | | | | | |
| | | (Adenylate cyclase-inhibiting G alpha | | | | | | |
| | | protein) | | | | | | |
| MmuSTS.508.1.S1_at | LOC709049 | similar to activating signal | Mmu.14734 | XP_001105587 | 145.0563 | A | 157.3825 | P |
| | | cointegrator 1 complex subunit 1 | | | | | | |
| MmugDNA.32637.1.S1_at | LOC709253 | similar to nucleosomal binding | Mmu.13802 | XP_001103630 | 80.94846 | A | 62.59929 | P |
| | | protein 1 | | | | | | |
| MmugDNA.38015.1.S1_at | LOC709468 | similar to terminal | Mmu.12395 | XP_001107131 | 75.45173 | A | 95.06112 | P |
| | | deoxynucleotidyltransferase | | | | | | |
| | | interacting factor 1 | | | | | | |
| Mmu.10346.1.S1_at | LOC709544 | hypothetical protein LOC709544 | Mmu.16556 | XP_001096738 | 507.0977 | A | 355.7336 | P |
| MmugDNA.39938.1.S1_at | LOC709882 | similar to Troponin T, cardiac muscle | Mmu.14101 | XP_001098451 | 129.6782 | A | 195.8546 | P |
| | | (TnTc) (Cardiac muscle troponin T) | | | | | | |
| | | (cTnT) | | | | | | |
| MmugDNA.26478.1.S1_at | LOC710518 | similar to mitochondrial solute | Mmu.11000 | XP_001105927 | 455.317 | A | 1481.092 | P |
| | | carrier-like protein | | | | | | |
| MmugDNA.33876.1.S1_s_at | LOC711336 | similar to olfactomedin 2 | Mmu.15445 | XP_001099531 | 105.1338 | A | 181.6893 | P |
| MmugDNA.17217.1.S1_at | LOC711485 | similar to acyl-Coenzyme A | Mmu.12959 | XP_001109931 | 123.1174 | A | 379.9161 | P |
| | | dehydrogenase, long chain precursor | | | | | | |
| MmugDNA.15166.1.S1_at | LOC711633 | similar to zinc finger CCCH-type | Mmu.15988 | XP_001114089 | 185.2786 | A | 231.699 | P |
| | | containing 10 | | | | | | |
| MmuSTS.4200.1.S1_at | LOC711691 | Similar to pre-B-cell leukemia | Mmu.4832 | XP_001100529 | 105.6631 | A | 901.0759 | P |
| | | transcription factor 3 | | | | | | |
| Mmu.13564.1.S1_at | LOC711742 | similar to kinesin family member 9 | Mmu.564 | XP_001113416 | 8.346619 | A | 81.96733 | P |
| | | isoform 2 | | | | | | |
| MmugDNA.43479.1.S1_at | LOC711908 | similar to transmembrane 4 | Mmu.2535 | XP_001108758 | 8.667226 | A | 94.85152 | P |
| | | superfamily member 1 | | | | | | |
| MmugDNA.30723.1.S1_at | LOC711984 | Similar to zinc finger CCCH-type | Mmu.12594 | XP_001106124 | 175.1743 | A | 338.943 | P |
| | | domain containing 7 | | | | | | |
| MmugDNA.20892.1.S1_at | LOC712033 | similar to PCTAIRE protein kinase 1 | Mmu.14735 | XP_001100911 | 120.9395 | A | 203.3945 | P |
| MmugDNA.12728.1.S1_at | LOC712272 | similar to fibronectin type 3 and SPRY | Mmu.13034 | XP_001109699 | 37.6952 | A | 88.71973 | P |
| | | domain-containing protein | | | | | | |
| MmugDNA.1324.1.S1_s_at | LOC712600 | similar to quaking homolog, KH | Mmu.2689 | XP_001101642 | 109.0662 | A | 111.2004 | P |
| | | domain RNA binding isoform HQK-5 | | | | | | |
| MmugDNA.12133.1.S1_at | LOC713035 | similar to BCL2-like 1 isoform 1 | Mmu.16103 | XP_001109879 | 159.8234 | A | 120.8184 | P |
| MmugDNA.2816.1.S1_s_at | LOC713680 | similar to Protein C9orf7 | Mmu.11422 | XP_001100751 | 178.9342 | A | 371.2914 | P |
| MmugDNA.23412.1.S1_x_at | LOC715553 | similar to Protein PP2447 | Mmu.16050 | XP_001111734 | 651.9257 | A | 974.2084 | P |
| MmuSTS.563.1.S1_at | LOC715706 | Similar to schlafen family member 13 | Mmu.11333 | XP_001114176 | 47.03052 | A | 109.2105 | P |
| MmugDNA.40257.1.S1_at | LOC716245 | similar to sphingosine-1-phosphate | Mmu.4100 | XP_001106861 | 122.3767 | A | 155.0102 | P |
| | | lyase 1 | | | | | | |
| MmugDNA.36455.1.S1_at | LOC716351 | similar to SEC22 vesicle trafficking | Mmu.14967 | XP_001115262 | 162.7943 | A | 203.9004 | P |
| | | protein homolog C isoform b | | | | | | |
| MmugDNA.2703.1.S1_at | LOC716903 | hypothetical protein LOC716903 | Mmu.15043 | XP_001115326 | 21.5953 | A | 72.00944 | P |
| Mmu.8945.1.S1_at | LOC717346 | similar to complement factor H | Mmu.4677 | XP_001111499 | 90.61079 | A | 186.3635 | P |
| | | isoform a precursor | | | | | | |
| MmugDNA.22554.1.S1_s_at | LOC717764 | similar to phospholipase C, beta 3 | Mmu.11823 | XP_001115104 | 294.9439 | A | 257.2195 | P |
| | | (phosphatidylinositol-specific) | | | | | | |
| MmugDNA.41138.1.S1_at | LOC717824 | similar to mago-nashi homolog | Mmu.2413 | XP_001115401 | 56.18679 | A | 108.1663 | P |
| MmugDNA.8485.1.S1_at | LOC719645 | similar to H326 | Mmu.153 | XP_001117421 | 55.48396 | A | 122.239 | P |
| MmuSTS.2495.1.S1_at | LOC719814 | similar to zinc finger protein 577 | Mmu.15382 | XP_001116472 | 105.0435 | A | 132.2027 | P |
| MmugDNA.11286.1.S1_at | LOC720186 | similar to immunoglobulin | Mmu.11138 | XP_001115307 | 385.697 | A | 486.4542 | P |
| | | superfamily, member 8 | | | | | | |
| MmugDNA.1499.1.S1_at | LOC721262 | similar to Pigment epithelium-derived | Mmu.12605 | XP_001117361 | 90.36726 | A | 283.4944 | P |
| | | factor precursor (PEDF) (EPC-1) | | | | | | |
| MmugDNA.16852.1.S1_at | LTB4R | leukotriene B4 receptor | — | XP_001114016 | 97.06477 | A | 397.0128 | P |
| MmugDNA.21143.1.S1_at | MAOA | monoamine oxidase A | Mmu.3283 | XP_001096840 | 127.6487 | A | 386.7567 | P |
| MmugDNA.20648.1.S1_at | MFAP3 | microfibrillar-associated protein 3 | Mmu.1139 | XP_001102567 | 209.1429 | A | 191.3753 | P |
| MmugDNA.17553.1.S1_at | MMP19 | matrix metallopeptidase 19 | — | XP_001111542 | 95.18174 | A | 126.3377 | P |
| MmuSTS.4170.1.S1_at | MRAS | muscle RAS oncogene homolog | Mmu.14559 | XP_001114261 | 150.9189 | A | 140.7781 | P |
| MmugDNA.13215.1.S1_at | MYO5B | myosin VB | Mmu.15475 | XP_001090434 | 76.57364 | A | 115.3176 | P |
| MmugDNA.26632.1.S1_at | NMNAT3 | nicotinamide nucleotide | Mmu.14941 | XP_001113328 | 100.2686 | A | 157.8669 | P |
| | | adenylyltransferase 3 | | | | | | |
| MmugDNA.28543.1.S1_at | NR4A2 | Nuclear receptor subfamily 4, group | Mmu.11507 | XP_001085438 | 67.00845 | A | 47.1474 | P |
| | | A, member 2 | | | | | | |
| Mmu.14635.1.S1_s_at | P11 | placental protein 11 | Mmu.1192 | XP_001097398 | 3.029125 | A | 85.42168 | P |
| MmugDNA.7677.1.S1_at | RBM34 | RNA binding motif protein 34 | Mmu.12653 | XP_001102020 | 139.6196 | A | 191.8863 | P |
| MmugDNA.17401.1.S1_at | SEC24A | SEC24 family, member A (S. cerevisiae) | Mmu.13387 | XP_001109569 | 32.50195 | A | 145.5821 | P |
| MmugDNA.34347.1.S1_s_at | SLC25A13 | solute carrier family 25, member 13 | Mmu.11416 | XP_001088340 | 83.63106 | A | 83.29261 | P |
| | | (citrin) | | | | | | |
| MmugDNA.15062.1.S1_at | SSBP1 | Single-stranded DNA binding protein 1 | Mmu.4481 | XP_001084058 | 129.9467 | A | 251.779 | P |
| MmugDNA.26812.1.S1_at | ST8SIA4 | ST8 alpha-N-acetyl-neuraminide | Mmu.13440 | XP_001097339 | 135.5056 | A | 100.6624 | P |
| | | alpha-2,8-sialyltransferase 4 | | | | | | |
| MmugDNA.9561.1.S1_at | TIMP-2 | tissue inhibitor of matrix | Mmu.1702 | XP_001106657 | 369.0166 | A | 579.6535 | P |
| | | metalloproteinase-2 | | | | | | |
| MmugDNA.86.1.S1_at | TRHDE | thyrotropin-releasing hormone | Mmu.130 | XP_001109391 | 53.7547 | A | 145.0238 | P |
| | | degrading enzyme | | | | | | |
| Mmu.5321.1.S1_at | TTC12 | tetratricopeptide repeat domain 12 | Mmu.13011 | XP_001084384 | 108.8476 | A | 283.5953 | P |
| MmugDNA.33980.1.S1_at | TTLL4 | tubulin tyrosine ligase-like family, | Mmu.15909 | XP_001094019 | 273.2361 | A | 188.5919 | P |
| | | member 4 | | | | | | |
| MmugDNA.20532.1.S1_at | WDR77 | WD repeat domain 77 | Mmu.4390 | XP_001105103 | 179.0304 | A | 608.6301 | P |
| MmugDNA.33665.1.S1_at | WDR8 | WD repeat domain 8 | Mmu.15190 | XP_001083578 | 178.8616 | A | 467.1802 | P |
| MmugDNA.19387.1.S1_at | ZDHHC3 | zinc finger, DHHC-type containing 3 | Mmu.11811 | XP_001114690 | 387.6217 | A | 769.4366 | P |
| MmugDNA.42763.1.S1_at | ZFAND5 | zinc finger, AN1-type domain 5 | Mmu.1639 | XP_001096078 | 2.943856 | A | 141.202 | P |

TABLE 3
Differentially expressed genes in PBMCs of NHPs 24-hr following MCAO

| Probe Set ID | Gene Symbol | Gene Title | UniGene ID | RefSeq Protein ID | Pre-O Signal | Pre-O detection | 24 hr Signal | 24-hr detection |
|---|---|---|---|---|---|---|---|---|
| MmuSTS.3785.1.S1_at | AGT | angiotensinogen | Mmu.11931 | XP_001107315 /// | 53.05984 | A | 121.1526 | P |
| | | | | XP_001107374 | | | | |
| MmugDNA.18724.1.S1_s_at | B4GALT6 | UDP-Gal:betaGlcNAc beta 1,4- | Mmu.604 | XP_001099215 /// | 58.75183 | A | 80.2387 | P |
| | | galactosyltransferase, | | XP_001099324 /// | | | | |
| | | polypeptide 6 | | XP_001099421 | | | | |
| Mmu.7394.1.A1_at | BXDC1 | Brix domain containing 1 | Mmu.2348 | XP_001087115 | 93.25996 | A | 98.8365 | P |
| MmugDNA.2736.1.S1_at | C1QA | Complement component 1, q | Mmu.4861 | XP_001101655 /// | 182.484 | A | 177.6559 | P |
| | | subcomponent, A chain | | XP_001101750 /// | | | | |
| | | | | XP_001101837 /// | | | | |
| | | | | XP_001101933 | | | | |
| MmugDNA.26392.1.S1_at | CCNF | cyclin F | Mmu.11675 | XP_001085466 | 114.6939 | A | 287.1454 | P |
| MmugDNA.25206.1.S1_at | CDC2 | cell division cycle 2, G1 to S and | Mmu.16170 | XP_001095697 /// | 83.30299 | A | 82.88945 | P |
| | | G2 to M | | XP_001095798 /// | | | | |
| | | | | XP_001095903 | | | | |
| Mmu.10362.1.S1_at | CLIC4 | chloride intracellular channel 4 | Mmu.4332 | XP_001106291 /// | 131.4646 | A | 115.3725 | P |
| | | | | XP_001106424 /// | | | | |
| | | | | XP_001106485 | | | | |
| MmuSTS.1884.1.S1_at | CREM | cAMP responsive element | Mmu.2805 | XP_001088641 /// | 144.2539 | A | 153.9597 | P |
| | | modulator | | XP_001088747 /// | | | | |
| | | | | XP_001088981 /// | | | | |
| | | | | XP_001089092 /// | | | | |
| | | | | XP_001089667 /// | | | | |
| | | | | XP_001090015 /// | | | | |
| | | | | XP_001090136 /// | | | | |
| | | | | XP_001090248 /// | | | | |
| | | | | XP_001090366 /// | | | | |
| | | | | XP_001090477 /// | | | | |
| | | | | XP_001090595 /// | | | | |
| | | | | XP_001090829 /// | | | | |
| | | | | XP_001091190 /// | | | | |
| | | | | XP_001091309 /// | | | | |
| | | | | XP_001091547 /// | | | | |
| | | | | XP_001091662 /// | | | | |
| | | | | XP_001092258 /// | | | | |
| | | | | XP_001092367 /// | | | | |
| | | | | XP_001092480 /// | | | | |
| | | | | XP_001092602 /// | | | | |
| | | | | XP_001092837 /// | | | | |
| | | | | XP_001092950 /// | | | | |
| | | | | XP_001093063 /// | | | | |
| | | | | XP_001093168 | | | | |
| MmugDNA.1202.1.S1_at | CRKL | V-crk sarcoma virus CT10 | Mmu.12764 | XP_001086006 /// | 92.14868 | A | 181.7126 | P |
| | | oncogene homolog (avian)-like | | XP_001086123 | | | | |
| MmunewRS.528.1.S1_at | CSF2RB | colony stimulating factor 2 | Mmu.1176 | XP_001086084 | 146.8577 | A | 237.7413 | P |
| | | receptor, beta, low-affinity | | | | | | |
| | | (granulocyte-macrophage) | | | | | | |
| Mmu.15973.2.S1_x_at | CSH-3 /// | chorionic somatommamotropin | Mmu.10849 | NP_001036203 /// | 122.2181 | A | 167.5703 | P |
| | CSH-4 /// | hormone 3 /// chorionic | | NP_001036207 /// | | | | |
| | GH1 /// | somatommamotropin hormone 4 | | NP_001040618 /// | | | | |
| | LOC718474 | /// growth hormone 1 /// similar | | NP_001040620 /// | | | | |
| | /// | to growth hormone 1 isoform 1 | | XP_001111200 /// | | | | |
| | LOC721256 | /// similar to growth hormone 1 | | XP_001116436 /// | | | | |
| | /// | isoform 1 /// chorionic | | XP_001117351 | | | | |
| | LOC735307 | somatomammotropin-1 | | | | | | |
| MmugDNA.5060.1.S1_at | CTNND1 | catenin (cadherin-associated | Mmu.1419 | XP_001094621 /// | 79.00558 | A | 247.8638 | P |
| | | protein), delta 1 | | XP_001095087 /// | | | | |
| | | | | XP_001095314 /// | | | | |
| | | | | XP_001095429 /// | | | | |
| | | | | XP_001095545 /// | | | | |
| | | | | XP_001095646 /// | | | | |
| | | | | XP_001095745 /// | | | | |
| | | | | XP_001095859 /// | | | | |
| | | | | XP_001095967 /// | | | | |
| | | | | XP_001096070 /// | | | | |
| | | | | XP_001096183 /// | | | | |
| | | | | XP_001096303 /// | | | | |
| | | | | XP_001096427 /// | | | | |
| | | | | XP_001096539 /// | | | | |
| | | | | XP_001096644 /// | | | | |
| | | | | XP_001096747 /// | | | | |
| | | | | XP_001096864 /// | | | | |
| | | | | XP_001096974 /// | | | | |
| | | | | XP_001097086 /// | | | | |
| | | | | XP_001097202 /// | | | | |
| | | | | XP_001097299 /// | | | | |
| | | | | XP_001097408 /// | | | | |
| | | | | XP_001097505 /// | | | | |
| | | | | XP_001097599 /// | | | | |
| | | | | XP_001097712 /// | | | | |
| | | | | XP_001097810 /// | | | | |
| | | | | XP_001097908 /// | | | | |
| | | | | XP_001097998 /// | | | | |
| | | | | XP_001098090 /// | | | | |
| | | | | XP_001098194 /// | | | | |
| | | | | XP_001098301 /// | | | | |
| | | | | XP_001098398 /// | | | | |
| | | | | XP_001098491 /// | | | | |
| | | | | XP_001098588 /// | | | | |
| | | | | XP_001098689 /// | | | | |
| | | | | XP_001098792 /// | | | | |
| | | | | XP_001098897 /// | | | | |
| | | | | XP_001098993 /// | | | | |
| | | | | XP_001099102 /// | | | | |
| | | | | XP_001099203 /// | | | | |
| | | | | XP_001099314 /// | | | | |
| | | | | XP_001099411 /// | | | | |
| | | | | XP_001099522 /// | | | | |
| | | | | XP_001099613 /// | | | | |
| | | | | XP_001099715 /// | | | | |
| | | | | XP_001099819 /// | | | | |
| | | | | XP_001099920 | | | | |
| MmugDNA.40586.1.S1_s_at | CYP3A5 /// | cytochrome P450, family 3, | Mmu.3065 | NP_001035309 /// | 176.8445 | A | 370.1329 | P |
| | CYP3A64 | subfamily A, polypeptide 5 /// | | NP_001035504 /// | | | | |
| | | cytochrome P450 3A64 | | XP_001096666 /// | | | | |
| | | | | XP_001097621 /// | | | | |
| | | | | XP_001097930 | | | | |
| MmugDNA.1267.1.S1_s_at | CYP3A64 /// | cytochrome P450 3A64 /// | Mmu.9706 | NP_001035504 /// | 89.83363 | A | 267.5797 | P |
| | LOC718917 | similar to Cytochrome P450 3A7 | | XP_001097930 /// | | | | |
| | | (CYPIIIA7) (P450-HFLA) | | XP_001112281 | | | | |
| MmugDNA.36232.1.S1_at | DAB2 | disabled homolog 2 | Mmu.12958 | XP_001084792 | 72.17534 | A | 157.0555 | P |
| MmugDNA.41396.1.S1_at | DAB2 | disabled homolog 2 | Mmu.12958 | XP_001084792 | 86.95129 | A | 159.1484 | P |
| MmugDNA.13802.1.S1_at | DCN | decorin | Mmu.1687 | XP_001103412 /// | 52.87949 | A | 70.88836 | P |
| | | | | XP_001103495 /// | | | | |
| | | | | XP_001103577 /// | | | | |
| | | | | XP_001103663 /// | | | | |
| | | | | XP_001103749 /// | | | | |
| | | | | XP_001103834 /// | | | | |
| | | | | XP_001103918 /// | | | | |
| | | | | XP_001104009 /// | | | | |
| | | | | XP_001104095 /// | | | | |
| | | | | XP_001104178 /// | | | | |
| | | | | XP_001104261 /// | | | | |
| | | | | XP_001104340 | | | | |
| MmugDNA.14528.1.S1_s_at | DCN | decorin | Mmu.1687 | XP_001103412 /// | 107.5135 | A | 179.9101 | P |
| | | | | XP_001103495 /// | | | | |
| | | | | XP_001103577 /// | | | | |
| | | | | XP_001103663 /// | | | | |
| | | | | XP_001103749 /// | | | | |
| | | | | XP_001103834 /// | | | | |
| | | | | XP_001103918 /// | | | | |
| | | | | XP_001104009 /// | | | | |
| | | | | XP_001104095 /// | | | | |
| | | | | XP_001104178 /// | | | | |
| | | | | XP_001104261 /// | | | | |
| | | | | XP_001104340 | | | | |
| MmugDNA.22700.1.S1_at | DCN | decorin | Mmu.1687 | XP_001103412 /// | 120.1081 | A | 301.5998 | P |
| | | | | XP_001103495 /// | | | | |
| | | | | XP_001103577 /// | | | | |
| | | | | XP_001103663 /// | | | | |
| | | | | XP_001103749 /// | | | | |
| | | | | XP_001103834 /// | | | | |
| | | | | XP_001103918 /// | | | | |
| | | | | XP_001104009 /// | | | | |
| | | | | XP_001104095 /// | | | | |
| | | | | XP_001104178 /// | | | | |
| | | | | XP_001104261 /// | | | | |
| | | | | XP_001104340 | | | | |
| MmugDNA.41798.1.S1_at | DHRS7 | dehydrogenase/reductase (SDR | Mmu.4703 | XP_001095590 /// | 122.1582 | A | 206.1407 | P |
| | | family) member 7 | | XP_001095688 /// | | | | |
| | | | | XP_001095790 | | | | |
| MmugDNA.30768.1.S1_at | DOCK6 | dedicator of cytokinesis 6 | Mmu.14293 | XP_001106785 | 19.43518 | A | 178.1556 | P |
| MmugDNA.26154.1.S1_at | DRD2 | dopamine receptor D2 | Mmu.3401 | XP_001085336 /// | 94.5069 | A | 196.3607 | P |
| | | | | XP_001085449 /// | | | | |
| | | | | XP_001085571 | | | | |
| Mmu.10947.1.A1_at | DUSP3 | Dual specificity phosphatase 3 | Mmu.11380 | XP_001113728 /// | 98.48205 | A | 192.5923 | P |
| | | | | XP_001113754 | | | | |
| MmugDNA.26188.1.S1_at | ECEL1 | endothelin converting enzyme- | Mmu.11796 | XP_001117070 | 178.3571 | A | 167.7575 | P |
| | | like 1 | | | | | | |
| MmugDNA.32487.1.S1_s_at | ELA3B | elastase 3B | Mmu.3417 | XP_001099601 | 53.45941 | A | 207.1332 | P |
| MmugDNA.38477.1.S1_at | ELK4 | ELK4, ETS-domain protein (SRF | — | XP_001087748 | 144.6712 | A | 96.148 | P |
| | | accessory protein 1) | | | | | | |
| MmugDNA.620.1.S1_at | ENPP1 | ectonucleotide | Mmu.14518 | XP_001103359 | 50.47231 | A | 103.8725 | P |
| | | pyrophosphatase/phosphodiesterase 1 | | | | | | |
| MmugDNA.27688.1.S1_at | EPOR | erythropoietin receptor | — | XP_001105833 | 188.3416 | A | 259.7 | P |
| MmugDNA.10912.1.S1_at | ERBB3 | v-erb-b2 erythroblastic leukemia | Mmu.758 | XP_001113794 /// | 103.4474 | A | 195.9001 | P |
| | | viral oncogene homolog 3 | | XP_001113900 /// | | | | |
| | | (avian) | | XP_001113928 /// | | | | |
| | | | | XP_001113953 | | | | |
| MmuSTS.4736.1.S1_at | ERBB3 | v-erb-b2 erythroblastic leukemia | Mmu.758 | XP_001113794 /// | 161.6664 | A | 289.3541 | P |
| | | viral oncogene homolog 3 | | XP_001113900 /// | | | | |
| | | (avian) | | XP_001113928 /// | | | | |
| | | | | XP_001113953 | | | | |
| MmuSTS.2627.1.S1_at | EXTL1 | exostoses (multiple)-like 1 | Mmu.15170 | XP_001108035 /// | 62.74446 | A | 177.0561 | P |
| | | | | XP_001108149 /// | | | | |
| | | | | XP_001108202 | | | | |
| MmugDNA.18128.1.S1_at | F7 | coagulation factor VII | Mmu.11080 | NP_001073605 /// | 95.21188 | A | 208.6877 | P |
| | | | | XP_001118128 | | | | |
| MmugDNA.11377.1.S1_s_at | FCAR | Fc fragment of IgA, receptor for | Mmu.3792 | NP_001035039 | 196.9096 | A | 296.4183 | P |
| MmugDNA.19608.1.S1_at | FGB | fibrinogen beta chain | Mmu.11581 | XP_001091417 /// | 35.28459 | A | 142.7827 | P |
| | | | | XP_001091760 /// | | | | |
| | | | | XP_001091882 /// | | | | |
| | | | | XP_001091998 | | | | |
| MmuSTS.4781.1.S1_at | FKBP10 | FK506 binding protein 10, 65 kDa | Mmu.14407 | XP_001107581 /// | 144.692 | A | 206.9655 | P |
| | | | | XP_001107638 /// | | | | |
| | | | | XP_001107696 /// | | | | |
| | | | | XP_001107756 /// | | | | |
| | | | | XP_001107821 | | | | |
| MmugDNA.33925.1.S1_s_at | FOXP2 | forkhead box P2 | Mmu.4784 | NP_001028193 | 204.0107 | A | 286.409 | P |
| MmuSTS.4804.1.S1_at | FPR2 | formyl peptide receptor 2 | Mmu.15383 | XP_001116437 /// | 314.0186 | A | 738.5731 | P |
| | | | | XP_001116445 /// | | | | |
| | | | | XP_001116452 | | | | |
| MmuSTS.4807.1.S1_at | FUT3 | fucosyltransferase 3 (galactoside | Mmu.11521 | XP_001085818 /// | 130.1834 | A | 194.3972 | P |
| | | 3(4)-L-fucosyltransferase, Lewis | | XP_001085924 | | | | |
| | | blood group) | | | | | | |
| MmuSTS.4814.1.S1_at | GABRE | gamma-aminobutyric acid | Mmu.15237 | XP_001093070 /// | 61.50348 | A | 144.2998 | P |
| | | (GABA) A receptor, epsilon | | XP_001093507 /// | | | | |
| | | | | XP_001093628 /// | | | | |
| | | | | XP_001093865 | | | | |
| MmugDNA.22053.1.S1_at | GGCX | gamma-glutamyl carboxylase | Mmu.13911 | XP_001086016 /// | 309.7049 | A | 434.7215 | P |
| | | | | XP_001086474 | | | | |
| MmugDNA.13746.1.S1_s_at | GK | glycerol kinase | Mmu.16427 | XP_001100778 /// | 21.59358 | A | 147.123 | P |
| | | | | XP_001100861 /// | | | | |
| | | | | XP_001100968 | | | | |
| MmugDNA.37125.1.S1_at | GLRB | glycine receptor, beta | Mmu.1658 | XP_001093718 /// | 140.4933 | A | 260.0455 | P |
| | | | | XP_001093832 | | | | |
| Mmu.8905.1.S1_at | GPD1 | glycerol-3-phosphate | Mmu.3010 | XP_001102726 | 70.93366 | A | 227.4083 | P |
| | | dehydrogenase 1 (soluble) | | | | | | |
| Mmu.11695.1.S1_at | GPR34 | G protein-coupled receptor 34 | Mmu.3224 | XP_001087742 /// | 146.0326 | A | 141.9543 | P |
| | | | | XP_001087858 /// | | | | |
| | | | | XP_001087981 /// | | | | |
| | | | | XP_001088105 | | | | |
| MmuSTS.2489.1.S1_at | GPR77 | G protein-coupled receptor 77 | Mmu.15399 | XP_001112895 /// | 85.30246 | A | 324.2143 | P |
| | | | | XP_001112923 /// | | | | |
| | | | | XP_001112956 | | | | |
| MmugDNA.19343.1.S1_s_at | HBG2 | hemoglobin, gamma G | Mmu.2134 | NP_001040611 /// | 36.8732 | A | 92.44987 | P |
| | | | | XP_001111590 | | | | |
| MmugDNA.7761.1.S1_at | HIPK2 | homeodomain interacting protein | Mmu.4514 | XP_001106719 | 112.2257 | A | 349.4229 | P |
| | | kinase 2 | | | | | | |
| MmugDNA.27045.1.S1_at | HOMER1 | homer homolog 1 (Drosophila) | Mmu.16400 | XP_001108808 /// | 59.80412 | A | 82.8224 | P |
| | | | | XP_001108859 /// | | | | |
| | | | | XP_001108899 /// | | | | |
| | | | | XP_001108946 /// | | | | |
| | | | | XP_001109003 /// | | | | |
| | | | | XP_001109058 /// | | | | |
| | | | | XP_001109116 /// | | | | |
| | | | | XP_001109170 | | | | |
| MmuSTS.1348.1.S1_at | HOXB7 | homeobox B7 | Mmu.16913 | XP_001088822 | 134.1311 | A | 124.6616 | P |
| MmugDNA.18776.1.S1_at | HPD | 4-hydroxyphenylpyruvate | Mmu.14575 | XP_001096061 /// | 81.20009 | A | 312.6377 | P |
| | | dioxygenase | | XP_001096175 | | | | |
| Mmu.15730.2.S1_at | HSP90AB1 | heat shock 90 kDa protein 1, | Mmu.142 | XP_001098219 /// | 97.19216 | A | 432.5437 | P |
| | | beta | | XP_001098426 /// | | | | |
| | | | | XP_001098520 /// | | | | |
| | | | | XP_001098716 /// | | | | |
| | | | | XP_001098925 /// | | | | |
| | | | | XP_001099027 /// | | | | |
| | | | | XP_001099135 /// | | | | |
| | | | | XP_001099237 /// | | | | |
| | | | | XP_001099339 /// | | | | |
| | | | | XP_001099439 | | | | |
| MmugDNA.28970.1.S1_at | IL1RN | interleukin 1 receptor antagonist | Mmu.15827 | XP_001091493 /// | 126.3157 | A | 202.4636 | P |
| | | | | XP_001091717 /// | | | | |
| | | | | XP_001091833 | | | | |
| MmuSTS.681.1.S1_at | IL1RN | interleukin 1 receptor antagonist | Mmu.15827 | XP_001091493 /// | 108.0968 | A | 347.2209 | P |
| | | | | XP_001091717 /// | | | | |
| | | | | XP_001091833 | | | | |
| MmugDNA.7719.1.S1_at | IL4I1 | interleukin 4 induced 1 | Mmu.15390 | XP_001115731 /// | 122.4228 | A | 134.9988 | P |
| | | | | XP_001115739 /// | | | | |
| | | | | XP_001115750 /// | | | | |
| | | | | XP_001115759 /// | | | | |
| | | | | XP_001115764 /// | | | | |
| | | | | XP_001115773 | | | | |
| MmugDNA.21962.1.S1_at | INHBA | inhibin, beta A | — | XP_001098421 /// | 5.461364 | A | 66.77403 | P |
| | | | | XP_001098514 | | | | |
| MmugDNA.41698.1.S1_at | ITGA6 | Integrin, alpha 6 | Mmu.11085 | XP_001086200 | 175.6256 | A | 326.1319 | P |
| MmuSTS.4395.1.S1_at | ITGA9 | integrin, alpha 9 | — | XP_001086555 | 12.68558 | A | 106.7995 | P |
| MmugDNA.36020.1.S1_at | ITGB5 | integrin, beta 5 | Mmu.1239 | XP_001113831 /// | 118.9835 | A | 287.9891 | P |
| | | | | XP_001113881 /// | | | | |
| | | | | XP_001113909 | | | | |
| MmugDNA.33524.1.S1_at | JAG1 | jagged 1 | Mmu.16088 | XP_001116216 | 33.29051 | A | 158.451 | P |
| MmugDNA.25725.1.S1_at | KCNE1 | potassium voltage-gated | Mmu.14910 | XP_001088840 /// | 8.312235 | A | 125.2664 | P |
| | | channel, Isk-related family, | | XP_001088944 /// | | | | |
| | | member 1 | | XP_001089061 /// | | | | |
| | | | | XP_001089178 | | | | |
| MmugDNA.2200.1.S1_s_at | KCNJ13 | potassium inwardly-rectifying | Mmu.11590 | XP_001114522 /// | 80.08498 | A | 118.0615 | P |
| | | channel, subfamily J, member 13 | | XP_001114542 | | | | |
| Mmu.7460.6.S1_x_at | KIR3DH5 | killer-cell Ig-like receptor | Mmu.17415 | NP_001098643 | 251.4261 | A | 142.5331 | P |
| | | KIR3DH5 | | | | | | |
| Mmu.7460.12.S1_x_at | KIR3DL10 | killer immunoglobulin-like | Mmu.17259 | NP_001098640 | 1.215597 | A | 128.9819 | P |
| | | receptor KIR3DL10 | | | | | | |
| Mmu.9316.1.S1_at | KLF11 | Kruppel-like factor 11 | Mmu.11355 | XP_001090608 | 501.1391 | A | 598.1434 | P |
| MmugDNA.33624.1.S1_at | KLK15 | kallikrein-related peptidase 15 | Mmu.15386 | XP_001116155 /// | 58.26991 | A | 165.5999 | P |
| | | | | XP_001116159 /// | | | | |
| | | | | XP_001116169 /// | | | | |
| | | | | XP_001116177 /// | | | | |
| | | | | XP_001116190 /// | | | | |
| | | | | XP_001116199 /// | | | | |
| | | | | XP_001116226 /// | | | | |
| | | | | XP_001116235 | | | | |
| MmugDNA.40811.1.S1_at | KSR1 | kinase suppressor of ras | Mmu.2049 | XP_001105629 | 92.95608 | A | 89.93006 | P |
| MmuSTS.385.1.S1_at | LMO1 | LIM domain only 1 (rhombotin 1) | Mmu.14044 | XP_001105032 /// | 84.15849 | A | 209.9162 | P |
| | | | | XP_001105198 /// | | | | |
| | | | | XP_001105265 | | | | |
| MmuSTS.1666.1.S1_at | LNX1 | ligand of numb-protein X 1 | Mmu.16462 | XP_001090921 /// | 104.5588 | A | 155.7875 | P |
| | | | | XP_001091044 /// | | | | |
| | | | | XP_001091164 | | | | |
| Mmu.7390.1.S1_at | LOC574134 | VAMP-2 | Mmu.3489 | NP_001027992 | 220.164 | A | 376.094 | P |
| Mmu.1906.5.S1_s_at | LOC574146 | NKG2-A | Mmu.3577 | NP_001028001 | 12.69617 | A | 90.53711 | P |
| MmugDNA.15100.1.S1_at | LOC693289 | Similar to ATP-binding cassette, | Mmu.12529 | XP_001082244 /// | 7.136568 | A | 109.7202 | P |
| | | sub-family A member 8 | | XP_001082492 /// | | | | |
| | | | | XP_001082622 | | | | |
| MmuSTS.1757.1.S1_at | LOC693556 | similar to ras responsive element | Mmu.14828 | XP_001082265 /// | 109.3152 | A | 122.6399 | P |
| | | binding protein 1 isoform 1 | | XP_001082516 /// | | | | |
| | | | | XP_001082652 /// | | | | |
| | | | | XP_001082784 /// | | | | |
| | | | | XP_001082911 | | | | |
| MmugDNA.20287.1.S1_at | LOC693696 | similar to longevity assurance | Mmu.16301 | XP_001082530 /// | 9.598577 | A | 92.75128 | P |
| | | homolog 2 | | XP_001082667 /// | | | | |
| | | | | XP_001082802 | | | | |
| MmugDNA.12459.1.S1_at | LOC693821 | similar to THAP domain | Mmu.11226 | XP_001086766 | 33.44794 | A | 97.39143 | P |
| | | containing 7 | | | | | | |
| MmugDNA.17057.1.S1_at | LOC694043 | Similar to RECK protein | Mmu.12983 | XP_001083242 /// | 36.28584 | A | 106.7528 | P |
| | | precursor | | XP_001083599 | | | | |
| MmugDNA.14488.1.S1_at | LOC694192 | similar to Protein C18orf54 | Mmu.15470 | XP_001082497 /// | 158.6407 | A | 146.8237 | P |
| | | precursor | | XP_001082628 /// | | | | |
| | | | | XP_001082761 | | | | |
| MmugDNA.32899.1.S1_at | LOC694220 | similar to glutathione S- | Mmu.16453 | XP_001083156 /// | 151.0824 | A | 165.4515 | P |
| | | transferase, C-terminal domain | | XP_001083273 /// | | | | |
| | | containing isoform 1 | | XP_001083401 | | | | |
| MmugDNA.35637.1.S1_at | LOC694461 | similar to RIO kinase 1 isoform 1 | Mmu.13193 | XP_001084416 /// | 195.3009 | A | 347.8302 | P |
| | | | | XP_001084537 /// | | | | |
| | | | | XP_001084661 | | | | |
| MmugDNA.32349.1.S1_at | LOC694620 | similar to regulator of G-protein | Mmu.16259 | XP_001084439 /// | 49.87419 | A | 116.6921 | P |
| | | signalling 6 | | XP_001084557 /// | | | | |
| | | | | XP_001084681 | | | | |
| MmugDNA.31733.1.S1_at | LOC694857 | similar to Ca2+-dependent | Mmu.14856 | XP_001083727 /// | 89.40633 | A | 66.00955 | P |
| | | activator protein for secretion 2 | | XP_001083836 /// | | | | |
| | | isoform b | | XP_001083951 /// | | | | |
| | | | | XP_001084057 /// | | | | |
| | | | | XP_001084178 /// | | | | |
| | | | | XP_001084294 /// | | | | |
| | | | | XP_001084413 | | | | |
| MmugDNA.8053.1.S1_at | LOC694961 | Similar to Scm-like with four mbt | Mmu.13251 | XP_001084282 /// | 111.6069 | A | 98.00121 | P |
| | | domains 1 | | XP_001084404 /// | | | | |
| | | | | XP_001084525 /// | | | | |
| | | | | XP_001084648 /// | | | | |
| | | | | XP_001084766 /// | | | | |
| | | | | XP_001084884 /// | | | | |
| | | | | XP_001084999 | | | | |
| MmugDNA.7498.1.S1_at | LOC695519 | hypothetical protein LOC695519 | Mmu.14586 | XP_001088156 | 103.4704 | A | 190.2334 | P |
| MmugDNA.23700.1.S1_at | LOC695660 | similar to glutamate receptor 6 | Mmu.14770 | XP_001086511 /// | 71.56487 | A | 175.5264 | P |
| | | isoform 1 precursor | | XP_001086640 /// | | | | |
| | | | | XP_001086762 | | | | |
| Mmu.12414.1.S1_at | LOC695807 | similar to positive cofactor 2, | Mmu.12304 | XP_001084461 | 334.7846 | A | 548.711 | P |
| | | glutamine/Q-rich-associated | | | | | | |
| | | protein isoform b | | | | | | |
| MmugDNA.30964.1.S1_at | LOC695817 | similar to FRG1 protein (FSHD | Mmu.5349 | XP_001084469 | 63.37233 | A | 129.4325 | P |
| | /// | region gene 1 protein) /// similar | | | | | | |
| | LOC721908 | to FRG1 protein (FSHD region | | | | | | |
| | | gene 1 protein) | | | | | | |
| MmugDNA.40736.1.S1_at | LOC695861 | similar to regulatory factor X3 | Mmu.15656 | XP_001086023 /// | 59.62864 | A | 127.9034 | P |
| | | isoform b | | XP_001086140 /// | | | | |
| | | | | XP_001086369 /// | | | | |
| | | | | XP_001086480 /// | | | | |
| | | | | XP_001086606 /// | | | | |
| | | | | XP_001086729 | | | | |
| MmugDNA.17800.1.S1_at | LOC695993 | similar to chromosome 18 open | Mmu.11030 | XP_001084399 /// | 114.231 | A | 191.3399 | P |
| | | reading frame 25 isoform b | | XP_001084520 /// | | | | |
| | | | | XP_001084644 | | | | |
| MmugDNA.25624.1.S1_at | LOC696061 | similar to Wilms tumor 1 isoform B | Mmu.4826 | XP_001084704 | 7.96631 | A | 115.5726 | P |
| MmunewRS.241.1.S1_at | LOC696130 | similar to lin-28 homolog B | Mmu.2034 | XP_001086990 | 38.80841 | A | 120.6038 | P |
| MmugDNA.27370.1.S1_at | LOC696199 | hypothetical protein LOC696199 | Mmu.15122 | XP_001085896 /// | 53.2388 | A | 143.1268 | P |
| | | | | XP_001085999 /// | | | | |
| | | | | XP_001086115 /// | | | | |
| | | | | XP_001086220 | | | | |
| MmuSTS.2262.1.S1_at | LOC696202 | similar to cathepsin E isoform a | Mmu.11543 | XP_001089941 /// | 5.594962 | A | 126.9847 | P |
| | | preproprotein | | XP_001090059 /// | | | | |
| | | | | XP_001090171 /// | | | | |
| | | | | XP_001090284 | | | | |
| MmugDNA.9916.1.S1_at | LOC696275 | similar to neuregulin 1 isoform | Mmu.16225 | XP_001085266 /// | 109.3953 | A | 329.6868 | P |
| | | HRG-beta1 | | XP_001085962 /// | | | | |
| | | | | XP_001086071 /// | | | | |
| | | | | XP_001086184 | | | | |
| MmugDNA.2716.1.S1_at | LOC696683 | similar to origin recognition | Mmu.12240 | XP_001085300 | 18.94276 | A | 94.6161 | P |
| | | complex subunit 5 isoform 1 | | | | | | |
| MmuSTS.2097.1.S1_s_at | LOC696726 | similar to Apolipoprotein C-III | Mmu.3042 | XP_001090312 | 296.7122 | A | 776.4622 | P |
| | | precursor (Apo-CIII) (ApoC-III) | | | | | | |
| MmugDNA.20065.1.S1_at | LOC696749 | similar to runt-related | Mmu.14911 | XP_001086266 /// | 216.7346 | A | 510.84 | P |
| | | transcription factor 1 isoform a | | XP_001086504 /// | | | | |
| | | | | XP_001086631 /// | | | | |
| | | | | XP_001086755 /// | | | | |
| | | | | XP_001086875 /// | | | | |
| | | | | XP_001086981 /// | | | | |
| | | | | XP_001087104 /// | | | | |
| | | | | XP_001087224 /// | | | | |
| | | | | XP_001087338 | | | | |
| MmugDNA.40590.1.S1_at | LOC696991 | similar to CG13322-PA, isoform A | Mmu.15342 | XP_001090213 /// | 151.6879 | A | 116.2028 | P |
| | | | | XP_001090328 /// | | | | |
| | | | | XP_001090443 /// | | | | |
| | | | | XP_001090556 | | | | |
| MmugDNA.23596.1.S1_at | LOC697028 | similar to dedicator of | Mmu.4738 | XP_001085641 | 42.86666 | A | 102.283 | P |
| | | cytokinesis 7 | | | | | | |
| MmugDNA.19935.1.S1_at | LOC697140 | similar to LIM domain binding 3 | Mmu.11452 | XP_001084921 /// | 127.76 | A | 128.6186 | P |
| | | | | XP_001085158 /// | | | | |
| | | | | XP_001085274 | | | | |
| MmugDNA.19209.1.S1_s_at | LOC697208 | similar to Neutrophil gelatinase- | Mmu.11352 | XP_001083008 | 87.27077 | A | 281.2362 | P |
| | | associated lipocalin precursor | | | | | | |
| | | (NGAL) (p25) (25 kDa alpha-2- | | | | | | |
| | | microglobulin-related subunit of | | | | | | |
| | | MMP-9) (Lipocalin-2) (Oncogene | | | | | | |
| | | 24p3) | | | | | | |
| MmugDNA.25784.1.S1_at | LOC697300 | Similar to DNA topoisomerase I | Mmu.12250 | XP_001088021 | 107.8373 | A | 191.1222 | P |
| Mmu.12394.1.S1_at | LOC697308 | similar to CG2453-PA | Mmu.3029 | XP_001089249 | 220.9762 | A | 178.96 | P |
| MmugDNA.33930.1.S1_s_at | LOC697557 | similar to LIN-7 homolog A (LIN- | Mmu.14198 | XP_001087299 | 17.28876 | A | 53.62199 | P |
| | | 7A) (mLin-7) (Mammalian LIN- | | | | | | |
| | | seven protein 1) (MALS-1) | | | | | | |
| | | (Vertebrate LIN 7 homolog 1) | | | | | | |
| | | (Veli-1 protein) | | | | | | |
| MmugDNA.27371.1.S1_at | LOC697751 | hypothetical protein LOC697751 | Mmu.14664 | XP_001086421 /// | 39.36357 | A | 72.29453 | P |
| | | | | XP_001086529 /// | | | | |
| | | | | XP_001086660 /// | | | | |
| | | | | XP_001086783 /// | | | | |
| | | | | XP_001086904 | | | | |
| MmugDNA.41587.1.S1_at | LOC697948 | similar to Apical Junction | Mmu.15719 | XP_001094873 /// | 231.1788 | A | 167.211 | P |
| | | Molecule family member (ajm-1) | | XP_001094990 /// | | | | |
| | | | | XP_001095091 /// | | | | |
| | | | | XP_001095202 /// | | | | |
| | | | | XP_001095318 /// | | | | |
| | | | | XP_001095432 /// | | | | |
| | | | | XP_001095548 | | | | |
| MmugDNA.2651.1.S1_s_at | LOC698116 | hypothetical protein LOC698116 | Mmu.17317 | XP_001089195 | 2.093363 | A | 121.8847 | P |
| MmugDNA.34368.1.S1_at | LOC698257 | hypothetical protein LOC698257 | Mmu.14406 | XP_001091064 | 35.19047 | A | 156.6444 | P |
| MmugDNA.6912.1.S1_at | LOC698488 | similar to glycoprotein M6A | Mmu.309 | XP_001088513 /// | 55.25916 | A | 91.12907 | P |
| | | isoform 1 | | XP_001088626 /// | | | | |
| | | | | XP_001088729 /// | | | | |
| | | | | XP_001088856 /// | | | | |
| | | | | XP_001088960 /// | | | | |
| | | | | XP_001089075 /// | | | | |
| | | | | XP_001089193 /// | | | | |
| | | | | XP_001089301 /// | | | | |
| | | | | XP_001089420 /// | | | | |
| | | | | XP_001089530 /// | | | | |
| | | | | XP_001089652 | | | | |
| MmugDNA.8688.1.S1_s_at | LOC698576 | similar to plakophilin 4 isoform a | Mmu.90 | XP_001090763 /// | 57.58762 | A | 130.691 | P |
| | | | | XP_001091487 /// | | | | |
| | | | | XP_001091595 /// | | | | |
| | | | | XP_001091710 /// | | | | |
| | | | | XP_001091829 | | | | |
| MmugDNA.4919.1.S1_at | LOC698583 | similar to transcobalamin I | Mmu.3193 | XP_001088817 | 6.806493 | A | 121.0638 | P |
| | | precursor | | | | | | |
| MmugDNA.25652.1.S1_at | LOC698610 | similar to netrin-G1 ligand | Mmu.14852 | XP_001090221 /// | 159.8217 | A | 460.219 | P |
| | | | | XP_001090333 /// | | | | |
| | | | | XP_001090447 | | | | |
| MmugDNA.7880.1.S1_at | LOC698922 | similar to transcription | Mmu.1273 | XP_001087401 | 67.81429 | A | 120.6219 | P |
| | | elongation factor B (SIII), | | | | | | |
| | | polypeptide 1 | | | | | | |
| MmugDNA.37713.1.S1_s_at | LOC698993 | similar to RAD1 homolog isoform 1 | Mmu.16416 | XP_001090119 /// | 10.74294 | A | 78.33199 | P |
| | | | | XP_001090232 /// | | | | |
| | | | | XP_001090346 | | | | |
| MmugDNA.26991.1.S1_x_at | LOC699049 | similar to SMT3 suppressor of | Mmu.10807 | XP_001082298 /// | 132.9624 | A | 151.3819 | P |
| | /// | mif two 3 homolog 2 /// similar | | XP_001093875 /// | | | | |
| | LOC705512 | to SMT3 suppressor of mif two 3 | | XP_001101180 | | | | |
| | /// | homolog 2 isoform b precursor | | | | | | |
| | LOC712242 | /// similar to SMT3 suppressor of | | | | | | |
| | /// SUMO2 | mif two 3 homolog 2 /// SMT3 | | | | | | |
| | | suppressor of mif two 3 homolog | | | | | | |
| | | 2 (S. cerevisiae) | | | | | | |
| MmugDNA.30038.1.S1_at | LOC699233 | similar to J domain protein | Mmu.14908 | XP_001091149 /// | 20.38795 | A | 105.1611 | P |
| | | C21orf55 | | XP_001091275 /// | | | | |
| | | | | XP_001091400 | | | | |
| MmugDNA.24540.1.S1_at | LOC699255 | similar to c-Mpl binding protein | Mmu.8566 | XP_001103235 | 97.93331 | A | 108.0194 | P |
| | /// | isoform c /// similar to c-Mpl | | | | | | |
| | LOC713773 | binding protein isoform a | | | | | | |
| MmugDNA.36570.1.S1_at | LOC699320 | similar to keratin 1 | Mmu.15995 | XP_001097706 /// | 82.466 | A | 155.1366 | P |
| | | | | XP_001097800 /// | | | | |
| | | | | XP_001097902 /// | | | | |
| | | | | XP_001097988 /// | | | | |
| | | | | XP_001098082 /// | | | | |
| | | | | XP_001098182 /// | | | | |
| | | | | XP_001098292 | | | | |
| MmugDNA.36148.1.S1_at | LOC699519 | similar to cytochrome P450, | Mmu.16164 | XP_001091192 /// | 6.762844 | A | 128.3562 | P |
| | | family 26, subfamily A, | | XP_001091311 /// | | | | |
| | | polypeptide 1 isoform 2 | | XP_001091435 | | | | |
| MmugDNA.34762.1.S1_at | LOC699606 | similar to YKT6 v-SNARE protein | Mmu.14457 | XP_001092458 /// | 352.2617 | A | 455.7151 | P |
| | | | | XP_001092567 | | | | |
| MmugDNA.5021.1.S1_at | LOC699884 | similar to aspartate beta- | Mmu.12462 | XP_001090473 /// | 74.45432 | A | 142.7363 | P |
| | | hydroxylase isoform b | | XP_001090590 /// | | | | |
| | | | | XP_001090710 /// | | | | |
| | | | | XP_001090824 | | | | |
| MmugDNA.35909.1.S1_at | LOC700246 | hypothetical protein LOC700246 | Mmu.16339 | XP_001091537 /// | 141.387 | A | 153.2613 | P |
| | | | | XP_001091653 /// | | | | |
| | | | | XP_001091770 | | | | |
| MmugDNA.2916.1.S1_at | LOC700561 | similar to Protein FAM61B | Mmu.4706 | XP_001090063 | 64.34468 | A | 237.9115 | P |
| MmugDNA.2141.1.S1_at | LOC700632 | similar to tumor protein p53 | Mmu.12749 | XP_001090474 /// | 103.4481 | A | 245.4697 | P |
| | | inducible nuclear protein 1 | | XP_001090592 | | | | |
| MmugDNA.6969.1.S1_s_at | LOC700634 | similar to PTK2 protein tyrosine | Mmu.3254 | XP_001091188 /// | 318.6551 | A | 503.8765 | P |
| | | kinase 2 isoform a | | XP_001092257 /// | | | | |
| | | | | XP_001092479 /// | | | | |
| | | | | XP_001092599 /// | | | | |
| | | | | XP_001092714 /// | | | | |
| | | | | XP_001092835 /// | | | | |
| | | | | XP_001092947 /// | | | | |
| | | | | XP_001093060 /// | | | | |
| | | | | XP_001093166 /// | | | | |
| | | | | XP_001093282 | | | | |
| MmugDNA.11282.1.S1_at | LOC700766 | similar to CG14299-PA, isoform A | Mmu.13220 | XP_001089105 | 102.762 | A | 180.8994 | P |
| MmugDNA.1940.1.S1_at | LOC700803 | LOC57821 | Mmu.10545 | XP_001093899 /// | 42.41242 | A | 65.8287 | P |
| | | | | XP_001094005 /// | | | | |
| | | | | XP_001094116 | | | | |
| MmugDNA.22818.1.S1_at | LOC700803 | LOC57821 | Mmu.10545 | XP_001093899 /// | 99.11937 | A | 161.2611 | P |
| | | | | XP_001094005 /// | | | | |
| | | | | XP_001094116 | | | | |
| MmugDNA.33647.1.S1_s_at | LOC700961 | similar to zinc finger protein 79 | Mmu.15647 | XP_001095865 | 124.1566 | A | 166.1214 | P |
| | | (pT7) | | | | | | |
| MmugDNA.43384.1.S1_at | LOC700988 | similar to androgen-induced 1 | Mmu.4494 | XP_001091992 /// | 50.12266 | A | 91.1585 | P |
| | | | | XP_001092111 /// | | | | |
| | | | | XP_001092349 /// | | | | |
| | | | | XP_001092577 | | | | |
| MmugDNA.13490.1.S1_at | LOC701093 | similar to calcitonin receptor | Mmu.14864 | XP_001099231 /// | 70.82004 | A | 141.6998 | P |
| | | | | XP_001099337 /// | | | | |
| | | | | XP_001099435 /// | | | | |
| | | | | XP_001099544 | | | | |
| MmuSTS.1092.1.S1_at | LOC701162 | similar to solute carrier family 6, | Mmu.11269 | XP_001099500 | 62.05202 | A | 183.6655 | P |
| | | member 17 | | | | | | |
| MmugDNA.10860.1.S1_s_at | LOC701186 | similar to serine | Mmu.12203 | XP_001096193 /// | 299.3282 | A | 568.1307 | P |
| | | hydroxymethyltransferase 1 | | XP_001096314 /// | | | | |
| | | (soluble) isoform 1 | | XP_001096436 /// | | | | |
| | | | | XP_001096547 /// | | | | |
| | | | | XP_001096653 /// | | | | |
| | | | | XP_001096755 /// | | | | |
| | | | | XP_001096871 /// | | | | |
| | | | | XP_001096982 | | | | |
| MmugDNA.9882.1.S1_at | LOC701376 | similar to NADPH oxidase 1 | Mmu.15254 | XP_001089791 /// | 59.80977 | A | 157.23 | P |
| | | isoform long variant | | XP_001089905 /// | | | | |
| | | | | XP_001090025 /// | | | | |
| | | | | XP_001090252 /// | | | | |
| | | | | XP_001090373 | | | | |
| MmugDNA.24847.1.S1_at | LOC701547 | similar to glycine-N- | Mmu.14222 | XP_001092072 /// | 104.8917 | A | 213.7509 | P |
| | | acyltransferase-like 1 | | XP_001092197 /// | | | | |
| | | | | XP_001092316 /// | | | | |
| | | | | XP_001092427 | | | | |
| Mmu.11429.1.A1_at | LOC701690 | Similar to T-cell activation Rho | Mmu.12230 | XP_001095239 /// | 125.3303 | A | 137.3554 | P |
| | | GTPase-activating protein | | XP_001095349 /// | | | | |
| | | isoform b | | XP_001095467 /// | | | | |
| | | | | XP_001095579 /// | | | | |
| | | | | XP_001095675 /// | | | | |
| | | | | XP_001095775 | | | | |
| MmugDNA.37160.1.S1_at | LOC701721 | Similar to X Kell blood group | Mmu.13648 | XP_001090721 | 93.54068 | A | 282.5066 | P |
| | | precursor-related, X-linked | | | | | | |
| MmugDNA.14409.1.S1_at | LOC701722 | similar to nucleoporin 62 | Mmu.12987 | XP_001094695 | 17.99913 | A | 83.21231 | P |
| Mmu.2615.1.S1_at | LOC702282 | similar to ADP-ribosylation factor 7 | Mmu.3100 | XP_001096998 | 45.92997 | A | 137.102 | P |
| MmugDNA.12356.1.S1_at | LOC702392 | similar to peroxin1 | Mmu.13262 | XP_001100851 /// | 267.6477 | A | 272.199 | P |
| | | | | XP_001101055 | | | | |
| MmugDNA.35938.1.S1_at | LOC702474 | similar to F46E10.1a /// similar | Mmu.11756 | XP_001093983 /// | 212.2583 | A | 374.0013 | P |
| | /// | to radical S-adenosyl methionine | | XP_001097818 | | | | |
| | LOC705626 | domain containing 1 | | | | | | |
| MmugDNA.22044.1.S1_at | LOC702523 | similar to plexin domain | Mmu.13851 | XP_001094684 /// | 56.80964 | A | 144.3693 | P |
| | | containing 2 precursor | | XP_001094803 | | | | |
| MmugDNA.30120.1.S1_at | LOC702596 | similar to nucleolar RNA- | Mmu.15669 | XP_001099108 /// | 517.206 | A | 624.0098 | P |
| | | associated protein alpha isoform | | XP_001099208 | | | | |
| MmugDNA.14645.1.S1_at | LOC702627 | similar to CNKSR family member 3 | Mmu.16800 | XP_001095122 | 112.3272 | A | 171.7359 | P |
| MmuSTS.4277.1.S1_at | LOC702809 | similar to eukaryotic translation | Mmu.7828 | XP_001096741 /// | 46.99945 | A | 91.84947 | P |
| | /// | elongation factor 1 alpha 2 /// | | XP_001107881 /// | | | | |
| | LOC703715 | similar to eukaryotic translation | | XP_001107938 /// | | | | |
| | /// | elongation factor 1 alpha 2 /// | | XP_001107997 /// | | | | |
| | LOC704199 | similar to eukaryotic translation | | XP_001108047 | | | | |
| | /// | elongation factor 1 alpha 2 /// | | | | | | |
| | LOC704438 | similar to eukaryotic translation | | | | | | |
| | /// | elongation factor 1 alpha 1 /// | | | | | | |
| | LOC709017 | similar to eukaryotic translation | | | | | | |
| | /// | elongation factor 1 alpha 1 /// | | | | | | |
| | LOC715351 | similar to eukaryotic translation | | | | | | |
| | | elongation factor 1 alpha 1 | | | | | | |
| Mmu.14402.2.A1_at | LOC702904 | Similar to T-cell receptor beta | Mmu.3323 | XP_001091216 | 43.79837 | A | 185.5729 | P |
| | | chain C region | | | | | | |
| MmugDNA.35794.1.S1_at | LOC703305 | similar to adrenomedullin | Mmu.1495 | XP_001100373 /// | 84.27942 | A | 185.7086 | P |
| | | | | XP_001100748 /// | | | | |
| | | | | XP_001100827 | | | | |
| MmugDNA.18234.1.S1_s_at | LOC703331 | similar to runt-related | Mmu.14787 | XP_001100592 /// | 58.99397 | A | 193.9611 | P |
| | | transcription factor 2 isoform b | | XP_001100682 /// | | | | |
| | | | | XP_001100772 /// | | | | |
| | | | | XP_001100855 /// | | | | |
| | | | | XP_001100961 /// | | | | |
| | | | | XP_001101058 | | | | |
| MmugDNA.42623.1.S1_at | LOC703350 | Similar to SLD5 | Mmu.12445 | XP_001094432 | 116.1626 | A | 230.3927 | P |
| MmugDNA.37293.1.S1_at | LOC703448 | similar to NHS-like 1 | Mmu.12755 | XP_001094901 /// | 38.42938 | A | 120.9677 | P |
| | | | | XP_001095017 /// | | | | |
| | | | | XP_001095123 | | | | |
| MmugDNA.28192.1.S1_at | LOC703449 | similar to solute carrier family 22 | Mmu.14760 | XP_001098823 /// | 85.29906 | A | 109.4904 | P |
| | | member 1 isoform a | | XP_001098927 /// | | | | |
| | | | | XP_001099030 | | | | |
| Mmu.9793.1.S1_at | LOC703475 | similar to chloride intracellular | Mmu.13655 | XP_001098953 /// | 113.1555 | A | 216.6789 | P |
| | | channel 2 | | XP_001099055 /// | | | | |
| | | | | XP_001099159 | | | | |
| MmugDNA.14145.1.S1_s_at | LOC703676 | similar to clusterin associated | Mmu.12076 | XP_001095007 | 9.514027 | A | 72.95879 | P |
| | | protein 1 isoform 1 | | | | | | |
| MmugDNA.32118.1.S1_at | LOC703699 | similar to N-acylsphingosine | Mmu.1534 | XP_001098236 /// | 49.51334 | A | 133.2948 | P |
| | | amidohydrolase (acid | | XP_001098342 | | | | |
| | | ceramidase) 1 preproprotein | | | | | | |
| | | isoform a | | | | | | |
| MmugDNA.5848.1.S1_at | LOC703707 | similar to type IV alpha 6 | Mmu.14512 | XP_001098247 /// | 118.7948 | A | 173.2802 | P |
| | | collagen isoform A precursor | | XP_001098348 /// | | | | |
| | | | | XP_001098448 | | | | |
| MmugDNA.6896.1.S1_at | LOC703747 | hypothetical protein LOC703747 | Mmu.11245 | XP_001097797 /// | 99.1186 | A | 166.5303 | P |
| | | | | XP_001097899 /// | | | | |
| | | | | XP_001097986 /// | | | | |
| | | | | XP_001098078 /// | | | | |
| | | | | XP_001098178 /// | | | | |
| | | | | XP_001098384 | | | | |
| MmugDNA.18415.1.S1_at | LOC703912 | similar to zinc-finger protein | Mmu.16177 | XP_001095490 /// | 66.7701 | A | 171.3356 | P |
| | | NOLZ1 | | XP_001095599 /// | | | | |
| | | | | XP_001095696 /// | | | | |
| | | | | XP_001095797 | | | | |
| MmugDNA.20879.1.S1_at | LOC703981 | similar to NOD9 protein isoform 1 | Mmu.15714 | XP_001104018 /// | 68.5675 | A | 247.2942 | P |
| | | | | XP_001104187 /// | | | | |
| | | | | XP_001104271 /// | | | | |
| | | | | XP_001104348 /// | | | | |
| | | | | XP_001104430 | | | | |
| MmugDNA.43058.1.S1_at | LOC704126 | similar to kinesin family member | Mmu.14823 | XP_001096456 /// | 240.8623 | A | 391.0729 | P |
| | | 13A | | XP_001096670 /// | | | | |
| | | | | XP_001096780 /// | | | | |
| | | | | XP_001096896 | | | | |
| MmugDNA.9461.1.S1_at | LOC704129 | similar to M-phase | Mmu.872 | XP_001097629 /// | 9.741516 | A | 150.9924 | P |
| | /// | phosphoprotein 6 /// similar to | | XP_001111939 | | | | |
| | LOC714335 | M-phase phosphoprotein 6 | | | | | | |
| MmugDNA.36952.1.S1_at | LOC704226 | similar to | Mmu.1901 | XP_001095099 /// | 181.1201 | A | 255.0666 | P |
| | | metallophosphoesterase 1 | | XP_001095213 /// | | | | |
| | | precursor | | XP_001095328 /// | | | | |
| | | | | XP_001095440 /// | | | | |
| | | | | XP_001095557 /// | | | | |
| | | | | XP_001095654 | | | | |
| MmugDNA.20355.1.S1_at | LOC704563 | hypothetical protein LOC704563 | Mmu.14834 | XP_001087946 /// | 43.39126 | A | 163.4751 | P |
| | | | | XP_001088073 /// | | | | |
| | | | | XP_001088182 /// | | | | |
| | | | | XP_001088296 /// | | | | |
| | | | | XP_001088391 | | | | |
| MmugDNA.32338.1.S1_at | LOC704620 | Hypothetical protein LOC704620 | Mmu.12860 | XP_001092983 | 484.9445 | A | 1003.579 | P |
| MmugDNA.26222.1.S1_at | LOC704779 | hypothetical protein LOC704779 | Mmu.1356 | XP_001088505 /// | 145.6491 | A | 229.6519 | P |
| | | | | XP_001088616 /// | | | | |
| | | | | XP_001088722 /// | | | | |
| | | | | XP_001088845 | | | | |
| MmugDNA.32288.1.S1_at | LOC705099 | similar to IBR domain containing 2 | Mmu.13564 | XP_001097834 | 169.2173 | A | 287.2967 | P |
| MmuSTS.753.1.S1_at | LOC705192 | similar to solute carrier family 34 | Mmu.4 | XP_001087672 /// | 399.9195 | A | 614.1758 | P |
| | | (sodium phosphate), member 3 | | XP_001087793 | | | | |
| MmugDNA.13807.1.S1_at | LOC705221 | similar to CD2-associated protein | Mmu.14706 | XP_001103871 | 101.1595 | A | 165.9324 | P |
| MmugDNA.32676.1.S1_at | LOC705267 | similar to mitochondrial | Mmu.4582 | XP_001093640 | 81.411 | A | 106.6216 | P |
| | | ribosomal protein S5 | | | | | | |
| MmugDNA.39707.1.S1_at | LOC705275 | similar to 5-azacytidine induced 2 | Mmu.2888 | XP_001093647 | 228.3279 | A | 445.0563 | P |
| MmugDNA.3813.1.S1_at | LOC705358 | similar to PDZ and LIM domain 5 | Mmu.11535 | XP_001102789 /// | 196.3453 | A | 217.9732 | P |
| | | isoform a | | XP_001103137 /// | | | | |
| | | | | XP_001103292 /// | | | | |
| | | | | XP_001103360 /// | | | | |
| | | | | XP_001103447 | | | | |
| MmugDNA.41256.1.S1_at | LOC705369 | similar to SET domain containing 3 | Mmu.12527 | XP_001103146 /// | 307.612 | A | 442.7356 | P |
| | | | | XP_001103224 /// | | | | |
| | | | | XP_001103370 | | | | |
| MmugDNA.28135.1.S1_at | LOC705773 | similar to wingless-type MMTV | Mmu.15115 | XP_001107735 /// | 53.23829 | A | 147.2127 | P |
| | | integration site family, member | | XP_001107795 /// | | | | |
| | | 2B isoform WNT-2B2 | | XP_001107861 | | | | |
| MmugDNA.32069.1.S1_at | LOC705781 | similar to CG10721-PA | Mmu.11312 | XP_001098386 | 139.6408 | A | 181.3665 | P |
| MmugDNA.7008.1.S1_at | LOC705781 | similar to CG10721-PA | Mmu.11312 | XP_001098386 | 257.4816 | A | 205.8675 | P |
| MmugDNA.20418.1.S1_at | LOC705794 | similar to Glutamate receptor 4 | Mmu.15725 | XP_001100749 /// | 49.4125 | A | 144.5466 | P |
| | | precursor (GluR-4) (GluR4) | | XP_001100828 /// | | | | |
| | | (GluR-D) (Glutamate receptor | | XP_001100934 /// | | | | |
| | | ionotropic, AMPA 4) (AMPA- | | XP_001101031 /// | | | | |
| | | selective glutamate receptor 4) | | XP_001101120 /// | | | | |
| | | | | XP_001101205 /// | | | | |
| | | | | XP_001101297 /// | | | | |
| | | | | XP_001101389 | | | | |
| MmugDNA.11389.1.S1_at | LOC705913 | similar to cytidine deaminase | Mmu.14713 | XP_001096632 | 157.2027 | A | 432.5854 | P |
| Mmu.8604.1.A1_at | LOC706093 | Similar to golgi-associated | Mmu.12976 | XP_001099454 /// | 105.2247 | A | 270.9851 | P |
| | | microtubule-binding protein | | XP_001099564 | | | | |
| | | HOOK3 | | | | | | |
| MmugDNA.31582.1.S1_at | LOC706263 | similar to potassium voltage- | Mmu.15899 | XP_001108426 | 64.51572 | A | 142.2389 | P |
| | | gated channel, Isk-related | | | | | | |
| | | family, member 4 | | | | | | |
| MmugDNA.6257.1.S1_at | LOC706392 | similar to Temporarily Assigned | Mmu.15492 | XP_001100027 /// | 28.65171 | A | 216.6125 | P |
| | | Gene name family member (tag- | | XP_001100112 /// | | | | |
| | | 241) | | XP_001100204 | | | | |
| MmugDNA.2951.1.S1_at | LOC706404 | similar to anillin, actin binding | Mmu.12004 | XP_001103129 /// | 41.87038 | A | 150.5943 | P |
| | | protein (scraps homolog, | | XP_001103356 /// | | | | |
| | | Drosophila) | | XP_001103440 | | | | |
| MmugDNA.16996.1.S1_at | LOC706417 | similar to rap2 interacting | Mmu.3060 | XP_001105219 /// | 77.67221 | A | 127.3368 | P |
| | | protein x isoform 2 | | XP_001105432 /// | | | | |
| | | | | XP_001105503 /// | | | | |
| | | | | XP_001105576 | | | | |
| MmugDNA.2760.1.S1_at | LOC706531 | similar to SRY (sex determining | Mmu.2172 | XP_001098923 | 494.1494 | A | 320.2222 | P |
| | | region Y)-box 4 | | | | | | |
| MmugDNA.41505.1.S1_at | LOC706541 | similar to PDZ domain-containing | Mmu.12847 | XP_001100504 /// | 60.10989 | A | 85.12571 | P |
| | | guanine nucleotide exchange | | XP_001100780 /// | | | | |
| | | factor I | | XP_001100863 /// | | | | |
| | | | | XP_001100970 /// | | | | |
| | | | | XP_001101236 /// | | | | |
| | | | | XP_001101336 /// | | | | |
| | | | | XP_001101427 /// | | | | |
| | | | | XP_001101518 | | | | |
| MmugDNA.4165.1.S1_at | LOC707050 | similar to caldesmon 1 isoform 4 | Mmu.1286 | XP_001103048 /// | 134.2276 | A | 113.0357 | P |
| | | | | XP_001103209 /// | | | | |
| | | | | XP_001103288 /// | | | | |
| | | | | XP_001103525 /// | | | | |
| | | | | XP_001103606 /// | | | | |
| | | | | XP_001103692 /// | | | | |
| | | | | XP_001103781 /// | | | | |
| | | | | XP_001103868 /// | | | | |
| | | | | XP_001103951 /// | | | | |
| | | | | XP_001104042 /// | | | | |
| | | | | XP_001104129 /// | | | | |
| | | | | XP_001104210 /// | | | | |
| | | | | XP_001104289 /// | | | | |
| | | | | XP_001104371 | | | | |
| MmugDNA.24929.1.S1_at | LOC707355 | similar to zinc finger protein 672 | Mmu.15300 | XP_001105495 /// | 160.5896 | A | 148.2761 | P |
| | | | | XP_001105637 /// | | | | |
| | | | | XP_001105777 | | | | |
| MmugDNA.21203.1.S1_x_at | LOC707383 | similar to amyloid beta (A4) | Mmu.669 | XP_001101993 | 428.4578 | A | 499.8212 | P |
| | | precursor protein-binding, family | | | | | | |
| | | B, member 1 interacting protein | | | | | | |
| MmugDNA.35674.1.S1_at | LOC707661 | similar to additional sex combs | Mmu.15484 | XP_001101400 /// | 110.2575 | A | 154.3551 | P |
| | | like 1 | | XP_001101495 /// | | | | |
| | | | | XP_001101584 /// | | | | |
| | | | | XP_001101677 | | | | |
| MmugDNA.14411.1.S1_at | LOC707686 | similar to angiogenic factor | Mmu.12022 | XP_001106588 /// | 312.2592 | A | 474.9281 | P |
| | | VG5Q | | XP_001106645 /// | | | | |
| | | | | XP_001106704 /// | | | | |
| | | | | XP_001106767 | | | | |
| MmuSTS.1472.1.S1_at | LOC707739 | Similar to serine/threonine | Mmu.12485 | XP_001096161 | 31.29481 | A | 81.42928 | P |
| | | protein kinase MASK | | | | | | |
| MmugDNA.6011.1.S1_at | LOC707982 | similar to rhomboid domain | Mmu.15898 | XP_001110015 /// | 253.2026 | A | 423.9781 | P |
| | /// | containing 1 /// hypothetical | | XP_001110070 /// | | | | |
| | LOC708189 | protein LOC708189 | | XP_001110119 /// | | | | |
| | | | | XP_001110165 /// | | | | |
| | | | | XP_001110210 | | | | |
| MmugDNA.37057.1.S1_at | LOC708094 | similar to mannosyl (alpha-1,3-)- | Mmu.15839 | XP_001102758 /// | 104.4101 | A | 147.5036 | P |
| | | glycoprotein beta-1,4-N- | | XP_001102852 | | | | |
| | | acetylglucosaminyltransferase, | | | | | | |
| | | isoenzyme A | | | | | | |
| MmugDNA.42069.1.S1_s_at | LOC708427 | similar to organic solute | Mmu.11865 | XP_001108335 | 78.81197 | A | 116.7262 | P |
| | | transporter beta | | | | | | |
| MmugDNA.30697.1.S1_at | LOC708649 | similar to leucine rich repeat | Mmu.13704 | XP_001101795 /// | 163.4347 | A | 221.4591 | P |
| | | containing 28 | | XP_001101886 /// | | | | |
| | | | | XP_001101987 /// | | | | |
| | | | | XP_001102074 | | | | |
| MmuSTS.1715.1.S1_at | LOC708906 | similar to wingless-type MMTV | Mmu.14255 | XP_001104947 /// | 130.317 | A | 218.3881 | P |
| | | integration site family, member | | XP_001105026 /// | | | | |
| | | 10B precursor | | XP_001105115 | | | | |
| MmuSTS.4837.1.S1_at | LOC708940 | similar to Guanine nucleotide- | Mmu.14862 | XP_001108555 /// | 67.89697 | A | 63.73149 | P |
| | | binding protein G(i), alpha-1 | | XP_001108609 /// | | | | |
| | | subunit (Adenylate cyclase- | | XP_001108656 | | | | |
| | | inhibiting G alpha protein) | | | | | | |
| MmuSTS.508.1.S1_at | LOC709049 | similar to activating signal | Mmu.14734 | XP_001105587 /// | 145.0563 | A | 167.4562 | P |
| | | cointegrator 1 complex subunit 1 | | XP_001105655 /// | | | | |
| | | | | XP_001105727 /// | | | | |
| | | | | XP_001105789 /// | | | | |
| | | | | XP_001105856 /// | | | | |
| | | | | XP_001105929 /// | | | | |
| | | | | XP_001106003 | | | | |
| MmugDNA.31920.1.S1_at | LOC709087 | similar to EF-hand domain (C- | Mmu.14530 | XP_001097580 | 36.69831 | A | 95.83981 | P |
| | | terminal) containing 2 | | | | | | |
| MmugDNA.32637.1.S1_at | LOC709253 | similar to nucleosomal binding | Mmu.13802 | XP_001103630 | 80.94846 | A | 106.7455 | P |
| | | protein 1 | | | | | | |
| MmugDNA.42508.1.S1_at | LOC709303 | similar to ATP binding protein | Mmu.1839 | XP_001104184 /// | 60.61469 | A | 122.8885 | P |
| | | associated with cell | | XP_001104266 | | | | |
| | | differentiation | | | | | | |
| MmugDNA.38015.1.S1_at | LOC709468 | similar to terminal | Mmu.12395 | XP_001107131 | 75.45173 | A | 118.2112 | P |
| | | deoxynucleotidyltransferase | | | | | | |
| | | interacting factor 1 | | | | | | |
| Mmu.10346.1.S1_at | LOC709544 | hypothetical protein LOC709544 | Mmu.16556 | XP_001096738 | 507.0977 | A | 619.4869 | P |
| Mmu.6678.1.S1_x_at | LOC709619 | similar to Metallothionein-1I | Mmu.11575 | XP_001098159 | 1740.248 | A | 2628.085 | P |
| | | (MT-1I) (Metallothionein-II) | | | | | | |
| MmugDNA.8461.1.S1_at | LOC709735 | similar to nuclear receptor | Mmu.15049 | XP_001110159 /// | 42.78323 | A | 100.8168 | P |
| | | subfamily 5, group A, member 2 | | XP_001110202 /// | | | | |
| | | isoform 2 | | XP_001110281 | | | | |
| MmugDNA.39938.1.S1_at | LOC709882 | similar to Troponin T, cardiac | Mmu.14101 | XP_001098451 | 129.6782 | A | 230.9345 | P |
| | | muscle (TnTc) (Cardiac muscle | | | | | | |
| | | troponin T) (cTnT) | | | | | | |
| MmugDNA.1217.1.S1_at | LOC709920 | similar to Helix-loop-helix protein | Mmu.15111 | XP_001112029 /// | 8.452376 | A | 99.66127 | P |
| | | 2 (HEN2) (Nescient helix loop | | XP_001112063 /// | | | | |
| | | helix 2) (NSCL-2) | | XP_001112098 | | | | |
| MmugDNA.19461.1.S1_at | LOC710132 | similar to p53 target zinc finger | Mmu.13485 | XP_001109218 | 348.8743 | A | 339.8408 | P |
| | | protein isoform 1 | | | | | | |
| MmugDNA.33092.1.S1_s_at | LOC710136 | similar to alpha-2-glycoprotein 1, | Mmu.2996 | XP_001098813 | 12.33912 | A | 216.8815 | P |
| | | zinc | | | | | | |
| MmugDNA.41451.1.S1_at | LOC710209 | similar to F33H2.6 | Mmu.12671 | XP_001108744 /// | 21.07946 | A | 115.8894 | P |
| | | | | XP_001108792 /// | | | | |
| | | | | XP_001108841 /// | | | | |
| | | | | XP_001108885 /// | | | | |
| | | | | XP_001108926 /// | | | | |
| | | | | XP_001108981 /// | | | | |
| | | | | XP_001109038 /// | | | | |
| | | | | XP_001109094 /// | | | | |
| | | | | XP_001109206 | | | | |
| MmugDNA.25032.1.S1_at | LOC710300 | similar to CDC14 homolog B | Mmu.15640 | XP_001106305 /// | 96.10104 | A | 163.648 | P |
| | | isoform 1 | | XP_001106374 /// | | | | |
| | | | | XP_001106436 | | | | |
| MmugDNA.26478.1.S1_at | LOC710518 | similar to mitochondrial solute | Mmu.11000 | XP_001105927 /// | 455.317 | A | 775.371 | P |
| | | carrier-like protein | | XP_001106001 /// | | | | |
| | | | | XP_001106076 /// | | | | |
| | | | | XP_001106141 /// | | | | |
| | | | | XP_001106211 | | | | |
| MmugDNA.17545.1.S1_at | LOC710681 | hypothetical protein LOC710681 | Mmu.16264 | XP_001106907 /// | 117.03 | A | 219.1418 | P |
| | | | | XP_001106971 /// | | | | |
| | | | | XP_001107040 | | | | |
| MmugDNA.28666.1.S1_at | LOC710731 | similar to palmdelphin | Mmu.12773 | XP_001105606 /// | 37.84584 | A | 84.9398 | P |
| | | | | XP_001105677 /// | | | | |
| | | | | XP_001105748 /// | | | | |
| | | | | XP_001105804 /// | | | | |
| | | | | XP_001105954 | | | | |
| MmugDNA.8258.1.S1_at | LOC710987 | similar to mucolipin 3 | Mmu.15135 | XP_001108095 /// | 29.74118 | A | 102.5425 | P |
| | | | | XP_001108302 /// | | | | |
| | | | | XP_001108412 | | | | |
| MmugDNA.34412.1.S1_at | LOC711115 | similar to mitogen-activated | Mmu.3822 | XP_001108716 /// | 22.68439 | A | 99.87075 | P |
| | | protein kinase 8 isoform 1 | | XP_001108770 /// | | | | |
| | | | | XP_001108815 /// | | | | |
| | | | | XP_001108865 | | | | |
| Mmu.5150.1.A1_at | LOC711124 | Similar to NCK-associated | Mmu.2403 | XP_001099879 | 88.93265 | A | 141.7522 | P |
| | | protein 1 isoform 2 | | | | | | |
| Mmu.5996.2.S1_x_at | LOC711141 | hypothetical protein LOC711141 | Mmu.10434 | XP_001099892 | 92.06384 | A | 109.7883 | P |
| MmugDNA.17269.1.S1_at | LOC711164 | similar to sulfite oxidase | Mmu.15990 | XP_001113374 /// | 171.6959 | A | 271.7326 | P |
| | | | | XP_001113406 /// | | | | |
| | | | | XP_001113430 /// | | | | |
| | | | | XP_001113456 | | | | |
| MmugDNA.4283.1.S1_at | LOC711279 | similar to leucine zipper, putative | Mmu.16155 | XP_001109524 /// | 156.9818 | A | 163.6825 | P |
| | | tumor suppressor 2 | | XP_001109574 /// | | | | |
| | | | | XP_001109628 /// | | | | |
| | | | | XP_001109674 /// | | | | |
| | | | | XP_001109715 /// | | | | |
| | | | | XP_001109770 /// | | | | |
| | | | | XP_001109817 | | | | |
| MmugDNA.33876.1.S1_s_at | LOC711336 | similar to olfactomedin 2 | Mmu.15445 | XP_001099531 /// | 105.1338 | A | 182.7413 | P |
| | | | | XP_001099628 /// | | | | |
| | | | | XP_001099726 /// | | | | |
| | | | | XP_001099831 | | | | |
| MmugDNA.17217.1.S1_at | LOC711485 | similar to acyl-Coenzyme A | Mmu.12959 | XP_001109931 | 123.1174 | A | 293.2802 | P |
| | | dehydrogenase, long chain | | | | | | |
| | | precursor | | | | | | |
| MmugDNA.15166.1.S1_at | LOC711633 | similar to zinc finger CCCH-type | Mmu.15988 | XP_001114089 /// | 185.2786 | A | 239.9244 | P |
| | | containing 10 | | XP_001114107 /// | | | | |
| | | | | XP_001114132 /// | | | | |
| | | | | XP_001114150 | | | | |
| MmuSTS.4200.1.S1_at | LOC711691 | Similar to pre-B-cell leukemia | Mmu.4832 | XP_001100529 | 105.6631 | A | 780.6314 | P |
| | | transcription factor 3 | | | | | | |
| Mmu.13564.1.S1_at | LOC711742 | similar to kinesin family member | Mmu.564 | XP_001113416 /// | 8.346619 | A | 230.6845 | P |
| | | 9 isoform 2 | | XP_001113441 | | | | |
| MmugDNA.22716.1.S1_at | LOC711901 | similar to TBC1 domain family, | Mmu.11462 | XP_001109803 /// | 130.5118 | A | 170.4844 | P |
| | | member 16 | | XP_001109846 /// | | | | |
| | | | | XP_001109890 | | | | |
| MmugDNA.43479.1.S1_at | LOC711908 | similar to transmembrane 4 | Mmu.2535 | XP_001108758 /// | 8.667226 | A | 84.54928 | P |
| | | superfamily member 1 | | XP_001108803 | | | | |
| MmugDNA.30723.1.S1_at | LOC711984 | Similar to zinc finger CCCH-type | Mmu.12594 | XP_001106124 /// | 175.1743 | A | 424.0466 | P |
| | | domain containing 7 | | XP_001106316 /// | | | | |
| | | | | XP_001106384 /// | | | | |
| | | | | XP_001106446 | | | | |
| MmugDNA.20892.1.S1_at | LOC712033 | similar to PCTAIRE protein | Mmu.14735 | XP_001100911 | 120.9395 | A | 256.8878 | P |
| | | kinase 1 | | | | | | |
| MmugDNA.15919.1.S1_at | LOC712047 | similar to V-set and | Mmu.11490 | XP_001109973 /// | 73.78225 | A | 138.8611 | P |
| | | immunoglobulin domain | | XP_001110168 | | | | |
| | | containing 2 | | | | | | |
| MmugDNA.10446.1.S1_at | LOC712115 | similar to ADP-ribosylation factor | Mmu.14062 | XP_001101005 | 75.08247 | A | 115.4225 | P |
| | | guanine nucleotide factor 6 | | | | | | |
| | | isoform a | | | | | | |
| MmugDNA.11597.1.S1_at | LOC712144 | similar to zinc finger protein 42 | Mmu.15367 | XP_001099728 /// | 138.5558 | A | 151.442 | P |
| | | isoform 2 | | XP_001099833 | | | | |
| MmugDNA.12728.1.S1_at | LOC712272 | similar to fibronectin type 3 and | Mmu.13034 | XP_001109699 | 37.6952 | A | 102.4023 | P |
| | | SPRY domain-containing protein | | | | | | |
| MmuSTS.849.1.S1_at | LOC712288 | similar to thrombospondin, type | Mmu.11407 | XP_001106832 /// | 58.88806 | A | 99.43536 | P |
| | | I, domain containing 2 | | XP_001106900 | | | | |
| MmugDNA.26929.1.S1_s_at | LOC712486 | similar to ficolin 2 isoform a | Mmu.11973 | XP_001098197 /// | 37.70898 | A | 184.9997 | P |
| | | precursor | | XP_001098304 | | | | |
| MmugDNA.1324.1.S1_s_at | LOC712600 | similar to quaking homolog, KH | Mmu.2689 | XP_001101642 | 109.0662 | A | 139.7951 | P |
| | | domain RNA binding isoform | | | | | | |
| | | HQK-5 | | | | | | |
| MmugDNA.22601.1.S1_at | LOC713029 | hypothetical protein LOC713029 | Mmu.14564 | XP_001110409 | 11.16738 | A | 138.9437 | P |
| MmugDNA.12133.1.S1_at | LOC713035 | similar to BCL2-like 1 isoform 1 | Mmu.16103 | XP_001109879 /// | 159.8234 | A | 150.4057 | P |
| | | | | XP_001109926 /// | | | | |
| | | | | XP_001109964 /// | | | | |
| | | | | XP_001110009 /// | | | | |
| | | | | XP_001110062 /// | | | | |
| | | | | XP_001110113 /// | | | | |
| | | | | XP_001110162 /// | | | | |
| | | | | XP_001110205 /// | | | | |
| | | | | XP_001110245 /// | | | | |
| | | | | XP_001110285 /// | | | | |
| | | | | XP_001110326 | | | | |
| MmugDNA.158.1.S1_at | LOC713216 | similar to minor | Mmu.719 | XP_001110535 /// | 235.1732 | A | 562.5519 | P |
| | | histocompatibility antigen 13 | | XP_001110638 /// | | | | |
| | | isoform 3 | | XP_001110677 | | | | |
| MmugDNA.21170.1.S1_at | LOC713289 | similar to sarcosine | Mmu.14226 | XP_001099822 /// | 50.93322 | A | 157.4154 | P |
| | | dehydrogenase | | XP_001100018 /// | | | | |
| | | | | XP_001100105 | | | | |
| MmugDNA.17300.1.S1_s_at | LOC713324 | similar to erythrocyte membrane | Mmu.14133 | XP_001102639 | 68.31221 | A | 90.16785 | P |
| | | protein band 4.1 like 4B isoform 2 | | | | | | |
| MmuSTS.2766.1.S1_at | LOC713353 | Similar to unactive progesterone | Mmu.4022 | XP_001115328 /// | 77.942 | A | 233.5481 | P |
| | | receptor, 23 kD | | XP_001115356 /// | | | | |
| | | | | XP_001115374 /// | | | | |
| | | | | XP_001115388 | | | | |
| MmugDNA.2816.1.S1_s_at | LOC713680 | similar to Protein C9orf7 | Mmu.11422 | XP_001100751 /// | 178.9342 | A | 450.418 | P |
| | | | | XP_001100830 | | | | |
| MmugDNA.32391.1.S1_at | LOC713786 | similar to hepatocellular | Mmu.14691 | XP_001103245 | 155.9434 | A | 269.8064 | P |
| | | carcinoma-associated antigen | | | | | | |
| | | 112 | | | | | | |
| MmugDNA.37205.1.S1_at | LOC713800 | similar to plexin A2 | Mmu.15066 | XP_001110877 /// | 40.86835 | A | 95.29446 | P |
| | | | | XP_001110951 /// | | | | |
| | | | | XP_001110980 | | | | |
| MmugDNA.33340.1.S1_at | LOC713882 | similar to smooth muscle myosin | Mmu.14493 | XP_001109108 /// | 7.818644 | A | 114.8105 | P |
| | | heavy chain 11 isoform SM1A | | XP_001109220 /// | | | | |
| | | | | XP_001109323 /// | | | | |
| | | | | XP_001109374 /// | | | | |
| | | | | XP_001109425 /// | | | | |
| | | | | XP_001109463 | | | | |
| MmuSTS.3126.1.S1_at | LOC713917 | similar to neuronal guanine | Mmu.4419 | XP_001114604 | 89.85418 | A | 191.6622 | P |
| | | nucleotide exchange factor | | | | | | |
| MmuSTS.1097.1.S1_at | LOC714170 | similar to progestin and adipoQ | Mmu.11914 | XP_001111860 | 62.66206 | A | 124.0636 | P |
| | | receptor family member IX | | | | | | |
| MmugDNA.26038.1.S1_at | LOC714370 | Similar to decay accelerating | Mmu.1237 | XP_001111953 /// | 10.7786 | A | 155.9851 | P |
| | | factor for complement | | XP_001111994 /// | | | | |
| | | | | XP_001112031 /// | | | | |
| | | | | XP_001112064 /// | | | | |
| | | | | XP_001112134 /// | | | | |
| | | | | XP_001112168 /// | | | | |
| | | | | XP_001112209 /// | | | | |
| | | | | XP_001112247 /// | | | | |
| | | | | XP_001112287 /// | | | | |
| | | | | XP_001112321 | | | | |
| MmugDNA.18856.1.S1_at | LOC714749 | similar to Rsb-66 protein | Mmu.15699 | XP_001104349 /// | 73.02561 | A | 90.52779 | P |
| | | | | XP_001104431 /// | | | | |
| | | | | XP_001104509 /// | | | | |
| | | | | XP_001104586 | | | | |
| MmugDNA.30186.1.S1_at | LOC714958 | similar to Protein C19orf15 | Mmu.15418 | XP_001106958 /// | 63.63976 | A | 198.4253 | P |
| | | precursor | | XP_001107205 /// | | | | |
| | | | | XP_001107270 | | | | |
| MmugDNA.41904.1.S1_at | LOC715138 | similar to transmembrane | Mmu.11792 | XP_001107212 /// | 20.71842 | A | 109.4672 | P |
| | | protease, serine 2 | | XP_001107275 | | | | |
| MmugDNA.43404.1.S1_at | LOC715295 | similar to p53-induced protein | — | XP_001105448 | 182.3128 | A | 279.0873 | P |
| MmugDNA.13984.1.S1_at | LOC715330 | Similar to beta-site APP-cleaving | Mmu.1613 | XP_001107950 /// | 31.46918 | A | 188.1045 | P |
| | | enzyme 2 isoform C | | XP_001108061 | | | | |
| | | preproprotein | | | | | | |
| MmugDNA.23412.1.S1_x_at | LOC715553 | similar to Protein PP2447 | Mmu.16050 | XP_001111734 /// | 651.9257 | A | 749.3463 | P |
| | | | | XP_001111774 /// | | | | |
| | | | | XP_001111813 /// | | | | |
| | | | | XP_001111850 /// | | | | |
| | | | | XP_001111878 /// | | | | |
| | | | | XP_001111918 /// | | | | |
| | | | | XP_001111956 | | | | |
| MmuSTS.563.1.S1_at | LOC715706 | Similar to schlafen family | Mmu.11333 | XP_001114176 /// | 47.03052 | A | 86.69991 | P |
| | | member 13 | | XP_001114192 /// | | | | |
| | | | | XP_001114211 | | | | |
| MmugDNA.23724.1.S1_at | LOC715752 | similar to zinc finger protein 44 | Mmu.13185 | XP_001107149 /// | 177.6898 | A | 141.1652 | P |
| | | | | XP_001107204 /// | | | | |
| | | | | XP_001107269 | | | | |
| MmugDNA.30744.1.S1_s_at | LOC715853 | similar to interphase | Mmu.13940 | XP_001113603 | 221.8494 | A | 178.2616 | P |
| | | cyctoplasmic foci protein 45 | | | | | | |
| MmugDNA.11990.1.S1_at | LOC716153 | similar to myelin gene | Mmu.13074 | XP_001106720 | 32.29033 | A | 95.01642 | P |
| | | expression factor 2 | | | | | | |
| MmugDNA.36922.1.S1_at | LOC716153 | similar to myelin gene | Mmu.13074 | XP_001106720 | 71.02611 | A | 102.504 | P |
| | | expression factor 2 | | | | | | |
| MmugDNA.40257.1.S1_at | LOC716245 | similar to sphingosine-1- | Mmu.4100 | XP_001106861 | 122.3767 | A | 123.3001 | P |
| | | phosphate lyase 1 | | | | | | |
| MmugDNA.36455.1.S1_at | LOC716351 | similar to SEC22 vesicle | Mmu.14967 | XP_001115262 /// | 162.7943 | A | 119.6179 | P |
| | | trafficking protein homolog C | | XP_001115277 /// | | | | |
| | | isoform b | | XP_001115291 /// | | | | |
| | | | | XP_001115333 /// | | | | |
| | | | | XP_001115349 /// | | | | |
| | | | | XP_001115363 /// | | | | |
| | | | | XP_001115380 | | | | |
| MmuSTS.2718.1.S1_at | LOC716439 | similar to filamin A interacting | Mmu.12823 | XP_001113081 /// | 20.86039 | A | 142.4841 | P |
| | | protein 1 | | XP_001113105 /// | | | | |
| | | | | XP_001113142 /// | | | | |
| | | | | XP_001113175 | | | | |
| MmugDNA.18150.1.S1_at | LOC716504 | similar to Cdc42 effector protein 2 | — | XP_001113902 /// | 45.00316 | A | 317.3692 | P |
| | | | | XP_001113931 | | | | |
| MmugDNA.10063.1.S1_at | LOC716601 | similar to UPF3 regulator of | Mmu.13132 | XP_001107426 | 35.87898 | A | 178.3976 | P |
| | | nonsense transcripts homolog A | | | | | | |
| | | isoform hUpf3p | | | | | | |
| MmugDNA.2703.1.S1_at | LOC716903 | hypothetical protein LOC716903 | Mmu.15043 | XP_001115326 /// | 21.5953 | A | 134.5116 | P |
| | | | | XP_001115372 /// | | | | |
| | | | | XP_001115386 /// | | | | |
| | | | | XP_001115400 | | | | |
| Mmu.8945.1.S1_at | LOC717346 | similar to complement factor H | Mmu.4677 | XP_001111499 /// | 90.61079 | A | 197.2676 | P |
| | | isoform a precursor | | XP_001111535 /// | | | | |
| | | | | XP_001111875 | | | | |
| MmugDNA.17230.1.S1_at | LOC717350 | similar to ets homologous factor | Mmu.15764 | XP_001115436 /// | 66.78262 | A | 86.82366 | P |
| | | | | XP_001115449 /// | | | | |
| | | | | XP_001115455 /// | | | | |
| | | | | XP_001115469 /// | | | | |
| | | | | XP_001115487 /// | | | | |
| | | | | XP_001115502 /// | | | | |
| | | | | XP_001115517 | | | | |
| MmugDNA.20406.1.S1_at | LOC717474 | similar to GLE1-like, RNA export | Mmu.12903 | XP_001110762 /// | 182.8572 | A | 225.2607 | P |
| | | mediator isoform 1 | | XP_001110844 | | | | |
| MmugDNA.22554.1.S1_s_at | LOC717764 | similar to phospholipase C, beta | Mmu.11823 | XP_001115104 | 294.9439 | A | 463.7735 | P |
| | | 3 (phosphatidylinositol-specific) | | | | | | |
| MmugDNA.41138.1.S1_at | LOC717824 | similar to mago-nashi homolog | Mmu.2413 | XP_001115401 /// | 56.18679 | A | 182.9515 | P |
| | | | | XP_001115410 | | | | |
| MmugDNA.478.1.S1_at | LOC718703 | similar to TEA domain family | Mmu.15394 | XP_001114796 /// | 18.20921 | A | 131.8721 | P |
| | | member 2 | | XP_001114808 /// | | | | |
| | | | | XP_001114818 /// | | | | |
| | | | | XP_001114832 | | | | |
| MmugDNA.40598.1.S1_at | LOC719284 | similar to Duffy blood group | Mmu.10996 | XP_001117188 /// | 79.40461 | A | 157.7951 | P |
| | | | | XP_001117194 /// | | | | |
| | | | | XP_001117200 | | | | |
| MmugDNA.8485.1.S1_at | LOC719645 | similar to H326 | Mmu.153 | XP_001117421 /// | 55.48396 | A | 132.2252 | P |
| | | | | XP_001117432 /// | | | | |
| | | | | XP_001117435 /// | | | | |
| | | | | XP_001117439 /// | | | | |
| | | | | XP_001117441 /// | | | | |
| | | | | XP_001117445 /// | | | | |
| | | | | XP_001117452 /// | | | | |
| | | | | XP_001117454 /// | | | | |
| | | | | XP_001117459 /// | | | | |
| | | | | XP_001117461 /// | | | | |
| | | | | XP_001117464 /// | | | | |
| | | | | XP_001117465 /// | | | | |
| | | | | XP_001117470 /// | | | | |
| | | | | XP_001117473 | | | | |
| MmuSTS.2495.1.S1_at | LOC719814 | similar to zinc finger protein 577 | Mmu.15382 | XP_001116472 /// | 105.0435 | A | 133.1272 | P |
| | | | | XP_001116485 /// | | | | |
| | | | | XP_001116492 | | | | |
| MmugDNA.11286.1.S1_at | LOC720186 | similar to immunoglobulin | Mmu.11138 | XP_001115307 | 385.697 | A | 457.8605 | P |
| | | superfamily, member 8 | | | | | | |
| MmugDNA.1499.1.S1_at | LOC721262 | similar to Pigment epithelium- | Mmu.12605 | XP_001117361 | 90.36726 | A | 288.4651 | P |
| | | derived factor precursor (PEDF) | | | | | | |
| | | (EPC-1) | | | | | | |
| MmunewRS.138.1.S1_at | LOC721825 | similar to cAMP responsive | Mmu.11710 | XP_001118020 | 87.49419 | A | 146.52 | P |
| | | element binding protein 3-like 3 | | | | | | |
| MmugDNA.39283.1.S1_at | LOC721914 | similar to olfactory receptor, | Mmu.13643 | XP_001118110 | 76.79032 | A | 189.9431 | P |
| | | family 2, subfamily A, member 7 | | | | | | |
| Mmu.15041.1.S1_at | MAOA | monoamine oxidase A | Mmu.3283 | XP_001096840 | 63.99507 | A | 84.97249 | P |
| MmugDNA.21143.1.S1_at | MAOA | monoamine oxidase A | Mmu.3283 | XP_001096840 | 127.6487 | A | 588.567 | P |
| MmugDNA.25736.1.S1_at | MDGA2 | MAM domain containing | Mmu.16277 | XP_001097241 /// | 18.25484 | A | 68.81326 | P |
| | | glycosylphosphatidylinositol | | XP_001097344 /// | | | | |
| | | anchor 2 | | XP_001097446 /// | | | | |
| | | | | XP_001097544 /// | | | | |
| | | | | XP_001097646 | | | | |
| MmugDNA.32119.1.S1_at | ME1 | malic enzyme 1, NADP(+)- | Mmu.11722 | XP_001084298 /// | 8.007969 | A | 80.68948 | P |
| | | dependent, cytosolic | | XP_001084419 /// | | | | |
| | | | | XP_001084539 /// | | | | |
| | | | | XP_001084663 /// | | | | |
| | | | | XP_001084781 /// | | | | |
| | | | | XP_001084901 | | | | |
| MmugDNA.20648.1.S1_at | MFAP3 | microfibrillar-associated protein 3 | Mmu.1139 | XP_001102567 | 209.1429 | A | 166.2625 | P |
| MmugDNA.27696.1.S1_at | MMP26 | matrix metallopeptidase 26 | Mmu.3669 | XP_001112141 | 53.29844 | A | 76.93641 | P |
| MmugDNA.22230.1.S1_s_at | MMP28 | matrix metallopeptidase 28 | Mmu.3666 | XP_001114445 | 123.8551 | A | 161.2347 | P |
| MmugDNA.15929.1.S1_at | MRAS | muscle RAS oncogene homolog | Mmu.14559 | XP_001114261 /// | 44.73249 | A | 123.0301 | P |
| | | | | XP_001114274 /// | | | | |
| | | | | XP_001114299 /// | | | | |
| | | | | XP_001114317 | | | | |
| MmuSTS.4170.1.S1_at | MRAS | muscle RAS oncogene homolog | Mmu.14559 | XP_001114261 /// | 150.9189 | A | 182.644 | P |
| | | | | XP_001114274 /// | | | | |
| | | | | XP_001114299 /// | | | | |
| | | | | XP_001114317 | | | | |
| Mmu.14436.1.S1_at | MRC1 | mannose receptor, C type 1 | Mmu.1302 | XP_001091661 /// | 42.83043 | A | 113.4529 | P |
| | | | | XP_001091901 | | | | |
| MmugDNA.31263.1.S1_at | MRGPRX4 | MAS-related GPR, member X4 | Mmu.9516 | NP_001035708 /// | 18.23305 | A | 173.069 | P |
| | | | | XP_001086954 | | | | |
| MmugDNA.13215.1.S1_at | MYO5B | myosin VB | Mmu.15475 | XP_001090434 /// | 76.57364 | A | 138.2834 | P |
| | | | | XP_001090545 /// | | | | |
| | | | | XP_001090668 | | | | |
| MmugDNA.42474.1.S1_at | NCAM1 | neural cell adhesion molecule 1 | Mmu.11207 | XP_001082236 /// | 145.3389 | A | 147.3262 | P |
| | | | | XP_001082361 /// | | | | |
| | | | | XP_001083241 /// | | | | |
| | | | | XP_001083366 /// | | | | |
| | | | | XP_001083486 /// | | | | |
| | | | | XP_001083597 /// | | | | |
| | | | | XP_001083697 /// | | | | |
| | | | | XP_001083809 /// | | | | |
| | | | | XP_001083924 | | | | |
| MmugDNA.3439.1.S1_at | NEB | nebulin | Mmu.10774 | XP_001084585 | 4.838272 | A | 107.4847 | P |
| MmugDNA.10723.1.S1_at | NECAP2 | NECAP endocytosis associated 2 | Mmu.3233 | XP_001086937 /// | 142.5812 | A | 201.7808 | P |
| | | | | XP_001087060 | | | | |
| Mmu.6763.1.S1_at | NEU1 | sialidase 1 (lysosomal sialidase) | Mmu.1002 | XP_001113418 /// | 414.9135 | A | 679.2563 | P |
| | | | | XP_001113467 /// | | | | |
| | | | | XP_001113496 | | | | |
| MmugDNA.26632.1.S1_at | NMNAT3 | nicotinamide nucleotide | Mmu.14941 | XP_001113328 /// | 100.2686 | A | 285.6383 | P |
| | | adenylyltransferase 3 | | XP_001113354 /// | | | | |
| | | | | XP_001113387 /// | | | | |
| | | | | XP_001113417 /// | | | | |
| | | | | XP_001113442 | | | | |
| MmugDNA.28543.1.S1_at | NR4A2 | Nuclear receptor subfamily 4, | Mmu.11507 | XP_001085438 /// | 67.00845 | A | 110.9794 | P |
| | | group A, member 2 | | XP_001085797 /// | | | | |
| | | | | XP_001085905 /// | | | | |
| | | | | XP_001086011 /// | | | | |
| | | | | XP_001086128 /// | | | | |
| | | | | XP_001086234 /// | | | | |
| | | | | XP_001086356 /// | | | | |
| | | | | XP_001086593 /// | | | | |
| | | | | XP_001086719 | | | | |
| MmugDNA.38502.1.S1_at | NRXN1 | neurexin 1 | Mmu.4429 | XP_001114208 | 18.97834 | A | 126.3031 | P |
| MmuSTS.4647.1.S1_at | NTNG2 | netrin G2 | Mmu.15124 | XP_001082464 /// | 3.059485 | A | 101.9615 | P |
| | | | | XP_001082588 /// | | | | |
| | | | | XP_001082730 /// | | | | |
| | | | | XP_001082854 | | | | |
| Mmu.2344.1.S1_at | NXPH4 | neurexophilin 4 | Mmu.3606 | XP_001099977 | 143.7606 | A | 166.9342 | P |
| Mmu.14635.1.S1_s_at | P11 | placental protein 11 | Mmu.1192 | XP_001097398 /// | 3.029125 | A | 102.4633 | P |
| | | | | XP_001097492 /// | | | | |
| | | | | XP_001097588 /// | | | | |
| | | | | XP_001097705 /// | | | | |
| | | | | XP_001097799 | | | | |
| MmuSTS.1070.1.S1_at | PAH | phenylalanine hydroxylase | Mmu.11888 | XP_001094859 | 24.82761 | A | 66.58179 | P |
| MmugDNA.3732.1.S1_at | PCGF2 | polycomb group ring finger 2 | Mmu.17122 | XP_001083603 /// | 7.621283 | A | 94.5854 | P |
| | | | | XP_001083705 /// | | | | |
| | | | | XP_001083817 | | | | |
| MmugDNA.30313.1.S1_at | PFKFB1 | 6-phosphofructo-2- | Mmu.15267 | XP_001091669 /// | 76.07756 | A | 92.32727 | P |
| | | kinase/fructose-2,6- | | XP_001091781 /// | | | | |
| | | biphosphatase 1 | | XP_001091907 | | | | |
| MmuSTS.2247.1.S1_at | PLG | plasminogen | Mmu.3024 | NP_001036540 /// | 48.69508 | A | 71.0714 | P |
| | | | | XP_001099441 | | | | |
| MmugDNA.5295.1.S1_s_at | PNLIPRP2 | pancreatic lipase-related protein 2 | Mmu.3256 | XP_001095293 | 9.050271 | A | 134.0069 | P |
| MmugDNA.15066.1.S1_at | PPM1F | protein phosphatase 1F | Mmu.13045 | XP_001089477 | 112.9894 | A | 349.5799 | P |
| MmugDNA.21620.1.S1_at | PPP5C | protein phosphatase 5, catalytic | Mmu.11271 | XP_001111636 /// | 604.6081 | A | 644.2534 | P |
| | | subunit | | XP_001111674 /// | | | | |
| | | | | XP_001111714 /// | | | | |
| | | | | XP_001111749 | | | | |
| MmugDNA.39032.1.S1_at | PRDX6 | Peroxiredoxin 6 | Mmu.4290 | XP_001101473 | 34.28012 | A | 142.5283 | P |
| MmugDNA.31823.1.S1_at | PRKACB | protein kinase, cAMP-dependent, | Mmu.1940 | XP_001105459 /// | 145.2954 | A | 122.2689 | P |
| | | catalytic, beta | | XP_001105534 /// | | | | |
| | | | | XP_001105605 /// | | | | |
| | | | | XP_001105676 /// | | | | |
| | | | | XP_001105747 /// | | | | |
| | | | | XP_001105803 /// | | | | |
| | | | | XP_001105875 /// | | | | |
| | | | | XP_001105953 /// | | | | |
| | | | | XP_001106026 /// | | | | |
| | | | | XP_001106097 /// | | | | |
| | | | | XP_001106164 | | | | |
| MmugDNA.36754.1.S1_at | PRR3 | proline rich 3 | Mmu.12623 | XP_001100535 | 92.55525 | A | 104.1183 | P |
| Mmu.12.1.S1_at | RASSF8 | Ras association (RalGDS/AF-6) | Mmu.13028 | XP_001102028 /// | 31.62563 | A | 82.27078 | P |
| | | domain family (N-terminal) | | XP_001102115 /// | | | | |
| | | member 8 | | XP_001102201 | | | | |
| MmuSTS.2090.1.S1_at | RBM11 | RNA binding motif protein 11 | Mmu.12892 | XP_001083260 | 81.05433 | A | 85.9953 | P |
| MmugDNA.7677.1.S1_at | RBM34 | RNA binding motif protein 34 | Mmu.12653 | XP_001102020 /// | 139.6196 | A | 60.82475 | P |
| | | | | XP_001102111 | | | | |
| MmugDNA.17401.1.S1_at | SEC24A | SEC24 family, member A (S. cerevisiae) | Mmu.13387 | XP_001109569 /// | 32.50195 | A | 160.1317 | P |
| | | | | XP_001109620 | | | | |
| MmugDNA.18877.1.S1_at | SF3B14 | similar to splicing factor 3B, 14 kDa | Mmu.942 | XP_001098393 | 96.04313 | A | 144.1895 | P |
| | | subunit | | | | | | |
| MmuSTS.3315.1.S1_at | SH3GLB2 | SH3-domain GRB2-like | Mmu.17131 | XP_001109262 | 415.9608 | A | 360.3215 | P |
| | | endophilin B2 | | | | | | |
| MmugDNA.28147.1.S1_at | SLC13A2 | solute carrier family 13 (sodium- | Mmu.11776 | XP_001106342 | 83.45712 | A | 172.6272 | P |
| | | dependent dicarboxylate | | | | | | |
| | | transporter), member 2 | | | | | | |
| MmugDNA.20608.1.S1_at | SLC25A11 | Solute carrier family 25 | Mmu.11522 | XP_001097307 /// | 159.8107 | A | 199.7657 | P |
| | | (mitochondrial carrier; | | XP_001097413 /// | | | | |
| | | oxoglutarate carrier), member | | XP_001097514 | | | | |
| | | 11 | | | | | | |
| MmugDNA.34347.1.S1_s_at | SLC25A13 | solute carrier family 25, member | Mmu.11416 | XP_001088340 | 83.63106 | A | 130.7075 | P |
| | | 13 (citrin) | | | | | | |
| MmugDNA.43541.1.S1_at | SLC25A17 | Solute carrier family 25 | Mmu.4154 | XP_001102293 | 135.3007 | A | 217.9323 | P |
| | | (mitochondrial carrier; | | | | | | |
| | | peroxisomal membrane protein, | | | | | | |
| | | 34 kDa), member 17 | | | | | | |
| MmugDNA.26126.1.S1_at | SOX2 | SRY (sex determining region Y)- | Mmu.10959 | NP_001136412 /// | 25.83881 | A | 100.8555 | P |
| | | box 2 | | XP_001106891 | | | | |
| MmuAffx.1.1.S1_at | SRY | sex determining region Y | Mmu.3544 | NP_001028008 | 56.02947 | A | 91.99885 | P |
| MmugDNA.15062.1.S1_at | SSBP1 | Single-stranded DNA binding | Mmu.4481 | XP_001084058 /// | 129.9467 | A | 146.7887 | P |
| | | protein 1 | | XP_001084179 /// | | | | |
| | | | | XP_001084295 /// | | | | |
| | | | | XP_001084414 /// | | | | |
| | | | | XP_001084536 | | | | |
| MmugDNA.17880.1.S1_at | ST3GAL1 | ST3 beta-galactoside alpha-2,3- | Mmu.12091 | XP_001088980 /// | 127.049 | A | 295.4928 | P |
| | | sialyltransferase 1 | | XP_001089209 /// | | | | |
| | | | | XP_001089316 /// | | | | |
| | | | | XP_001089439 /// | | | | |
| | | | | XP_001089550 | | | | |
| MmugDNA.26812.1.S1_at | ST8SIA4 | ST8 alpha-N-acetyl-neuraminide | Mmu.13440 | XP_001097339 | 135.5056 | A | 84.99597 | P |
| | | alpha-2,8-sialyltransferase 4 | | | | | | |
| MmugDNA.27466.1.S1_at | TAF11 | similar to Transcription initiation | Mmu.9248 | — | 94.84025 | A | 93.78352 | P |
| | | factor TFIID subunit 11 | | | | | | |
| | | (Transcription initiation factor | | | | | | |
| | | TFIID 28 kDa subunit) | | | | | | |
| | | (TAF(II)28) (TAFII-28) | | | | | | |
| | | (TAFII28) (TFIID subunit p30- | | | | | | |
| | | beta) | | | | | | |
| MmugDNA.17390.1.S1_at | TAR1 | trace amine receptor 1 | Mmu.16488 | NP_001074234 /// | 43.22684 | A | 83.6374 | P |
| | | | | XP_001102243 | | | | |
| MmugDNA.27658.1.S1_at | TCEA2 | transcription elongation factor A | Mmu.229 | XP_001082344 /// | 73.96481 | A | 92.49038 | P |
| | | (SII), 2 | | XP_001082860 /// | | | | |
| | | | | XP_001082986 /// | | | | |
| | | | | XP_001083100 /// | | | | |
| | | | | XP_001083220 /// | | | | |
| | | | | XP_001083344 | | | | |
| MmugDNA.8481.1.S1_at | TES | Testis derived transcript (3 LIM | Mmu.4028 | XP_001095286 | 52.37125 | A | 122.8702 | P |
| | | domains) | | | | | | |
| MmugDNA.9561.1.S1_at | TIMP-2 | tissue inhibitor of matrix | Mmu.1702 | XP_001106657 | 369.0166 | A | 888.6364 | P |
| | | metalloproteinase-2 | | | | | | |
| MmugDNA.502.1.S1_at | TIRAP | toll-interleukin 1 receptor (TIR) | Mmu.17493 | NP_001123907 /// | 205.38 | A | 386.3123 | P |
| | | domain containing adaptor | | XP_001112918 /// | | | | |
| | | protein | | XP_001112950 | | | | |
| MmugDNA.31314.1.S1_at | TMLHE | trimethyllysine hydroxylase, | Mmu.15229 | XP_001099467 /// | 183.9673 | A | 362.3624 | P |
| | | epsilon | | XP_001099575 /// | | | | |
| | | | | XP_001099674 | | | | |
| MmugDNA.86.1.S1_at | TRHDE | thyrotropin-releasing hormone | Mmu.130 | XP_001109391 | 53.7547 | A | 144.0242 | P |
| | | degrading enzyme | | | | | | |
| MmugDNA.13477.1.S1_at | TSHR | thyroid stimulating hormone | Mmu.3677 | XP_001104765 /// | 3.757645 | A | 64.06719 | P |
| | | receptor | | XP_001104839 | | | | |
| Mmu.5321.1.S1_at | TTC12 | tetratricopeptide repeat domain | Mmu.13011 | XP_001084384 /// | 108.8476 | A | 237.5948 | P |
| | | 12 | | XP_001084508 /// | | | | |
| | | | | XP_001084630 /// | | | | |
| | | | | XP_001084748 /// | | | | |
| | | | | XP_001084868 /// | | | | |
| | | | | XP_001084979 | | | | |
| MmugDNA.39789.1.S1_at | TTC7A | tetratricopeptide repeat domain | Mmu.15863 | XP_001113285 /// | 151.4908 | A | 248.7907 | P |
| | | 7A | | XP_001113315 /// | | | | |
| | | | | XP_001113346 | | | | |
| MmugDNA.33980.1.S1_at | TTLL4 | tubulin tyrosine ligase-like | Mmu.15909 | XP_001094019 /// | 273.2361 | A | 328.0809 | P |
| | | family, member 4 | | XP_001094615 /// | | | | |
| | | | | XP_001094742 /// | | | | |
| | | | | XP_001094864 | | | | |
| MmugDNA.29140.1.S1_s_at | UGCGL1 | UDP-glucose ceramide | Mmu.12163 | XP_001091001 /// | 404.7524 | A | 510.9632 | P |
| | | glucosyltransferase-like 1 | | XP_001091120 /// | | | | |
| | | | | XP_001091250 /// | | | | |
| | | | | XP_001091373 /// | | | | |
| | | | | XP_001091494 | | | | |
| MmugDNA.13424.1.S1_at | USP21 | Ubiquitin specific peptidase 21 | Mmu.11289 | XP_001117752 /// | 43.01239 | A | 94.55185 | P |
| | | | | XP_001117766 /// | | | | |
| | | | | XP_001117768 /// | | | | |
| | | | | XP_001117772 /// | | | | |
| | | | | XP_001117777 /// | | | | |
| | | | | XP_001117779 /// | | | | |
| | | | | XP_001117782 /// | | | | |
| | | | | XP_001117786 | | | | |
| MmugDNA.20532.1.S1_at | WDR77 | WD repeat domain 77 | Mmu.4390 | XP_001105103 | 179.0304 | A | 536.4213 | P |
| MmugDNA.33665.1.S1_at | WDR8 | WD repeat domain 8 | Mmu.15190 | XP_001083578 /// | 178.8616 | A | 306.8982 | P |
| | | | | XP_001083787 /// | | | | |
| | | | | XP_001083904 /// | | | | |
| | | | | XP_001084011 | | | | |
| MmugDNA.26867.1.S1_at | XG | Xg blood group | Mmu.11499 | XP_001086077 /// | 29.58194 | A | 136.8591 | P |
| | | | | XP_001086193 | | | | |
| MmugDNA.19387.1.S1_at | ZDHHC3 | zinc finger, DHHC-type | Mmu.11811 | XP_001114690 /// | 387.6217 | A | 714.7256 | P |
| | | containing 3 | | XP_001114737 /// | | | | |
| | | | | XP_001114753 | | | | |
| MmugDNA.42763.1.S1_at | ZFAND5 | zinc finger, AN1-type domain 5 | Mmu.1639 | XP_001096078 /// | 2.943856 | A | 138.3536 | P |
| | | | | XP_001096191 /// | | | | |
| | | | | XP_001096312 /// | | | | |
| | | | | XP_001096433 /// | | | | |
| | | | | XP_001096545 | | | | |
| MmugDNA.29066.1.S1_at | ZNRD1 | zinc ribbon domain containing 1 | Mmu.17470 | NP_001108417 /// | 87.50885 | A | 120.7655 | P |
| | | | | XP_001105355 /// | | | | |
| | | | | XP_001105431 | | | | |

TABLE 4
Immediate early (1-hr post stroke) biomarkers identified by RNA analysis.

| Gene identifier | Gene name | Amino Acid Sequence |
|---|---|---|
| NP_006546.1 | synaptobrevin homolog YKT6 | SEQ ID NO: 1 |
| NP_001776.1 | cytidine deaminase | SEQ ID NO: 2 |
| EAW60338.1 | hypothetical protein FLJ20232 | SEQ ID NO: 3 |
| EAW67321.1 | centrosomal protein 164 kDa | SEQ ID NO: 4 |
| EAW71459.1 | WD repeat domain 8 | SEQ ID NO: 5 |
| EAW85140.1 | zinc finger CCCH-type containing 7A | SEQ ID NO: 6 |
| EAW89539.1 | TIMP metallopeptidase inhibitor 2 | SEQ ID NO: 7 |
| EAW96888.1 | zinc finger CCCH-type containing 10 | SEQ ID NO: 8 |
| BAB47442.1 | KIAA1813 protein | SEQ ID NO: 9 |
| BAB59061.1 | Pex1p-634del690 | SEQ ID NO: 10 |
| AAH09411.1 | WDR77 protein | SEQ ID NO: 11 |
| NP_001078929.1 | catenin delta-1 isoform 1A | SEQ ID NO: 12 |
| NP_071426.1 | leucine-rich repeat-containing protein 4 precursor | SEQ ID NO: 13 |
| NP_001099038.1 | kinesin-like protein KIF13A isoform d | SEQ ID NO: 14 |
| BAG53572.1 | unnamed protein product | SEQ ID NO: 15 |
| BAG54027.1 | unnamed protein product | SEQ ID NO: 16 |
| BAG57307.1 | unnamed protein product | SEQ ID NO: 17 |
| BAG63947.1 | unnamed protein product | SEQ ID NO: 18 |
| BAG64130.1 | unnamed protein product | SEQ ID NO: 19 |
| BAG58900.1 | unnamed protein product | SEQ ID NO: 20 |
| NP_148978.2 | cyclin-dependent kinase 16 isoform 2 | SEQ ID NO: 21 |
| NP_001135741.1 | vitamin K-dependent gamma-carboxylase isoform 2 | SEQ ID NO: 22 |
| NP_631981.2 | nucleolar protein 6 gamma isoform | SEQ ID NO: 23 |
| AAM94901.1 | AF311906_1 LIR-D1 precursor | SEQ ID NO: 24 |
| AAH35404.1 | PTK2 protein | SEQ ID NO: 25 |
| NP_733840.1 | NLR family member X1 isoform 2 | SEQ ID NO: 26 |
| AAN75525.1 | Rap1-interacting adaptor molecule | SEQ ID NO: 27 |
| NP_001171187.1 | Krueppel-like factor 11 isoform b | SEQ ID NO: 28 |
| NP_001171726.1 | trimethyllysine dioxygenase, mitochondrial isoform 2 | SEQ ID NO: 29 |
| | precursor | |
| CAD91343.1 | cytochrome P450 | SEQ ID NO: 30 |
| NP_115652.2 | rhomboid domain-containing protein 1 | SEQ ID NO: 31 |
| NP_079480.2 | traB domain-containing protein | SEQ ID NO: 32 |
| NP_954574.1 | histone-lysine N-methyltransferase setd3 isoform b | SEQ ID NO: 33 |
| NP_000112.1 | erythropoietin receptor precursor | SEQ ID NO: 34 |
| NP_001453.1 | N-formyl peptide receptor 2 | SEQ ID NO: 35 |
| NP_002420.1 | matrix metalloproteinase-19 isoform rasi-1 preproprotein | SEQ ID NO: 36 |
| NP_003098.1 | transcription factor SOX-4 | SEQ ID NO: 37 |
| NP_003134.1 | single-stranded DNA-binding protein, mitochondrial | SEQ ID NO: 38 |
| | precursor | |
| AAH16977.2 | PBX3 protein | SEQ ID NO: 39 |
| NP_001973.2 | receptor tyrosine-protein kinase erbB-3 isoform 1 precursor | SEQ ID NO: 40 |
| CAH72013.1 | RIO kinase 1 (yeast) | SEQ ID NO: 41 |
| 2IRT | A Chain, Initial Crystallographic Analyses Of A | SEQ ID NO: 42 |
| | Recombinant Interleukin-1 Receptor Antagonist Protein | |
| BAA91249.1 | unnamed protein product | SEQ ID NO: 43 |
| NP_000447.2 | sulfite oxidase, mitochondrial precursor | SEQ ID NO: 44 |
| CAB86601.1 | xce | SEQ ID NO: 45 |
| NP_057692.1 | armadillo repeat-containing X-linked protein 1 | SEQ ID NO: 46 |
| NP_057696.2 | mitoferrin-1 | SEQ ID NO: 47 |
| NP_064530.1 | endophilin-B2 | SEQ ID NO: 48 |

TABLE 5
Early biomarkers (2-hr post stroke) biomarkers identified by RNA analysis.

| Gene identifier | Gene name | Amino Acid Sequence |
|---|---|---|
| NP_060288.3 | WD repeat-containing protein WRAP73 | SEQ ID NO: 49 |
| NP_001776.1 | cytidine deaminase | SEQ ID NO: 50 |
| NP_001128651.1 | palmitoyltransferase ZDHHC3 isoform 1 | SEQ ID NO: 51 |
| NP_004197.1 | vesicle-trafficking protein SEC22c isoform b | SEQ ID NO: 52 |
| NP_001001890.1 | runt-related transcription factor 1 isoform AML1b | SEQ ID NO: 53 |
| NP_000457.1 | peroxisome biogenesis factor 1 | SEQ ID NO: 54 |
| NP_071426.1 | leucine-rich repeat-containing protein 4 precursor | SEQ ID NO: 13 |
| NP_003134.1 | single-stranded DNA-binding protein, mitochondrial | SEQ ID NO: 55 |
| NP_001003700.1 | ras-responsive element-binding protein 1 isoform 3 | SEQ ID NO: 56 |
| NP_001099038.1 | kinesin-like protein KIF13A isoform d | SEQ ID NO: 57 |
| NP_877434.2 | E3 ubiquitin-protein ligase RNF144B | SEQ ID NO: 58 |
| NP_031381.2 | heat shock protein HSP 90-beta | SEQ ID NO: 59 |
| NP_001011516.1 | PDZ and LIM domain protein 5 isoform e | SEQ ID NO: 60 |
| NP_000815.1 | glycine receptor subunit beta isoform A precursor | SEQ ID NO: 61 |
| NP_060516.2 | angiogenic factor with G patch and FHA domains 1 | SEQ ID NO: 62 |
| NP_057113.1 | dehydrogenase/reductase SDR family member 7 precursor | SEQ ID NO: 63 |
| NP_954574.1 | histone-lysine N-methyltransferase setd3 isoform b | SEQ ID NO: 33 |
| NP_057696.2 | mitoferrin-1 | SEQ ID NO: 47 |
| NP_039258.1 | pro-neuregulin-1, membrane-bound isoform isoform HRG- | SEQ ID NO: 64 |
| | alpha | |
| NP_005598.3 | focal adhesion kinase 1 isoform b | SEQ ID NO: 65 |

TABLE 6
Late biomarkers (24-hr post stroke) biomarkers identified by RNA analysis.

| Gene identifier | Gene name | Amino Acid Sequence |
|---|---|---|
| NP_005944.1 | metallothionein-2 | SEQ ID NO: 66 |
| NP_057692.1 | armadillo repeat-containing X-linked protein 1 | SEQ ID NO: 46 |
| NP_001128250.1 | pre-B-cell leukemia transcription factor 3 isoform 2 | SEQ ID NO: 67 |
| NP_000031.1 | apolipoprotein C-III precursor | SEQ ID NO: 68 |
| NP_057696.2 | mitoferrin-1 | SEQ ID NO: 47 |
| NP_079480.2 | traB domain-containing protein | SEQ ID NO: 69 |
| NP_001128651.1 | palmitoyltransferase ZDHHC3 isoform 1 | SEQ ID NO: 70 |
| NP_000425.1 | sialidase-1 precursor | SEQ ID NO: 71 |
| NP_006238.1 | serine/threonine-protein phosphatase 5 isoform 1 | SEQ ID NO: 72 |
| NP_631981.2 | nucleolar protein 6 gamma isoform | SEQ ID NO: 73 |
| NP_057427.3 | centromere protein F | SEQ ID NO: 74 |
| \|NP_543153.1 | sodium-dependent phosphate transport protein 2C | SEQ ID NO: 75 |
| NP_001171187.1 | Krueppel-like factor 11 isoform b | SEQ ID NO: 28 |
| NP_000231.1 | amine oxidase | SEQ ID NO: 76 |
| NP_004160.3 | serine hydroxymethyltransferase, cytosolic isoform 1 | SEQ ID NO: 77 |
| NP_848695.1 | minor histocompatibility antigen H13 isoform 2 | SEQ ID NO: 78 |
| NP_056973.2 | mediator of RNA polymerase II transcription subunit 15 | SEQ ID NO: 79 |
| | isoform b | |
| NP_077007.1 | methylosome protein 50 | SEQ ID NO: 80 |
| NP_064505.1 | UDP-glucose:glycoprotein glucosyltransferase 1 precursor | SEQ ID NO: 81 |
| NP_001001890.1 | runt-related transcription factor 1 isoform AML1b | SEQ ID NO: 82 |
| NP_005598.3 | focal adhesion kinase 1 isoform b | SEQ ID NO: 83 |
| NP_061916.3 | amyloid beta A4 precursor protein-binding family B member | SEQ ID NO: 84 |
| | 1-interacting protein | |
| NP_060516.2 | angiogenic factor with G patch and FHA domains 1 | SEQ ID NO: 62 |
| NP_000923.1 | 1-phosphatidylinositol-4,5-bisphosphate phosphodiesterase | SEQ ID NO: 85 |
| | beta-3 isoform 1 | |
| NP_071426.1 | leucine-rich repeat-containing protein 4 precursor | SEQ ID NO: 13 |
| NP_443100.1 | immunoglobulin superfamily member 8 | SEQ ID NO: 86 |
| NP_006546.1 | synaptobrevin homolog YKT6 | SEQ ID NO: 1 |
| NP_001129247.1 | calcium channel flower homolog isoform b | SEQ ID NO: 87 |
| NP_071906.1 | 5-azacytidine-induced protein 2 isoform a | SEQ ID NO: 88 |
| NP_954574.1 | histone-lysine N-methyltransferase setd3 isoform b | SEQ ID NO: 33 |
| NP_001135741.1 | vitamin K-dependent gamma-carboxylase isoform 2 | SEQ ID NO: 22 |
| NP_001776.1 | cytidine deaminase | SEQ ID NO: 2 |
| \|NP_031381.2 | heat shock protein HSP 90-beta | SEQ ID NO: 59 |
| NP_054872.2 | zinc finger CCCH domain-containing protein 7A | SEQ ID NO: 6 |
| NP_115652.2 | rhomboid domain-containing protein 1 | SEQ ID NO: 31 |
| NP_001034750.1 | toll/interleukin-1 receptor domain-containing adapter protein | SEQ ID NO: 89 |
| | isoform a | |
| NP_055047.2 | vesicle-associated membrane protein 2 | SEQ ID NO: 90 |
| NP_000568.1 | interleukin-1 receptor antagonist protein isoform 3 | SEQ ID NO: 91 |
| NP_039258.1 | pro-neuregulin-1, membrane-bound isoform isoform HRG- | SEQ ID NO: 64 |
| | alpha | |
| NP_060288.3 | WD repeat-containing protein WRAP73 | SEQ ID NO: 49 |
| NP_598012.1 | decorin isoform c precursor | SEQ ID NO: 92 |
| NP_001973.2 | receptor tyrosine-protein kinase erbB-3 isoform 1 precursor | SEQ ID NO: 40 |
| NP_001166237.1 | forkhead box protein P2 isoform V | SEQ ID NO: 93 |
| NP_000457.1 | peroxisome biogenesis factor 1 | SEQ ID NO: 54 |
| NP_000112.1 | erythropoietin receptor precursor | SEQ ID NO: 34 |
| NP_001078929.1 | catenin delta-1 isoform 1A | SEQ ID NO: 12 |
| \|NP_001176.1 | zinc-alpha-2-glycoprotein precursor | SEQ ID NO: 94 |
| NP_001171597.1 | immunoglobulin lambda-like polypeptide 5 | SEQ ID NO: 95 |
| NP_077278.1 | matrix metalloproteinase-28 isoform 1 preproprotein | SEQ ID NO: 96 |

TABLE 7
Proteins differentially expressed in the NHP model following MCAO

| | | | pre | |
|---|---|---|---|---|
| 1-HR | Sequence ID | Sequence Name | Occlusion | 1 h |
| 47 | XP_001112525 | PREDICTED: similar to CD163 antigen isoform a [Macaca mulatta] | 0 | 12 |
| 146 | XP_001112106 | PREDICTED: similar to thyroid hormone receptor associated protein 2 [Macaca mulatta] | 0 | 5 |
| 145 | XP_001083301 | PREDICTED: cytochrome b-245, beta polypeptide (chronic granulomatous disease) isoform 1 [Macaca | 0 | 4 |
| | | mulatta] | | |
| 104 | XP_001083320 | PREDICTED: similar to protein kinase C, delta [Macaca mulatta] | 0 | 3 |
| 51 | XP_001090238 | PREDICTED: pyruvate kinase, muscle isoform 1 [Macaca mulatta] | 0 | 3 |
| 139 | XP_001111401 | PREDICTED: golgi autoantigen, golgin subfamily b, macrogolgin (with transmembrane signal), 1 | 0 | 3 |
| | | isoform 1 [Macaca mulatta] | | |
| 50 | NP_001038203 | pancreatic ribonuclease [Macaca mulatta] | 0 | 3 |
| 184 | XP_001117349 | PREDICTED: similar to alpha-2-plasmin inhibitor [Macaca mulatta] | 0 | 3 |
| 172 | XP_001104736 | PREDICTED: general transcription factor IIIC, polypeptide 4, 90 kDa [Macaca mulatta] | 0 | 3 |
| | | | pre | |
| 2-HR | Sequence ID | Sequence Name | Occlusion | 2 h |
| 47 | XP_001112525 | PREDICTED: similar to CD163 antigen isoform a [Macaca mulatta] | 0 | 6 |
| 150 | XP_001082953 | PREDICTED: similar to alpha 2 type IX collagen [Macaca mulatta] | 0 | 4 |
| 42 | XP_001115663 | PREDICTED: similar to AKT1 substrate 1 (proline-rich) isoform 1 [Macaca mulatta] | 0 | 4 |
| 104 | XP_001083320 | PREDICTED: similar to protein kinase C, delta [Macaca mulatta] | 0 | 3 |
| 50 | NP_001038203 | pancreatic ribonuclease [Macaca mulatta] | 0 | 3 |
| 5 | XP_001111499 | PREDICTED: similar to complement factor H isoform a precursor isoform 1 [Macaca mulatta] | 0 | 3 |
| 184 | XP_001117349 | PREDICTED: similar to alpha-2-plasmin inhibitor [Macaca mulatta] | 0 | 2 |
| 87 | XP_001096042 | PREDICTED: similar to serine palmitoyltransferase, long chain base subunit 2 [Macaca mulatta] | 0 | 2 |
| 142 | XP_001111185 | PREDICTED: transforming growth factor, beta-induced, 68 kDa isoform 1 [Macaca mulatta] | 0 | 2 |
| 17 | XP_001090341 | PREDICTED: similar to WD repeat domain 19 [Macaca mulatta] | 0 | 2 |
| 117 | XP_001088176 | PREDICTED: similar to CG32699-PA [Macaca mulatta] | 0 | 2 |
| 161 | XP_001084230 | PREDICTED: similar to chemokine-like factor superfamily 3 isoform a [Macaca mulatta] | 0 | 2 |
| 128 | XP_001114075 | PREDICTED: similar to T-cell immunomodulatory protein [Macaca mulatta] | 0 | 2 |
| 129 | XP_001113081 | PREDICTED: similar to filamin A interacting protein 1 isoform 1 [Macaca mulatta] | 0 | 2 |
| 137 | XP_001109018 | PREDICTED: zinc finger protein 443 [Macaca mulatta] | 0 | 2 |
| 26 | XP_001083469 | PREDICTED: similar to apolipoprotein L2 [Macaca mulatta] | 0 | 2 |
| 32 | XP_001086062 | PREDICTED: similar to MADS box transcription enhancer factor 2, polypeptide C (myocyte enhancer | 0 | 2 |
| | | factor 2C) isoform 1 [Macaca mulatta] | | |
| 49 | XP_001105319 | PREDICTED: similar to phosphorylated CTD interacting factor 1 isoform 1 [Macaca mulatta] | 0 | 2 |
| 59 | XP_001109556 | PREDICTED: delta-tubulin [Macaca mulatta] | 0 | 2 |
| 124 | XP_001082131 | PREDICTED: similar to receptor interacting protein 140 [Macaca mulatta] | 0 | 2 |
| 166 | XP_001088368 | PREDICTED: carbonic anhydrase IX isoform 1 [Macaca mulatta] | 0 | 2 |
| 24- | | | pre | |
| HR | Sequence ID | Sequence Name | Occlusion | 24 h |
| 104 | XP_001083320 | PREDICTED: similar to protein kinase C, delta [Macaca mulatta] | 0 | 12 |
| 134 | XP_001104018 | PREDICTED: similar to NOD9 protein isoform 2 isoform 1 [Macaca mulatta] | 0 | 6 |
| 109 | XP_001095858 | PREDICTED: aspartyl-tRNA synthetase [Macaca mulatta] | 0 | 4 |
| 136 | XP_001114645 | PREDICTED: RAS p21 protein activator 4 [Macaca mulatta] | 0 | 4 |
| 161 | XP_001084230 | PREDICTED: similar to chemokine-like factor superfamily 3 isoform a [Macaca mulatta] | 0 | 4 |
| 51 | XP_001090238 | PREDICTED: pyruvate kinase, muscle isoform 1 [Macaca mulatta] | 0 | 3 |
| 58 | NP_001107821 | NLR family, pyrin domain containing 1 [Macaca mulatta] | 0 | 3 |
| 78 | XP_001089332 | PREDICTED: similar to Proteasome subunit alpha type 1 (Proteasome component C2) (Macropain | 0 | 3 |
| | | subunit C2) (Multicatalytic endopeptidase complex subunit C2) (Proteasome nu chain) (30 kDa | | |
| | | prosomal protein) (PROS-30) [Macaca mulatta] | | |
| 95 | XP_001102929 | PREDICTED: chitotriosidase isoform 1 [Macaca mulatta] | 0 | 3 |
| 116 | XP_001098308 | PREDICTED: L-plastin isoform 1 [Macaca mulatta] | 0 | 3 |
| 139 | XP_001111401 | PREDICTED: golgi autoantigen, golgin subfamily b, macrogolgin (with transmembrane signal), 1 | 0 | 3 |
| | | isoform 1 [Macaca mulatta] | | |
| 150 | XP_001082953 | PREDICTED: similar to alpha 2 type IX collagen [Macaca mulatta] | 0 | 3 |
| 17 | XP_001090341 | PREDICTED: similar to WD repeat domain 19 [Macaca mulatta] | 0 | 2 |
| 18 | XP_001093056 | PREDICTED: similar to solute carrier family 24 member 4 isoform 1 precursor isoform 1 [Macaca | 0 | 2 |
| | | mulatta] | | |
| 36 | XP_001097844 | PREDICTED: similar to Histidine acid phosphatase domain containing 1 isoform 1 [Macaca mulatta] | 0 | 2 |
| 38 | XP_001104918 | PREDICTED: similar to Transcription factor E3 isoform 1 [Macaca mulatta] | 0 | 2 |
| 50 | NP_001038203 | pancreatic ribonuclease [Macaca mulatta] | 0 | 2 |
| 57 | XP_001100504 | PREDICTED: similar to PDZ domain-containing guanine nucleotide exchange factor I isoform 1 | 0 | 2 |
| | | [Macaca mulatta] | | |
| 62 | XP_001116691 | PREDICTED: suppressin [Macaca mulatta] | 0 | 2 |
| 63 | XP_001085867 | PREDICTED: similar to cofactor required for Sp1 transcriptional activation, subunit 2, 150 kDa [Macaca | 0 | 2 |
| | | mulatta] | | |
| 75 | XP_001117192 | PREDICTED: similar to thyroid hormone receptor-associated protein 5 [Macaca mulatta] | 0 | 2 |
| 80 | XP_001092767 | PREDICTED: 2-deoxyribose-5-phosphate aldolase homolog [Macaca mulatta] | 0 | 2 |
| 87 | XP_001096042 | PREDICTED: similar to serine palmitoyltransferase, long chain base subunit 2 [Macaca mulatta] | 0 | 2 |
| 113 | XP_001100715 | PREDICTED: zinc finger protein 639 [Macaca mulatta] | 0 | 2 |
| 128 | XP_001114075 | PREDICTED: similar to T-cell immunomodulatory protein [Macaca mulatta] | 0 | 2 |
| 129 | XP_001113081 | PREDICTED: similar to filamin A interacting protein 1 isoform 1 [Macaca mulatta] | 0 | 2 |
| 145 | XP_001083301 | PREDICTED: cytochrome b-245, beta polypeptide (chronic granulomatous disease) isoform 1 [Macaca | 0 | 2 |
| | | mulatta] | | |
| 146 | XP_001112106 | PREDICTED: similar to thyroid hormone receptor associated protein 2 [Macaca mulatta] | 0 | 2 |
| 148 | XP_001087105 | PREDICTED: hypothetical protein [Macaca mulatta] | 0 | 2 |
| 163 | NP_001123907 | toll-interleukin 1 receptor (TIR) domain containing adaptor protein [Macaca mulatta] | 0 | 2 |
| 184 | XP_001117349 | PREDICTED: similar to alpha-2-plasmin inhibitor [Macaca mulatta] | 0 | 2 |

TABLE 8
Biomarkers identified by the proteomics study

| | | Amino Acid |
|---|---|---|
| Gene identifier | Gene name | Sequence |
| | Immediate early stroke biomarkers, 1-hr post stroke | |
| NP_004235.3 | scavenger receptor cysteine-rich type 1 protein M130 isoform | SEQ ID NO: 97 |
| NP_056150.1 | mediator of RNA polymerase II transcription subunit 13-like | SEQ ID NO: 98 |
| NP_000388.2 | cytochrome b-245 heavy chain | SEQ ID NO: 99 |
| NP_006245.2 | protein kinase C delta type | SEQ ID NO: 100 |
| NP_001193726.1 | pyruvate kinase isozymes M1/M2 isoform d | SEQ ID NO: 101 |
| NP_004478.3 | Golgin subfamily B member 1 | SEQ ID NO: 102 |
| NP_002924.1 | ribonuclease pancreatic precursor | SEQ ID NO: 103 |
| NP_001157281.1 | WD repeat-containing protein 81 isoform 1 | SEQ ID NO: 104 |
| NP_036336.2 | general transcription factor 3C polypeptide 4 | SEQ ID NO: 105 |
| | Early stroke biomarkers, 2-hr post stroke | |
| NP_004235.3 | scavenger receptor cysteine-rich type 1 protein M130 isoform | SEQ ID NO: 97 |
| NP_001843.1 | collagen alpha-2(IX) chain precursor | SEQ ID NO: 106 |
| NP_001092103.1 | proline-rich AKT1 substrate 1 | SEQ ID NO: 107 |
| NP_006245.2 | protein kinase C delta type | SEQ ID NO: 100 |
| NP_002924.1 | ribonuclease pancreatic precursor | SEQ ID NO: 103 |
| NP_000177.2 | complement factor H isoform a precursor | SEQ ID NO: 108 |
| NP_001157281.1 | WD repeat-containing protein 81 isoform 1 | SEQ ID NO: 104 |
| NP_004854.1 | serine palmitoyltransferase 2 | SEQ ID NO: 109 |
| NP_000349.1 | transforming growth factor-beta-induced protein ig-h3 precursor | SEQ ID NO: 110 |
| NP_079408.3 | WD repeat-containing protein 19 | SEQ ID NO: 111 |
| NP_060309.2 | lysophosphatidylcholine acyltransferase 2 | SEQ ID NO: 112 |
| NP_653202.1 | CKLF-like MARVEL transmembrane domain-containing protein 3 | SEQ ID NO: 113 |
| NP_110417.2 | T-cell immunomodulatory protein precursor | SEQ ID NO: 114 |
| NP_056502.1 | filamin-A-interacting protein 1 | SEQ ID NO: 115 |
| NP_001074290.1 | zinc finger protein 799 | SEQ ID NO: 116 |
| NP_112092.1 | apolipoprotein L2 | SEQ ID NO: 117 |
| NP_001124477.1 | myocyte-specific enhancer factor 2C isoform 2 | SEQ ID NO: 118 |
| NP_071387.1 | phosphorylated CTD-interacting factor 1 | SEQ ID NO: 119 |
| NP_001180539.1 | tubulin delta chain isoform 3 | SEQ ID NO: 120 |
| NP_003480.2 | nuclear receptor-interacting protein 1 | SEQ ID NO: 121 |
| NP_001207.2 | carbonic anhydrase 9 precursor | SEQ ID NO: 122 |
| | Late stroke biomarkers, 24-hr post stroke | |
| NP_006245.2 | protein kinase C delta type | SEQ ID NO: 100 |
| NP_733840.1 | NLR family member X1 isoform 2 [Homo sapiens] | SEQ ID NO: 26 |
| NP_001340.2 | aspartyl-tRNA synthetase, cytoplasmic | SEQ ID NO: 123 |
| XP_003119104.1 | PREDICTED: putative Ras GTPase-activating protein 4B isoform 5 | SEQ ID NO: 124 |
| NP_653202.1 | CKLF-like MARVEL transmembrane domain-containing protein 3 | SEQ ID NO: 113 |
| NP_001193726.1 | pyruvate kinase isozymes M1/M2 isoform | SEQ ID NO: 101 |
| NP_127497.1 | NACHT, LRR and PYD domains-containing protein 1 isoform 1 | SEQ ID NO: 125 |
| NP_002777.1 | proteasome subunit alpha type-1 isoform 2 | SEQ ID NO: 126 |
| NP_003456.1 | chitotriosidase-1 precursor | SEQ ID NO: 127 |
| NP_002289.2 | plastin-2 | SEQ ID NO: 128 |
| NP_004478.3 | Golgin subfamily B member 1 | SEQ ID NO: 129 |
| NP_001843.1 | collagen alpha-2(IX) chain precursor | SEQ ID NO: 130 |
| NP_079408.3 | WD repeat-containing protein 19 | SEQ ID NO: 111 |
| NP_705932.2 | sodium/potassium/calcium exchanger 4 isoform 1 precursor | SEQ ID NO: 131 |
| NP_056031.2 | inositol hexakisphosphate and diphosphoinositol-pentakisphosphate | SEQ ID NO: 132 |
| | kinase 2 | |
| NP_006512.2 | transcription factor E3 | SEQ ID NO: 133 |
| NP_002924.1 | ribonuclease pancreatic precursor | SEQ ID NO: 103 |
| NP_001157861.1 | rap guanine nucleotide exchange factor 6 isoform 5 | SEQ ID NO: 134 |
| NP_066288.2 | deformed epidermal autoregulatory factor 1 homolog | SEQ ID NO: 135 |
| NP_004220.2 | mediator of RNA polymerase II transcription subunit 14 | SEQ ID NO: 136 |
| NP_005472.2 | mediator of RNA polymerase II transcription subunit 16 | SEQ ID NO: 137 |
| NP_057038.2 | putative deoxyribose-phosphate aldolase | SEQ ID NO: 138 |
| NP_004854.1 | serine palmitoyltransferase 2 | SEQ ID NO: 139 |
| NP_057415.1 | zinc finger protein 639 | SEQ ID NO: 140 |
| NP_110417.2 | T-cell immunomodulatory protein precursor | SEQ ID NO: 114 |
| NP_056502.1 | filamin-A-interacting protein 1 | SEQ ID NO: 115 |
| NP_000388.2 | cytochrome b-245 heavy chain | SEQ ID NO: 99 |
| NP_056150.1 | mediator of RNA polymerase II transcription subunit 13-like | SEQ ID NO: 98 |
| NP_689956.2 | BRCA1-associated ATM activator 1 | SEQ ID NO: 141 |
| NP_001034750.1 | toll/interleukin-1 receptor domain-containing adapter protein isoform a | SEQ ID NO: 142 |
| NP_001157281.1 | WD repeat-containing protein 81 isoform 1 | SEQ ID NO: 104 |

Sequence of Stroke Biomarkers

>gi|5730120|ref|NP_006546.1| synaptobrevin homolog YKT6 [Homo sapiens], SEQ ID NO: 1
MKLYSLSVLYKGEAKVVLLKAAYDVSSFSFFQRSSVQEFMTFTSQLIVERSSKGTRASVKEQDYLCHVYVRNDSLAGVVIADNEYPSRVA
FTLLEKVLDEFSKQVDRIDWPVGSPATIHYPALDGHLSRYQNPREADPMTKVQAELDETKIILHNTMESLLERGEKLDDLVSKSEVLGTQS
KAFYKTARKQNSCCAIM
>gi|11386157|ref|NP_001776.1| cytidine deaminase [Homo sapiens], SEQ ID NO: 2
MAQKRPACTLKPECVQQLLVCSQEAKKSAYCPYSHFPVGAALLTQEGRIFKGCNIENACYPLGICAERTAIQKAVSEGYKDFRAIAIASDM
QDDFISPCGACRQVMREFGTNWPVYMTKPDGTYIVMTVQELLPSSFGPEDLQKTQ
>gi|119580742|gb|EAW60338.1| hypothetical protein FLJ20232, isoform CRA_b [Homo sapiens],
SEQ ID NO: 3
MAGAGERKGKKDDNGIGTAIDFVLSNARLVLGVGGAAMLGIATLAVKRMYDRAISAPTSPTRLSHSGKRSWEEPNWMGSPRLLNRDMK
TGLSRSLQTLPTDSSTFDTDTFCPPRPKPVARKGQVDLKKSRLRMSLQEKLLTYYRNRAAIPAGEQARAKQAAVDICAELRSFLRAKLPDM
PLRDMYLSGSLYDDLQVVTADHIQLIVPLVLEQNLWSCIPGEDTIMNVPGFFLVRRENPEYFPRGSSYWDRCVVGGYLSPKTVADTFEKVV
AGSINWPAIGSLLDYVIRPAPPPEALTLEVQYERDKHLFIDFLPSVTLGDTVLVAKPHRLAQYDNLWRLSLRPAETARLRALDQADSGCRSL
CLKILKAICKSTPALGHLTASQLTNVILHLAQEEADWSPDMLADRFLQALRGLISYLEAGVLPSALNPKDKALKERSLRHQVRMMEVDSRL
NVSWKIQSLVGVWWPCFLPRQALLF
>gi|119587725|gb|EAW67321.1| centrosomal protein 164kDa, isoform CRA_d [Homo sapiens], SEQ ID NO: 4
MAGRPLRIGDQLVLEEDYDETYIPSEQEILEFAREIGIDPIKEPELMWLAREGIVAPLPGEWKPCQDITGDIYYFNFANGQSMWDHPCDEHY
RSLVIQERAKLSTSGAIKKKKKKKEKKDKKDRDPPKSSLALGSSLAPVHVPLGGLAPLRGLVDTPPSALRGSQSVSLGSSVESGRQLGELML
PSQGLKTSAYTKGLLGSIYEDKTALSLLGLGEETNEEDEEESDNQSVHSSSEPLRNLHLDIGALGGDFEYEESLRTSQPEEKKDVSLDSDAA
GPPTPCKPSSPGADSSLSSAVGKGRQGSGARPGLPEKEENEKSEPKICRNLVTPKADPTGSEPAKASEKEAPEDTVDAGEEGSRREEAAKEP
KKKASALEEGSSDASQELEISEHMKEPQLSDSIASDPKSFHGLDFGFRSRISEHLLDVDVLSPVLGGACRQAQQPLGIEDKDDSQSSQDELQS
KQSKGLEERYHRLSPPLPHEERAQSPPRSLATEEEPPQGPEGQPEWKEAEELGEDSAASLSLQLSLQREQAPSPPAACEKGKEQHSQAEELG
PGQEEAEDPEEKVAVSPTPPVSPEVRSTEPVAPPEQLSEAALKAMEEAVAQVLEQDQRHLLESKQEKMQQLREKLCQEEEEEILRLHQQKE
QSLSSLRERLQKAIEEEEARMREEESQRLSWLRAQVQSSTQADEDQIRAEQEASLQKLREELESQQKAERASLEQKNRQMLEQLKEEIEAS
EKSEQAALNAAKEKALQQLREQLEGERKEAVATLEKEHSAELERLCSSLEAKHREVVSSLQKKIQEAQQKEEAQLQKCLGQVEHRVHQK
SYHVAGYEHELSSLLREKRQEVEGEHERRLDKMKEEHQQVMAKAREQYEAEERKQRAELLGHLTGELERLQRAHERELETVRQEQH
KRLEDLRRRHREQERKLQDLELDLETRAKDVKARLALLEVQEETARREKQQLLDVQRQVALKSEEATATHQQLEEAQKEHTHLLQSNQQ
LREILDELQARKLKLESQVDLLQAQSQQLQKHFSSLEAEAQKKQHLLREVTVEENNASPHFEPDLHIEDLRKSLGTNQTKEVSSSLSQSKED
LYLDSLSSHNVWHLLSAEGVALRSAKEFLVRQTRSMRRRQTALKAAQQHWRHELASAQEVAKDPPGIKALEDMRKNLEKETRHLDEMK
SAMRKGHNLLKKKEEKLNQLESSLWEEASDEGTLGGSPTKKAVTFDLSDMDSLSSESSESFSPPHLDSTPSLTSRKIHGLSHSLRQISSQLSS
VLSILDSLNPQSPPPLLASMPAQLPPRDPKSTPTPTYYGSLARFSALSSATPTSTQWAWDSGQGPRLPSSVAQTVDDFLLEKWRKYFPSGIPL
LSNSPTPLESRLGYMSASEQLRLLQHSHSQVPEAGSTTFQGIIEANRRWLERVKNDPRLPLFSSTPKPKATLSLLQLGLDEHNRVKVYRF
>gi|119591865|gb|EAW71459.1| WD repeat domain 8, isoform CRA_a [Homo sapiens], SEQ ID NO: 5
MNFSEVFKLSSLLCKFSPDGKYLASCVQYRLVVRDVNTLQILQLYTCLDQIQHIEWSADSLFILCAMYKRGLVQVWSLEQPEWHCKIDEGS
AGLVASCWSPDGRHILNTTEFHLRITVWSLCTKSVSYIKYPKACLQGITFTRDGRYMALAERRDCKDYVSIFVCSDWQLLRHFDTDTQDLT
GIEWAPNGCVLAVWDTCLEVRILNHVTWKMITEFGHPAAINDPKIVVYKEAEKSPQLGLGCLSFPPPRAGAGPLPSSESKYEIASVPVSLQT
LKPVTDRANPKIGIGMLAFSPDSYFLATRNDNIPNAVWVWDIQKLRLFAVLEQLSPVRAFQWDPQQPRLAICTGGS
RLYLWSPAGCMSVQVPGEGDFAVLSLCWHLSGDSMALLSKDHFCLCFLETEAVVGTACRQLGGHT
>gi|119605546|gb|EAW85140.1| zinc finger CCCH-type containing 7A, isoform CRA_b [Homo sapiens],
SEQ ID NO: 6
MSNVSEERRKRQQNIKEGLQFIQSPLSYPGTQEQYAVYLRALVRNLFNEGNDVYREHDWNNSISQYTEALNIADYAKSEEILIPKEIIEKLYI
NRIACYSNMGFHDKVLEDCNIVLSLNASNCKALYRKSKALSDLGRYKKAYDAVAKCSLAVPQDEHVIKLTQELAQKLGFKIRKAYVRAE
LSLKSVPGDGATKALNHSVEDIEPDLLTPRQEAVPVVSLPAPSFSHEVGSELASVPVMPLTSILPLQVEESALPSAVLANGGKMPFTMPEAFL
DDGDMVLGDELDDLLDSAPETNETVMMNGPGSLFASENFLGISSQPRNDFGNFFGSAVTKPSSSVTPRHPLEGTHELRQACQICFVKSGPK
LMDFTYHANIDHKCKKDILIGRIKNVEDKSWKKIRPRPTKTNYEGPYYICKDVAAEEECRYSGHCTFAYCQEEIDVWTLERKGAFSREAFF
GGNGKINLTVFKLLQEHLGEFIFLCEKCFDHKPRMISKRNKDNSTACSHPVTKHEFEDNKCLVHILRETTVKYSKIRSFHGQCQLDLCRHEV
RYGCLREDECFYAHSLVELKVWIMQNETGISHDAIAQESKRYWQNLEANVPGAQVLGNQIMPGFLNMKIKFVCAQCLRNGQVIEPDKNR
KYCSAKARHSWTKDRRAMRVMSIERKKWMNIRPLPTKKQMPLQFDLCNHIASGKKCQYVGNCSFAHSPEEREVWTYMKENGIQDMEQ
FYELWLKS
>gi|119609945|gb|EAW89539.1| TIMP metallopeptidase inhibitor 2, isoform CRA_b [Homo sapiens],
SEQ ID NO: 7
MPVIRAKAVSEKEVDSGNDIYGNPIKRIQYEIKQIKMFKGPEKDIEFIYTAPSSAVCGVSLDVGGKKEYLIAGKAEGDGKMHITLCDFIVPW
DTLSTTQKKSLNHRYQMGCECKITRCPMIPCYISSPDECLWMDWVTEKNINGHQAKFFACIKRSDGSCAWYRGAAPPKQEFLDIEDP
>gi|119617294|gb|EAW96888.1| zinc finger CCCH-type containing 10, isoform CRA_a [Homo sapiens],
SEQ ID NO: 8
MSHAYFIHHRVWFRVCWLERHFFRRKRKSDRVGGSLSKLEDRQAAAATTAAAAEPSSDVEVETHRSPGLHLSYLSGHQEWQDKENPELG
GTRMPDRDSYANGTGSSGGGPGGGGSEEASGAGVGSGGASSDAICRDFLRNVCKRGKRCRYRHPDMSEVSNLGVSKNEFIFCHDFQNKE
CSRPNCRFIHGSKEDEDGYKKTGELPPRLRQKVAAGLGLSPADLPNGKEEVPICRDFLKGDCQRGAKCKFRHLQRDFEFDARGGGGTGGG
STGSVLPGRRHDLYDIYDLPDRGFEDHEPGPKRRRGGCCPPDGPHFESYEYSLAPPRGVECRLLEEENAMLRKRVEELKKQVSNLLATNEV
LLEQNAQFRNQAKVITLSSTAPATEQTLAPTVGTVATFNHGIAQTHTTLSSQALQPRPVSQQELVAPAGAPAAPPTNAAPPAAPPPPPPHLT
PEITPLSAALAQTIAQGMAPPPVSMAPVAVSVAPVAPVAVSMAQPLAGITMSHTTTPMVTYPIASQSMRITAMPH
>gi|14017843|dbj|BAB47442.1| KIAA1813 protein [Homo sapiens], SEQ ID NO: 9
ASATMAIVQTLPVPLEPAPEAATAPQAPVMGSVSSLISGRPCPGGPAPPRHHGPPGPTFFRQQDGLLRGGYEAQEPLCPAVPPRKAVPVTSF
TYINEDFRTESPPSPSSDVEDAREQRAHNAHLRGPPPKLIPVSGKLEKNMEKILIRPTAFKPVLPKPRGAPSLPSFMGPRATGLSGSQGSLTQL
FGGPASSSSSSSSSSAADKPLAFSGWASGCPSGTLSDSGRNSLSSLPTYSTGGAEPTTSSPGGHLPSHGSGRGALPGPARGVPTGPSHSDSGR
SSSSKSTGSLGGRVAGGLLGSGTRASPDSSSCGERSPPPPPPPPSDEALLHCVLEGKLRDREAELQQLRDSLDENEATMCQAYEERQRHWQ
REREALREDCAAQAQRAQRAQQLLQLQVFQLQQEKRQLQDDFAQLLQEREQLERRCATLEREQRELGPRLEETKWEVCQKSGEISLLKQ
QLKESQAELVQKGSELVALRVALREARATLRVSEGRARGLQEAARARELELEACSQELQRHRQEAEQLREKAGQLDAEAAGLREPPVPPA
TADPFLLAESDEAKVQRAAAGVGGSLRAQVERLRVELQRERRRGEEQRDSFEGERLAWQAEKEQVIRYQKQLQHNYIQMYRRNRQLEQ
ELQQLSLELEARELADLGLAEQAPCICLEEITATEI
>gi|14289171|dbj|BAB59061.1| Pex1p-634del690 [Homo sapiens], SEQ ID NO: 10
MWGSDRLAGAGGGGAAVTVAFTNARDCFLHLPRRLVAQLHLLQNQAIEVVWSHQPAFLSWVEGRHFSDQGENVAEINRQVGQKLGLSN
GGQVFLKPCSHVVSCQQVEVEPLSADDWEILELHAVSLEQHLLDQIRIVFPKAIFPVWVDQQTYIFIQIVALIPAASYGRLETDTKLLIQPKTR
RAKENTFSKADAEYKKLHSYGRDQKGMMKELQTKQLQSNTVGITESNENESEIPVDSSSVASLWTMIGSIFSFQSEKKQETSWGLTEINAF
KNMQSKVVPLDNIFRVCKSQPPSIYNASATSVFHKHCAIHVFPWDQEYFDVEPSFTVTYGKLVKLLSPKQQQSKTKQNVLSPEKEKQMSE
PLDQKKIRSDHNEEDEKACVLQVVWNGLEELNNAIKYTKNVEVLHLGKVWIPDDLRKRLNIEMHAVVRITPVEVTPKIPRSLKLQPRENLP
KDISEEDIKTVFYSWLQQSTTTMLPLVISEEEFIKLETKDGLKEFSLSIVHSWEKEKDKNIFLLSPNLLQKTTIQVLLDPMVKEENSEEIDFILPF
LKLSSLGGVNSLGVSSLEHITHSLLGRPLSRQLMSLVAGLRNGALLLTGGKGSGKSTLAKAICKEAFDKLDAHVERVDCKALRALNDMIKE
FISMGSLVALIATSQSQQSLHPLLVSAQGVHIFQCVQHIQPPNQEQRCEILCNVIKNKLDCDINKFTDLDLQHVAKETGGFVARDFTVLVDR
AIHSRLSRQSISTREKLVLTTLDFQKALRGFLPASLRSVNLHKPRDLGWDKIGGLHEVRQILMDTIQLPAKYPELFANLPIRQRTGILLYGPPG
TGKTLLAGVIARESRMNFISVKGPELLSKYIGASEQAVRDIFIRAQAAKPCILFFDEFESIAPRRGHDNTGVTDRVVNQLLTQLDGVEGLQGV
YVLAATSRPDLIDPALLRPGRLDKCVYCPPPDQVSRLEILNVLSDSLPLADDVDLQHVASVTDSFTGADLKALLYNAQLEALHGMLLSSGL
QDGSSSSDSDLSLSSMVFLNHSSGSDDSAGDGECGLDQSLVSLEMSEILPDESKFNMYRLYFGSSYESELGNGTSSDLSSQCLSAPSSMTQD
LPGVPGKDQLFSQPPVLRTASQEGCQELTQEQRDQLRADISIIKGRYRSQSGEDESMNQPGPIKTRLAISQSHLMTALGHTRPSISEDDWKN
FAELYESFQNPKRRKNQSGTMFRPGQKVTLA
>gi|14495615|gb|AAH09411.1| WDR77 protein [Homo sapiens], SEQ ID NO: 11
MRKETPPPLVPPAAREWNLPPNAPACMERQLEAARYRSDGALLLGASSLSGRCWAGSLWLFKDPCAAPNEGFCSAGVQTEAGVADLTW
VGERGILVASDSGAVELWELDENETLIVSKFCKYEHDDIVSTVSVLSSGTQAVSGSKDICIKVWDLAQQVVLSSYRAHAAQVTCVAASPHK
DSVFLSCSEDNRILLWDTRCPKPASQIGCSAPGYLPTSLAWHPQQSEVFVFGDENGTVSLVDTKSTSCVLSSAVHSQCVTGLVFSPHSVPFL
ASLSEDCSLAVLDSSLSELFRSQAHRDFVRDATWSPLNHSLDLQVLLSRLDLRQKASPP
>gi|146231938|ref|NP_001078929.1| catenin delta-1 isoform 1A [Homo sapiens], SEQ ID NO: 12
MDDSEVESTASILASVKEQEAQFEKLTRALEEERRHVSAQLERVRVSPQDANPLMANGTLTRRHQNGRFVGDADLERQKFSDLKLNGPQ
DHSHLLYSTIPRMQEPGQIVETYTEEDPEGAMSVVSVETSDDGTTRRTETTVKKVVKTVTTRTVQPVAMGPDGLPVDASSVSNNYIQTLG
RDFRKNGNGGPGPYVGQAGTATLPRNFHYPPDGYSRHYEDGYPGGSDNYGSLSRVTRIEERYRPSMEGYRAPSRQDVYGPQPQVRVGGS
SVDLHRFHPEPYGLEDDQRSMGYDDLDYGMMSDYGTARRTGTPSDPRRRLRSYEDMIGEEVPSDQYYWAPLAQHERGSLASLDSLRKG
GPPPPNWRQPELPEVIAMLGFRLDAVKSNAAAYLQHLCYRNDKVKTDVRKLKGIPVLVGLLDHPKKEVHLGACGALKNISFGRDQDNKIA
IKNCDGVPALVRLLRKARDMDLTEVITGTLWNLSSHDSIKMEIVDHALHALTDEVIIPHSGWEREPNEDCKPRHIEWESVLTNTAGCLRNV
SSERSEARRKLRECDGLVDALIFIVQAEIGQKDSDSKLVENCVCURNLSYQVHREIPQAERYQEAAPNVANNTGPHAASCFGAKKGKGK
KPIEDPANDTVDFPKRTSPARGYELLFQPEVVRIYISLLKESKTPAILEASAGAIQNLCAGRWTYGRYIRSALRQEKALSAIADLLTNEHERV
VKAASGALRNLAVDARNKELIGKHAIPNLVKNLPGGQQNSSWNFSEDTVISILNTINEVIAENLEAAKKLRETQGIEKLVLINKSGNRSEKEV
RAAALVLQTIWGYKELRKPLEKEGWKKSDFQVNLNNASRSQSSHSYDDSTLPLIDRNQKSDKKPDREEIQMSNMGSNTKSLDNNYSTPNE
RGDHNRTLDRSGDLGDMEPLKGTTPLMQKI
>gi|15029530|ref|NP_071426.1| leucine-rich repeat-containing protein 4 precursor [Homo sapiens],
SEQ ID NO: 13
MKLLWQVTVHHHTWNAILLPFVYLTAQVWILCAAIAAAASAGPQNCPSVCSCSNQFSKVVCTRRGLSEVPQGIPSNTRYLNLMENNIQMI
QADTFRHLHHLEVLQLGRNSIRQIEVGAFNGLASLNTLELFDNWLTVIPSGAFEYLSKLRELWLRNNPIESIPSYAFNRVPSLMRLDLGELKK
LEYISEGAFEGLFNLKYLNLGMCNIKDMPNLTPLVGLEELEMSGNHFPEIRPGSFHGLSSLKKLWVMNSQVSLIERNAFDGLASLVELNLAH
NNLSSLPHDLFTPLRYLVELHLHHNPWNCDCDILWLAWWLREYIPTNSTCCGRCHAPMHMRGRYLVEVDQASFQCSAPFIMDAPRDLNIS
EGRMAELKCRTPPMSSVKWLLPNGTVLSHASRHPRISVLNDGTLNFSHVLLSDTGVYTCMVTNVAGNSNASAYLNVSTAELNTSNYSFFT
TVTVETTEISPEDTTRKYKPVPTTSTGYQPAYTTSTTVLIQTTRVPKQVAVPATDTTDKMQTSLDEVMKTTKIIIGCFVAVTLLAAAMLIVFY
KLRKRHQQRSTVTAARTVEIIQVDEDIPAATSAAATAAPSGVSGEGAVVLPTIHDHINYNTYKPAHGAHWTENSLGNSLHPTVTTISEPYIIQ
THTKDKVQETQI
>gi|157738629|ref|NP_001099038.1| kinesin-like protein KIF13A isoform d [Homo sapiens],
SEQ ID NO: 14
MSDTKVKVAVRVRPMNRRELELNTKCVVEMEGNQTVLHPPPSNTKQGERKPPKVFAFDYCFWSMDESNTTKYAGQEVVFKCLGEGILE
KAFQGYNACIFAYGQTGSGKSFSMMGHAEQLGLIPRLCCALFKRISLEQNESQTFKVEVSYMEIYNEKVRDLLDPKGSRQSLKVREHKVLG
PYVDGLSQLAVTSFEDIESLMSEGNKSRTVAATNMNEESSRSHAVFNIIITQTLYDLQSGNSGEKVSKVSLVDLAGSERVSKTGAAGERLKE
GSNINKSLTTLGLVISSLADQAAGKGKSKFVPYRDSVLTWLLKDNLGGNSQTSMIATISPAADNYEETLSTLRYADRAKRIVNHAVVNEDP
NAKVIRELREEVEKLREQLSQAEAMKAPELKEKLEESEKLIKELTVTWEEKLRKTEEIAQERQRQLESMGISLEMSGIKVGDDKCYLVNLN
ADPALNELLVYYLKDHTRVGADTSQDIQLFGIGIQPQHCEIDIASDGDVTLTPKENARSCVNGTLVCSTTQLWHGDRILWGNNHFFRINLPK
RKRRDWLKDFEKETGPPEHDLDAASEASSEPDYNYEFAQMEVIMKTLNSNDPVQNVVQVLEKQYLEEKRSALEEQRLMYERELEQLRQQ
LSPDRQPQSSGPDRLAYSSQTAQQKVTQWAEERDELFRQSLAKLREQLVKANTLVREANFLAEEMSKLTDYQVTLQIPAANLSANRKRG
AIVSEPAIQVRRKGKSTQVWTIEKLENKLIDMRDLYQEWKEKVPEAKRLYGKRGDPFYEAQENHNLIGVANVFLECLFCDVKLQYAVPIIS
QQGEVAGRLHVEVMRVTGAVPERVVEDDSSENSSESGSLEVVDSSGEIIHRVKKLTCRVKIKEATGLPLNLSNFVFCQYTFWDQCESTVA
APVVDPEVPSPQSKDAQYTVTFSHCKDYVVNVTEEFLEFISDGALAIEVWGHRCAGNGSSIWEVDSLHAKTRTLHDRWNEVTRRIEMWISI
LELNELGEYAAVELHQAKDVNTGGIFQLRQGHSRRVQVTVKPVQHSGTLPLMVEAILSVSIGCVTARSTKLQRGLDSYQEEDLNCVRERW
SDALIKRREYLDEQIKKVSNKTEKTEDDVEREAQLVEQWVGLTEERNAVLVPAPGSGIPGAPADWIPPPGMETHIPVLFLDLNADDLSANE
QLVGPHASGVNSILPKEHGSQFFYLPIIKHSDDEVSATASWDSSVHDSVHLNRVTPQNERIYLIVKTTVQLSHPAAMELVLRKRIAANIYNK
QSFTQSLKRRISLKNIFYSCGVTYEIVSNIPKATEEIEDRETLALLAARSENEGTSDGETYIEKYTRGVLQVENILSLERLRQAVTVKEALSTK
ARHIRRSLSTPNVHNVSSSRPDLSGFDEDDKGWPENQLDMSDYSSSYQDVACYGTLPRDSPRRNKEGCTSETPHALTVSPFKAFSPQPPKF
FKPLMPVKEEHKKRIALEARPLLSQEDSEEEENELEAINRKLISSQPYVPVEFADFSVYNASLENREWFSSKVDLSNSRVLEKEVSRSPTTSSI
TSGYFSHSASNATLSDMVVPSSDSSDQLAIQTKDADSTEHSTPSLVHDFRPSSNKELTEVEKGLVKDKIIVVPLKENSALAKGSPSSQSIPEK
NSKSLCRTGSCSELDACPSKISQPARGFCPREVTVEHTTNILEDHSFTEFMGVSEGKDFDGLTDSSAGELSSRRSLPNKTGGKTVSDGLHHP
SQLHSKLENDQGGTTSR
>gi|193783661|dbjBAG53572.1| unnamed protein product [Homo sapiens], SEQ ID NO: 15
MDPPAEKPGEAGGLQITPQLLKSRTGEFSLESILLLKLRGLGLADLGCLGECLGLEWLDLSGNALTHLGPLASLRQLAVLNVSNNRLTGLEP
LATCENLQSLNAAGNLLATPGQLQCLAGLPCLEYLRLRDPLARLSNPLCASPSYWAAVRELLPGLKVIDGERVIGRGSEFYQLCRDLDSSLR
PSSSPGPRATEAQPWVEPGYWESWPSRSSSILEEACRQFQDTLQECWDLDRQASDSLAQAEQVLSSAGPTSSFVF
>gi|193784874|dbjBAG54027.1| unnamed protein product [Homo sapiens], SEQ ID NO: 16
MASSTSLPAPGSRPKKPLGKMADWFRQTLLKKPKKRPNSPESTSSDASQPTSQDNPLPPSLSSVTSPSLPPTHASDSGSSRWSKDYDVCVC
HSEEDLVAAQDLVSYLEGSTASLRCFLQLRDATPGGAIVSELCQALSSSHCRVLLITPGFLQDPWCKYQMLQALTEAPGAEGCTIPLLSGLS
RAAYPPELRFMYYVDGRGPDGGFRQVKEAVMRYLQTLS
>gi|194375926|dbjBAG57307.1| unnamed protein product [Homo sapiens], SEQ ID NO: 17
MGKEIPTDAPWEAQHADKWDKMTMKELIDKICWTKTARRFAYLFVNINVTSEPHEVSALWFLWYVKQCGGTTRIFSVTNGGQERKFVG
GSGQVSERIMDLLGDQVKLNHPVTHVDQSSDNIIIETLNHEHYECKYVINAIPPTLTAKIHFRPELPAERNQLIQRLPMGAVIKCMMYYKEA
FWKKKDYCGCMIIEDEDAPISITLDDTKPDGSLPAIMGFILARKADRLAKLHKEIRKKKICELYAKVLGSQEALHPVHYEEKNWCEEQYSG
GCYTAYFPPGIMTQYGRVIRQPVGRIFFAGTETATKWSGYMEGAVEAGERAAREVLNGLGKVTEKDIWVQEPESKDVPAVEITHTFWERN
LPSVSGLLKIIGFSTSVTALGFVLYKYKLLPRS
>gi|194380360|dbjBAG63947.1| unnamed protein product [Homo sapiens], SEQ ID NO: 18
MVFSMAQPMASSSTEPYLCLAAAGENPSGKSLASAISGKIPSPLSSSYKPMLNNNSFMWPNSTPVPLLQTTQGLKPVSPPKIQPVSWHHSG
GTGDCAPQPVDHKVPKSIGTVPADASAHIALSTASSHDTSTTSVASSWYNRNNLAMRAEPLSCALDDSSDSQDPTKEIRFTEAVRKLTARG
FEKMPRQGCQLEQSSFLNPSFQWNVLNRSRRWKPPAVNQQFPQEDAGSVRRVLPGASDTLGLDNTVFCTKRISIHLLASHASGLNHNPAC
ESVIDSSAFGEGKAPGPPFPQTLGIANVATRLSSIQLGQSEKERPEEARELDSSDRDISSATDLQPDQAETEDTEEELVDGLEDCCSRDENEEE
EGDSECSSLSAVSPGESVAMISRSCMEILTKPLSNHEKVVRPALIYSLFPNVPPTIYFGTRDERVEKLPWEQRKLLRWKMSTVTPNIVKQTIG
RSHFKISKRNDDWLGCWGHHMKSPSFRSIREHQKLNHFPGSFQIGRKDRLWRNLSRMQSRFGKKEFSFFPQSFILPQDAKLLRKAWESSSR
QKWIVKPPASARGIGIQVIHKWSQLPKRRPLLVQRYLHKPYLISGSKFDLRIYVYVTSYDPLRIYLFSDGLVRFASCKYSPSMKSLGNKFMH
LTNYSVNKKNAEYQANADEMACQGHKWALKALWNYLSQKGVNSDAIWEKIKDVVVKTIISSEPYVTSLLKMYVRRPYSCHELFGFDIML
DENLKPWVLEVNISPSLHSSSPLDISIKGQMIRDLLNLAGFVLPNAEDIISSPSSCSSSTTSLPTSPGDKCRMAPEHVTAQKMKKAYYLTQKIP
DQDFYASVLDVLTPDDVRILVEMEDEFSRRGQFERIFPSHISSRYLRFFEQPRYFNILTTQWEQKYHGNKLKGVDLLRSWCYKGFHMGVVS
DSAPVWSLPTSLLTISKDDVILNAFSKSETSKLGNPVPQRTVRTPAKSPAFLPRRYL
>gi|194381124|dbj|BAG64130.1| unnamed protein product [Homo sapiens], SEQ ID NO: 19
MGKLRPWRDLLEPVSCDLSDDMPWSACPHPRCVPRRCVIPCQSFVVTDVDYFSFQPDRPLGTRLTVTLTQHVQPPEPRDLQISTDQDHFLL
TWSVALGSPQSHWLSPGDLEFEVVYKRLQDSWEDAAILLSNTSQATLGPEHLMPSSTYVARVRTRLAPGSRLSGRPSKWSPEVCWDSQPG
DEAQPQNLECFFDGAAVLSCSWEVRKEVASSVSFGLFYKPSPDAGSAVLLREEECSPVLREGLGSLHTRHHCQIPVPDPATHGQYIVSVQP
RRAEKHIKSSVNIQMAPPSLNVTKDGDSYSLRWETMKMRYEHIDHTFEIQYRKDTATWKDSKTETLQNAHSMALPALEPSTRYWARVRV
RTSRTGYNGIWSEWSEARSWDTESVLPMWVLALIVIFLTIAVLLALRFCGIYGYRLRRKWEEKIPNPSKSHLFQNGSAELWPPGSMSAFTSG
SPPHQGPWGSRFPELEGVFPVGFGDSEVSPLTIEDPKHVCDPPSGPDTTPAASDLPTEQPPSPQPGPPAASHTPEKQASSFDFNGPYLGPPHS
RSLPDILGQPEPPQEGGSQKSPPPGSLEYLCLPAGGQVQLVPLAQAMGPGQAVEVERRPSQGAAGSPSLESGGGPAPPALGPRVGGQDQKD
SPVAIPMSSGDTEDPGVASGYVSSADLVFTPNSGASSVSLVPSLGLPSDQTPSLCPGLASGPPGAPGPVKSGFEGYVELPPIEGRSPRSPRNNP
VPPEAKSPVLNPGERPADVSPTSPQPEGLLVLQQVGDYCFLPGLGPGPLSLRSKPSSPGPGPEIKNLDQAFQVKKPPGQAVPQVPVIQLFKAL
KQQDYLSLPPWEVNKPGEVC
>gi|194382290|dbj|BAG58900.1| unnamed protein product [Homo sapiens], SEQ ID NO: 20
MAARLLRGSLRVLGGHRAPRQLPAARCSHSGGEERLETPSAKKLTDIGIRRIFSPEHDIFRKSVRKFFQEEVIPHHSEWEKAGEVSREVWEK
AGKQGLLGVNIAEHLGGIGGDLYSAAIVWEEQ
>gi|197333744|ref|NP_148978.2|cyclin-dependent kinase 16 isoform 2 [Homo sapiens], SEQ ID NO: 21
MQSEIAMDRMKKIKRQLSMTLRGGRGIDKTNGAPEQIGLDESGGGGGSDPGEAPTRAAPGELRSARGPLSSAPEIVHEDLKMGSDGESDQ
ASATSSDEVQSPVRVRMRNHPPRKISTEDINKRLSLPADIRLPEGYLEKLTLNSPIFDKPLSRRLRRVSLSEIGFGKLETYIKLDKLGEGTYAT
VYKGKSKLTDNLVALKEIRLEHEEGAPCTAIREVSLLKDLKHANIVTLHDIIHTEKSLTLVFEYLDKDLKQYLDDCGNIINMHNVKLFLFQLL
RGLAYCHRQKVLHRDLKPQNLLINERGELKLADFGLARAKSIPTKTYSNEVVTLWYRPPDILLGSTDYSTQIDMWGVGCIFYEMATGRPLF
PGSTVEEQLHFIFRILGTPTEETWPGILSNEEFKTYNYPKYRAEALLSHAPRLDSDGADLLTKLLQFEGRNRISAEDAMKHPFFLSLGERIHKL
PDTTSIFALKEIQLQKEASLRSSSMPDSGRPAFRVVDTEF
>gi|214010149|ref|NP_001135741.1| vitamin K-dependent gamma-carboxylase isoform 2 [Homo sapiens],
SEQ ID NO: 22
MAVSAGSARTSPSSGFLMVLDIPQERGLSSLDRKYLDGLDVCRFPLLDALRPLPLDWMYLVYTIMFLGALGMMLGLCYRISCVLFLLPYW
YVFLLDKTSWNNHSYLYGLLAFQLTFMDANHYWSVDGLLNAHRRNAHVPLWNYAVLRGQIFIVYFIAGVKKLDADWVEGYSMEYLSR
HWLFSPFKLLLSEELTSLLVVHWGGLLLDLSAGFLLFFDVSRSIGLFFVSYFHCMNSQLFSIGMFSYVMLASSPLFCSPEWPRKLVSYCPRRL
QQLLPLKAAPQPSVSCVYKRSRGKSGQKPGLRHQLGAAFTLLYLLEQLFLPYSHFLTQGYNNWTNGLYGYSWDMMVHSRSHQHVKITY
RDGRTGELGYLNPGVFTQSRRWKDHADMLKQYATCLSRLLPKYNVTEPQIYFDIWVSINDRFQQRIFDPRVDIVQAAWSPFQRTSWVQPL
LMDLSPWRAKLQEIKSSLDNHTEVVFIADFPGLHLENFVSEDLGNTSIQLLQGEVTVELVAEQKNQTLREGEKMQLPAGEYHKVYTTSPSP
SCYMYVYVNTTELALEQDLAYLQELKEKVENGSETGPLPPELQPLLEGEVKGGPEPTPLVQTFLRRQQRLQEIERRRNTPFHERFFRFLLRK
LYVFRRSFLMTCISLRNLILGRPSLEQLAQEVTYANLRPFEAVGELNPSNTDSSHSNPPESNPDPVHSEF
>gi|22212931|ref|NP_631981.2| nucleolar protein 6 gamma isoform [Homo sapiens], SEQ ID NO: 23
MGPAPAGEQLRGATGEPEVMEPALEGTGKEGKKASSRKRTLAEPPAKGLLQPVKLSRAELYKEPTNEELNRLRETEILFHSSLLRLQVEELL
KEVRLSEKKKDRIDAFLREVNQRVVRVPSVPETELTDQAWLPAGVRVPLHQVPYAVKGCFRFLPPAQVTVVGSYLLGTCIRPDINVDVAL
TMPREILQDKDGLNQRYFRKRALYLAHLAHHLAQDPLFGSVCFSYTNGCHLKPSLLLRPRGKDERLVTVRLHPCPPPDFFRPCRLLPTKNN
VRSAWYRGQSPAGDGSPEPPTPRYNTWVLQDTVLESHLQLLSTILSSAQGLKDGVALLKVWLRQRELDKGQGGFTGFLVSMLVVFLVSTR
KIHTTMSGYQVLRSVLQFLATTDLTVNGISLCLSSDPSLPALADFHQAFSVVFLDSSGHLNLCADVTASTYHQVQHEARLSMMLLDSRADD
GFHLLLMTPKPMIRAFDHVLHLRPLSRLQAACHRLKLWPELQDNGGDYVSAALGPLTTLLEQGLGARLNLLAHSRPPVPEWDISQDPPKH
KDSGTLTLGLLLRPEGLTSVLELGPEADQPEAAKFRQFWGSRSELRRFQDGAIREAVVWEAASMSQKRLIPHQVVTHLLALHADIPETCVH
YVGGPLDALIQGLKEASSTKGRMVMSRGGELVMVPNVEAILEDFAVLGEGLVQTVEARSERWTV
>gi|22266726|gb|AAM94901.1| AF311906_1 LIR-D1 precursor [Homo sapiens], SEQ ID NO: 24
MGALRPTLLPPSLPLLLLLMLGMGCWAREVLVPEGPLYRVAGTAVSISCNVTGYEGPAQQNFEWFLYRPEAPDTALGIVSTKDTQFSYAVF
KSRVVAGEVQVQRLQGDAVVLKIARLQAQDAGIYECHTPSTDTRYLGSYSGKVELRVLPDVLQVSAAPPGPRGRQAPTSPPRMTVHEGQ
ELALGCLARTSTQKHTHLAVSFGRSVPEAPVGRSTLQEVVGIRSDLAVEAGAPYAERLAAGELRLGKEGTDRYRMVVGGAQAGDAGTYH
CTAAEWIQDPDGSWAQIAEKRAVLAHVDVQTLSRQLPVTVGPGERRIGPGEPLELLCNVSGALPPAGRHAAYSVGWEMAPAGAPGPGRL
VAQLDTEGVGSLGPGYEGRHIAMEKVASRTYRLRLEAARPGDAGTYRCLAKAYVRGSGTRLREAASARSRPLPVHVREEGVVLEAVAWL
AGGTVYRGETASLLCNISVRGGPPGLRSARSGPVTVYPYMHALDTLFVPLLVGTGVALVTGATVLGTITCCFMKRLRKR
>gi|23273417|gb|AAH35404.1| PTK2 protein [Homo sapiens], SEQ ID NO: 25
MAAAYLDPNLNHTPNSSTKTHLGTGMERSPGAMERVLKWHYFESNSEPTTWASIIRHGDATDVRGIIQKIVDSHKVKHVACYGFRLSHL
RSEEVHWLHVDMGVSSVREKYELAHPPEEWKYELRIRYLPKGFLNQFTEDKPTLNFFYQQVKSDYMLEIADQVDQEIALKLGCLEIRRSY
WEMRGNALEKKSNYEVLEKDVGLKRFFPKSLLDSVKAKTLRKLIQQTFRQFANLNREESILKFFEILSPVYRFDKECFKCALGSSWIISVELA
IGPEEGISYLTDKGCNPTHLADFTQVQTIQYSNSEDKDRKGMLQLKIAGAPEPLTVTAPSLTIAENMADLIDGYCRLVNGTSQSFIIRPQKEG
ERALPSIPKLANSEKQGMRTHAVSVSETDDYAEIIDEEDTYTMPSTRDYEIQRERIELGRCIGEGQFGDVHQGIYMSPENPALAVAIKTCKN
CTSDSVREKFLQEALTMRQFDHPHIVKLIGVITENPVWIIMELCTLGELRSFLQVRKYSLDLASLILYAYQLSTALAYLESKRFVHRDIAARN
VLVSSNDCVKLGDFGLSRYMEDSTYYKASKGKLPIKWMAPESINFRRFTSASDVWMFGVCMWEILMHGVKPFQGVKNNDVIGRIENGER
LPMPPNCPPTLYSLMTKCWAYDPSRRPRFTELKAQLSTILEEEKAQQEERMRMESRRQATVSWDSGGSDEAPPKPSRPGYPSPRSSEGFYP
SPQHMVQTNHYQDSTVLDLRGIGQVLPTHLMEERLIRQQQEMEEDQRWLEKEERFLKPDVRLSRGSIDREDGSLQGPIGNQHIYQPVGKP
DPAAPPKKPPRPGAPGHLGSLASLSSPADSYNEGVKLQPQEISPPPTANLDRSNDKVYENVTGLVKAVIEMSSKIQPAPPEEYVPMVKEVGL
ALRTLLATVDETIPLLPASTHREIEMAQKLLNSDLGELINKMKLAQQYVMTSLQQEYKKQMLTAAHALAVDAKNLLDVIDQARLKMLGQ
TRPH
>gi|25777610|ref|NP_733840.1| NLR family member X1 isoform 2 [Homo sapiens], SEQ ID NO: 26
MRWGHHLPRASWGSGFRRALQRPDDRIPFLIHWSWPLQGERPFGPPRAFIRHHGSSVDSAPPPGRHGRLFPSASATEAIQRHRRNLAEWFS
RLPREERQFGPTFALDTVHVDPVIRESTPDELLRPPAELALEHQPPQAGLPPLALSQLFNPDACGRRVQTVVLYGTVGTGKSTLVRKMVLD
WCYGRLPAFELLIPFSCEDLSSLGPAPASLCQLVAQRYTPLKEVLPLMAAAGSHLLFVLHGLEHLNLDFRLAGTGLCSDPEEPQEPAAIIVNL
LRKYMLPQASILVTTRPSAIGRIPSKYVGRYGEICGFSDTNLQKLYFQLRLNQPYCGYAVGGSGVSATPAQRDHLVQMLSRNLEGHHQIAA
ACFLPSYCWLVCATLHFLHAPTPAGQTLTSIYTSFLRLNFSGETLDSTDPSNLSLMAYAARTMGKLAYEGVSSRKTYFSEEDVCGCLEAGIR
TEEEFQLLHIFRRDALRFFLAPCVEPGRAGTFVFTVPAMQEYLAALYIVLGLRKTTLQKVGKEVAELVGRVGEDVSLVLGIMAKLLPLRALP
LLFNLIKVVPRVFGRMVGKSREAVAQAMVLEMFREEDYYNDDVLDQMGASILGVEGPRRHPDEPPEDEVFELFPMFMGGLLSAHNRAVL
AQLGCPIKNLDALENAQAIKKKLGKLGRQVLPPSELLDHLFFHYEFQNQRFSAEVLSSLRQLNLAGVRMTPVKCTVVAAVLGSGRHALDE
VNLASCQLDPAGLRTLLPVFLRARKLGLQLNSLGPEACKDLRDLLLHDQCQITTLRLSNNPLTAAGVAVLMEGLAGNTSVTHLSLLHTGLG
DEGLELLAAQLDRNRQLQELNVAYNGAGDTAALALARAAREHPSLELLQGVAIQMCWKLPLLPYAHLWTPRMPSHWCFLLILMPPLPQW
YDGLVAPRGRCT
>gi|26000235|gb|AAN75525.1| Rap1-interacting adaptor molecule [Homo sapiens], SEQ ID NO: 27
MGESSEDIDQMFSTLLGEMDLLTQSLGVDTLPPPDPNPPRAEFNYSVGFKDLNESLNALEDQDLDALMADLVADISEAEQRTIQAQKESLQ
NQHHSASLQASIFSGAASLGYGTNVAATGISQYEDDLPPPPADPVLDLPLPPPPPEPLSQEEEEAQAKADKIKLALEKLKEAKVKKLVVKVH
MNDNSTKSLMVDERQLARDVLDNLFEKTHCDCNVDWCLYEIYPELQIERFFEDHENVVEVLSDWTRDTENKILFLEKEEKYAVFKNPQNF
YLDNRGKKESKETNEKMNAKNKESLLEESFCGTSIIVPELEGALYLKEDGKKSWKRRYFLLRASGIYYVPKGKTKTSRDLACFIQFENVNIY
YGTQHKMKYKAPTDYCFVLKHPQIQKESQYIKYLCCDDTRTLNQWVMGIRIAKYGKTLYDNYQRAVAKAGLASRWTNLGTVNAAAPA
QPSTGPKTGTTQPNGQIPQATHSVSAVLQEAQRHAETSKDKKPALGNHHDPAVPRAPHAPKSSLPPPPPVRRSSDTSGSPATPLKAKGTGG
GGLPAPPDDFLPPPPPPPPLDDPELPPPPPDFMEPPPDFVPPPPPSYAGIAGSELPPPPPPPAPAPAPVPDSARPPPAVAKRPPVPPKRQENPGH
PGGAGGGEQDFMSDLMKALQKKRGNVS
>gi|295148161|ref|NP_001171187.1| Krueppel-like factor 11 isoform b [Homo sapiens], SEQ ID NO: 28
MDICESILERKRHDSERSTCSILEQTDMEAVEALVCMSSWGQRSQKGDLLRIRPLTPVSDSGDVTTTVHMDAATPELPKDFHSLSTLCITPP
QSPDLVEPSTRTPVSPQVTDSKACTATDVLQSSAVVARALSGGAERGLLGLEPVPSSPCRAKGTSVIRHTGESPAACFPTIQTPDCRLSDSRE
GEEQLLGHFETLQDTHLTDSLLSTNLVSCQPCLHKSGGLLLTDKGQQAGWPGAVQTCSPKNYENDLPRKTTPLISVSVPAPPVLCQMIPVT
GQSSMLPAFLKPPPQLSVGTVRPILAQAAPAPQPVFVGPAVPQGAVMLVLPQGALPPPAPCAANVMAAGNTKLLPLAPAPVFITSSQNCVP
QVDFSRRRNYVCSFPGCRKTYFKSSHLKAHLRTHTGEKPFNCSWDGCDKKFARSDELSRHRRTHTGEKKFVCPVCDRRFMRSDHLTKHA
RRHMTTKKIPGWQAEVGKLNRIASAESPGSPLVSMPASA
>gi|296317249|ref|NP_001171726.1| trimethyllysine dioxygenase, mitochondrial isoform 2 precursor
[Homo sapiens] SEQ ID NO: 29
MWYHRLSHLHSRLQDLLKGGVIYPALPQPNFKSLLPLAVHWHHTASKSLTCAWQQHEDHFELKYANTVMRFDYVWLRDHCRSASCYNS
KTHQRSLDTASVDLCIKPKTIRLDETTLFFTWPDGHVTKYDLNWLVKNSYEGQKQKVIQPRILWNAEIYQQAQVPSVDCQSFLETNEGLKK
FLQNFLLYGIAFVENVPPTQEHTEKLAERISLIRETIYGRMWYFTSDFSRGDTAYTKLALDRHTDTTYFQEPCGIQVFHCLKHEGTGGRTLL
VDGFYAAEQVLQKAPEEFELLSKVPLKHEYIEDVGECHNHMIGIGPVLNIYPWNKELYLIRLFKEKQNTVNRQWNSSLQCDIPERILTYRHF
VSGTSIEHRGSLI
>gi|30840237|emb|CAD91343.1| cytochrome P450 [Homo sapiens], SEQ ID NO: 30
MDLIPNFAMETWVLVATSLVLLYIYGTHSHGLFKKLGIPGPTPLPFLGNILSYHKGFCMFDMECHKKYGKVWGFYDGQQPVLAITDPDMI
KTVLVKECYSVFTNRRPFGPVGFMKSAISIAEDEEWKRLRSLLSPTFTSGKLKEMVPIIAQYGDVLVRNLRREAETGKPVTLKDVFGAYSM
DVITSTSFGVNIDSLNNPQDPFVENTKKLLRFDFLDPFFLSITVFPFLIPILEVLNICVFPREVTNFLRKSVKRMKESRLEDTQKHRVDFLQLMI
DSQNSKETESHKALSDLELVAQSIIFIFAGYETTSSVLSFIMYELATHPDVQQKLQEEIDAVLPNKAPPTYDTVLQMEYLDMVVNETLRLFPI
AMRLERVCKKDVEINGMFIPKGVVVMIPSYALHRDPKYWTEPEKFLPERFSKKNKDNIDPYIYTPFGSGPRNCIGMRFALMNMKLALIRVL
QNFSFKPCKETQIPLKLSLGGLLQPEKPVVLKVESRDGTVSGA
>gi|33300639|ref|NP_115652.2| rhomboid domain-containing protein 1 [Homo sapiens], SEQ ID NO: 31
MQRRSRGINTGLILLLSQIFHVGINNIPPVTLATLALNIWFFLNPQKPLYSSCLSVEKCYQQKDWQRLLLSPLHEIADDWHLYFNMASMLWK
GINLERRLGSRWFAYVITAFSVLTGVVYLLLQFAVAEFMDEPDFKRSCAVGFSGVLFALKVLNNHYCPGGFVNILGFPVPNRFACWVELVA
IHLFSPGTSFAGHLAGILVGLMYTQGPLKKIMEACAGGFSSSVGYPGRQYYFNSSGSSGYQDYYPHGRPDHYEEAPRNYDTYTAGLSEEE
QLERALQASLWDRGNTRNSPPPYGFHLSPEEMRRQRLHRFDSQ
>gi|33342278|ref|NP_079480.2| traB domain-containing protein [Homo sapiens], SEQ ID NO: 32
MDGEEQQPPHEANVEPVVPSEASEPVPRVLSGDPQNLSDVDAFNLLLEMKLKRRRQRPNLPRTVTQLVAEDGSRVYVVGTAHFSDDSKR
DVVKTIREVQPDVVVVELCQYRVSMLKMDESTLLREAQELSLEKLQQAVRQNGLMSGLMQMLLLKVSAHITEQLGMAPGGEFREAFKEA
SKVPFCKFHLGDRPIPVTFKRAIAALSFWQKVRLAWGLCFLSDPISKDDVERCKQKDLLEQMMAEMIGEFPDLHRTIVSERDVYLTYMLRQ
AARRLELPRASDAEPRKCVPSVVVGVVGMGHVPGIEKNWSTDLNIQEIMTVPPPSVSGRVSRLAVKAAFFGLLGYSLYWMGRRTASLVLS
LPAAQYCLQRVTEARHK
>gi|40068483|ref|NP_954574.1| histone-lysine N-methyltransferase setd3 isoform b [Homo sapiens],
SEQ ID NO: 33
MGKKSRVKTQKSGTGATATVSPKEILNLTSELLQKCSSPAPGPGKEWEEYVQIRTLVEKIRKKQKGLSVTFDGKREDYFPDLMKWASENG
ASVEGFEMVNFKEEGFGLRATRDIKAEELFLWVPRKLLMTVESAKNSVLGPLYSQDRILQAMGNIALAFHLLCERASPNSFWQPYIQTLPSE
YDTPLYFEEDEVRYLQSTQAIHDVFSQYKNTARQYAYFYKVIQTHPHANKLPLKDSFTYEDYRWAVSSVMTRQNQIPTEDGSRVTLALIP
LWDMCNHTNGLTPEDSFALAVASA
>gi|4503591|ref|NP_000112.1| erythropoietin receptor precursor [Homo sapiens], SEQ ID NO: 34
MDHLGASLWPQVGSLCLLLAGAAWAPPPNLPDPKFESKAALLAARGPEELLCFTERLEDLVCFWEEAASAGVGPGNYSFSYQLEDEPWKL
CRLHQAPTARGAVRFWCSLPTADTSSFVPLELRVTAASGAPRYHRVIHINEVVLLDAPVGLVARLADESGHVVLRWLPPPETPMTSHIRYE
VDVSAGNGAGSVQRVEILEGRTECVLSNLRGRTRYTFAVRARMAEPSFGGFWSAWSEPVSLLTPSDLDPLILTLSLILVVILVLLTVLALLSH
RRALKQKIWPGIPSPESEFEGLFTTHKGNFQLWLYQNDGCLWWSPCTPFTEDPPASLEVLSERCWGTMQAVEPGTDDEGPLLEPVGSEHA
QDTYLVLDKWLLPRNPPSEDLPGPGGSVDIVAMDEGSEASSCSSALASKPSPEGASAASFEYTILDPSSQLLRPWTLCPELPPTPPHLKYLYL
VVSDSGISTDYSSGDSQGAQGGLSDGPYSNPYENSLIPAAEPLPPSYVACS
>gi|4503781|ref|NP_001453.1| N-formyl peptide receptor 2 [Homo sapiens], SEQ ID NO: 35
METNFSTPLNEYEEVSYESAGYTVLRILPLVVLGVTFVLGVLGNGLVIWVAGFRMTRTVTTICYLNLALADFSFTATLPFLIVSMAMGEKW
PFGWFLCKLIHIVVDINLFGSVFLIGFIALDRCICVLHPVWAQNHRTVSLAMKVIVGPWILALVLTLPVFLFLTTVTIPNGDTYCTFNFASWGG
TPEERLKVAITMLTARGIIRFVIGFSLPMSIVAICYGLIAAKIFIKKGMIKSSRPLRVLTAVVASFFICWFPFQLVALLGTVWLKEMLFYGKYKII
DILVNPTSSLAFFNSCLNPMLYVFVGQDFRERLIHSLPTSLERALSEDSAPTNDTAANSASPPAETELQAM
>gi|4505213|ref|NP_002420.1| matrix metalloproteinase-19 isoform rasi-1 preproprotein [Homo
sapiens], SEQ ID NO: 36
MNCQQLWLGFLLPMTVSGRVLGLAEVAPVDYLSQYGYLQKPLEGSNNFKPEDITEALRAFQEASELPVSGQLDDATRARMRQPRCGLED
PFNQKTLKYLLLGRWRKKHLTFRILNLPSTLPPHTARAALRQAFQDWSNVAPLTFQEVQAGAADIRLSFHGRQSSYCSNTFDGPGRVLAH
ADIPELGSVHFDEDEFWTEGTYRGVNLRIIAAHEVGHALGLGHSRYSQALMAPVYEGYRPHFKLHPDDVAGIQALYGKKSPVIRDEEEEET
ELPTVPPVPTEPSPMPDPCSSELDAMMLGPRGKTYAFKGDYVWTVSDSGPGPLFRVSALWEGLPGNLDAAVYSPRTQWIHFFKGDKVWR
YINFKMSPGFPKKLNRVEPNLDAALYWPLNQKVFLFKGSGYWQWDELARTDFSSYPKPIKGLFTGVPNQPSAAMSWQDGRVYFFKGKV
YWRLNQQLRVEKGYPRNISHNWMHCRPRTIDTTPSGGNTTPSGTGITLDTTLSATETTFEY
>gi|4507163|ref|NP_003098.1| transcription factor SOX-4 [Homo sapiens], SEQ ID NO: 37
MVQQTNNAENTEALLAGESSDSGAGLELGIASSPTPGSTASTGGKADDPSWCKTPSGHIKRPMNAFMVWSQIERRKIMEQSPDMHNAEIS
KRLGKRWKLLKDSDKIPFIREAERLRLKHMADYPDYKYRPRKKVKSGNANSSSSAAASSKPGEKGDKVGGSGGGGHGGGGGGGSSNAG
GGGGGASGGGANSKPAQKKSCGSKVAGGAGGGVSKPHAKLILAGGGGGGKAAAAAAASFAAEQAGAAALLPLGAAADHHSLYKARTP
SASASASSAASASAALAAPGKHLAEKKVKRVYLFGGLGTSSSPVGGVGAGADPSDPLGLYEEEGAGCSPDAPSLSGRSSAASSPAAGRSPA
DHRGYASLRAASPAPSSAPSHASSSASSHSSSSSSSGSSSSDDEFEDDLLDLNPSSNFESMSLGSFSSSSALDRDLDFNFEPGSGSHFEFPDYC
TPEVSEMISGDWLESSISNLVFTY
>gi|4507231|ref|NP_003134.1| single-stranded DNA-binding protein, mitochondrial precursor [Homo
sapiens], SEQ ID NO: 38
MFRRPVLQVLRQFVRHESETTTSLVLERSLNRVHLLGRVGQDPVLRQVEGKNPVTIFSLATNEMWRSGDSEVYQLGDVSQKTTWHRISVF
RPGLRDVAYQYVKKGSRIYLEGKIDYGEYMDKNNVRRQATTIIADNIIFLSDQTKEKE
>gi|48257122|gb|AAH16977.2| PBX3 protein [Homo sapiens], SEQ ID NO: 39
EKTGLSIRGAQEEDPPDPQLMRLDNMLLAEGVSGPEKGGGSAAAAAAAAASGGSSDNSIEHSDYRAKLTQIRQIYHTELEKYEQACNEFTT
HVMNLLREQSRTRPISPKEIERMVGIIHRKFSSIQMQLKQSTCEAVMILRSRFLDARRKRRNFSKQATEILNEYFYSHLSNPYPSEEAKEELA
KKCSITVSQSLVKDPKERGSKGSDIQPTSVVSNWFGNKRIRYKKNIGKFQEEANLYAAKTAVTAAHAVAAAVQNNQTNSPTTPNSGSSGS
FNLPNSGDMFMNMQSLNGDSYQGSQVGANVQSQVDTLRHVINQTGGYSDGLGGNSLYSPHNLNANGGWQDATTPSSVTSPTEGPGSVH
SDTSN
>gi|547921001ref|NP_001973.2| receptor tyrosine-protein kinase erbB-3 isoform 1 precursor [Homo
sapiens], SEQ ID NO: 40
MRANDALQVLGLLFSLARGSEVGNSQAVCPGTLNGLSVTGDAENQYQTLYKLYERCEVVMGNLEIVLTGHNADLSFLQWIREVTGYVLV
AMNEFSTLPLPNLRVVRGTQVYDGKFAIFVMLNYNTNSSHALRQLRLTQLTEILSGGVYIEKNDKLCHMDTIDWRDIVRDRDAEIVVKDN
GRSCPPCHEVCKGRCWGPGSEDCQTLTKTICAPQCNGHCFGPNPNQCCHDECAGGCSGPQDTDCFACRHFNDSGACVPRCPQPLVYNKL
TFQLEPNPHTKYQYGGVCVASCPHNFVVDQTSCVRACPPDKMEVDKNGLKMCEPCGGLCPKACEGTGSGSRFQTVDSSNIDGFVNCTKI
LGNLDFLITGLNGDPWHKIPALDPEKLNVFRTVREITGYLNIQSWPPHMHNFSVFSNLTTIGGRSLYNRGFSLLIMKNLNVTSLGFRSLKEIS
AGRIYISANRQLCYHHSLNWTKVLRGPTEERLDIKHNRPRRDCVAEGKVCDPLCSSGGCWGPGPGQCLSCRNYSRGGVCVTHCNFLNGEP
REFAHEAECFSCHPECQPMEGTATCNGSGSDTCAQCAHFRDGPHCVSSCPHGVLGAKGPIYKYPDVQNECRPCHENCTQGCKGPELQDC
LGQTLVLIGKTHLTMALTVIAGLVVIFMMLGGTFLYWRGRRIQNKRAMRRYLERGESIEPLDPSEKANKVLARIFKETELRKLKVLGSGVF
GTVHKGVWIPEGESIKIPVCIKVIEDKSGRQSFQAVTDHMLAIGSLDHAHIVRLLGLCPGSSLQLVTQYLPLGSLLDHVRQHRGALGPQLLL
NWGVQIAKGMYYLEEHGMVHRNLAARNVLLKSPSQVQVADFGVADLLPPDDKQLLYSEAKTPIKWMALESIHFGKYTHQSDVWSYGVT
VWELMTFGAEPYAGLRLAEVPDLLEKGERLAQPQICTIDVYMVMVKCWMIDENIRPTFKELANEFTRMARDPPRYLVIKRESGPGIAPGPE
PHGLTNKKLEEVELEPELDLDLDLEAEEDNLATTTLGSALSLPVGTLNRPRGSQSLLSPSSGYMPMNQGNLGESCQESAVSGSSERCPRPVS
LHPMPRGCLASESSEGHVTGSEAELQEKVSMCRSRSRSRSPRPRGDSAYHSQRHSLLTPVTPLSPPGLEEEDVNGYVMPDTHLKGTPSSRE
GTLSSVGLSSVLGTEEEDEDEEYEYMNRRRRHSPPHPPRPSSLEELGYEYMDVGSDLSASLGSTQSCPLHPVPIMPTAGTTPDEDYEYMNR
QRDGGGPGGDYAAMGACPASEQGYEEMRAFQGPGHQAPHVHYARLKTLRSLEATDSAFDNPDYWHSRLFPKANAQRT
>gi|55661618|emb|CAH72013.1| RIO kinase 1 (yeast) [Homo sapiens], SEQ ID NO: 41
MSRVVPGQFDDADSSDSENRDLKTVKEKDDILFEDLQDNVNENGEGEIEDEEEEGYDDDDDDWDWDEGVGKLAKGYVWNGGSNPQAN
RQTSDSSSAKMSTPADKVLRKFENKINLDKLNVTDSVINKVTEKSRQKEADMYRIKDKADRATVEQVLDPRTRMILFKMLTRGIITEINGCI
STGKEANVYHASTANGESRAIKIYKTSILVFKDRDKYVSGEFRFRHGYCKGNPRKMVKTWAEKEMRNLIRLNTAEIPCPEPIMLRSHVLVM
SFIGKDDMPAPLLKNVQLSESKARELYLQVIQYMRRMYQDARLVHADLSEFNMLYHGGGVYBDVSQSVEHDHPHALEFLRKDCANVND
FFMRHSVAVMTVRELFEFVTDPSITHENMDAYLSKAMEIASQRTKEERSSQDHVDEEVFKRAYIPRTLNEVKNYERDMDBMKLKEEDMA
MNAQQDNILYQTVTGLKKDLSGVQKVPALLENQVEERTCSDSEDIGSSECSDTDSEEQGDHARPKKHTTDPDIDKKERKKMVKEAQREK
RKNKIPKHVKKRKEKTAKTKKGK
>gi|640248|pdb|2IRT| A Chain A, Initial Crystallographic Analyses Of A Recombinant Interleukin-1
Receptor Antagonist Protein, SEQ ID NO: 42
RPSGRKSSKMQAFRIWDVNQKTFYLRNNQLVAGYLQGPNVNLEEKIDVVPIEPHALFLGIHGGKMCLSCVKSGDETRLQLEAVNITDLSEN
RKQDKRFAFIRSDSGPTTSFESAACPGWFLCTAMEADQPVSLTNMPDEGVMVTKFYFQEDE
>gi|7020726|dbj|BAA91249.1| unnamed protein product [Homo sapiens], SEQ ID NO: 43
MWGACKVKVHDSLATISITLRRYLRLGATMAKSKFEYVRDFEADDTCLAHCWVVVRLDGRNFHRFAEKHNFAKPNDSRALQLMTKCAQ
TVMEELEDIVIAYGQSDEYSFVFKRKTNWFKRRASKFMTHVASQFASSYVFYWRDYFEDQPLLYPPGFDGRVVVYPSNQTLKDYLSWRQ
ADCHINNLYNTVFWALIQQSGLTPVQAQGRLQGTLAADKNEILFSEFNINYNNEPPMYRKGTVLIWQKVDEVMTKEIKLPTEMEGKKMA
VTRTRTKPVPLHCDIIGDAFWKEHPEILDEDS
>gi|74099694|ref|NP_000447.2| sulfite oxidase, mitochondrial precursor [Homo sapiens],
SEQ ID NO: 44
MLLLHRAVVLRLQQACRLKSIPSRICIQACSTNDSFQPQRPSLTFSGDNSSTQGWRVMGTLLGLGAVLAYQDHRCRAAQESTHIYTKEEVS
SHTSPETGIWVTLGSEVFDVTEFVDLHPGGPSKLMLAAGGPLEPFWALYAVHNQSHVRELLAQYKIGELNPEDKVAPTVETSDPYADDPV
RHPALKVNSQRPFNAEPPPELLTENYITPNPIFFTRNHLPVPNLDPDTYRLHVVGAPGGQSLSLSLDDLHNFPRYEITVTLQCAGNRRSEMT
QVKEVKGLEWRTGAISTARWAGARLCDVLAQAGHQLCETEAHVCFEGLDSDPTGTAYGASIPLARAMDPEAEVLLAYEMNGQPLPRDH
GFPVRVVVPGVVGARHVKWLGRVSVQPEESYSHWQRRDYKGFSPSVDWETVDFDSAPSIQELPVQSAITEPRDGETVESGEVTIKGYAWS
GGGRAVIRVDVSLDGGLTWQVAKLDGEEQRPRKAWAWRLWQLKAPVPAGQKELNIVCKAVDDGYNVQPDTVAPIWNLRGVLSNAWH
RVHVYVSP
>gl17529553|emb|CAB86601.1| xce [Homo sapiens], SEQ ID NO: 45
MEPPYSLTAHYDEFQEVKYVSRCGAGGARGASLPPGFPLGAARSATGARSGLPRWNRREVCLLSGLVFAAGLCAILAAMLALKYLGPVA
AGGGACPEGCPERKAFARAARFLAANLDASIDPCQDFYSFACGGWLRRHAIPDDKLTYGTIAAIGEQNEERLRRLLARPGGGPGGAAQRK
VRAFFRSCLDMREIERLGPRPMLEVIEDCGGWDLGGAEERPGVAARWDLNRLLYKAQGVYSAAALFSLTVSLDDRNSSRYVIRIDQDGLT
LPERTLYLAQDEDSEKILAAYRVFMERVLSLLGADAVEQKAQEILQVEQQLANITVSEYDDLRRDVSSMYNKVTLGQLQKITPHLRWKWL
LDQIFQEDFSEEEEVVLLATDYMQQVSQLIRSTPHRVLHNYLVWRVVVVLSEHLSPPFREALHELAQEMEGSDKPQELARVCLGQANRHF
GMALGALFVHEHFSAASKAKVQQLVEDIKYILGQRLEELDWMDAETRAAARAKLQYMMVMVGYPDFLLKPDAVDKEYEFEVHEKTYF
KNILNSIRFSIQLSVKKIRQEVDKSTWLLPPQALNAYYLPNKNQMVFPAGILQPTLYDPDFPQSLNYGGIGTIIGHELTHGYDDWGGQYDRS
GNLLHWWTEASYSRFLRKAECIVRLYDNFTVYNQRVNGKHTLGENIADMGGLKLAYHAYQKWVREHGPEHPLPRLKYTHDQLFFIAFA
QNWCIKRRSQSIYLQVLTDKHAPEHYRVLGSVSQFEEFGRAFHCPKDSPMNPAHKCSVW
>gi|7706143|ref|NP_057692.1| armadillo repeat-containing X-linked protein 1 [Homo sapiens],
SEQ ID NO: 46
MGRTREAGCVAAGVVIGAGACYCVYRLAWGRDENEKIWDEDEESTDTSEIGVETVKGAKTNAGAGSGAKLQGDSEVKPEVSLGLEDCP
GVKEKAHSGSHSGGGLEAKAKALFNTLKEQASAKAGKGARVGTISGNRTLAPSLPCPGGRGGGCHPTRSGSRAGGRASGKSKGKARSKS
TRAPATTWPVRRGKFNFPYKIDDILSAPDLQKVLNILERTNDPFIQEVALVTLGNNAAYSFNQNAIRELGGVPIIAKLIKTKDPIIREKTYNAL
NNLSVNAENQGKIKTYISQVCDDTMVCRLDSAVQMAGLRLLTNMTVTNHYQHLLSYSFPDFFALLFLGNHFTKIQIMKLIINFTENPAMTR
ELVSCKVPSELISLFNKEWDREILLNILTLFENINDNIKNEGLASSRKEFSRSSLFFLFKESGVCVKKIKALANHNDLVVKVKVLKVLTKL
>gi|82775373|ref|NP_057696.2| mitoferrin-1 [Homo sapiens], SEQ ID NO: 47
MELRSGSVGSQAVARRMDGDSRDGGGGKDATGSEDYENLPTSASVSTHMTAGAMAGILEHSVMYPVDSVKTRMQSLSPDPKAQYTSIY
GALKKIMRTEGFWRPLRGVNVMIMGAGPAHAMYFACYENMKRTLNDVFHHQGNSHLANGIAGSMATLLHDAVMNPAEVVKQRLQMY
NSQHRSAISCIRTVWRTEGLGAFYRSYTTQLTMNIPFQSIHFITYEFLQEQVNPHRTYNPQSHIISGGLAGALAAAATTPLDVCKTLLNTQEN
VALSLANISGRLSGMANAFRTVYQLNGLAGYFKGIQARVIYQMPSTAISWSVYEFFKYFLTKRQLENRAPY
>gi|9910352|ref|NP_064530.1| endophilin-B2 [Homo sapiens], SEQ ID NO: 48
MDFNMKKLASDAGIFFTRAVQFTEEKFGQAEKTELDAHFENLLARADSTKNWTEKILRQTEVLLQPNP_SARVEEFLYEKLDRKVPSRVTN
GELLAQYMADAASELGPTTPYGKTLIKVAEAEKQLGAAERDFIHTASISFLTPLRNFLEGDWKTISKERRLLQNRRLDLDACKARLKKAKA
AEAKATTVPDFQETRPRNYILSASASALWNDEVDKAEQELRVAQTEFDRQAEVTRLLLEGISSTHVNHLRCLHEFVKSQTTYYAQCYRHM
LDLQKQLGRFPGTFVGTTEPASPPLSSTSPTTAAATMPVVPSVASLAPPGEASLCLEEVAPPASGTRKARVLYDYEAADSSELALLADELITV
YSLPGMDPDWLIGERGNKKGKVPVTYLELLS
>gi|224586777|ref|NP_060288.3| WD repeat-containing protein WRAP73 [Homo sapiens], SEQ ID NO: 49
MNFSEVFKLSSLLCKFSPDGKYLASCVQYRLVVRDVNTLQILQLYTCLDQIQHIEWSADSLFILCAMYKRGLVQVWSLEQPEWHCKIDEGS
AGLVASCWSPDGRHILNTTEFHLRITVWSLCTKSVSYIKYPKACLQGITFTRDGRYMALAERRDCKDYVSIFVCSDWQLLRHFDTDTQDLT
GIEWAPNGCVLAVWDTCLEYKILLYSLDGRLLSTYSAYEWSLGIKSVAWSPSSQFLAVGSYDGKVRILNHVTWKMITEFGHPAAINDPKIV
VYKEAEKSPQLGLGCLSFPPPRAGAGPLPSSESKYEIASVPVSLQTLKPVTDRANPKIGIGMLAFSPDSYFLATRNDNIPNAVWVWDIQKLR
LFAVLEQLSPVRAFQWDPQQPRLAICTGGSRLYLWSPAGCMSVQVPGEGDFAVLSLCWHLSGDSMALLSKDHFCLCFLETEAVVGTACR
QLGGHT
>gi|11386157|ref|NP_001776.1| cytidine deaminase [Homo sapiens], SEQ ID NO: 50
MAQKRPACTLKPECVQQLLVCSQEAKKSAYCPYSHFPVGAALLTQEGRIFKGCNIENACYPLGICAERTAIQKAVSEGYKDFRAIAIASDM
QDDFISPCGACRQVMREFGTNWPVYMTKPDGTYIVMTVQELLPSSFGPEDLQKTQ
>gi|206597477|ref|NP_001128651.1| palmitoyltransferase ZDHHC3 isoform 1 [Homo sapiens],
SEQ ID NO: 51
MMLIPTHHFRNIERKPEYLQPEKCVPPPYPGPVGTMWFIRDGCGIACAIVTWFLVLYAEFVVLFVMLIPSRDYVYSIINGIVFNLLAFLALAS
HCRAMLTDPGAVPKGNATKEFIESLQLKPGQVVYKCPKCCSIKPDRAHHCSVCKRCIRKMDHHCPWVNNCVGENNQKYFVLFTMYIALI
SLHALIMVGFHFLHCFEEDWTKCSSFSPPTTVILLILLCFEGLLFLIFTSVMFGTQVHSICTDETGIEQLKKEERRWAKKTKWMNMKAVFGH
PFSLGWASPFATPDQGKADPYQYVV
>gi|4759088|ref|NP_004197.1| vesicle-trafficking protein SEC22c isoform b [Homo sapiens],
SEQ ID NO: 52
MSVIFFACVVRVRDGLPLSASTDFYHTQDFLEWRRRLKSLALRLAQYPGRGSAEGCDFSIHFSSFGDVACMAICSCQCPAAMAFCFLETLW
WEFTASYDTTCIGLASRPYAFLEFDSIIQKVKWHFNYVSSSQMECSLEKIQEELKLQPPAVLTLEDTDVANGVMNGHTPMHLEPAPNFRME
PVTALGILSLILNIMCAALNLIRGVHLAEHSLQVAHEEIGNILAFLVPFVACIFQDPRSWFCWLDQTS
>gi|49574546|ref|NP_001001890.1| runt-related transcription factor 1 isoform AML1b [Homo sapiens],
SEQ ID NO: 53
MRIPVDASTSRRFTPPSTALSPGKMSEALPLGAPDAGAALAGKLRSGDRSMVEVLADHPGELVRTDSPNFLCSVLPTHWRCNKTLPIAFKV
VALGDVPDGTLVTVMAGNDENYSAELRNATAAMKNQVARFNDLRFVGRSGRGKSFTLTITVFTNPPQVATYHRAIKITVDGPREPRRHR
QKLDDQTKPGSLSFSERLSELEQLRRTAMRVSPHHPAPTPNPRASLNHSTAFNPQPQSQMQDTRQIQPSPPWSYDQSYQYLGSIASPSVHP
ATPISPGRASGMTTLSAELSSRLSTAPDLTAFSDPRQFPALPSISDPRMHYPGAFTYSPTPVTSGIGIGMSAMGSATRYHTYLPPPYPGSSQA
QGGPFQASSPSYHLYYGASAGSYQFSMVGGERSPPRILPPCTNASTGSALLNPSLPNQSDVVEAEGSHSNSPTNMAPSARLEEAVWRPY
>gi|4505725|ref|NP_000457.1| peroxisome biogenesis factor 1 [Homo sapiens], SEQ ID NO: 54
MWGSDRLAGAGGGGAAVTVAFTNARDCFLHLPRRLVAQLHLLQNQAIEVVWSHQPAFLSWVEGRHFSDQGENVAEINRQVGQKLGLSN
GGQVFLKPCSHVVSCQQVEVEPLSADDWEILELHAVSLEQHLLDQIRIVFPKAIFPVWVDQQTYIFIQIVALIPAASYGRLETDTKLLIQPKTR
RAKENTFSKADAEYKKLHSYGRDQKGMMKELQTKQLQSNTVGITESNENESEIPVDSSSVASLWTMIGSIFSFQSEKKQETSWGLTEINAF
KNMQSKVVPLDNIFRVCKSQPPSIYNASATSVFHKHCAIHVFPWDQEYFDVEPSFTVTYGKLVKLLSPKQQQSKTKQNVLSPEKEKQMSE
PLDQKKIRSDHNEEDEKACVLQVVWNGLEELNNAIKYTKNVEVLHLGKVWIPDDLRKRLNIEMHAVVRITPVEVTPKIPRSLKLQPRENLP
KDISEEDIKTVFYSWLQQSTTTMLPLVISEEEFIKLETKDGLKEFSLSIVHSWEKEKDKNIFLLSPNLLQKTTIQVLLDPMVKEENSEEIDFILPF
LKLSSLGGVNSLGVSSLEHITHSLLGRPLSRQLMSLVAGLRNGALLLTGGKGSGKSTLAKAICKEAFDKLDAHVERVDCKALRGKRLENIQ
KTLEVAFSEAVWMQPSVVLLDDLDLIAGLPAVPEHEHSPDAVQSQRLAHALNDMIKEFISMGSLVALIATSQSQQSLHPLLVSAQGVHIFQ
CVQHIQPPNQEQRCEILCNVIKNKLDCDINKFTDLDLQHVAKETGGFVARDFTVLVDRAIHSRLSRQSISTREKLVLTTLDFQKALRGFLPAS
LRSVNLHKPRDLGWDKIGGLHEVRQILMDTIQLPAKYPELFANLPIRQRTGILLYGPPGTGKTLLAGVIARESRMNFISVKGPELLSKYIGAS
EQAVRDIFIRAQAAKPCILFFDEFESIAPRRGHDNTGVTDRVVNQLLTQLDGVEGLQGVYVLAATSRPDLIDPALLRPGRLDKCVYCPPPDQ
VSRLEILNVLSDSLPLADDVDLQHVASVTDSFTGADLKALLYNAQLEALHGMLLSSGLQDGSSSSDSDLSLSSMVFLNHSSGSDDSAGDGE
CGLDQSLVSLEMSEILPDESKFNMYRLYFGSSYESELGNGTSSDLSSQCLSAPSSMTQDLPGVPGKDQLFSQPPVLRTASQEGCQELTQEQR
DQLRADISIIKGRYRSQSGEDESMNQPGPIKTRLAISQSHLMTALGHTRPSISEDDWKNFAELYESFQNPKRRKNQSGTMFRPGQKVTLA
>gi|4507231|ref|NP_003134.1| single-stranded DNA-binding protein, mitochondrial precursor [Homo
sapiens] SEQ ID NO: 55
MFRRPVLQVLRQFVRHESETTTSLVLERSLNRVHLLGRVGQDPVLRQVEGKNPVTIFSLATNEMWRSGDSEVYQLGDVSQKTTWHRISVF
RPGLRDVAYQYVKKGSRIYLEGKIDYGEYMDKNNVRRQATTIIADNIIFLSDQTKEKE
>gi|270132935|ref|NP_001003700.1| ras-responsive element-binding protein 1 isoform 3 [Homo
sapiens], SEQ ID NO: 56
MTSSSPAGLEGSDLSSINTMMSAVMSVGKVTENGGSPQGIKSPSKPPGPNRIGRRNQETKEEKSSYNCPLCEKICTTQHQLTMHIRQHNTD
TGGADHSCSICGKSLSSASSLDRHMLVHSGERPYKCTVCGQSFTTNGNMHRHMKIHEKDPNSATATAPPSPLKRRRLSSKRKLSHDAESE
REDPAPAKKMVEDGQSGDLEKKADEVFHCPVCFKEFVCKYGLETHMETHSDNPLRCDICCVTFRTHRGLLRHNALVHKQLPRDAMGRPF
IQNNPSIPAGFHDLGFTDFSCRKFPRISQAWCETNLRRCISEQHRFVCDTCDKAFPMLCSLALHKQTHVAADQGQEKPQATPLPGDALDQK
GFLALLGLQHTKDVRPAPAEEPLPDDNQAIQLQTLKCQLPQDPGCTNLLSLSPFEAASLGGSLTVLPATKDSIKHLSLQPFQKGFIIQPDSSIV
VKPISGESAIELADIQQILKMAASAPPQISLPPFSKAPAAPLQAIFKHMPPLKPKPLVTPRTVVATSTPPPLINAQQASPGCISPSLPPPPLKLLK
GSVEAASNAHLLQSKSGTQPHAATRLSLQQPRAELPGQPEMKTQLEQDSBEALLPLSMEAKIKQEITEGELKAFMTAPGGKKTPAMRKVL
YPCRFCNQVFAFSGVLRAHVRSHLGISPYQCNICDYIAADKAALIRHLRTHSGERPYICKICHYPFTVKANCERHLRKKHLKATRKDIEKNI
EYVSSSAAELVDAFCAPDTVCRLCGEDLKHYRALRIHMRTHCGRGLGGGHKGRKPFECKECSAAFAAKRNCIHHILKQHLHVPEQDIESY
VLAADGLGPAEAPAAEASGRGEDSGCAALGDCKPLTAFLEPQNGFLHRGPTQPPPPHVSIKLEPASSFAVDFNEPLDFSQKGLALVQVKQE
NISFLSPSSLVPYDCSMEPIDLSIPKNFRKGDKDLATPSEAKKPEEEAGSSEQPSPCPAPGPSLPVTLGPSGILESPMAPAPAATPEPPAQPLQG
PVQLAVPIYSSALVSSPPLVGSSALLSGTALLRPLRPKPPLLLPKPPVTEELPPLASIAQIISSVSSAPTLLKTKVADPGPASTGSNTTASDSLGG
SVPKAATTATPAATTSPKESSEPPAPASSPEAASPTEQGPAGTSKKRGRKRGMRSRPRANSGGVDLDSSGEFASIEKMLATTDTNKFSPFLQ
TAEDNTQDEVAGAPADHHGPSDEEQGSPPEDKLLRAKRNSYTNCLQKITCPHCPRVFPWASSLQRHMLTHTGQKPFPCQKCDAFFSTKSN
CERHQLRKHGVTTCSLRRNGLIPQSKESDVGSHDSTGERPYKCQTCERTFTLKHSLVRHQRIHQKARHAKHHGKDSDKEERGEEDSENES
THSGNNAVSENEAELAPNASNHMAVTRSRKEGLASATKDCSHREEKVTAGWPSEPGQGDLNPESPAALGQDLLEPRSKRPAHPILATADG
ASQLVGME
>gi|157738629|ref|NP_001099038.1| kinesin-like protein KIF13A isoform d [Homo sapiens],
SEQ ID NO: 57
MSDTKVKVAVRVRPMNRRELELNTKCVVEMEGNQTVLHPPPSNTKQGERKPPKVFAFDYCFWSMDESNTTKYAGQEVVFKCLGEGILE
KAFQGYNACIFAYGQTGSGKSFSMMGHAEQLGLIPRLCCALFKRISLEQNESQTFKVEVSYMEIYNEKVRDLLDPKGSRQSLKVREHKVLG
PYVDGLSQLAVTSFEDIESLMSEGNKSRTVAATNMNEESSRSHAVFNIIITQTLYDLQSGNSGEKVSKVSLVDLAGSERVSKTGAAGERLKE
GSNINKSLTTLGLVISSLADQAAGKGKSKFVPYRDSVLTWLLKDNLGGNSQTSMIATISPAADNYEETLSTLRYADRAKRIVNHAVVNEDP
NAKVIRELREEVEKLREQLSQAEAMKAPELKEKLEESEKLIKELTVTWEEKLRKTEEIAQERQRQLESMGISLEMSGIKVGDDKCYLVNLN
ADPALNELLVYYLKDHTRVGADTSQDIQLFGIGIQPQHCEIDIASDGDVTLTPKENARSCVNGTLVCSTTQLWHGDRILWGNNHFFRINLPK
RKRRDWLKDFEKETGPPEHDLDAASEASSEPDYNYEFAQMEVIMKTLNSNDPVQNVVQVLEKQYLEEKRSALEEQRLMYERELEQLRQQ
LSPDRQPQSSGPDRLAYSSQTAQQKVTQWAEERDELFRQSLAKLREQLVKANTLVREANFLAEEMSKLTDYQVTLQIPAANLSANRKRG
AIVSEPAIQVRRKGKSTQVWTIEKLENKLIDMRDLYQEWKEKVPEAKRLYGKRGDPFYEAQENHNLIGVANVFLECLFCDVKLQYAVPIIS
QQGEVAGRLHVEVMRVTGAVPERVVEDDSSENSSESGSLEVVDSSGEIIHRVKKLTCRVKIKEATGLPLNLSNFVFCQYTFWDQCESTVA
APVVDPEVPSPQSKDAQYTVTFSHCKDYVVNVTEEFLEFISDGALAIEVWGHRCAGNGSSIWEVDSLHAKTRTLHDRWNEVTRRIEMWISI
LELNELGEYAAVELHQAKDVNTGGIFQLRQGHSRRVQVTVKPVQHSGTLPLMVEAILSVSIGCVTARSTKLQRGLDSYQEEDLNCVRERW
SDALIKRREYLDEQIKKVSNKTEKTEDDVEREAQLVEQWVGLTEERNAVLVPAPGSGIPGAPADWIPPPGMETHIPVLFLDLNADDLSANE
QLVGPHASGVNSILPKEHGSQFFYLPIIKHSDDEVSATASWDSSVHDSVHLNRVTPQNERIYLIVKTTVQLSHPAAMELVLRKRIAANIYNK
QSFTQSLKRRISLKNIFYSCGVTYEIVSNIPKATEEIEDRETLALLAARSENEGTSDGETYIEKYTRGVLQVENILSLERLRQAVTVKEALSTK
ARHIRRSLSTPNVHNVSSSRPDLSGFDEDDKGWPENQLDMSDYSSSYQDVACYGTLPRDSPRRNKEGCTSETPHALTVSPFKAFSPQPPKF
FKPLMPVKEEHKKRIALEARPLLSQEDSEEEENELEAINRKLISSQPYVPVEFADFSVYNASLENREWFSSKVDLSNSRVLEKEVSRSPTTSSI
TSGYFSHSASNATLSDMVVPSSDSSDQLAIQTKDADSTEHSTPSLVHDFRPSSNKELTEVEKGLVKDKIIVVPLKENSALAKGSPSSQSIPEK
NSKSLCRTGSCSELDACPSKISQPARGFCPREVTVEHTTNILEDHSFTEFMGVSEGKDFDGLTDSSAGELSSRRSLPNKTGGKTVSDGLHHP
SQLHSKLENDQGGTTSR
>gi|50284696|ref|NP_877434.2| E3 ubiquitin-protein ligase RNF144B [Homo sapiens], SEQ ID NO: 58
MGSAGRLHYLAMTAENPTPGDLAPAPLITCKLCLCEQSLDKMTTLQECQCIFCTACLKQYMQLAIREGCGSPITCPDMVCLNHGTLQEAEI
ACLVPVDQFQLYQRLKFEREVHLDPYRTWCPVADCQTVCPVASSDPGQPVLVECPSCHLKFCSCCKDAWHAEVSCRDSQPIVLPTEHRAL
FGTDAEAPIKQCPVCRVYIERNEGCAQMMCKNCKHTFCWYCLQNLDNDIFLRHYDKGPCRNKLGHSRASVMWNRTQVVGILVGLGIIAL
VTSPLLLLASPCIICCVCKSCRGKKKKHDPSTT
>gi|20149594|ref|NP_031381.2| heat shock protein HSP 90-beta [Homo sapiens], SEQ ID NO: 59
MPEEVHHGEEEVETFAFQAEIAQLMSLIINTFYSNKEIFLRELISNASDALDKIRYESLTDPSKLDSGKELKIDIIPNPQERTLTLVDTGIGMTK
ADLINNLGTIAKSGTKAFMEALQAGADISMIGQFGVGFYSAYLVAEKVVVITKHNDDEQYAWESSAGGSFTVRADHGEPIGRGTKVILHLK
EDQTEYLEERRVKEVVKKHSQFIGYPITLYLEKEREKEISDDEAEEEKGEKEEEDKDDEEKPKIEDVGSDEEDDSGKDKKKKTKKIKEKYID
QEELNKTKPIWTRNFDDITQEEYGEFYKSLTNDWEDHLAVKHFSVEGQLEFRALLFIPRRAPFDLFENKKKKNNIKLYVRRVFIMDSCDELI
PEYLNFIRGVVDSEDLPLNISREMLQQSKILKVIRKNIVKKCLELFSELAEDKENYKKFYEAFSKNLKLGIHEDSTNRRRLSELLRYHTSQSG
DEMTSLSEYVSRMKETQKSIYYITGESKEQVANSAFVERVRKRGFEVVYMTEPIDEYCVQQLKEFDGKSLVSVTKEGLELPEDEEEKKKM
EESKAKFENLCKLMKEILDKKVEKVTISNRLVSSPCCIVTSTYGWTANMERIMKAQALRDNSTMGYMMAKKHLEINPDHPIVETLRQKAE
ADKNDKAVKDLVVLLFETALLSSGFSLEDPQTHSNRIYRMIKLGLGIDEDEVAAEEPNAAVPDEIPPLEGDEDASRMEEVD
>gi|58533161|ref|NP_001011516.1| PDZ and LIM domain protein 5 isoform e [Homo sapiens],
SEQ ID NO: 60
MSNYSVSLVGPAPWGFRLQGGKDFNMPLTISSLKDGGKAAQANVRIGDVVLSIDGINAQGMTHLEAQNKIKGCTGSLNMTLQRASAAPK
PEPVPVQKKTQVTNNPGTVKIPPKRPPRKHIVERYTEFYHVPTHSDASKKRLIEDTEDWRPRTGTTQSRSFRILAQITGTEHLKESEADNTK
KAKEKIPLHVFSPKYTKLRDWHHEVSARALNVQ
>gi|4504023|ref|NP_000815.1| glycine receptor subunit beta isoform A precursor [Homo sapiens],
SEQ ID NO: 61
MKFLLTTAFLILISLWVEEAYSKEKSSKKGKGKKKQYLCPSQQSAEDLARVPANSTSNILNRLLVSYDPRIRPNFKGIPVDVVVNIFINSFGSI
QETTMDYRVNIFLRQKWNDPRLKLPSDFRGSDALTVDPTMYKCLWKPDLFFANEKSANFHDVTQENILLFIFRDGDVLVSMRLSITLSCPL
DLTLFPMDTQRCKMQLESFGYTTDDLRFIWQSGDPVQLEKIALPQFDIKKEDIEYGNCTKYYKGTGYYTCVEVIFTLRRQVGFYMMGVYA
PTLLIVVLSWLSFWINPDASAARVPLGIFSVLSLASECTTLAAELPKVSYVKALDVWLIACLLFGFASLVEYAVVQVMLNNPKRVEAEKARI
AKAEQADGKGGNVAKKNTVNGTGTPVHISTLQVGETRCKKVCTSKSDLRSNDFSIVGSLPRDFELSNYDCYGKPIEVNNGLGKSQAKNN
KKPPPAKPVIPTAAKRIDLYARALFPFCFLFFNVIYWSIYL
>gi|39725952|ref|NP_060516.2| angiogenic factor with G patch and FHA domains 1 [Homo sapiens],
SEQ ID NO: 62
MASEAPSPPRSPPPPTSPEPELAQLRRKVEKLERELRSCKRQVREIEKLLHHTERLYQNAESNNQELRTQVEELSKILQRGRNEDNKKSDVE
VQTENHAPWSISDYFYQTYYNDVSLPNKVTELSDQQDQAIETSILNSKDHLQVENDAYPGTDRTENVKYRQVDHFASNSQEPASALATED
TSLEGSSLAESLRAAAEAAVSQTGFSYDENTGLYFDHSTGFYYDSENQLYYDPSTGIYYYCDVESGRYQFHSRVDLQPYPTSSTKQSKDK
KLKKKRKDPDSSATNEEKDLNSEDQKAFSVEHTSCNEEENFANMKKKAKIGIHHKNSPPKVTVPTSGNTIESPLHENISNSTSFKDEKIMET
DSEPEEGEITDSQTEDSYDEAITSEGNVTAEDSEDEDEDKIWPPCIRVIVIRSPVLQIGSLFIITAVNPATIGREKDMEHTLRIPEVGVSKFHAEI
YFDHDLQSYVLVDQGSQNGTIVNGKQILQPKTKCDPYVLEHGDEVKIGETVLSFHIHPGSDTCDGCEPGQVRAHLRLDKKDESFVGPTLSK
EEKELERRKELKKIRVKYGLQNTEYEDEKTLKNPKYKDRAGKRREQVGSEGTFQRDDAPASVHSEITDSNKGRKMLEKMGWKKGEGLG
KDGGGMKTPIQLQLRRTHAGLGTGKPSSFEDVHLLQNKNKKNWDKARERFTENFPETKPQKDDPGTMPWVKGTLE
>gi|7706318|ref|NP_057113.1| dehydrogenase/reductase SDR family member 7 precursor [Homo sapiens],
SEQ ID NO: 63
MNWELLLWLLVLCALLLLLVQLLRFLRADGDLTLLWAEWQGRRPEWELTDMVVWVTGASSGIGEELAYQLSKLGVSLVLSARRVHELER
VKRRCLENGNLKEKDILVLPLDLTDTGSHEAATKAVLQEFGRIDILVNNGGMSQRSLCMDTSLDVYRKLIELNYLGTVSLTKCVLPHMIER
KQGKIVTVNSILGIISVPLSIGYCASKHALRGFFNGLRTELATYPGIIVSNICPGPVQSNIVENSLAGEVTKTIGNNGDQSHKMTTSRCVRLMLI
SMANDLKEVWISEQPFLLVTYLWQYMPTWAWWITNKMGKKRIENFKSGVDADSSYFKIFKTKHD
>gi|7669526|ref|NP_039258.1| pro-neuregulin-1, membrane-bound isoform isoform HRG-alpha [Homo
sapiens], SEQ ID NO: 64
MSERKEGRGKGKGKKKERGSGKKPESAAGSQSPALPPRLKEMKSQESAAGSKLVLRCETSSEYSSLRFKWFKNGNELNRKNKPQNIKIQK
KPGKSELRINKASLADSGEYMCKVISKLGNDSASANITIVESNEIITGMPASTEGAYVSSESPIRISVSTEGANTSSSTSTSTTGTSHLVKCAEK
EKTFCVNGGECFMVKDLSNPSRYLCKCQPGFTGARCTENVPMKVQNQEKAEELYQKRVLTITGICIALLVVGIMCVVAYCKTKKQRKKL
HDRLRQSLRSERNNMMNIANGPHHPNPPPENVQLVNQYVSKNVISSEHIVEREAETSFSTSHYTSTAHHSTTVTQTPSHSWSNGHTESILSE
SHSVIVMSSVENSRHSSPTGGPRGRLNGTGGPRECNSFLRHARETPDSYRDSPHSERYVSAMTTPARMSPVDFHTPSSPKSPPSEMSPPVSS
MTVSMPSMAVSPFMEEERPLLLVTPPRLREKKFDHHPQQFSSFHHNPAHDSNSLPASPLRIVEDEEYETTQEYEPAQEPVKKLANSRRAKR
TKPNGHIANRLEVDSNTSSQSSNSESETEDERVGEDTPFLGIQNPLAASLEATPAFRLADSRTNPAGRFSTQEEIQARLSSVIANQDPIAV
>gi|27886593|ref|NP_005598.3| focal adhesion kinase 1 isoform b [Homo sapiens], SEQ ID NO: 65
MISADCNLCLPEYDRYLASSKIMAAAYLDPNLNHTPNSSTKTHLGTGMERSPGAMERVLKVFHYFESNSEPTTWASIIRHGDATDVRGIIQ
KIVDSHKVKHVACYGFRLSHLRSEEVHWLHVDMGVSSVREKYELAHPPEEWKYELRIRYLPKGFLNQFTEDKPTLNFFYQQVKSDYMLEI
ADQVDQEIALKLGCLEIRRSYWEMRGNALEKKSNYEVLEKDVGLKRFFPKSLLDSVKAKTLRKLIQQTFRQFANLNREESILKFFEILSPVY
RFDKECFKCALGSSWIISVELAIGPEEGISYLTDKGCNPTHLADFTQVQTIQYSNSEDKDRKGMLQLKIAGAPEPLTVTAPSLTIAENMADLI
DGYCRLVNGTSQSFIIRPQKEGERALPSIPKLANSEKQGMRTHAVSVSETDDYAEIIDEEDTYTMPSTRDYEIQRERIELGRCIGEGQFGDVH
QGIYMSPENPALAVAIKTCKNCTSDSVREKFLQEALTMRQFDHPHIVKLIGVITENPVWIIMELCTLGELRSFLQVRKYSLDLASLILYAYQL
STALAYLESKRFVHRDIAARNVLVSSNDCVKLGDFGLSRYMEDSTYYKASKGKLPIKWMAPESINFRRFTSASDVWMFGVCMWEILMHG
VKPFQGVKNNDVIGRIENGERLPMPPNCPPTLYSLMTKCWAYDPSRRPRFTELKAQLSTILEEEKAQQEERMRMESRRQATVSWDSGGSD
EAPPKPSRPGYPSPRSSEGFYPSPQHMVQTNHYQVSGYPGSHGITAMAGSIYPGQASLLDQTDSWNHRPQEIAMWQPNVEDSTVLDLRGI
GQVLPTHLMEERLIRQQQEMEEDQRWLEKEERFLKPDVRLSRGSIDREDGSLQGPIGNQHIYQPVGKPDPAAPPKKPPRPGAPGHLGSLAS
LSSPADSYNEGVKLQPQEISPPPTANLDRSNDKVYENVTGLVKAVIEMSSKIQPAPPEEYVPMVKEVGLALRTLLATVDETIPLLPASTHREI
EMAQKLLNSDLGELINKMKLAQQYVMTSLQQEYKKQMLTAAHALAVDAKNLLDVIDQARLKMLGQTRPH
>gi|5174764|ref|NP_005944.1| metallothionein-2 [Homo sapiens], SEQ ID NO: 66
MDPNCSCAAGDSCTCAGSCKCKECKCTSCKKSCCSCCPVGCAKCAQGCICKGASDKCSCCA
>gi|198041755|ref|NP_001128250.1| pre-B-cell leukemia transcription factor 3 isoform 2 [Homo
sapiens], SEQ ID NO: 67
MKPALFSVLCEIKEKTGLSIRGAQEEDPPDPQLMRLDNMLLAEGVSGPEKGGGSAAAAAAAAASGGSSDNSIEHSDYRAKLTQIRQIYHTE
LEKYEQACNEFTTHVMNLLREQSRTRPISPKEIERMVGIIHRKFSSIQMQLKQSTCEAVMILRSRFLDARRKRRNFSKQATEILNEYFYSHLS
NPYPSEEAKEELAKKCSITVSQVSNWFGNKRIRYKKNIGKFQEEANLYAAKTAVTAAHAVAAAVQNNQTNSPTTPNSGSSGSFNLPNSGD
MFMNMQSLNGDSYQGSQVGANVQSQVDTLRHVINQTGGYSDGLGGNSLYSPHNLNANGGWQDATTPSSVTSPTEGPGSVHSDTSN
>gi|4557323|ref|NP_000031.1| apolipoprotein C-III precursor [Homo sapiens], SEQ ID NO: 68
MQPRVLLVVALLALLASARASEAEDASLLSFMQGYMKHATKTAKDALSSVQESQVAQQARGWVTDGFSSLKDYWSTVKDKFSEFWDL
DPEVRPTSAVAA
>gi|33342278|ref|NP_079480.2| traB domain-containing protein [Homo sapiens], SEQ ID NO: 69
MDGEEQQPPHEANVEPVVPSEASEPVPRVLSGDPQNLSDVDAFNLLLEMKLKRRRQRPNLPRTVTQLVAEDGSRVYVVGTAHFSDDSKR
DVVKTIREVQPDVVVVELCQYRVSMLKMDESTLLREAQELSLEKLQQAVRQNGLMSGLMQMLLLKVSAHITEQLGMAPGGEFREAFKEA
SKVPFCKFHLGDRPIPVTFKRAIAALSFWQKVRLAWGLCFLSDPISKDDVERCKQKDLLEQMMAEMIGEFPDLHRTIVSERDVYLTYMLRQ
AARRLELPRASDAEPRKCVPSVVVGVVGMGHVPGIEKNWSTDLNIQEIVITVPPPSVSGRVSRLAVKAAFFGLLGYSLYWMGRRTASLVLS
LPAAQYCLQRVTEARHK
>gi|206597477|ref|NP_001128651.1| palmitoyltransferase ZDHHC3 isoform 1 [Homo sapiens],
SEQ ID NO: 70
MMLIPTHHFRNIERKPEYLQPEKCVPPPYPGPVGTMWFIRDGCGIACAIVTWFLVLYAEFVVLFVMLIPSRDYVYSIINGIVFNLLAFLALAS
HCRAMLTDPGAVPKGNATKEFIESLQLKPGQVVYKCPKCCSIKPDRAHHCSVCKRCIRKMDHHCPWVNNCVGENNQKYFVLFTMYIALI
SLHALIMVGFHFLHCFEEDWTKCSSFSPPTTVILLILLCFEGLLFLIFTSVMFGTQVHSICTDETGIEQLKKEERRWAKKTKWMNMKAVFGH
PFSLGWASPFATPDQGKADPYQYVV
>gi|4557791|ref|NP_000425.1| sialidase-1 precursor [Homo sapiens], SEQ ID NO: 71
MTGERPSTALPDRRWGPRILGFWGGCRVWVFAAIFLLLSLAASWSKAENDFGLVQPLVTMEQLLWVSGRQIGSVDTFRIPLITATPRGTLL
AFAEARKMSSSDEGAKFIALRRSMDQGSTWSPTAFIVNDGDVPDGLNLGAVVSDVETGVVFLFYSLCAHKAGCQVASTMLVWSKDDGV
SWSTPRNLSLDIGTEVFAPGPGSGIQKQREPRKGRLIVCGHGTLERDGVFCLLSDDHGASWRYGSGVSGIPYGQPKQENDFNPDECQPYEL
PDGSVVINARNQNNYHCHCRIVLRSYDACDTLRPRDVTFDPELVDPVVAAGAVVTSSGIVFFSNPAHPEFRVNLTLRWSFSNGTSWRKET
VQLWPGPSGYSSLATLEGSMDGEEQAPQLYVLYEKGRNHYTESISVAKISVYGTL
>gi|5453958|ref|NP_006238.1| serine/threonine-protein phosphatase 5 isoform 1 [Homo sapiens],
SEQ ID NO: 72
MAMAEGERTECAEPPRDEPPADGALKRAEELKTQANDYFKAKDYENAIKFYSQAIELNPSNAIYYGNRSLAYLRTECYGYALGDATRAIE
LDKKYIKGYYRRAASNMALGKFRAALRDYETVVKVKPHDKDAKMKYQECNKIVKQKAFERAIAGDEHKRSVVDSLDIESMTIEDEYSGP
KLEDGKVTISFMKELMQWYKDQKKLHRKCAYQILVQVKEVLSKLSTLVETTLKETEKITVCGDTHGQFYDLLNIFELNGLPSETNPYIFNG
DFVDRGSFSVEVILTLFGFKLLYPDHFHLLRGNHETDNMNQIYGFEGEVKAKYTAQMYELFSEVFEWLPLAQCINGKVLIMHGGLFSEDGV
TLDDIRKIERNRQPPDSGPMCDLLWSDPQPQNGRSISKRGVSCQFGPDVTKAFLEENNLDYIIRSHEVKAEGYEVAHGGRCVTVFSAPNYC
DQMGNKASYIHLQGSDLRPQFHQFTAVPHPNVKPMAYANTLLQLGMM
>gi|22212931|ref|NP_631981.2| nucleolar protein 6 gamma isoform [Homo sapiens], SEQ ID NO: 73
MGPAPAGEQLRGATGEPEVMEPALEGTGKEGKKASSRKRTLAEPPAKGLLQPVKLSRAELYKEPTNEELNRLRETEILFHSSLLRLQVEELL
KEVRLSEKKKDRIDAFLREVNQRVVRVPSVPETELTDQAWLPAGVRVPLHQVPYAVKGCFRFLPPAQVTVVGSYLLGTCIRPDINVDVAL
TMPREILQDKDGLNQRYFRKRALYLAHLAHHLAQDPLFGSVCFSYTNGCHLKPSLURPRGKDERLVTVRLHPCPPPDFFRPCRLLPTKNN
VRSAWYRGQSPAGDGSPEPPTPRYNTWVLQDTVLESHLQLLSTILSSAQGLKDGVALLKVWLRQRELDKGQGGFTGFLVSMLVVFLVSTR
KIHTTMSGYQVLRSVLQFLATTDLTVNGISLCLSSDPSLPALADFHQAFSVVFLDSSGHLNLCADVTASTYHQVQHEARLSMMLLDSRADD
GFHLLLMTPKPMIRAFDHVLHLRPLSRLQAACHRLKLWPELQDNGGDYVSAALGPLTTLLEQGLGARLNLLAHSRPPVPEWDISQDPPKH
KDSGTLTLGLLLRPEGLTSVLELGPEADQPEAAKFRQFWGSRSELRRFQDGAIREAVVWEAASMSQKRLIPHQVVTHLLALHADIPETCVH
YVGGPLDALIQGLKEASSTKGRMVMSRGGELVMVPNVEAILEDFAVLGEGLVQTVEARSERWTV
>gi|55770834|ref|NP_057427.3| centromere protein F [Homo sapiens], SEQ ID NO: 74
MSWALEEWKEGLPTRALQKIQELEGQLDKLKKEKQQRQFQLDSLEAALQKQKQKVENEKTEGTNLKRENQRLMEICESLEKTKQKISHEL
QVKESQVNFQEGQLNSGKKQIEKLEQELKRCKSELERSQQAAQSADVSLNPCNTPQKIFTTPLTPSQYYSGSKYEDLKEKYNKEVEERKRL
EAEVKALQAKKASQTLPQATMNHRDIARHQASSSVFSWQQEKTPSHLSSNSQRTPIRRDFSASYFSGEQEVTPSRSTLQIGKRDANSSFFD
NSSSPHLLDQLKAQNQELRNKINELELRLQGHEKEMKGQVNKFQELQLQLEKAKVELIEKEKVLNKCRDELVRTTAQYDQASTKYTALEQ
KLKKLTEDLSCQRQNAESARCSLEQKIKEKEKEFQEELSRQQRSFQTLDQECIQMKARLTQELQQAKNMHNVLQAELDKLTSVKQQLENN
LEEFKQKLCRAEQAFQASQIKENELRRSMEEMKKENNLLKSHSEQKAREVCHLEAELKNIKQCLNQSQNFAEEMKAKNTSQETMLRDLQ
EKINQQENSLTLEKLKLAVADLEKQRDCSQDLLKKREHHIEQLNDKLSKTEKESKALLSALELKKKEYEELKEEKTLFSCWKSENEKLLTQ
MESEKENLQSKINHLETCLKTQQIKSHEYNERVRTLEMDRENLSVEIRNLHNVLDSKSVEVETQKLAYMELQQKAEFSDQKHQKEIENMC
LKTSQLTGQVEDLEHKLQLLSNEIMDKDRCYQDLHAEYESLRDLLKSKDASLVTNEDHQRSLLAFDQQPAMHHSFANIIGEQGSMPSERSE
CRLEADQSPKNSAILQNRVDSLEFSLESQKQMNSDLQKQCEELVQIKGEIEENLMKAEQMHQSFVAETSQRISKLQEDTSAHQNVVAETLS
ALENKEKELQLLNDKVETEQAEIQELKKSNHLLEDSLKELQLLSETLSLEKKEMSSIISLNKREIEELTQENGTLKEINASLNQEKMNLIQKSE
SFANYIDEREKSISELSDQYKQEKLILLQRCEETGNAYEDLSQKYKAAQEKNSKLECLLNECTSLCENRKNELEQLKEAFAKEHQEFLTKLA
FAEERNQNLMLELETVQQALRSEMTDNQNNSKSEAGGLKQEIMTLKEEQNKMQKEVNDLLQENEQLMKVMKTKHECQNLESEPIRNSV
KERESERNQCNFKPQMDLEVKEISLDSYNAQLVQLEAMLRNKELKLQESEKEKECLQHELQTIRGDLETSNLQDMQSQEISGLKDCEIDAE
EKYISGPHELSTSQNDNAHLQCSLQTTMNKLNELEKICEILQAEKYELVTELNDSRSECITATRKMAEEVGKLLNEVKILNDDSGLLHGELV
EDIPGGEFGEQPNEQHPVSLAPLDESNSYEHLTLSDKEVQMHFAELQEKFLSLQSEHKILHDQHCQMSSKMSELQTYVDSLKAENLVLSTN
LRNFQGDLVKEMQLGLEEGLVPSLSSSCVPDSSSLSSLGDSSFYRALLEQTGDMSLLSNLEGAVSANQCSVDEVFCSSLQEENLTRKETPSA
PAKGVEELESLCEVYRQSLEKLEEKMESQGIMKNKEIQELEQLLSSERQELDCLRKQYLSENEQWQQKLTSVTLEMESKLAAEKKQTEQLS
LELEVARLQLQGLDLSSRSLLGIDTEDAIQGRNESCDISKEHTSETTERTPKHDVHQICDKDAQQDLNLDIEKITETGAVKPTGECSGEQSPD
TNYEPPGEDKTQGSSECISELSFSGPNALVPMDFLGNQEDIHNLQLRVKETSNENLRLLHVIEDRDRKVESLLNEMKELDSKLHLQEVQLM
TKIEACIELEKIVGELKKENSDLSEKLEYFSCDHQELLQRVETSEGLNSDLEMHADKSSREDIGDNVAKVNDSWKERFLDVENELSRIRSEK
ASIEHEALYLEADLEVVQTEKLCLEKDNENKQKVIVCLEEELSVVTSERNQLRGELDTMSKKTTALDQLSEKMKEKTQELESHQSECLHCI
QVAEAEVKEKTELLQTLSSDVSELLKDKTHLQEKLQSLEKDSQALSLTKCELENQIAQLNKEKELLVKESESLQARLSESDYEKLNVSKALE
AALVEKGEFALRLSSTQEEVHQLRRGIEKLRVRIEADEKKQLHIAEKLKERERENDSLKDKVENLERELQMSEENQELVILDAENSKAEVET
LKTQIEEMARSLKVFELDLVTLRSEKENLTKQIQEKQGQLSELDKLLSSFKSLLEEKEQAEIQIKEESKTAVEMLQNQLKELNEAVAALCGD
QEIMKATEQSLDPPIEEEHQLRNSIEKLRARLEADEKKQLCVLQQLKESEHHADLLKGRVENLERELEIARTNQEHAALEAENSKGEVETLK
AKIEGMTQSLRGLELDVVTIRSEKENLTNELQKEQERISELEIINSSFENILQEKEQEKVQMKEKSSTAMEMLQTQLKELNERVAALHNDQE
ACKAKEQNLSSQVECLELEKAQLLQGLDEAKNNYIVLQSSVNGLIQEVEDGKQKLEKKDEEISRLKNQIQDQEQLVSKLSQVEGEHQLWK
EQNLELRNLTVELEQKIQVLQSKNASLQDTLEVLQSSYKNLENELELTKMDKMSFVEKVNKMTAKETELQREMHEMAQKTAELQEELSG
EKNRLAGELQLLLEEIKSSKDQLKELTLENSELKKSLDCMHKDQVEKEGKVREEIAEYQLRLHEAEKKHQALLLDTNKQYEVEIQTYREKL
TSKEECLSSQKLEIDLLKSSKEELNNSLKATTQILEELKKTKMDNLKYVNQLKKENERAQGKMKLLIKSCKQLEEEKEILQKELSQLQAAQE
KQKTGTVMDTKVDELTTEIKELKETLEEKTKEADEYLDKYCSLLISHEKLEKAKEMLETQVAHLCSQQSKQDSRGSPLLGPVVPGPSPIPSV
TEKRLSSGQNKASGKRQRSSGIWENGRGPTPATPESFSKKSKKAVMSGIHPAEDTEGTEFEPEGLPEVVKKGFADIPTGKTSPYILRRTTMA
TRTSPRLAAQKLALSPLSLGKENLAESSKPTAGGSRSQKVKVAQRSPVDSGTILREPTTKSVPVNNLPERSPTDSPREGLRVKRGRLVPSPK
AGLESNGSENCKVQ
>gi|25014088|ref|NP_543153.1| sodium-dependent phosphate transport protein 2C [Homo sapiens],
SEQ ID NO: 75
MPSSLPGSQVPHPTLDAVDLVEKTLRNEGTSSSAPVLEEGDTDPWTLPQLKDTSQPWKELRVAGRLRRVAGSVLKACGLLGSLYFFICSLD
VLSSAFQLLGSKVAGDIFKDNVVLSNPVAGLVIGVLVTALVQSSSTSSSIVVSMVAAKLLTVRVSVPIIMGVNVGTSITSTLVSMAQSGDREF
QRAFSGSAVHGIFNWLTVLVLLPLESATALLERLSELALGAASLTPRAQAPDILKVLTKPLTHLIVQLDSDMIMSSATGNATNSSLIKHWCGT
TGQPTQENSSCGAFGPCTEKNSTAPADRLPCRHLFAGTELTDLAVGCILLAGSLLVLCGCLVLIVKLLNSVLRGRVAQVVRTVINADFPFPL
GWLGGYLAVLAGAGLTFALQSSSVFTAAVVPLMGVGVISLDRAYPLLLGSNIGTTTTALLAALASPADRMLSALQVALIHFFFNLAGILLW
YLVPALRLPIPLARHFGVVTARYRWVAGVYLLLGFLLLPLAAFGLSLAGGMVLAAVGGPLVGLVLLVILVTVLQRRRPAWLPVRLRSWAW
LPVWLHSLEPWDRLVTRCCPCNVCSPPKATTKEAYCYENPEILASQQL
>gi|4557735|ref|NP_000231.1| amine oxidase [flavin-containing] A [Homo sapiens], SEQ ID NO: 76
MENQEKASIAGHMFDVVVIGGGISGLSAAKLLTEYGVSVLVLEARDRVGGRTYTIRNEHVDYVDVGGAYVGPTQNRILRLSKELGIETYK
VNVSERLVQYVKGKTYPFRGAFPPVWNPIAYLDYNNLWRTIDNMGKEIPTDAPWEAQHADKWDKMTMKELIDKICWTKTARRFAYLFV
NINVTSEPHEVSALWFLWYVKQCGGTTRIFSVTNGGQERKFVGGSGQVSERIMDLLGDQVKLNHPVTHVDQSSDNIIIETLNHEHYECKYV
INAIPPTLTAKIFIFRPELPAERNQLIQRLPMGAVIKCMMYYKEAFWKKKDYCGCMIIEDEDAPISITLDDTKPDGSLPAIMGFILARKADRLA
KLHKEIRKKKICELYAKVLGSQEALHPVHYEEKNWCEEQYSGGCYTAYFPPGIMTQYGRVIRQPVGRIFFAGTETATKWSGYMEGAVEAG
ERAAREVLNGLGKVTEKDIWVQEPESKDVPAVEITHTFWERNLPSVSGLLKIIGFSTSVTALGFVLYKYKLLPRS
>gi|22547186|ref|NP_004160.3| serine hydroxymethyltransferase, cytosolic isoform 1 [Homo sapiens],
SEQ ID NO: 77
MTMPVNGAHKDADLWSSHDKMLAQPLKDSDVEVYNIIKKESNRQRVGLELIASENFASRAVLEALGSCLNNKYSEGYPGQRYYGGTEFI
DELETLCQKRALQAYKLDPQCWGVNVQPYSGSPANFAVYTALVEPHGRIMGLDLPDGGHLTHGFMTDKKKISATSIFFESMPYKVNPDT
GYINYDQLEENARLFHPKLIIAGTSCYSRNLEYARLRKIADENGAYLMADMAHISGLVAAGVVPSPFEHCHVVTTTTHKTLRGCRAGMIFY
RKGVKSVDPKTGKEILYNLESLINSAVFPGLQGGPHNHAIAGVAVALKQAMTLEFKVYQHQVVANCRALSEALTELGYKIVTGGSDNHLIL
VDLRSKGTDGGRAEKVLEACSIACNKNTCPGDRSALRPSGLRLGTPALTSRGLLEKDFQKVAHFIHRGIELTLQIQSDTGVRATLKEFKERL
AGDKYQAAVQALREEVESFASLFPLPGLPDF
>gi|30581109|ref|NP_848695.1| minor histocompatibility antigen H13 isoform 2 [Homo sapiens],
SEQ ID NO: 78
MDSALSDPHNGSAEAGGPTNSTTRPPSTPEGIALAYGSLLLMALLPIFFGALRSVRCARGKNASDMPETITSRDAARFPIIASCTLLGLYLFFK
IFSQEYINLLLSMYFFVLGILALSHTISPFMNKFFPASFPNRQYQLLFTQGSGENKEEIINYEFDTKDLVCLGLSSIVGVWYLLRKHWIANNLF
GLAFSLNGVELLHLNNVSTGCILLGGLFIYDVFWVFGTNVMVTVAKSFEAPIKLVFPQDLLEKGLEANNFAMLGLGDVVIPGIFIALLLRFDI
SLKKNTHTYFYTSFAAYIFGLGLTIFIMHIFKHAQPALLYLVPACIGFPVLVALAKGEVTEMFSYESSAEILPHTPRLTHFPTVSGSPASLADS
MQQKLAGPRRRRPQNPSAM
>gi|21312134|ref|NP_056973.2| mediator of RNA polymerase II transcription subunit 15 isoform b
[Homo sapiens], SEQ ID NO: 79
MDVSGQETDWRSTAFRQKLVSQIEDAMRKAGVAHSKSSKDMESHVFLKAKTRDEYLSLVARLIIHFRDIHNKKSQASVSDPMNALQSLT
GGPAAGAAGIGMPPRGPGQSLGGMGSLGAMGQPMSLSGQPPPGTSGMAPHSMAVVSTATPQTQLQLQQVALQQQQQQQQFQQQQQAA
LQQQQQQQQQQQFQAQQSAMQQQFQAVVQQQQQLQQQQQQQQHLIKLHHQNQQQIQQQQQQLQRIAQLQLQQQQQQQQQQQQQQQ
QALQAQPPIQQPPMQQPQPPPSQALPQQLQQMHHTQHHQPPPQPQQPPVAQNQPSQLPPQSQTQPLVSQAQALPGQMLYTQPPLKFVRAP
MVVQQPPVQPQVQQQQTAVQTAQAAQMVAPGVQVSQSSLPMLSSPSPGQQVQTPQSMPPPPQPSPQPGQPSSQPNSNVSSGPAPSPSSFL
PSPSPQPSQSPVTARTPQNFSVPSPGPLNTPVNPSSVMSPAGSSQAEEQQYLDKLKQLSKYIEPLRRMINKIDKNEDRKKDLSKMKSLLDILT
DPSKRCPLKTLQKCEIALEKLKNDMAVPTPPPPPVPPTKQQYLCQPLLDAVLANIRSPVFNHSLYRTFVPAMTAIHGPPITAPVVCTRKRRLE
DDERQSIPSVLQGEVARLDPKFLVNLDPSHCSNNGTVHLICKLDDKDLPSVPPLELSVPADYPAQSPLWIDRQWQYDANPFLQSVHRCMTS
RLLQLPDKHSVTALLNTWAQSVHQACLSAA
>gi|13129110|ref|NP_077007.1| methylosome protein 50 [Homo sapiens], SEQ ID NO: 80
MRKETPPPLVPPAAREWNLPPNAPACMERQLEAARYRSDGALLLGASSLSGRCWAGSLWLFKDPCAAPNEGFCSAGVQTEAGVADLTW
VGERGILVASDSGAVELWELDENETLIVSKFCKYEHDDIVSTVSVLSSGTQAVSGSKDICIKVWDLAQQVVLSSYRAHAAQVTCVAASPHK
DSVFLSCSEDNRILLWDTRCPKPASQIGCSAPGYLPTSLAWHPQQSEVFVFGDENGTVSLVDTKSTSCVLSSAVHSQCVTGLVFSPHSVPFL
ASLSEDCSLAVLDSSLSELFRSQAHRDFVRDATWSPLNHSLLTTVGWDHQVVHHVVPTEPLPAPGPASVTE
>gi|9910280|ref|NP_064505.1| UDP-glucose:glycoprotein glucosyltransferase 1 precursor [Homo
sapiens], SEQ ID NO: 81
MGCKGDASGACAAGALPVTGVCYKMGVLVVLTVLWLFSSVKADSKAITTSLTTKWFSTPLLLEASEFLAEDSQEKFWNFVEASQNIGSSD
HDGTDYSYYHAILEAAFQFLSPLQQNLFKFCLSLRSYSATIQAFQQIAADEPPPEGCNSFFSVHGKKTCESDTLEALLLTASERPKPLLFKGD
HRYPSSNPESPVVIFYSEIGSEEFSNFHRQLISKSNAGKINYVFRHYIFNPRKEPVYLSGYGVELAIKSTEYKAKDDTQVKGTEVNTTVIGEN
DPIDEVQGFLFGKLRDLHPDLEGQLKELRKHLVESTNEMAPLKVWQLQDLSFQTAARILASPVELALVVMKDLSQNFPTKARAITKTAVSS
ELRTEVEENQKYFKGTLGLQPGDSALFINGLHMDLDTQDIFSLFDVLRNEARVMEGLHRLGIEGLSLHNVLKLNIQPSEADYAVDIRSPAIS
WVNNLEVDSRYNSWPSSLQELLRPTFPGVIRQIRKNLHNMVFIVDPAHETTAELMNTAEMFLSNHIPLRIGFIFVVNDSEDVDGMQDAGVA
VLRAYNYVAQEVDDYHAFQTLTHIYNKVRTGEKVKVEHVVSVLEKKYPYVEVNSILGIDSAYDRNRKEARGYYEQTGVGPLPVVLFNG
MPFEREQLDPDELETITMHKILETTTFFQRAVYLGELPHDQDVVEYIMNQPNVVPRINSRILTAERDYLDLTASNNFFVDDYARFTILDSQG
KTAAVANSMNYLTKKGMSSKEIYDDSFIRPVTFWIVGDFDSPSGRQLLYDAIKHQKSSNNVRISMINNPAKEISYENTQISRAIWAALQTQT
SNAAKNFITKMAKEGAAEALAAGADIAEFSVGGMDFSLFKEVFESSKMDFILSHAVYCRDVLKLKKGQRAVISNGRIIGPLEDSELFN
QDDFHLLENIILKTSGQKIKSHIQQLRVEEDVASDLVMKVDALLSAQPKGDPRIEYQFFEDRHSAIKLRPKEGETYFDVVAVVDPVTREAQR
LAPLLLVLAQLINMNLRVFMNCQSKLSDMPLKSFYRYVLEPEISFTSDNSFAKGPIAKFLDMPQSPLFTLNLNTPESWMVESVRTPYDLDNI
YLEEVDSVVAAEYELEYLLLEGHCYDITTGQPPRGLQFTLGTSANPVIVDTIVMANLGYFQLKANPGAWILRLRKGRSEDIYRIYSHDGTDS
PPDADEVVIVLNNFKSKIIKVKVQKKADMVNEDLLSDGTSENESGFWDSFKWGFTGQKTEEVKQDKDDIINIFSVASGHLYERFLRIMMLS
VLKNTKTPVKFWFLKNYLSPTFKEFIPYMANEYNFQYELVQYKWPRWLHQQTEKQRIIWGYKILFLDVLFPLVVDKFLFVDADQIVRTDL
KELRDFNLDGAPYGYTPFCDSRREMDGYRFWKSGYWASHLAGRKYHISALYVVDLKKFRKIAAGDRLRGQYQGLSQDPNSLSNLDQDL
PNNMIHQVPIKSLPQEWLWCETWCDDASKKRAKTIDLCNNPMTKEPKLEAAVRIVPEWQDYDQEIKQLQIRFQKEKETGALYKEKTKEPS
REGPQKREEL
>gi|49574546|ref|NP_001001890.1| runt-related transcription factor 1 isoform AML1b [Homo sapiens],
SEQ ID NO: 82
MRIPVDASTSRRFTPPSTALSPGKMSEALPLGAPDAGAALAGKLRSGDRSMVEVLADHPGELVRTDSPNFLCSVLPTHWRCNKTLPIAFKV
VALGDVPDGTLVTVMAGNDENYSAELRNATAAMKNQVARFNDLRFVGRSGRGKSFTLTITVFTNPPQVATYHRAIKITVDGPREPRRHR
QKLDDQTKPGSLSFSERLSELEQLRRTAMRVSPHHPAPTPNPRASLNHSTAFNPQPQSQMQDTRQIQPSPPWSYDQSYQYLGSIASPSVHP
ATPISPGRASGMTTLSAELSSRLSTAPDLTAFSDPRQFPALPSISDPRMHYPGAFTYSPTPVTSGIGIGMSAMGSATRYHTYLPPPYPGSSQA
QGGPFQASSPSYHLYYGASAGSYQFSMVGGERSPPRILPPCTNASTGSALLNPSLPNQSDVVEAEGSHSNSPTNMAPSARLEEAVWRPY
>gi|27886593|ref|NP_005598.3| focal adhesion kinase 1 isoform b [Homo sapiens], SEQ ID NO: 83
MISADCNLCLPEYDRYLASSKIMAAAYLDPNLNHTPNSSTKTHLGTGMERSPGAMERVLKVFHYFESNSEPTTWASIIRHGDATDVRGIIQ
KIVDSHKVKHVACYGFRLSHLRSEEVHWLHVDMGVSSVREKYELAHPPEEWKYELRIRYLPKGFLNQFTEDKPTLNFFYQQVKSDYMLEI
ADQVDQEIALKLGCLEIRRSYWEMRGNALEKKSNYEVLEKDVGLKRFFPKSLLDSVKAKTLRKLIQQTFRQFANLNREESILKFFEILSPVY
RFDKECFKCALGSSWIISVELAIGPEEGISYLTDKGCNPTHLADFTQVQTIQYSNSEDKDRKGMLQLKIAGAPEPLTVTAPSLTIAENMADLI
DGYCRLVNGTSQSFIIRPQKEGERALPSIPKLANSEKQGMRTHAVSVSETDDYAEIIDEEDTYTMPSTRDYEIQRERIELGRCIGEGQFGDVH
QGIYMSPENPALAVAIKTCKNCTSDSVREKFLQEALTMRQFDHPHIVKLIGVITENPVWIIMELCTLGELRSFLQVRKYSLDLASLILYAYQL
STALAYLESKRFVHRDIAARNVLVSSNDCVKLGDFGLSRYMEDSTYYKASKGKLPIKWMAPESINFRRFTSASDVWMFGVCMWEILMHG
VKPFQGVKNNDVIGRIENGERLPMPPNCPPTLYSLMTKCWAYDPSRRPRFTELKAQLSTILEEEKAQQEERMRMESRRQATVSWDSGGSD
EAPPKPSRPGYPSPRSSEGFYPSPQHMVQTNHYQVSGYPGSHGITAMAGSIYPGQASLLDQTDSWNHRPQEIAMWQPNVEDSTVLDLRGI
GQVLPTHLMEERLIRQQQEMEEDQRWLEKEERFLKPDVRLSRGSIDREDGSLQGPIGNQHIYQPVGKPDPAAPPKKPPRPGAPGHLGSLAS
LSSPADSYNEGVKLQPQEISPPPTANLDRSNDKVYENVTGLVKAVIEMSSKIQPAPPEEYVPMVKEVGLALRTLLATVDETIPLLPASTHREI
EMAQKLLNSDLGELINKMKLAQQYVMTSLQQEYKKQMLTAAHALAVDAKNLLDVIDQARLKMLGQTRPH
>gi|56118221|ref|NP_061916.3| amyloid beta A4 precursor protein-binding family B member 1-
interacting protein [Homo sapiens], SEQ ID NO: 84
MGESSEDIDQMFSTLLGEMDLLTQSLGVDTLPPPDPNPPRAEFNYSVGFKDLNESLNALEDQDLDALMADLVADISEAEQRTIQAQKESLQ
NQHHSASLQASIFSGAASLGYGTNVAATGISQYEDDLPPPPADPVLDLPLPPPPPEPLSQEEEEAQAKADKIKLALEKLKEAKVKKLVVKVH
MNDNSTKSLMVDERQLARDVLDNLFEKTHCDCNVDWCLYEIYPELQIERFFEDHENVVEVLSDWTRDTENKILFLEKEEKYAVFKNPQNF
YLDNRGKKESKETNEKMNAKNKESLLEESFCGTSIIVPELEGALYLKEDGKKSWKRRYFLLRASGIYYVPKGKTKTSRDLACFIQFENVNIY
YGTQHKMKYKAPTDYCFVLKHPQIQKESQYIKYLCCDDTRTLNQWVMGIRIAKYGKTLYDNYQRAVAKAGLASRWTNLGTVNAAAPA
QPSTGPKTGTTQPNGQIPQATHSVSAVLQEAQRHAETSKDKKPALGNHHDPAVPRAPHAPKSSLPPPPPVRRSSDTSGSPATPLKAKGTGG
GGLPAPPDDFLPPPPPPPPLDDPELPPPPPDFMEPPPDFVPPPPPSYAGIAGSELPPPPPPPPAPAPAPVPDSARPPPAVAKRPPVPPKRQENPG
HPGGAGGGEQDFMSDLMKALQKKRGNVS
>gi|11386139|ref|NP_000923.1| 1-phosphatidylinositol-4,5-bisphosphate phosphodiesterase beta-3
isoform 1 [Homo sapiens], SEQ ID NO: 85
MAGAQPGVHALQLEPPTVVETLRRGSKFIKWDEETSSRNLVTLRVDPNGFFLYWTGPNMEVDTLDISSIRDTRTGRYARLPKDPKIREVLG
FGGPDARLEEKLMTVVSGPDPVNTVFLNFMAVQDDTAKVWSEELFKLAMNILAQNASRNTFLRKAYTKLKLQVNQDGRIPVKNILKMFS
ADKKRVETALESCGLKFNRSESIRPDEFSLEIFERFLNKLCLRPDIDKILLEIGAKGKPYLTLEQLMDFINQKQRDPRLNEVLYPPLRPSQARL
LIEKYEPNQQFLERDQMSMEGFSRYLGGEENGILPLEALDLSTDMTQPLSAYFINSSHNTYLTAGQLAGTSSVEMYRQALLWGCRCVELD
VWKGRPPEEEPFITHGFTMTTEVPLRDVLEAIAETAFKTSPYPVILSFENHVDSAKQQAKMAEYCRSIFGDALLIEPLDKYPLAPGVPLPSPQ
DLMGRILVKNKKRHRPSAGGPDSAGRKRPLEQSNSALSESSAATEPSSPQLGSPSSDSCPGLSNGEEVGLEKPSLEPQKSLGDEGLNRGPYV
LGPADREDEEEDEEEEEQTDPKKPTTDEGTASSEVNATEEMSTLVNYIEPVKFKSFEAARKRNKCFEMSSFVETKAMEQLTKSPMEFVEY
NKQQLSRIYPKGTRVDSSNYMPQLFWNVGCQLVALNFQTLDVAMQLNAGVFEYNGRSGYLLKPEFMRRPDKSFDPFTEVIVDGIVANAL
RVKVISGQFLSDRKVGIYVEVDMFGLPVDTRRKYRTRTSQGNSFNPVWDEEPFDFPKVVLPTLASLRIAAFEEGGKFVGHRILPVSAIRSGY
HYVCLRNEANQPLCLPALLIYTEASDYIPDDHQDYAEALINPIKHVSLMDQRARQLAALIGESEAQAGQETCQDTQSQQLGSQPSSNPTPSP
LDASPRRPPGPTTSPASTSLSSPGQRDDLIASILSEVAPTPLDELRGHKALVKLRSRQERDLRELRKKHQRKAVTLTRRLLDGLAQAQAEGR
CRLRPGALGGAADVEDTKEGEDEAKRYQEFQNRQVQSLLELREAQVDAEAQRRLEHLRQALQRLREVVLDANTTQFKRLKEMNEREKK
ELQKILDRKRHNSISEAKMRDKHKKEAELTEINRRHITESVNSIRRLEEAQKQRHDRLVAGQQQVLQQLAEEEPKLLAQLAQECQEQRARL
PQEIRRSLLGEMPEGLGDGPLVACASNGHAPGSSGHLSGADSESQEENTQL
>gi|16445029|ref|NP_443100.1| immunoglobulin supelfamily member 8 [Homo sapiens], SEQ ID NO: 86
MGALRPTLLPPSLPLLLLLMLGMGCWAREVLVPEGPLYRVAGTAVSISCNVTGYEGPAQQNFEWFLYRPEAPDTALGIVSTKDTQFSYAVF
KSRVVAGEVQVQRLQGDAVVLKIARLQAQDAGIYECHTPSTDTRYLGSYSGKVELRVLPDVLQVSAAPPGPRGRQAPTSPPRMTVHEGQ
ELALGCLARTSTQKHTHLAVSFGRSVPEAPVGRSTLQEVVGIRSDLAVEAGAPYAERLAAGELRLGKEGTDRYRMVVGGAQAGDAGTYH
CTAAEWIQDPDGSWAQIAEKRAVLAHVDVQTLSSQLAVTVGPGERRIGPGEPLELLCNVSGALPPAGRHAAYSVGWEMAPAGAPGPGRL
VAQLDTEGVGSLGPGYEGRHIAMEKVASRTYRLRLEAARPGDAGTYRCLAKAYVRGSGTRLREAASARSRPLPVHVREEGVVLEAVAWL
AGGTVYRGETASLLCNISVRGGPPGLRLAASWWVERPEDGELSSVPAQLVGGVGQDGVAELGVRPGGGPVSVELVGPRSHRLRLHSLGPE
DEGVYHCAPSAWVQHADYSWYQAGSARSGPVTVYPYMHALDTLFVPLLVGTGVALVTGATVLGTITCCFMKRLRKR
>gi|209413763|ref|NP_001129247.1| calcium channel flower homolog isoform b [Homo sapiens],
SEQ ID NO: 87
MSSSGGAPGASASSAPPAQEEGMTWWYRWLCRLSGVLGAVSCATSGLFNCITIHPLNIAAGVWMMMAVVPIVISLTLTTLLGNAIAFATGV
LYGLSALGKKGDAISYARIQQQRQQADEEKLAETLEGEL
>gi|11968003|ref|NP_071906.1| 5-azacytidine-induced protein 2 isoform a [Homo sapiens],
SEQ ID NO: 88
MDALVEDDICILNHEKAHKRDTVTPVSIYSGDESVASHFALVTAYEDIKKRLKDSEKENSLLKKRIRFLEEKLIARFEEETSSVGREQVNKA
YHAYREVCIDRDNLKSKLDKMNKDNSESLKVLNEQLQSKEVELLQLRTEVETQQVMRNLNPPSSNWEVEKLSCDLKIHGLEQELELMRK
ECSDLKIELQKAKQTDPYQEDNLKSRDLQKLSISSDNMQHAYWELKREMSNLHLVTQVQAELLRKLKTSTAIKKACAPVGCSEDLGRDST
KLHLMNFTATYTRHPPLLPNGKALCHTTSSPLPGDVKVLSEKAILQSWTDNERSIPNDGTCFQEHSSYGRNSLEDNSWVFPSPPKSSETAFG
ETKTKTLPLPNLPPLHYLDQHNQNCLYKN
>gi|89111122|ref|NP_001034750.1| toll/interleukin-1 receptor domain-containing adapter protein
isoform a [Homo sapiens], SEQ ID NO: 89
MASSTSLPAPGSRPKKPLGKMADWFRQTLLKKPKKRPNSPESTSSDASQPTSQDSPLPPSLSSVTSPSLPPTHASDSGSSRWSKDYDVCVCH
SEEDLVAAQDLVSYLEGSTASLRCFLQLRDATPGGAIVSELCQALSSSHCRVLLITPGFLQDPWCKYQMLQALTEAPGAEGCTIPLLSGLSR
AAYPPELRFMYYVDGRGPDGGFRQVKEAVMRYLQTLS
>gi|172072620|ref|NP_055047.2| vesicle-associated membrane protein 2 [Homo sapiens], SEQ ID NO: 90
MSATAATAPPAAPAGEGGPPAPPPNLTSNRRLQQTQAQVDEVVDIMRVNVDKVLERDQKLSELDDRADALQAGASQFETSAAKLKRKY
WWKNLKMMELGVICABLIIIIVYFST
>gi|10835147|ref|NP_000568.1| interleukin-1 receptor antagonist protein isoform 3 [Homo sapiens],
SEQ ID NO: 91
MALETICRPSGRKSSKMQAFRIWDVNQKTFYLRNNQLVAGYLQGPNVNLEEKIDVVPIEPHALFLGIHGGKMCLSCVKSGDETRLQLEAV
NITDLSENRKQDKRFAFIRSDSGPTTSFESAACPGWFLCTAMEADQPVSLTNMPDEGVMVTKFYFQEDE
>gi|19743850|ref|NP_598012.1| decorin isoform c precursor [Homo sapiens], SEQ ID NO: 92
MKATIILLLLAQVSWAGPFQQRGLFDFMLEDEASGIGPEVPDDRDFEPSLGPVCPFRCQCHLRVVQCSDLGLPPSLTELHLDGNKISRVDAA
SLKGLNNLAKLGLSFNSISAVDNGSLANTPHLRELHLDNNKLTRVPGGLAEHKYIQVVYLHNNNISVVGSSDFCPPGHNTKKASYSGVSLF
SNPVQYWEIQPSTFRCVYVRSAIQLGNYK
>gi|289666732|ref|NP_001166237.1| forkhead box protein P2 isoform V [Homo sapiens], SEQ ID NO: 93
MMQESATETISNSSMNQNGMSTLSSQLDAGSRDGRSSGDTSSEVSTVELLHLQQQQALQAARQLLLQQQTSGLKSPKSSDKQRPLQVPVS
VAMMTPQVITPQQMQQILQQQVLSPQQLQALLQQQQAVMLQQQQLQEFYKKQQEQLHLQLLQQQQQQQQQQQQQQQQQQQQQQQQ
QQQQQQQQQQQQQQQQHPGKQAKEQQQQQQQQQLAAQQLVFQQQLLQMQQLQQQQHLLSLQRQGLISIPPGQAALPVQSLPQAGLSP
AEIQQLWKEVTGVHSMEDNGIKHGGLDLTTNNSSSTTSSNTSKASPPITHHSIVNGQSSVLSARRDSSSHEETGASHTLYGHGVCKWPGCE
SICEDFGQFLKHLNNEHALDDRSTAQCRVQMQVVQQLEIQLSKERERLQAMMTHLHMRPSEPKPSPKPLNLVSSVTMSKNMLETSPQSLP
QTPTTPTAPVTPITQGPSVITPASVPNVGAIRRRHSDKYNIPMSSEIAPNYEFYKNADVRPPFTYATLIRQAIMESSDRQLTLNEIYSWFTRTF
AYFRRNAATWKNAVRHNLSLHKCFVRVENVKGAVWTVDEVEYQKRRSQKITGSPTLVKNIPTSLGYGAALNASLQAALAESSLPLLSNP
GLINNASSGLLQAVHEDLNGSLDHIDSNGNSSPGCSPQPHIHSIHVKEEPVIAEDEDCPMSLVTTANHSPELEDDREIEEEPLSEDLE
>gi|4502337|ref|NP_001176.1| zinc-alpha-2-glycoprotein precursor [Homo sapiens], SEQ ID NO: 94
MVRMVPVLLSLLLLLGPAVPQENQDGRYSLTYIYTGLSKHVEDVPAFQALGSLNDLQFFRYNSKDRKSQPMGLWRQVEGMEDWKQDSQ
LQKAREDIFMETLKDIVEYYNDSNGSHVLQGRFGCEIENNRSSGAFWKYYYDGKDYIEFNKEIPAWVPFDPAAQITKQKWEAEPVYVQRA
KAYLEEECPATLRKYLKYSKNILDRQDPPSVVVTSHQAPGEKKKLKCLAYDFYPGKIDVHWTRAGEVQEPELRGDVLHNGNGTYQSWV
VVAVPPQDTAPYSCHVQHSSLAQPLVVPWEAS
>gi|295986608|ref|NP_001171597.1| immunoglobulin lambda-like polypeptide 5 [Homo sapiens],
SEQ ID NO: 95
MRPKTGQVGCETPEELGPGPRQRWPLLLLGLAMVAHGLLRPMVAPQSGDPDPGASVGSSRSSLRSLWGRLLLQPSPQRADPRCWPRGFW
SEPQSLCYVFGTGTKVTVLGQPKANPTVTLFPPSSEELQANKATLVCLISDFYPGAVTVAWKADGSPVKAGVETTKPSKQSNNKYAASSY
LSLTPEQWKSHRSYSCQVTHEGSTVEKTVAPTECS
>gi|13236530|ref|NP_077278.1| matrix metalloproteinase-28 isoform 1 preproprotein [Homo sapiens],
SEQ ID NO: 96
MVARVGLLLRALQLLLWGHLDAQPAERGGQELRKEAEAFLEKYGYLNEQVPKAPTSTRFSDAIRAFQWVSQLPVSGVLDRATLRQMTRP
RCGVTDTNSYAAWAERISDLFARHRTKMRRKKRFAKQGNKWYKQHLSYRLVNWPEHLPEPAVRGAVRAAFQLWSNVSALEFWEAPAT
GPADIRLTFFQGDHNDGLGNAFDGPGGALAHAFLPRRGEAHFDQDERWSLSRRRGRNLFVVLAHEIGHTLGLTHSPAPRALMAPYYKRLG
RDALLSWDDVLAVQSLYGKPLGGSVAVQLPGKLFTDFETWDSYSPQGRRPETQGPKYCHSSFDAITVDRQQQLYIFKGSHFWEVAADGN
VSEPRPLQERWVGLPPNIEAAAVSLNDGDFYFFKGGRCWRFRGPKPVWGLPQLCRAGGLPRHPDAALFFPPLRRLILFKGARYYVLARGG
LQVEPYYPRSLQDWGGIPEEVSGALPRPDGSIIFFRDDRYWRLDQAKLQATTSGRWATELPWMGCWHANSGSALF
>gi|44662834|ref|NP_004235.3| scavenger receptor cysteine-rich type 1 protein M130 isoform a
[Homo sapiens], SEQ ID NO: 97
MSKLRMVLLEDSGSADFRRHFVNLSPFTITVVLLLSACFVTSSLGGTDKELRLVDGENKCSGRVEVKVQEEWGTVCNNGWSMEAVSVICN
QLGCPTAIKAPGWANSSAGSGRIWMDHVSCRGNESALWDCKHDGWGKHSNCTHQQDAGVTCSDGSNLEMRLTRGGNMCSGRIEIKFQ
GRWGTVCDDNFNIDHASVICRQLECGSAVSFSGSSNFGEGSGPIWFDDLICNGNESALWNCKHQGWGKHNCDHAEDAGVICSKGADLSL
RLVDGVTECSGRLEVRFQGEWGTICDDGWDSYDAAVACKQLGCPTAVTAIGRVNASKGFGHIWLDSVSCQGHEPAVWQCKHEIEWGKH
YCNHNEDAGVTCSDGSDLELRLRGGGSRCAGTVEVEIQRLLGKVCDRGWGLKEADVVCRQLGCGSALKTSYQVYSKIQATNTWLFLSSC
NGNETSLWDCKNWQWGGLTCDHYEEAKITCSAHREPRLVGGDIPCSGRVEVKHGDTWGSICDSDFSLEAASVLCRELQCGTVVSILGGA
HFGEGNGQIWAEEFQCEGHESHLSLCPVAPRPEGTCSHSRDVGVVCSRYTEIRLVNGKTPCEGRVELKTLGAWGSLCNSHWDIEDAHVLC
QQLKCGVALSTPGGARFGKGNGQIWRHMFHCTGTEQHMGDCPVTALGASLCPSEQVASVICSGNQSQTLSSCNSSSLGPTRPTIPEESAVA
CIESGQLRLVNGGGRCAGRVEIYHEGSWGTICDDSWDLSDAHVVCRQLGCGEAINATGSAHFGEGTGPIWLDEMKCNGKESRIWQCHSH
GWGQQNCRHKEDAGVICSEFMSLRLTSEASREACAGRLEVFYNGAWGTVGKSSMSETTVGVVCRQLGCADKGKINPASLDKAMSIPMW
VDNVQCPKGPDTLWQCPSSPWEKRLASPSEETWITCDNKIRLQEGPTSCSGRVEIWHGGSWGTVCDDSWDLDDAQVVCQQLGCGPALK
AFKEAEFGQGTGPIWLNEVKCKGNESSLWDCPARRWGHSECGHKEDAAVNCTDISVQKTPQKATTGRSSRQSSFIAVGILGVVLLAIFVAL
FFLTKKRRQRQRLAVSSRGENLVHQIQYREMNSCLNADDLDLMNSSENSHESADFSAAELISVSKFLPISGMEKEAILSHTEKENGNL
>gi|44771211|ref|NP_056150.1| mediator of RNA polymerase II transcription subunit 13-like [Homo
sapiens], SEQ ID NO: 98
MTAAANWVANGASLEDCHSNLFSLAELTGIKWRRYNFGGHGDCGPIISAPAQDDPILLSFIRCLQANLLCVWRRDVKPDCKELWIFWWGD
EPNLVGVIHHELQVVEEGLWENGLSYECRTLLFKAIFINLLERCLMDKNFVRIGKWFVRPYEKDEKPVNKSEHLSCAFTFFLHGESNVCTSV
EIAQHQPIYLINEEHIHMAQSSPAPFQVLVSPYGLNGTLTGQAYKMSDPATRKLIEEWQYFYPMVLKKKEESKEEDELGYDDDFPVAVEVI
VGGVRMVYPSAFVLISQNDIPVPQSVASAGGHIAVGQQGLGSVKDPSNCGMPLTPPTSPEQAILGESGGMQSAASHLVSQDGGMITMHSP
KRSGKIPPKLHNHMVHRVWKECILNRTQSKRSQMSTPTLEEEPASNPATWDFVDPTQRVSCSCSRHKLLKRCAVGPNRPPTVSQPGFSAG
PSSSSSLPPPASSKHKTAERQEKGDKLQKRPLIPFHHRPSVAEELCMEQDTPGQKLGLAGIDSSLEVSSSRKYDKQMAVPSRNTSKQMNLN
PMDSPHSPISPLPPTLSPQPRGQETESLDPPSVPVNPALYGNGLELQQLSTLDDRTVLVGQRLPLMAEVSETALYCGIRPSNPESSEKWWHS
YRLPPSDDAEFRPPELQGERCDAKMEVNSESTALQRLLAQPNKRFKIWQDKQPQLQPLHFLDPLPLSQQPGDSLGEVNDPYTFEDGDIKYIF
TANKKCKQGTEKDSLKKNKSEDGFGTKDVTTPGHSTPVPDGKNAMSIFSSATKTDVRQDNAAGRAGSSSLTQVTDLAPSLHDLDNIFDNS
DDDELGAVSPALRSSKMPAVGTEDRPLGKDGRAAVPYPPTVADLQRMFPTPPSLEQHPAFSPVMNYKDGISSETVTALGMMESPMVSMV
STQLTEFKMEVEDGLGSPKPEEIKDFSYVHKVPSFQPFVGSSMFAPLKMLPSHCLLPLKIPDACLFRPSWAIPPKIEQLPMPPAATFIRDGYN
NVPSVGSLADPDYLNTPQMNTPVTLNSAAPASNSGAGVLPSPATPRFSVPTPRTPRTPRTPRGGGTASGQGSVKYDSTDQGSPASTPSTTR
PLNSVEPATMQPIPEAHSLYVTLILSDSVMNIFKDRNFDSCCICACNMNIKGADVGLYIPDSSNEDQYRCTCGFSAIMNRKLGYNSGLFLED
ELDIFGKNSDIGQAAERRLMMCQSTFLPQVEGTKKPQEPPISLLLLLQNQHTQPFASLNFLDYISSNNRQTLPCVSWSYDRVQADNNDYWT
ECFNALEQGRQYVDNPTGGKVDEALVRSATVHSWPHSNVLDISMLSSQDVVRMLLSLQPFLQDAIQKKRTGRTWENIQHVQGPLTWQQF
HKMAGRGTYGSEESPEPLPIPTLLVGYDKDFLTISPFSLPFWERLLLDPYGGHRDVAYIVVCPENEALLEGAKTFFRDLSAVYEMCRLGQHK
PICKVLRDGIMRVGKTVAQKLTDELVSEWFNQPWSGEENDNHSRLKLYAQVCRHHLAPYLATLQLDSSLLIPPKYQTPPAAAQGQATPGN
AGPLAPNGSAAPPAGSAFNPTSNSSSTNPAASSSASGSSVPPVSSSASAPGISQISTTSSSGFSGSVGGQNPSTGGISADRTQGNIGCGGDTDP
GQSSSQPSQDGQESVTERERIGIPTEPDSADSHAHPPAVVIYMVDPFTYAAEEDSTSGNFWLLSLMRCYTEMLDNLPEHMRNSFILQIVPCQ
YMLQTMKDEQVFYIQYLKSMAFSVYCQCRRPLPTQIHIKSLTGFGPAASIEMTLKNPERPSPIQLYSPPFILAPIKDKQTELGETFGEASQKY
NVLFVGYCLSHDQRWLLASCTDLHGELLETCVVNIALPNRSRRSKVSARKIGLQKLWEWCIGIVQMTSLPWRVVIGRLGRLGHGELKDWS
ILLGECSLQTISKKLKDVCRMCGISAADSPSILSACLVAMEPQGSFVVMPDAVTMGSVFGRSTALNMQSSQLNTPQDASCTHILVFPTSSTIQ
VAPANYPNEDGFSPNNDDMFVDLPFPDDMDNDIGILMTGNLHSSPNSSPVPSPGSPSGIGVGSHFQHSRSQGERLLSREAPEELKQQPLALG
YFVSTAKAENLPQWFWSSCPQAQNQCPLFLKASLHHHISVAQTDELLPARNSQRVPHPLDSKTTSDVLRFVLEQYNALSWLTCNPATQDR
TSCLPVHFVVLTQLYNAIMNIL
>gi|6996021|ref|NP_000388.2| cytochrome b-245 heavy chain [Homo sapiens], SEQ ID NO: 99
MGNWAVNEGLSIFVILVWLGLNVFLFVWYYRVYDIPPKFFYTRKLLGSALALARAPAACLNFNCMLILLPVCRNLLSFLRGSSACCSTRVR
RQLDRNLTFHKMVAWMIALHSAIHTIAHLFNVEWCVNARVNNSDPYSVALSELGDRQNESYLNFARKRIKNPEGGLYLAVTLLAGITGVV
ITLCLILIITSSTKTIRRSYFEVFWYTHHLFVIFFIGLAIHGAERIVRGQTAESLAVHNITVCEQKISEWGKIKECPIPQFAGNPPMTWKWIVGPM
FLYLCERLVRFWRSQQKVVITKVVTHPFKTIELQMKKKGFKMEVGQYIFVKCPKVSKLEWHPFTLTSAPEEDFFSIHIRIVGDWTEGLFNAC
GCDKQEFQDAWKLPKIAVDGPFGTASEDVFSYEVVMLVGAGIGVTPFASILKSVWYKYCNNATNLKLKKIYFYWLCRDTHAFEWFADLL
QLLESQMQERNNAGFLSYNIYLTGWDESQANHFAVHHDEEKDVITGLKQKTLYGRPNWDNEFKTIASQHPNTRIGVFLCGPEALAETLSK
QSISNSESGPRGVHFIFNKENF
>gi|31377782|ref|NP_006245.2| protein kinase C delta type [Homo sapiens], SEQ ID NO: 100
MAPFLRIAFNSYELGSLQAEDEANQPFCAVKMKEALSTERGKTLVQKKPTMYPEWKSTFDAHIYEGRVIQIVLMRAAEEPVSEVTVGVSV
LAERCKKNNGKAEFWLDLQPQAKVLMSVQYFLEDVDCKQSMRSEDEAKFPTMNRRGAIKQAKIHYIKNHEFIATFFGQPTFCSVCKDFV
WGLNKQGYKCRQCNAAIHKKCIDKIIGRCTGTAANSRDTIFQKERFNIDMPHRFKVHNYMSPTFCDHCGSLLWGLVKQGLKCEDCGMNV
HHKCREKVANLCGINQKLLAEALNQVTQRASRRSDSASSEPVGIYQGFEKKTGVAGEDMQDNSGTYGKIWEGSSKCNINNFIFHKVLGKG
SFGKVLLGELKGRGEYFAIKALKKDVVLIDDDVECTMVEKRVLTLAAENPFLTHLICTFQTKDHLFFVMEFLNGGDLMYHIQDKGRFELYA
TFYAAEIMCGLQFLHSKGIIYRDLKLDNVLLDRDGHIKIADFGMCKENIFGESRASTFCGTPDYIAPEILQGLKYTFSVDWWSFGVLLYEMLI
GQSPFHGDDEDELFESIRVDTPHYPRWITKESKDILEKLFEREPTKRLGVTGNIKIHPFFKTINWTLLEKRRLEPPFRPKVKSPRDY
SNFDQEFLNEKARLSYSDKNLIDSMDQSAFAGFSFVNPKFEHLLED
>gi|332164777|ref|NP_001193726.1| pyruvate kinase isozymes M1/M2 isoform d [Homo sapiens],
SEQ ID NO: 101
MSKPHSEAGTAFIQTQQLHAAMADTFLEHMCRLDIDSPPITARNTGIICTIGPASRSVELKKGATLKITLDNAYMEKCDENILWLDYKNICK
VVEVGSKIYVDDGLISLQVKQKGADFLVTEVENGGSLGSKKGVNLPGAAVDLPAVSEKDIQDLKFGVEQDVDMVFASFIRKASDVHEVRK
VLGEKGKNIKIISKIENHEGVRRFDEILEASDGIMVARGDLGIEIPAEKVFLAQKMMIGRCNRAGKPVICATQMLESMIKKPRPTRAEGSDVA
NAVLDGADCIMLSGETAKGDYPLEAVRMQHLIAREAEAAIYHLQLFEELRRLAPITSDPTEATAVGAVEASFKCCSGAIIVLTKSGRSAHQV
ARYRPRAPIIAVTRNPQTARQAHLYRGIFPVLCKDPVQEAWAEDVDLRVNFAMNVGKARGFFKKGDVVIVLTGWRPGSGFTNTMRVVPV
P
>gi|148596984|ref|NP_004478.3| Golgin subfamily B member 1 [Homo sapiens], SEQ ID NO: 102
MLSRLSGLANVVLHELSGDDDTDQNMRAPLDPELHQESDMEFNNTTQEDVQERLAYAEQLVVELKDBRQKDVQLQQKDEALQEERKAA
DNKIKKLKLHAKAKLTSLNKYIEEMKAQGGTVLPTEPQSEEQLSKHDKSSTEEEMEIEKIKHKLQEKEELISTLQAQLTQAQAEQPAQSSTE
MEEFVMMKQQLQEKEEFISTLQAQLSQTQAEQAAQQVVREKDARFETQVRLHEDELLQLVTQADVETEMQQKLRVLQRKLEEHEESLV
GRAQVVDLLQQELTAAEQRNQILSQQLQQMEAEHNTLRNTVETEREESKILLEKMELEVAERKLSFHNLQEEMHHLLEQFEQAGQAQAEL
ESRYSALEQKHKAEMEEKTSHILSLQKTGQELQSACDALKDQNSKLLQDKNEQAVQSAQTIQQLEDQLQQKSKEISQFLNRLPLQQHETAS
QTSFPDVYNEGTQAVTEENIASLQKRVVELENEKGALLLSSIELEELKAENEKLSSQITLLEAQNRTGEADREVSEISIVDIANKRSSSAEESG
QDVLENTFSQKHKELSVLLLEMKEAQEEIAFLKLQLQGKRAEEADHEVLDQKEMKQMEGEGIAPIKMKVFLEDTGQDFPLMPNEESSLPA
VEKEQASTEHQSRTSEEISLNDAGVELKSTKQDGDKSLSAVPDIGQCHQDELERLKSQ1LELELNFHKAQE1YEKNLDEKAKEISNLNQUEE
FKKNADNNSSAFTALSEERDQLLSQVKELSMVTELRAQVKQLEMNLAEAERQRRLDYESQTAHDNLLTEQIHSLSIEAKSKDVKIEVLQNE
LDDVQLQFSEQSTLIRSLQSQLQNKESEVLEGAERVRHISSKVEELSQALSQKELEITKMDQLLLEKKRDVETLQQTIEEKDQQVTEISFSM
TEKMVQLNEEKFSLGVEIKTLKEQLNLLSRAEEAKKEQVEEDNEVSSGLKQNYDEMSPAGQISKEELQHEFDLLKKENEQRKRKLQAALIN
RKELLQRVSRLEEELANLKDESKKEIPLSETERGEVEEDKENKEYSEKCVTSKCQEIEIYLKQTISEKEVELQHIRKDLEEKLAAEEQFQALV
KQMNQTLQDKTNQIDLLQAEISENQAIIQKLITSNTDASDGDSVALVKETVVISPPCTGSSEHWKPELEEKILALEKEKEQLQKKLQEALTSR
KAILKKAQEKERHLREELKQQKDDYNRLQEQFDEQSKENENIGDQLRQLQIQVRESIDGKLPSTDQQESCSSTPGLEEPLFKATEQHHTQP
VLESNLCPDWPSHSEDASALQGGTSVAQIKAQLKEIEAEKVELELKVSSTTSELTKKSEEVFQLQEQINKQGLEIESLKTVSHEAEVHAESLQ
QKLESSQLQIAGLEHLRELQPKLDELQKLISKKEEDVSYLSGQLSEKEAALTKIQTEBEQEDLIKALHTQLEMQAKEHDERIKQLQVELCEM
KQKPEEIGEESRAKQQIQRKLQAALISRKEALKENKSLQEELSLARGTIERLTKSLADVESQVSAQNKEKDTVLGRLALLQEERDKLITEMD
RSLLENQSLSSSCESLKLALEGLTEDKEKLVKEIESLKSSKIAESTEWQEKHKELQKEYEILLQSYENVSNEAERIQHVVEAVRQEKQELYGK
LRSTEANKKETEKQLQEAEQEMEEMKEKMRKFAKSKQQKILELEEENDRLRAEVHPAGDTAKECMETLLSSNASMKEELERVKMEYETL
SKKFQSLMSEKDSLSEEVQDLKHQIEGNVSKQANLEATEKHDNQTNVTEEGTQSIPGETEEQDSLSMSTRPTCSESVPSAKSANPAVSKDF
SSHDEINNYLQQIDQLKERIAGLEEEKQKNKEFSQTLENEKNTLLSQISTKDGELKMLQEEVTKMNLLNQQIQEELSRVTKLKETAEEEKDD
LEERLMNQLAELNGSIGNYCQDVTDAQIKNELLESEMKNLKKCVSELEEEKQQLVKEKTKVESEIRKEYLEKIQGAQKEPGNKSHAKELQ
ELLKEKQQEVKQLQKDCIRYQEKISALERTVKALEFVQTESQKDLEITKENLAQAVEHRKKAQAELASFKVLLDDTQSEAARVLADNLKL
KKELQSNKESVKSQMKQKDEDLERRLEQAEEKHLKEKKNMQEKLDALRREKVHLEETIGEIQVTLNKKDKEVQQLQENLDSTVTQLAAF
TKSMSSLQDDRDRVIDEAKKWERKFSDAIQSKEEEIRLKEDNCSVLKDQLRQMSIHMEELKINISRLEHDKQIWESKAQTEVQLQQKVCDT
LQGENKELLSQLEETRHLYHSSQNELAKLESELKSLKDQLTDLSNSLEKCKEQKGNLEGIIRQQEADIQNSKFSYEQLETDLQASRELTSRLH
EEINMKEQKIISLLSGKEEAIQVAIAELRQQHDKEIKELENLLSQEEEENIVLEEENKKAVDKTNQLMETLKTIKKENIQQKAQLDSFVKSMS
SLQNDRDRIVGDYQQLEERHLSIILEKDQLIQEAAAENNKLKEEIRGLRSHMDDLNSENAKLDAELIQYREDLNQVITIKDSQQKQLLEVQL
QQNKELENKYAKLEEKLKESEEANEDLRRSFNALQEEKQDLSKEIESLKVSISQLTRQVTALQEEGTLGLYHAQLKVKEEEVHRLSALFSSS
QKRIAELEEELVCVQKEAAKKVGEIEDKLKKELKHLHHDAGIMRNETETAEERVAELARDLVEMEQKLLMVTKENKGLTAQIQSFGRSMS
SLQNSRDHANEELDELKRKYDASLKELAQLKEQGLLNRERDALLSETAFSMNSTEENSLSHLEKLNQQLLSKDEQLLHLSSQLEDSYNQV
QSFSKAMASLQNERDHLWNELEKFRKSEEGKQRSAAQPSTSPAEVQSLKKAMSSLQNDRDRLLKELKNLQQQYLQINQEITELHPLKAQL
QEYQDKTKAFQINIQEELRQENLSWQHELHQLRMEKSSWEIHERRMKEQYLMAISDKDQQLSHLQNLIRELRSSSSQTQPLKVQYQRQAS
PETSASPDGSQNLVYETELLRTQLNDSLKEIHQKELRIQQLNSNFSQLLEEKNTLSIQLCDTSQSLRENQQHYGDLLNHCAVLEKQVQELQA
GPLNIDVAPGAPQEKNGVHRKSDPEELREPQQSFSEAQQQLCNTRQEVNELRKLLEEERDQRVAAENALSVAEEQIRRLEHSEWDSSRTPII
GSCGTQEQALLIDLTSNSCRRTRSGVGWKRVLRSLCHSRTRVPLLAAIYFLMIHVLLILCFTGHL
>gi|4506547|ref|NP_002924.1| ribonuclease pancreatic precursor [Homo sapiens], SEQ ID NO: 103
MALEKSLVRLLLLVLILLVLGWVQPSLGKESRAKKFQRQHMDSDSSPSSSSTYCNQMMRRRNMTQGRCKPVNTFVHEPLVDVQNVCFQE
KVTCKNGQGNCYKSNSSMHITDCRLTNGSRYPNCAYRTSPKERHIIVACEGSPYVPVHFDASVEDST
>gi|255759952|ref|NP_001157281.1| WD repeat-containing protein 81 isoform 1 [Homo sapiens],
SEQ ID NO: 104
MAQGSGGREGALRTPAGGWHSPPSPDMQELLRSVERDLSIDPRQLAPAPGGTHVVALVPARWLASLRDRRLPLGPCPRAEGLGEAEVRTL
LQRSVQRLPAGWTRVEVHGLRKRRLSYPLGGGLPFEDGSCGPETLTRFMQEVAAQNYRNLWRHAYHTYGQPYSHSPAPSAVPALDSVR
QALQRVYGCSFLPVGETTQCPSYAREGPCPPRGSPACPSLLRAEALLESPEMLYVVHPYVQFSLHDVVTFSPAKLTNSQAKVLFILFRVLRA
MDACHRQGLACGALSLYHIAVDEKLCSELRLDLSAYERPEEDENEEAPVARDEAGIVSQEEQGGQPGQPTGQEELRSLVLDWVHGRISNF
HYLMQLNRLAGRRQGDPNYHPVLPWVVDFTTPHGRFRDLRKSKFRLNKGDKQLDFTYEMTRQAFVAGGAGGGEPPHVPHHISDVLSDIT
YYVYKARRTPRSVLCGHVRAQWEPHEYPASMERMQNWTPDECIPEFYTDPSIFRSIHPDMPDLDVPAWCSSSQEFVAAHRALLESREVSR
DLHHWIDLTFGYKLQGKEAVKEKNVCLHLVDAHTHLASYGVVQLFDQPHPQRLAGAPALAPEPPLIPKLLVQTIQETTGREDFTENPGQLP
NGVGRPVLEATPCEASWTRDRPVAGEDDLEQATEALDSISLAGKAGDQLGSSSQASPGLLSFSVASASRPGRRNKAAGADPGEGEEGRILL
PEGFNPMQALEELEKTGNFLAKGLGGLLEVPEQPRVQPAVPLQCLLHRDMQALGVLLAEMVFATRVRTLQPDAPLWVRFQAVRGLCTRH
PKEVPVSLQPVLDTLLQMSGPEVPMGAERGKLDQLFEYRPVSQGLPPPCPSQLLSPFSSVVPFPPYFPALHRFILLYQARRVEDEAQGRELV
FALWQQLGAVLKDITPEGLEILLPFVLSLMSEEHTAVYTAWYLFEPVAKALGPKNANKYLLKPLIGAYESPCQLHGRFYLYTDCFVAQLMV
RLGLQAFLTHLLPHVLQVLAGAEASQEESKDLAGAAEEEESGLPGAGPGSCAFGEEIPMDGEPPASSGLGLPDYTSGVSFHDQADLPETEDF
QAGLYVTESPQPQEAEAVSLGRLSDKSSTSETSLGEERAPDEGGAPVDKSSLRSGDSSQDLKQSEGSEEEEEEEDSCVVLEEEEGEQEEVTG
ASELTLSDTVLSMETVVAGGSGGDGEEEEEALPEQSEGKEQKILLDTACKMVRWLSAKLGPTVASRHVARNLLRLLTSCYVGPTRQQFTV
SSGESPPLSAGNIYQKRPVLGDIVSGPVLSCLLHIARLYGEPVLTYQYLPYISYLVAPGSASGPSRLNSRKEAGLLAAVTLTQKIIVYLSDTTL
MDILPRISHEVLLPVLSFLTSLVTGFPSGAQARTILCVKTISLIALICLRIGQEMVQQHLSEPVATFFQVFSQLHELRQQDLKLDPAGRGEGQLP
QVVFSDGQQRPVDPALLDELQKVFTLEMAYTIYVPFSCLLGDIIRKIIPNHELVGELAALYLESISPSSRNPASVEPTMPGTGPEWDPHGGGC
PQDDGHSGTFGSVLVGNRIQIPNDSRPENPGPLGPISGVGGGGLGSGSDDNALKQELPRSVHGLSGNWLAYWQYEIGVSQQDAHFHFHQI
RLQSFPGHSGAVKCVAPLSSEDFFLSGSKDRTVRLWPLYNYGDGTSETAPRLVYTQHRKSVFFVGQLEAPQHVVSCDGAVHVWDPFTGK
TLRTVEPLDSRVPLTAVAVMPAPHTSITMASSDSTLRFVDCRKPGLQHEFRLGGGLNPGLVRALAISPSGRSVVAGFSSGFMVLLDTRTGLV
LRGWPAHEGDILQIKAVEGSVLVSSSSDHSLTVWKELEQKPTHHYKSASDPIHTFDLYGSEVVTGTVSNKIGVCSLLEPPSQATTKLSSENF
RGTLTSLALLPTKRHLLLGSDNGVIRLLA
>gi|156119605|ref|NP_036336.2| general transcription factor 3C polypeptide 4 [Homo sapiens],
SEQ ID NO: 105
MNTADQARVGPADDGPAPSGEEEGEGGGEAGGKEPAADAAPGPSAAFRLMVTRREPAVKLQYAVSGLEPLAWSEDHRVSVSTARSIAVL
ELICDVHNPGQDLVIHRTSVPAPLNSCLLKVGSKTEVAECKEKFAASKDPTVSQTFMLDRVFNPEGKALPPMRGFKYTSWSPMGCDANGR
CLLAALTMDNRLTIQANLNRLQWVQLVDLTEIYGERLYETSYRLSKNEAPEGNLGDFAEFQRRHSMQTPVRMEWSGICTTQQVKHNNEC
RDVGSVLLAVLFENGNIAVWQFQLPFVGKESISSCNTIESGITSPSVLFWWEYEHNNRKMSGLIVGSAFGPIKILPVNLKAVKGYFTLRQPVI
LWKEMDQLPVHSIKCVPLYHPYQKCSCSLVVAARGSYVFWCLLLISKAGLNVHNSHVTGLHSLPIVSMTADKQNGTVYTCSSDGKVRQLI
PIFTDVALKFEHQLIKLSDVFGSVRTHGIAVSPCGAYLAIITTEGMINGLHPVNKNYQVQFVTLKTFEEAAAQLLESSVQNLFKQVDLIDLVR
WKILKDKHIPQFLQEALEKKIESSGVTYFWRFKLFLLRILYQSMQKTPSEALWKPTHEDSKILLVDSPGMGNADDEQQEEGTSSKQVVKQG
LQERSKEGDVEEPTDDSLPTTGDAGGREPMEEKLLEIQGKIEAVEMHLTREHMKRVLGEVYLHTWITENTSIPTRGLCNFLMSDEEYDDRT
ARVLIGHISKKMNKQTFPEHCSLCKEILPFTDRKQAVCSNGHIWLRCFLTYQSCQSLIYRRCLLHDSIARHPAPEDPDWIKRLLQSPCPFCDS
PVF
>gi|11386161|ref|NP_001843.1| collagen alpha-2(IX) chain precursor [Homo sapiens], SEQ ID NO: 106
MAAATASPRSLLVLLQVVVLALAQIRGPPGERGPPGPPGPPGVPGSDGIDGDNGPPGKAGPPGPKGEPGKAGPDGPDGKPGIDGLTGAKGE
PGPMGIPGVKGQPGLPGPPGLPGPGFAGPPGPPGPVGLPGEIGIRGPKGDPGPDGPSGPPGPPGKPGRPGTIQGLEGSADFLCPTNCPPGMKG
PPGLQGVKGHAGKRGILGDPGHQGKPGPKGDVGASGEQGIPGPPGPQGIRGYPGMAGPKGETGPHGYKGMVGAIGATGPPGEEGPRGPP
GRAGEKGDEGSPGIRGPQGITGPKGATGPPGINGKDGTPGTPGMKGSAGQAGQPGSPGHQGLAGVPGQPGTKGGPGDQGEPGPQGLPGFS
GPPGKEGEPGPRGEIGPQGIMGQKGDQGERGPVGQPGPQGRQGPKGEQGPPGIPGPQGLPGVKGDKGSPGKTGPRGKVGDPGVAGLPGE
KGEKGESGEPGPKGQQGVRGEPGYPGPSGDAGAPGVQGYPGPPGPRGLAGNRGVPGQPGRQGVEGRDATDQHIVDVALKMLQEQLAEV
AVSAKREALGAVGMMGPPGPPGPPGYPGKQGPHGHPGPRGVPGIVGAVGQIGNTGPKGKRGEKGDPGEVGRGHPGMPGPPGIPGLPGRP
GQAINGKDGDRGSPGAPGEAGRPGLPGPVGLPGFCEPAACLGASAYASARLTEPGSIKGP
>gi|148806884|ref|NP_001092103.1| proline-rich AKT1 substrate 1 [Homo sapiens], SEQ ID NO: 107
MASGRPEELWEAVVGAAERFRARTGTELVLLTAAPPPPPRPGPCAYAAHGRGALAEAARRCLHDIALAHRAATAARPPAPPPAPQPPSPTP
SPPRPTLAREDNEEDEDEPTETETSGEQLGISDNGGLFVMDEDATLQDLPPFCESDPESTDDGSLSEETPAGPPTCSVPPASALPTQQYAKSL
PVSVPVWGFKEKRTEARSSDEENGPPSSPDLDRIAASMRALVLREAEDTQVFGDLPRPRLNTSDFQKLKRKY
>gi|62739186|ref|NP_000177.2| complement factor H isoform a precursor [Homo sapiens],
SEQ ID NO: 108
MRLLAKIICLMLWAICVAEDCNELPPRRNTEILTGSWSDQTYPEGTQAIYKCRPGYRSLGNVIMVCRKGEWVALNPLRKCQKRPCGHPGD
TPFGTFTLTGGNVFEYGVKAVYTCNEGYQLLGEINYRECDTDGWTNDIPICEVVKCLPVTAPENGKIVSSAMEPDREYHFGQAVRFVCNS
GYKIEGDEEMHCSDDGFWSKEKPKCVEISCKSPDVINGSPISQKIIYKENERFQYKCNMGYEYSERGDAVCTESGWRPLPSCEEKSCDNPYI
PNGDYSPLRIKHRTGDEITYQCRNGFYPATRGNTAKCTSTGWIPAPRCTLKPCDYPDIKHGGLYHENMRRPYFPVAVGKYYSYYCDEHFE
TPSGSYWDHIHCTQDGWSPAVPCLRKCYFPYLENGYNQNHGRKFVQGKSIDVACHPGYALPKAQTTVTCMENGWSPTPRCIRVKTCSKS
SIDIENGFISESQYTYALKEKAKYQCKLGYVTADGETSGSITCGKDGWSAQPTCIKSCDIPVFMNARTKNDFTWFKLNDTLDYECHDGYES
NTGSTTGSIVCGYNGWSDLPICYERECELPKIDVHLVPDRKKDQYKVGEVLKFSCKPGFTIVGPNSVQCYHFGLSPDLPICKEQVQSCGPPP
ELLNGNVKEKTKEEYGHSEVVEYYCNPRFLMKGPNKIQCVDGEWTTLPVCIVEESTCGDIPELEHGWAQLSSPPYYYGDSVEFNCSESFT
MIGHRSITCIHGVWTQLPQCVAIDKLKKCKSSNLIILEEHLKNKKEFDHNSNIRYRCRGKEGWIHTVCINGRWDPEVNCSMAQIQLCPPPPQI
PNSHNMTTTLNYRDGEKVSVLCQENYLIQEGEEITCKDGRWQSIPLCVEKIPCSQPPQIEHGTINSSRSSQESYAHGTKLSYTCEGGFRISEE
NETTCYMGKWSSPPQCEGLPCKSPPEISHGVVAHMSDSYQYGEEVTYKCFEGFGIDGPAIAKCLGEKWSHPPSCIKTDCLSLPSFENAIPMG
EKKDVYKAGEQVTYTCATYYKMDGASNVTCINSRWTGRPTCRDTSCVNPPTVQNAYIVSRQMSKYPSGERVRYQCRSPYEMFGDEEVM
CLNGNWTEPPQCKDSTGKCGPPPPIDNGDITSFPLSVYAPASSVEYQCQNLYQLEGNKRITCRNGQWSEPPKCLHPCVISREIMENYNIALR
WTAKQKLYSRTGESVEFVCKRGYRLSSRSHTLRTTCWDGKLEYPTCAKR
>gi|4758668|ref|NP_004854.1| serine palmitoyltransferase 2 [Homo sapiens], SEQ ID NO: 109
MRPEPGGCCCRRTVRANGCVANGEVRNGYVRSSAAAAAAAAAGQIHHVTQNGGLYKRPFNEAFEETPMLVAVLTYVGYGVLTLFGYLR
DFLRYWRIEKCHHATEREEQKDFVSLYQDFENFYTRNLYMRIRDNWNRPICSVPGARVDIMERQSHDYNWSFKYTGNIIKGVINMGSYN
YLGFARNTGSCQEAAAKVLEEYGAGVCSTRQEIGNLDKHEELEELVARFLGVEAAMAYGMGFATNSMNIPALVGKGCLILSDELNHASLV
LGARLSGATIRIFKHNNMQSLEKLLKDAIVYGQPRTRRPWKKILILVEGIYSMEGSIVRLPEVIALKKKYKAYLYLDEAHSIGALGPTGRGVV
EYFGLDPEDVDVMMGTFTKSFGASGGYIGGKKELIDYLRTHSHSAVYATSLSPPVVEQIITSMKCIMGQDGTSLGKECVQQLAENTRYFRR
RLKEMGFIIYGNEDSPVVPLMLYMPAKIGAFGREMLKRNIGVVVVGFPATPIIESRARFCLSAAHTKEILDTALKEIDEVGDLLQLKYSRHRL
VPLLDRPFDETTYEETED
>gi|4507467|ref|NP_000349.1| transforming growth factor-beta-induced protein ig-h3 precursor
[Homo sapiens], SEQ ID NO: 110
MALFVRLLALALALALGPAATLAGPAKSPYQLVLQHSRLRGRQHGPNVCAVQKVIGTNRKYFTNCKQWYQRKICGKSTVISYECCPGYE
KVPGEKGCPAALPLSNLYETLGVVGSTTTQLYTDRTEKLRPEMEGPGSFTIFAPSNEAWASLPAEVLDSLVSNVNIELLNALRYHMVGRRV
LTDELKHGMTLTSMYQNSNIQIHHYPNGIVTVNCARLLKADHHATNGVVHLIDKVISTITNNIQQIIEIEDTFETLRAAVAASGLNTMLEGN
GQYTLLAPTNEAFEKIPSETLNRILGDPEALRDLLNNHILKSAMCAEAIVAGLSVETLEGTTLEVGCSGDMLTINGKAIISNKDILATNGVIHY
IDELLIPDSAKTLFELAAESDVSTAIDLFRQAGLGNHLSGSERLTLLAPLNSVFKDGTPPIDAHTRNLLRNHIIKDQLASKYLYHGQTLETLGG
KKLRVFVYRNSLCIENSCIAAHDKRGRYGTLFTMDRVLTPPMGTVMDVLKGDNRFSMLVAAIQSAGLTETLNREGVYTVFAPTNEAFRAL
PPRERSRLLGDAKELANILKYHIGDEILVSGGIGALVRLKSLQGDKLEVSLKNNVVSVNKEPVAEPDIMATNGVVHVITNVLQPPANRPQER
GDELADSALEIFKQASAFSRASQRSVRLAPVYQKLLERMKH
>gi|55743151|ref|NP_079408.3| WD repeat-containing protein 19 [Homo sapiens], SEQ ID NO: 111
MKRIFSLLEKTWLGAPIQFAWQKTSGNYLAVTGADYIVKIFDRHGQKRSEINLPGNCVAMDWDKDGDVLAVIAEKSSCIYLWDANTNKTS
QLDNGMRDQMSFLLWSKVGSFLAVGTVKGNLLIYNHQTSRKIPVLGKHTKRITCGCWNAENLLALGGEDKMITVSNQEGDTIRQTQVRSE
PSNMQFFLMKMDDRTSAAESMISVVLGKKTLFFLNLNEPDNPADLEFQQDFGNIVCYNWYGDGRIMIGFSCGHFVVISTHTGELGQEIFQA
RNHKDNLTSIAVSQTLNKVATCGDNCIKIQDLVDLKDMYVILNLDEENKGLGTLSWTDDGQLLALSTQRGSLHVFLTKLPILGDACSTRIA
YLTSLLEVTVANPVEGELPITVSVDVEPNFVAVGLYHLAVGMNNRAWFYVLGENAVKKLKDMEYLGTVASICLHSDYAAALFEGKVQLH
LIESEILDAQEERETRLFPAVDDKCRILCHALTSDFLIYGTDTGVVQYFYIEDWQFVNDYRHPVSVKKIFPDPNGTRLVFIDEKSDGFVYCPV
NDATYEIPDFSPTIKGVLWENWPMDKGVFIAYDDDKVYTYVFHKDTIQGAKVILAGSTKVPFAHKPLLLYNGELTCQTQSGKVNNIYLST
HGFLSNLKDTGPDELRPMLAQNLMLKRFSDAWEMCRILNDEAAWNELARACLHHMEVEFAIRVYRRIGNVGIVMSLEQIKGIEDYNLLAG
HLAMFTNDYNLAQDLYLASSCPIAALEMRRDLQHWDSALQLAKHLAPDQIPFISKEYAIQLEFAGDYVNALAHYEKGITGDNKEHDEACL
AGVAQMSIRMGDIRRGVNQALKHPSRVLKRDCGAILENMKQFSEAAQLYEKGLYYDKAASVYIRSKNWAKVGDLLPHVSSPKIHLQYAK
AKEADGRYKEAVVAYENAKQWQSVIRIYLDHLNNPEKAVNIVRETQSLDGAKMVARFFLQLGDYGSAIQFLVMSKCNNEAFTLAQQHN
KMEIYADIIGSEDTTNEDYQSIALYFEGEKRYLQAGKFFLLCGQYSRALKHFLKCPSSEDNVAIEMAIETVGQAKDELLTNQLIDHLLGEND
GMPKDAKYLFRLYMALKQYREAAQTAIIIAREEQSAGNYRNAHDVLFSMYAELKSQKIKIPSEMATNLMILHSYILVKIFIVKNGDHMKGA
RMLIRVANNISKFPSHIVPILTSTVIECHRAGLKNSAFSFAAMLMRPEYRSKIDAKYKKKIEGMVRRPDISEIEEATTPCPFCKFLLPECELLCP
GCKNSIPYCIATGRHMLKDDWTVCPHCDFPALYSELKIMLNTESTCPMCSERLNAAQLKKISDCTQYLRTEEEL
>gi|47086907|ref|NP_060309.2| lysophosphatidylcholine acyltransferase 2 [Homo sapiens],
SEQ ID NO: 112
MSRCAQAAEVAATVPGAGVGNVGLRPPMVPRQASFFPPPVPNPFVQQTQIGSARRVQIVLLGIILLPIRVLLVALILLLAWPFAAISTVCCPE
KLTHPITGWRRKITQTALKFLGRAMFFSMGFIVAVKGKIASPLEAPVFVAAPHSTFFDGIACVVAGLPSMVSRNENAQVPLIGRLLRAVQPV
LVSRVDPDSRKNTINEIIKRTTSGGEWPQILVFPEGTCTNRSCLITFKPGAFIPGVPVQPVLLRYPNKLDTVTWTWQGYTFIQLCMLTFCQLF
TKVEVEFMPVQVPNDEEKNDPVLFANKVRNLMAEALGIPVTDHTYEDCRLMISAGQLTLPMEAGLVEFTKISRKLKLDWDGVRKHLDEY
ASIASSSKGGRIGIEEFAKYLKLPVSDVLRQLFALFDRNHDGSIDFREYVIGLAVLCNPSNTEEIIQVAFKLFDVDEDGYITEEEFSTILQASLG
VPDLDVSGLFKEIAQGDSISYEEFKSFALKHPEYAKIFTTYLDLQTCHVFSLPKEVQTTPSTASNKVSPEKHEESTSDKKDD
>gi|21389401|ref|NP_653202.1| CKLF-like MARVEL transmembrane domain-containing protein 3 [Homo
sapiens], SEQ ID NO: 113
MWPPDPDPDPDPEPAGGSRPGPAVPGLRALLPARAFLCSLKGRLLLAESGLSFITFICYVASSASAFLTAPLLEFLLALYFLFADAMQLNDK
WQGLCWPMMDFLRCVTAALIYFAISITAIAKYSDGASKAAGVFGFFATIVFATDFYLIFNDVAKFLKQGDSADETTAHKTEEENSDSDSD
>gi|21361933|ref|NP_110417.2| T-cell immunomodulatory protein precursor [Homo sapiens],
SEQ ID NO: 114
MAAAGRLPSSWALFSPLLAGLALLGVGPVPARALHNVTAELFGAEAWGTLAAFGDLNSDKQTDLFVLRERNDLIVFLADQNAPYFKPKV
KVSFKNHSALITSVVPGDYDGDSQMDVLLTYLPKNYAKSELGAVIFWGQNQTLDPNNMTILNRTFQDEPLIMDFNGDLIPDIFGITNESNQP
QILLGGNLSWHPALTTTSKMRIPHSHAFIDLTEDFTADLFLTTLNATTSTFQFEIWENLDGNFSVSTILEKPQNMMVVGQSAFADFDGDGH
MDHLLPGCEDKNCQKSTIYLVRSGMKQWVPVLQDFSNKGTLWGFVPFVDEQQPTEIPIPITLHIGDYNMDGYPDALVILKNTSGSNQQAFL
LENVPCNNASCEEARRMFKVYWELTDLNQIKDAMVATFFDIYEDGILDIVVLSKGYTKNDFAIHTLKNNFEADAYFVKVIVLSGLCSNDCP
RKITPFGVNQPGPYIMYTTVDANGYLKNGSAGQLSQSAHLALQLPYNVLGLGRSANFLDHLYVGIPRPSGEKSIRKQEWTAIIPNSQLIVIPY
PHNVPRSWSAKLYLTPSNIVLLTAIALIGVCVFILAIIGILHWQEKKADDREKRQEAHRFHFDAM
>gi|31542634|ref|NP_056502.1| filamin-A-interacting protein 1 [Homo sapiens], SEQ ID NO: 115
MRSRNQGGESASDGHISCPKPSIIGNAGEKSLSEDAKKKKKSNRKEDDVMASGTVKRHLKTSGECERKTKKSLELSKEDLIQLLSIMEGEL
QAREDVIHMLKTEKTKPEVLEAHYGSAEPEKVLRVLHRDAILAQEKSIGEDVYEKPISELDRLEEKQKETYRRMLEQLLLAEKCHRRTVYE
LENEKHKHTDYMNKSDDFTNLLEQERERLKKLLEQEKAYQARKEKENAKRLNKLRDELVKLKSFALMLVDERQMHIEQLGLQSQKVQD
LTQKLREEEEKLKAITSKSKEDRQKLLKLEVDFEHKASRFSQEHEEMNAKLANQESHNRQLRLKLVGLTQRIEELEETNKNLQKAEEELQE
LRDKIAKGECGNSSLMAEVENLRKRVLEMEGKDEEITKTESQCRELRKKLQEEEHHSKELRLEVEKLQKRMSELEKLEEAFSKSKSECTQL
HLNLEKEKNLTKDLLNELEVVKSRVKELECSESRLEKAELSLKDDLTKLKSFTVMLVDERKNMMEKIKQEERKVDGLNKNFKVEQGKVM
DVTEKLIEESKKLLKLKSEMEEKVYNLTRERDELIGKLKSEEEKSSELSCSVDLLKKRLDGIEEVEREITRGRSRKGSELTCPEDNKIKELTLEI
ERLKKRLQQLEVVEGDLMKTEDEYDQLEQKFRTEQDKANFLSQQLEEIKHQIAKNKAIEKGEVVSQEAELRHRFRLEEAKSRDLKAEVQA
LKEKIHELMNKEDQLSQLQVDYSVLQQRFMEEENKNKNMGQEVLNLTKELELSKRYSRALRPSVNGRRMVDVPVTSTGVQTDAVSGEA
AEEETPAVFIRKSFQEENHIMSNLRQVGLKKPVERSSVLDRYPPAANELTMRKSWIPWMRKRENGPSITQEKGPRTNSSPGHPGEVVLSPK
QGQPLHIRVTPDHENSTATLEITSPTSEEFFSSTTVIPTLGNQKPRITIIPSPNVMPQKQKSGDTTLGPERAMSPVTITTFSREKTPESGRGAFA
DRPTSPIQIMTVSTSAAPAEIAVSPESQEMPMGRTILKVTPEKQTVPTPVRKYNSNANIITTEDNKIHIHLGSQFKRSPGTSGEGVSPVITVRPV
NVTAEKEVSTGTVLRSPRNHLSSRPGASKVTSTITITPVTTSSARGTQSVSGQDGSSQRPTPTRIPMSKGMKAGKPVVAAPGAGNLTKFEPR
AETQSMKIELKKSAASSTTSLGGGKG
>gi|153791560|ref|NP_001074290.1| zinc finger protein 799 [Homo sapiens], SEQ ID NO: 116
MASVALEDVAVNFTREEWALLGPCQKNLYKDVMQETIRNLDCVGMKWKDQNIEDQYRYPRKNLRCRMLERFVESKDGTQCGETSSQIQ
DSIVTKNTLPGVGPYESRMSGEVIMGHSSLNCYIRVGAGHKPYEYHECGEKPDTHKQRGKAFSYHNSLQTHERLHTGKKPYNCKECGKSF
SSLGNLQRHMAVQRGDGPYKCKLCGKAFFWPSLLHMHERTHTGEKPYECKQCSKAFSFYSSYLRHERTHTGEKLYECKQCSKAFPDYSS
CLRHERTHTGKKPYTCKQCGKAFSASTSLRRHETTHTDEKPYACQQCGKAFHHLGSFQRHMVMHTRDGPHKCKICGKGFDCPSSLKSHE
RTHTGEKLYECKQCGKALSHSSSFRRHMTMHTGDGPHKCKICGKAFVYPSVFQRHEKTHTAEKPYKCKQCGKAYRISSSLRRHETTHTG
EKPYKCKCGKAFIDFYSFQNHKTTHAGEKPYECKECGKAFSCFQYLSQHRRTHTGEKPYECNTCKKAFSHFGNLKVHERIHSGEKPYECK
ECGKAFSWLTCFLRHERIHMREKPYECQQCGKAFTHSRFLQGHEKTHTGENPYECKECGKAFASLSSLHRHKKTHWKKTHTGENPYGCK
ECGKAFASLSSLHRHKKTH
>gi|13562090|ref|NP_112092.1| apolipoprotein L2 [Homo sapiens], SEQ ID NO: 117
MNPESSIFIEDYLKYFQDQVSRENLLQLLTDDEAWNGFVAAAELPRDEADELRKALNKLASHMVMKDKNRHDKDQQHRQWFLKEFPRL
KRELEDHIRKLRALAEEVEQVHRGTTIANVVSNSVGTTSGILTLLGLGLAPFTEGISFVLLDTGMGLGAAAAVAGITCSVVELVNKLRARAQ
ARNLDQSGTNVAKVMKEFVGGNTPNVLTLVDNWYQVTQGIGRNIRAIRRARANPQLGAYAPPPHIIGRISAEGGEQVERVVEGPAQAMSR
GTMIVGAATGGILLLLDVVSLAYESKHLLEGAKSESAEELKKRAQELEGKLNFLTKIHEMLQPGQDQ
>gi|196114945|ref|NP_001124477.1| myocyte-specific enhancer factor 2C isoform 2 [Homo sapiens],
SEQ ID NO: 118
MGRKKIQITRIMDERNRQVTFTKRKFGLMKKAYELSVLCDCEIALEFNSTNKLFQYASTDMDKVLLKYTEYNEPHESRTNSDIVEALNKKE
NKGCESPDPDSSYALTPRTEEKYKKINEEFDNMIKSHKIPAVPPPNFEMPVSIPVSSHNSLVYSNPVSSLGNPNLLPLAHPSLQRNSMSPGVT
HRPPSAGNTGGLMGGDLTSGAGTSAGNGYGNPRNSPGLLVSPGNLNKNMQAKSPPPMNLGMNNRKPDLRVLIPPGSKNTMPSVNQRINN
SQSAQSLATPVVSVATPTLPGQGMGGYPSAISTTYGTEYSLSSADLSSLSGFNTASALHLGSVTGWQQQHLHNMPPSALSQLGACTSTHLS
QSSNLSLPSTQSLNIKSEPVSPPRDRTTTPSRYPQHTRHEAGRSPVDSLSSCSSSYDGSDREDHRNEFHSPIGLTRPSPDERESPSVKRMRLSE
GWAT
>gi|18034767|ref|NP_071387.1| phosphorylated CTD -interacting factor 1 [Homo sapiens],
SEQ ID NO: 119
MANENHGSPREEASLLSHSPGTSNQSQPCSPKPIRLVQDLPEELVHAGWEKCWSRRENRPYYFNRFTNQSLWEMPVLGQHDVISDPLGLN
ATPLPQDSSLVETPPAENKPRKRQLSEEQPSGNGVKKPKIEIPVTPTGQSVPSSPSIPGTPTLKMWGTSPEDKQQAALLRPTEVYWDLDIQTN
AVIKHRGPSEVLPPHPEVELLRSQLILKLRQHYRELCQQREGIEPPRESFNRWMLERKVVDKGSDPLLPSNCEPVVSPSMFREIMNDIPIRLS
RIKFREEAKRLLFKYAEAARRLIESRSASPDSRKVVKWNVEDTFSWLRKDHSASKEDYMDRLEHLRRQCGPHVSAAAKDSVEGICSKIYHI
SLEYVKRIREKHLAILKENNISEEVEAPEVEPRLVYCYPVRLAVSAPPMPSVEMHMENNVVCIRYKGEMVKVSRNYFSKLWLLYRYSCID
DSAFERFLPRVWCLLRRYQMMFGVGLYEGTGLQGSLPVHVFEALHRLFGVSFECFASPLNCYFRQYCSAFPDTDGYFGSRGPCLDFAPLS
GSFEANPPFCEELMDAMVSHFERLLESSPEPLSFIVFIPEWREPPTPALTRMEQSRFKRHQL1LPAFEHEYRSGSQHICKKEEMHYKAVHNTA
VLFLQNDPGFAKWAPTPERLQELSAAYRQSGRSHSSGSSSSSSSEAKDRDSGREQGPSREPHPT
>gi|302191702|ref|NP_001180539.1| tubulin delta chain isoform 3 [Homo sapiens], SEQ ID NO: 120
MSIVTVQLGQCGNQIGFEVFDALLSDSHSSQGLCSMRENEAYQASCKERFFSEEENGVPIARAVLVDMEPKVINQMLSKAAQSGQWKYG
QHACFCQKQGSGNNWAYGYSVHGPRHEESIMNIIRKEVEKCDSFSGFFIIMSMAGGTGSGLGAFVTQNLEDQYSNSLKMNQIIWPYGTGE
VIVQNYNSILTLSHLYRSSDALLLHENDAIHKICAKLMNIKQISFSDINQVLAHQLGSVFQPTYSAESSFHYRRNPLGDLMEHLVPHPEFKML
SVRNIPHMSENSLAYTTFTWAGLLKHLRQMLISNAKMEEGIDRHVWPPLSGLPPLSKMSLNKDLHFNTSIANLVILRGKDVQSADVGKKFR
AYIHQYTKFGIEEEDFLDSFTSLEQVVASYCNL
>gi|57232746|ref|NP_003480.2| nuclear receptor-interacting protein 1 [Homo sapiens],
SEQ ID NO: 121
MTHGEELGSDVHQDSIVLTYLEGLLMHQAAGGSGTAVDKKSAGHNEEDQNFNISGSAFPTCQSNGPVLNTHTYQGSGMLHLKKARLLQS
SEDWNAAKRKRLSDSIMNLNVKKEALLAGMVDSVPKGKQDSTLLASLLQSFSSRLQTVALSQQIRQSLKEQGYALSHDSLKVEKDLRCYG
VASSHLKTLLKKSKVKDQKPDTNLPDVTKNLIRDRFAESPHHVGQSGTKVMSEPLSCAARLQAVASMVEKRASPATSPKPSVACSQLALL
LSSEAHLQQYSREHALKTQNANQAASERLAAMARLQENGQKDVGSYQLPKGMSSHLNGQARTSSSKLMASKSSATVFQNPMGIIPSSPK
NAGYKNSLERNNIKQAANNSLLLHLLKSQTIPKPMNGHSHSERGSIFEESSTPTTIDEYSDNNPSFTDDSSGDESSYSNCVPIDLSCKHRTEK
SESDQPVSLDNFTQSLLNTWDPKVPDVDIKEDQDTSKNSKLNSHQKVTLLQLLLGHKNEENVEKNTSPQGVHNDVSKFNTQNYARTSVIE
SPSTNRFlPVSTPPLLTSSKAGSPINLSQHSLVIKWNSPPYVCSTQSEKLTNTASNHSMDLTKSKDPPGEKPAQNEGAQNSATFSASKLLQN
LAQCGMQSSMSVEEQRPSKQLLTGNTDKPIGMIDRLNSPLLSNKTNAVEENKAFSSQPTGPEPGLSGSEIENLLERRTVLQLLLGNPNKGKS
EKKEKTPLRDESTQEHSERALSEQILMVKIKSEPCDDLQIPNTNVHLSHDAKSAPFLGMAPAVQRSAPALPVSEDFKSEPVSPQDFSFSKNG
LLSRLLRQNQDSYLADDSDRSHRNNEMALLESKNLCMVPKKRKLYTEPLENPFKKMKNNIVDAANNHSAPEVLYGSLLNQEELKFSRND
LEFKYPAGHGSASESEHRSWARESKSFNVLKQLLLSENCVRDLSPHRSNSVADSKKKGHKNNVTNSKPEFSISSLNGLMYSSTQPSSCMDN
RTFSYPGVVKTPVSPTFPEHLGCAGSRPESGLLNGCSMPSEKGPIKWVITDAEKNEYEKDSPRLTKTNPILYYMLQKGGNSVTSRETQDKDI
WREASSAESVSQVTAKEELLPTAETKASFFNLRSPYNSHMGNNASRPHSANGEVYGLLGSVLTIKKESE
>gi|169636420|ref|NP_001207.2| carbonic anhydrase 9 precursor [Homo sapiens], SEQ ID NO: 122
MAPLCPSPWLPLLIPAPAPGLTVQLLLSLLLLVPVHPQRLPRMQEDSPLGGGSSGEDDPLGEEDLPSEEDSPREEDPPGEEDLPGEEDLPGEE
DLPEVKPKSEEEGSLKLEDLPTVEAPGDPQEPQNNAHRDKEGDDQSHWRYGGDPPWPRVSPACAGRFQSPVDIRPQLAAFCPALRPLELL
GFQLPPLPELRLRNNGHSVQLTLPPGLEMALGPGREYRALQLHLHWGAAGRPGSEHTVEGHRFPAEIHVVHLSTAFARVDEALGRPGGLA
VLAAFLEEGPEENSAYEQLLSRLEEIAEEGSETQVPGLDISALLPSDFSRYFQYEGSLTTPPCAQGVIWTVFNQTVMLSAKQLHTLSDTLWG
PGDSRLQLNFRATQPLNGRVIEASFPAGVDSSPRAAEPVQLNSCLAAGDILALVFGLLFAVTSVAFLVQMRRQHRRGTKGGVSYRPAEVAE
TGA
>gi|45439306|ref|NP_001340.2| aspartyl-tRNA synthetase, cytoplasmic [Homo sapiens], SEQ ID NO: 123
MPSASASRKSQEKPREIMDAAEDYAKERYGISSMIQSQEKPDRVLVRVRDLTIQKADEVVWVRARVHTSRAKGKQCFLVLRQQQFNVQA
LVAVGDHASKQMVKFAANINKESIVDVEGVVRKVNQKIGSCTQQDVELHVQKIYVISLAEPRLPLQLDDAVRPEAEGEEEGRATVNQDTR
LDNRVIDLRTSTSQAVFRLQSGICHLFRETLINKGFVEIQTPKIISAASEGGANVFTVSYFKNNAYLAQSPQLYKQMCICADFEKVFSIGPVFR
AEDSNTHRHLTEFVGLDIEMAFNYHYHEVMEEIADTMVQIFKGLQERFQTEIQTVNKQFPCEPFKFLEPTLRLEYCEALAMLREAGVEMG
DEDDLSTPNEKLLGHLVKEKYDTDFYILDKYPLAVRPFYTMPDPRNPKQSNSYDMFMRGEEILSGAQRIHDPQLLTERALHHGIDLEKIKA
YIDSFRFGAPPHAGGGIGLERVTMLFLGLHNVRQTSMFPRDPKRLTP
>gi|1310118388|ref|XP_003119104.1| PREDICTED: putative Ras GTPase-activating protein 4B isoform 5
[Homo sapiens],SEQ ID NO: 124
MIQSSSTPTRDLAPKDRNGTSDPFVRVRYKGRTRETSIVKKSCYPRWNETFEFELQEGAMEALCVEAWDWDLVSRNDFLGKVVIDVQRLR
VVQQEEGWFRLQPDQSKSRRHDEGNLGSLQLEVRLRDETVLPSSYYQPLVHLLCHEVKLGMQGPGQLIPLIEETTSTECRQDVATNLLKLF
LGQGLAKDFLDLLFQLELSRTSETNTLFRSNSLASKSVESFLKVAGMQYLHGVLGPIINKVFEEKKYVELDPSKVEVKDVGCSGLHRPQTEA
EVLEQSAQTLRAHLGALLSALSRSVRACPAVVRATFRQLFRRVRERFPGAQHENVPFIAVTSFLCLRFFSPAIMSPKLFHLRERHADARTSR
TLLLLAKAVQNVGNMDTPASRAKEAWMEPLQPTVHQGVAQLKDFITKLVDIEEKDELDLQRTLSLQAPPVKEGPLFIHRTKGKGPLMSSSF
KKLYFSLTTEALSFAKTPSSKKSALIKLANIRAAEKVEEKSFGGSHVMQVIYTDDAGRPQTAYLQCKCVNELNQWLSALRKVSINNTGLLG
SYHPGVFRGDKWSCCHQKEKTGQGCDKTRSRVTLQEWNDPLDHDLEAQLIYRHLLGVEAMLWERHRELSGGAEAGTVPTSPGKVPEDS
LARLLRVLQDLREAHSSSPAGSPPSEPNCLLELQT
>gi|14719829|refINP_127497.1| NACHT, LRR and PYD domains-containing protein 1 isoform 1 [Homo
sapiens], SEQ ID NO: 125
MAGGAWGRLACYLEFLKKEELKEFQLLLANKAHSRSSSGETPAQPEKTSGMEVASYLVAQYGEQRAWDLALHTWEQMGLRSLCAQAQ
EGAGHSPSFPYSPSEPHLGSPSQPTSTAVLMPWIHELPAGCTQGSERRVLRQLPDTSGRRWREISASLLYQALPSSPDHESPSQESPNAPTST
AVLGSWGSPPQPSLAPREQEAPGTQWPLDETSGIYYTEIREREREKSEKGRPPWAAVVGTPPQAHTSLQPHHHPWEPSVRESLCSTWPWK
NEDFNQKFTQLLLLQRPHPRSQDPLVKRSWPDYVEENRGHLIEIRDLFGPGLDTQEPRIVILQGAAGIGKSTLARQVKEAWGRGQLYGDRF
QHVFYFSCRELAQSKVVSLAELIGKDGTATPAPIRQILSRPERLLFILDGVDEPGWVLQEPSSELCLHWSQPQPADALLGSLLGKTILPEASFLI
TARTTALQNLIPSLEQARWVEVLGFSESSRKEYFYRYFTDERQAIRAFRLVKSNKELWALCLVPWVSWLACTCLMQQMKRKEKLTLTSKT
TTTLCLHYLAQALQAQPLGPQLRDLCSLAAEGIWQKKTLFSPDDLRKHGLDGAIISTFLKMGILQEHPIPLSYSFIHLCFQEFFAAMSYVLED
EKGRGKHSNCIIDLEKTLEAYGIHGLFGASTTRFLLGLLSDEGEREMENIFHCRLSQGRNLMQWVPSLQLLLQPHSLESLHCLYETRNKTFLT
QVMAHFEEMGMCVETDMELLVCTFCIKFSRHVKKLQLIEGRQHRSTWSPTMVVLFRWVPVTDAYWQILFSVLKVTRNLKELDLSGNSLS
HSAVKSLCKTLRRPRCLLETLRLAGCGLTAEDCKDLAFGLRANQTLTELDLSFNVLTDAGAKHLCQRLRQPSCKLQRLQLVSCGLTSDCCQ
DLASVLSASPSLKELDLQQNNLDDVGVRLLCEGLRHPACKLIRLGLDQTTLSDEMRQELRALEQEKPQLLIFSRRKPSVMTPTEGLDTGEM
SNSTSSLKRQRLGSERAASHVAQANLKLLDVSKIFPIAEIAEESSPEVVPVELLCVPSPASQGDLHTKPLGTDDDFWGPTGPVATEVVDKEK
NLYRVHFPVAGSYRWPNTGLCFVMREAVTVEIEFCVWDQFLGEINPQHSWMVAGPLLDIKAEPGAVEAVHLPHFVALQGGHVDTSLFQM
AHFKEEGMLLEKPARVELHHIVLENPSFSPLGVLLKMIHNALRFIPVTSVVLLYHRVHPEEVTFHLYLIPSDCSIRKAIDDLEMKFQFVRIHKP
PPLTPLYMGCRYTVSGSGSGMLEILPKELELCYRSPGEDQLFSEFYVGHLGSGIRLQVKDKKDETLVWEALVKPGDLMPATTLIPPARIAVP
SPLDAPQLLHFVDQYREQLIARVTSVEVVLDKLHGQVLSQEQYERVLAENTRPSQMRKLFSLSQSWDRKCKDGLYQALKETHPHLIMEL
WEKGSKKGLLPLSS
>gi|4506179|ref|NP_002777.1| proteasome subunit alpha type-1 isoform 2 [Homo sapiens],
SEQ ID NO: 126
MFRNQYDNDVTVWSPQGRIHQIEYAMEAVKQGSATVGLKSKTHAVLVALKRAQSELAAHQKKILHVDNHIGISIAGLTADARLLCNFMR
QECLDSRFVFDRPLPVSRLVSLIGSKTQIPTQRYGRRPYGVGLLIAGYDDMGPHIFQTCPSANYFDCRAMSIGARSQSARTYLERHMSEFME
CNLNELVKHGLRALRETLPAEQDLTTKNVSIGIVGKDLEFTIYDDDDVSPFLEGLEERPQRKAQPAQPADEPAEKADEPMEH
>gi|4502809|ref|NP_003456.1| chitotriosidase-1 precursor [Homo sapiens], SEQ ID NO: 127
MVRSVAWAGFMVLLMIPWGSAAKLVCYFTNWAQYRQGEARFLPKDLDPSLCTHLIYAFAGMTNHQLSTTEWNDETLYQEFNGLKKMN
PKLKTLLAIGGWNFGTQKFTDMVATANNRQTFVNSAIRFLRKYSFDGLDLDWEYPGSQGSPAVDKERFTTLVQDLANAFQQEAQTSGKE
RLLLSAAVPAGQTYVDAGYEVDKIAQNLDFVNLMAYDFHGSWEKVTGHNSPLYKRQEESGAAASLNVDAAVQQWLQKGTPASKLILGM
PTYGRSFTLASSSDTRVGAPATGSGTPGPFTKEGGMLAYYEVCSWKGATKQRIQDQKVPY1FRDNQWVGFDDVESFKTKVSYLKQKGLG
GAMVWALDLDDFAGFSCNQGRYPLIQTLRQELSLPYLPSGTPELEVPKPGQPSEPEHGPSPGQDTFCQGKADGLYPNPRERSSFYSCAAGR
LFQQSCPTGLVFSNSCKCCTWN
>gi|167614506|ref|NP_002289.2| plastin-2 [Homo sapiens], SEQ ID NO: 128
MARGSVSDEEMMELREAFAKVDTDGNGYISFNELNDLFKAACLPLPGYRVREITENLMATGDLDQDGRISFDEFIKIFHGLKSTDVAKTFR
KAINKKEGICAIGGTSEQSSVGTQHSYSEEEKYAFVNWINKALENDPDCRHVIPMNPNTNDLFNAVGDGIVLCKMINLSVPDTIDERTINKK
KLTPFTIQENLNLALNSASAIGCHVVNIGAEDLKEGKPYLVLGLLWQVIKIGLFADIELSRNEALIALLREGESLEDLMKLSPEELLLRWANY
HLENAGCNKIGNFSTDIKDSKAYYHLLEQVAPKGDEEGVPAVVIDMSGLREKDDIQRAECMLQQAERLGCRQFVTATDVVRGNPKLNLA
FIANLFNRYPALHKPENQDIDWGALEGETREERTFRNWMNSLGVNPRVNHLYSDLSDALVIFQLYEKIKVPVDWNRVNKPPYPKLGGNM
KKLENCNYAVELGKNQAKFSLVGIGGQDLNEGNRTLTLALIWQLMRRYTLNILEEIGGGQKVNDDIIVNWVNETLREAKKSSSISSFKDPKI
STSLPVLDLIDAIQPGSINYDLLKTENLNDDEKLNNAKYAISMARKIGARVYALPEDLVEVNPKMVMTVFACLMGKGMKRV
>gi|148596984|ref|NP_004478.3| Golgin subfamily B member 1 [Homo sapiens], SEQ ID NO: 129
MLSRLSGLANVVLHELSGDDDTDQNMRAPLDPELHQESDMEFNNTTQEDVQERLAYAEQLVVELKDIIRQKDVQLQQKDEALQEERKAA
DNKIKKLKLHAKAKLTSLNKYIEEMKAQGGTVLPTEPQSEEQLSKHDKSSTEEEMEIEKIKHKLQEKEELISTLQAQLTQAQAEQPAQSSTE
MEEFVMMKQQLQEKEEFISTLQAQLSQTQAEQAAQQVVREKDARFETQVRLHEDELLQLVTQADVETEMQQKLRVLQRKLEEHEESLV
GRAQVVDLLQQELTAAEQRNQILSQQLQQMEAEHNTLRNTVETEREESKILLEKMELEVAERKLSFHNLQEEMHHLLEQFEQAGQAQAEL
ESRYSALEQKHKAEMEEKTSHILSLQKTGQELQSACDALKDQNSKLLQDKNEQAVQSAQTIQQLEDQLQQKSKEISQFLNRLPLQQHETAS
QTSFPDVYNEGTQAVTEENIASLQKRVVELENEKGALLLSSIELEELKAENEKLSSQITLLEAQNRTGEADREVSEISIVDIANKRSSSAEESG
QDVLEN'TFSQKHKELSVLLLEMKEAQEEIAFLKLQLQGKRAEEADHEVLDQKEMKQMEGEGIAPIKMKVFLEDTGQDFPLMPNEESSLPA
VEKEQASTEHQSRTSEEISLNDAGVELKSTKQDGDKSLSAVPDIGQCHQDELERLKSQILELELNFHKAQEIYEKNLDEKAKEISNLNQUEE
FKKNADNNSSAFTALSEERDQLLSQVKELSMVTELRAQVKQLEMNLAEAERQRRLDYESQTAHDNLLTEQIHSLSIEAKSKDVKIEVLQNE
LDDVQLQFSEQSTLIRSLQSQLQNKESEVLEGAERVRHISSKVEELSQALSQKELEITKMDQLLLEKKRDVETLQQTIEEKDQQVTEISFSMT
EKMVQLNEEKFSLGVEIKTLKEQLNLLSRAEEAKKEQVEEDNEVSSGLKQNYDEMSPAGQISKEELQHEFDLLKKENEQRKRKLQAALIN
RKELLQRVSRLEEELANLKDESKKEIPLSETERGEVEEDKENKEYSEKCVTSKCQEIEIYLKQTISEKEVELQHIRKDLEEKLAAEEQFQALV
KQMNQTLQDKTNQIDLLQAEISENQAIIQKLITSNTDASDGDSVALVKETVVISPPCTGSSEHWKPELEEKILALEKEKEQLQKKLQEALTSR
KAILKKAQEKERHLREELKQQKDDYNRLQEQFDEQSKENENIGDQLRQLQIQVRESIDGKLPSTDQQESCSSTPGLEEPLFKATEQHHTQP
VLESNLCPDWPSHSEDASALQGGTSVAQIKAQLKEIEAEKVELELKVSSTTSELTKKSEEVFQLQEQINKQGLEIESLKTVSHEAEVHAESLQ
QKLESSQLQIAGLEHLRELQPKLDELQKLISKKEEDVSYLSGQLSEKEAALTKIQTEBEQEDLIKALHTQLEMQAKEHDERIKQLQVELCEM
KQKPEEIGEESRAKQQIQRKLQAALISRKEALKENKSLQEELSLARGTIERLTKSLADVESQVSAQNKEKDTVLGRLALLQEERDKLITEMD
RSLLENQSLSSSCESLKLALEGLTEDKEKLVKEIESLKSSKIAESTEWQEKHKELQKEYEILLQSYENVSNEAERIQHVVEAVRQEKQELYGK
LRSTEANKKETEKQLQEAEQEMEEMKEKMRKFAKSKQQKILELEEENDRLRAEVHPAGDTAKECMETLLSSNASMKEELERVKMEYETL
SKKFQSLMSEKDSLSEEVQDLKHQIEGNVSKQANLEATEKHDNQTNVTEEGTQSIPGETEEQDSLSMSTRPTCSESVPSAKSANPAVSKDF
SSHDEINNYLQQIDQLKERIAGLEEEKQKNKEFSQTLENEKNTLLSQISTKDGELKMLQEEVTKMNLLNQQIQEELSRVTKLKETAEEEKDD
LEERLMNQLAELNGSIGNYCQDVTDAQIKNELLESEMKNLKKCVSELEEEKQQLVKEKTKVESEIRKEYLEKIQGAQKEPGNKSHAKELQ
ELLKEKQQEVKQLQKDCIRYQEKISALERTVKALEFVQTESQKDLEITKENLAQAVEHRKKAQAELASFKVLLDDTQSEAARVLADNLKL
KKELQSNKESVKSQMKQKDEDLERRLEQAEEKHLKEKKNMQEKLDALRREKVHLEETIGEIQVTLNKKDKEVQQLQENLDSTVTQLAAF
TKSMSSLQDDRDRVIDEAKKWERKFSDAIQSKEEEIRLKEDNCSVLKDQLRQMSIHMEELKINISRLEHDKQIWESKAQTEVQLQQKVCDT
LQGENKELLSQLEETRHLYHSSQNELAKLESELKSLKDQLTDLSNSLEKCKEQKGNLEGIIRQQEADIQNSKFSYEQLETDLQASRELTSRLH
EEINMKEQKIISLLSGKEEAIQVAIAELRQQHDKEIKELENLLSQEEEENIVLEEENKKAVDKTNQLMETLKTIKKENIQQKAQLDSFVKSMS
SLQNDRDRIVGDYQQLEERHLSBLEKDQLIQEAAAENNKLKEEIRGLRSHMDDLNSENAKLDAELIQYREDLNQVITIKDSQQKQLLEVQL
QQNKELENKYAKLEEKLKESEEANEDLRRSFNALQEEKQDLSKEIESLKVSISQLTRQVTALQEEGTLGLYHAQLKVKEEEVHRLSALFSSS
QKRIAELEEELVCVQKEAAKKVGEIEDKLKKELKHLHHDAGIMRNETETAEERVAELARDLVEMEQKLLMVTKENKGLTAQIQSFGRSMS
SLQNSRDHANEELDELKRKYDASLKELAQLKEQGLLNRERDALLSETAFSMNSTEENSLSHLEKLNQQLLSKDEQLLHLSSQLEDSYNQV
QSFSKAMASLQNERDHLWNELEKFRKSEEGKQRSAAQPSTSPAEVQSLKKAMSSLQNDRDRLLKELKNLQQQYLQINQEITELHPLKAQL
QEYQDKTKAFQINIQEELRQENLSWQHELHQLRMEKSSWEIHERRMKEQYLMAISDKDQQLSHLQNLIRELRSSSSQTQPLKVQYQRQAS
PETSASPDGSQNLVYETELLRTQLNDSLKEIHQKELRIQQLNSNFSQLLEEKNTLSIQLCDTSQSLRENQQHYGDLLNHCAVLEKQVQELQA
GPLNIDVAPGAPQEKNGVHRKSDPEELREPQQSFSEAQQQLCNTRQEVNELRKLLEEERDQRVAAENALSVAEEQIRRLEHSEWDSSRTPII
GSCGTQEQALLIDLTSNSCRRTRSGVGWKRVLRSLCHSRTRVPLLAAIYFLMIHVLLILCFTGHL
>gi|11386161|ref|NP_001843.1| collagen alpha-2(IX) chain precursor [Homo sapiens], SEQ ID NO: 130
MAAATASPRSLLVLLQVVVLALAQIRGPPGERGPPGPPGPPGVPGSDGIDGDNGPPGKAGPPGPKGEPGKAGPDGPDGKPGIDGLTGAKGE
PGPMGIPGVKGQPGLPGPPGLPGPGFAGPPGPPGPVGLPGEIGIRGPKGDPGPDGPSGPPGPPGKPGRPGTIQGLEGSADFLCPTNCPPGMKG
PPGLQGVKGHAGKRGILGDPGHQGKPGPKGDVGASGEQGIPGPPGPQGIRGYPGMAGPKGETGPHGYKGMVGAIGATGPPGEEGPRGPP
GRAGEKGDEGSPGIRGPQGITGPKGATGPPGINGKDGTPGTPGMKGSAGQAGQPGSPGHQGLAGVPGQPGTKGGPGDQGEPGPQGLPGFS
GPPGKEGEPGPRGEIGPQGIMGQKGDQGERGPVGQPGPQGRQGPKGEQGPPGIPGPQGLPGVKGDKGSPGKTGPRGKVGDPGVAGLPGE
KGEKGESGEPGPKGQQGVRGEPGYPGPSGDAGAPGVQGYPGPPGPRGLAGNRGVPGQPGRQGVEGRDATDQHIVDVALKMLQEQLAEV
AVSAKREALGAVGMMGPPGPPGPPGYPGKQGPHGHPGPRGVPGIVGAVGQIGNTGPKGKRGEKGDPGEVGRGHPGMPGPPGIPGLPGRP
GQAINGKDGDRGSPGAPGEAGRPGLPGPVGLPGFCEPAACLGASAYASARLTEPGSIKGP
>gi|300797277|ref|NP_705932.2| sodium/potassium/calcium exchanger 4 isoform 1 precursor [Homo
sapiens], SEQ ID NO: 131
MALRGTLRPLKVRRRREMLPQQVGFVCAVLALVCCASGLFGSLGHKTASASKRVLPDTWRNRKLMAPVNGTQTAKNCTDPAIHEFPTDL
FSNKERQHGAVLLHILGALYMFYALAIVCDDFFVPSLEKICERLHLSEDVAGATFMAAGSSTPELFASVIGVFITHGDVGVGTIVGSAVFNIL
CIIGVCGLFAGQVVRLTWWAVCRDSVYYTISVIVLIVFIYDEQIVWWEGLVLIILYVFYILIMKYNVKMQAFFTVKQKSIANGNPVNSELEA
GNDFYDGSYDDPSVPLLGQVKEKPQYGKNPVVMVDEIMSSSPPKFTFPEAGLRIMITNKFGPRTRLRMASRIIINERQRLINSANGVSSKPLQ
NGRHENIENGNVPVENPEDPQQNQEQQPPPQPPPPEPEPVEADFLSPFSVPEARGDKVKWVFTWPLIFLLCVTIPNCSKPRWEKFFMVTFIT
ATLWIAVFSYIMVWLVTIIGYTLGIPDVIMGITFLAAGTSVPDCMASLIVARQGLGDMAVSNTIGSNVFDILVGLGVPWGLQTMVVNYGST
VKINSRGLVYSVVLLLGSVALTVLGIHLNKWRLDRKLGVYVLVLYAIFLCFSIMIEFNVFTFVNLPMCREDD
>gi|41281583|ref|NP_056031.2| inositol hexakisphosphate and diphosphoinositol-pentakisphosphate
kinase 2 [Homo sapiens], SEQ ID NO: 132
MSEAPRFFVGPEDTEINP_GNYRHFFHHADEDDEEEDDSPPERQIVVGICSMAKKSKSKPMKEILERISLFKYITVVVFEEEVILNEPVENWPL
CDCLISFHSKGFPLDKAVAYAKLRNPFVINDLNMQYLIQDRREVYSILQAEGILLPRYAILNRDPNNPKECNLIEGEDHVEVNGEVFQKPFV
EKPVSAEDHNVYIYYPTSAGGGSQRLFRKIGSRSSVYSPESNVRKTGSYIYEEFMPTDGTDVKVYTVGPDYAHAEARKSPALDGKVERDS
EGKEVRYPVILNAREKLIAWKVCLAFKQTVCGFDLLRANGQSYVCDVNGFSFVKNSMKYYDDCAKILGNIVMRELAPQFHIPWSIPLEAE
DIPIVPTTSGTMMELRCVIAVIRHGDRTPKQKMKMEVRHQKFFDLFEKCDGYKSGKLKLKKPKQLQEVLDIARQLLMELGQNNDSEIEEN
KPKLEQLKTVLEMYGHFSGINRKVQLTYLPHGCPKTSSEEEDSRREEPSLLLVLKWGGELTPAGRVQAEELGRAFRCMYPGGQGDYAGFP
GCGLLRLHSTYRHDLKIYASDEGRVQMTAAAFAKGLLALEGELTPILVQMVKSANMNGLLDSDSDSLSSCQQRVKARLHEILQKDRDFTA
EDYEKLTPSGSISLIKSMHLIKNPVKTCDKVYSLIQSLTSQIRHRMEDPKSSDIQLYHSETLELMLRRWSKLEKDFKTKNGRYDISKIPDIYDC
IKYDVQHNGSLKLENTMELYRLSKALADIVIPQEYGITKAEKLEIAKGYCTPLVRKIRSDLQRTQDDDTVNKLHPVYSRGVLSPERHVRTRL
YFTSESHVHSLLSILRYGALCNESKDEQWKRAMDYLNVVNELNYMTQIVIMLYEDPNKDLSSEERFHVELHFSPGAKGCEEDKNLPSGY
GYRPASRENEGRRPFKIDNDDEPHTSKRDEVDRAVILFKPMVSEPIHIHRKSPLPRSRKTATNDEESPLSVSSPEGTGTWLHYTSGVGTGRR
RRRSGEQITSSPVSPKSLAFTSSIFGSWQQVVSENANYLRTPRTLVEQKQNPTVGSHCAGLFSTSVLGGSSSAPNLQDYARTHRKKLTSSGCI
DGFELYSMVPSICPLETLHNALSLKQVDEFLASIASPSSDVPRKTAEISSTALRSSPIMRKKVSLNTYTPAKILPTPPATLKSTKASSKPATSGP
SSAVVPNTSSRKKNITSKTETHEHKKNTGKKK
>gi|8659574|ref|NP_006512.2| transcription factor E3 [Homo sapiens], SEQ ID NO: 133
MSHAAEPARDGVEASAEGPRAVFVLLEERRPADSAQLLSLNSLLPESGIVADIELENVLDPDSFYELKSQPLPLRSSLPISLQATPATPATLSA
SSSAGGSRTPAMSSSSSSRVLLRQQLMRAQAQEQERRERREQAAAAPFPSPAPASPAISVVGVSAGGHTLSRPPPAQVPREVLKVQTHLEN
PTRYHLQQARRQQVKQYLSTTLGPKLASQALTPPPGPASAQPLPAPEAAHTTGPTGSAPNSPMALLTIGSSSEKEIDDVIDEIISLESSYNDE
MLSYLPGGTTGLQLPSTLPVSGNLLDVYSSQGVATPAITVSNSCPAELPNIKREISETEAKALLKERQKKDNHNLIERRRRFNINDRIKELGTL
IPKSSDPEMRWNKGTILKASVDYIRKLQKEQQRSKDLESRQRSLEQANRSLQLRIQELELQAQIHGLPVPPTPGLLSLATTSASDSLKPEQLDI
EEEGRPGAATFHVGGGPAQNAPHQQPPAPPSDALLDLHFPSDHLGDLGDPFHLGLEDILMEEEEGVVGGLSGGALSPLRAASDPLLSSVSP
AVSKASSRRSSFSMEEES
>gi|256600202|ref|NP_001157861.1| rap guanine nucleotide exchange factor 6 isoform 5
[Homo sapiens], SEQ ID NO: 134
MNSPVDPGARQALRKKPPERTPEDLNTIYSYLHGMEILSNLREHQLRLMSARARYERYSGNQVLFCSETIARCWYILLSGSVLVKGSMVLP
PCSFGKQFGGKRGCDCLVLEPSEMIVVENAKDNEDSILQREIPARQSRRRFRKINYKGERQTITDDVEVNSYLSLPADLTKMHLTENPHPQV
THVSSSQSGCSIASDSGSSSLSDIYQATESEVGDVDLTRLPEGPVDSEDDEEEDEEIDRTDPLQGRDLVRECLEKEPADKTDDDIEQLLEFMH
QLPAFANMTMSVRRELCSVMIFEVVEQAGAIILEDGQELDSWYVILNGTVEISHPDGKVENLFMGNSFGITPTLDKQYMHGIVRTKVDDCQ
FVCIAQQDYWRILNHVEKNTHKVEEEGEIVMVHEHRELDRSGTRKGHIVIKATPERLIMHLIEEHSIVDPTYIEDFLLTYRTFLESPLDVGIKL
LEWFKIDSLRDKVTRIVLLWVNNHFNDFEGDPAMTRFLEEFEKNLEDTKMNGHLRLLNIACAAKAKWRQVVLQKASRESPLQFSLNGGSE
KGFGIFVEGVEPGSKAADSGLKRGDQINEVNGQNFENITFMKAVEILRNNTHLALTVKTNIFVFKELLFRTEQEKSGVPHIPKIAEKKSNRHS
IQHVPGDIEQTSQEKGSKKVKANTVSGGRNKIRKILDKTRFSILPPKLFSDGGLSQSQDDSIVGTRHCRHSLAIMPIPGTLSSSSPDLLQPTTS
MLDFSNPSDIPDQVIRVFKVDQQSCYIIISKDTTAKEVVFHAVHEFGLTGASDTYSLCEVSVTPEGVIKQRRLPDQFSKLADRIQLNGRYYLK
NNMETETLCSDEDAQELVKESQLSMLQLSTIEVATQLSMRDFDLFRNIEPTEYIDDLFKLNSKTGNTHLKRFEDIVNQETFWVASEILTEAN
QLKRMKIIKHFIKIALHCRECKNFNSMFAIISGLNLASVARLRGTWEKLPSKYEKHLQDLQDIFDPSRNMAKYRNILSSQSMQPPIIPLFPVVK
KDMTFLHEGNDSKVDGLVNFEKLRMISKEIRQVVRMTSANMDPAMMFRQRSLSQGSTNSNMLDVQGGAHKKRARRSSLLNAKKLYED
AQMARKVKQYLSSLDVETDEEKFQMMSLQWEPAYGTLTKNLSEKRSAKSSEMSPVPMRSAGQTTKAHLHQPHRVSQVLQVPAVNLH
PIRKKGQTKDPALNTSLPQKVLGTTEEISGKKHTEDTISVASSLHSSPPASPQGSPHKVGSIISDHSSKISGQSCPGIGGAYLQKKILQITRSTA
KRTDSTEKATEENRDRTSCENTTRKRMTSPFRRLRERMLSRERLVNSQKEDTDHNQATESCEKVKDVGSNIKDEKGSAIFNSNSQGNSNT
LNCFYTRFKSKRRKTL
>gi|38016945|ref|NP_066288.2| deformed epidermal autoregulatory factor 1 homolog [Homo sapiens],
SEQ ID NO: 135
MEDSDSAAKQLGLAEAAAVAAAAAVAAAAAAAAGGEAEEPVLSRDEDSEEDADSEAERETPRVTAVAVMAAEPGHMDMGAEALPGPD
EAAAAAAFAEVTTVTVANVGAAADNVFTTSVANAASISGHVLSGRTALQIGDSLNTEKATLIVVHTDGSIVETTGLKGPAAPLTPGPQSPPT
PLAPGQEKGGTKYNWDPSVYDSELPVRCRNISGTLYKNRLGSGGRGRCIKQGENWYSPTEFEAMAGRASSKDWKRSIRYAGRPLQCLIQD
GILNPHAASCTCAACCDDMTLSGPVRLFVPYKRRKKENELPTTPVKKDSPKNITLLPATAATTFTVTPSGQITTSGALTFDRASTVEATAVIS
ESPAQGDVFAGATVQEASVQPPCRASHPEPHYPGYQDSCQIAPFPEAALPTSHPKIVLTSLPALAVPPPTPTKAAPPALVNGLELSEPRSWL
YLEEMVNSLLNTAQQLKTLFEQAKHASTYREAATNQAKIFIADAERKEQSCVNCGREAMSECTGCHKVNYCSTFCQRKDWKDHQHICGQ
SAAVTVQADEVHVAESVMEKVTV
>gi|28558973|ref|NP_004220.2| mediator of RNA polymerase II transcription subunit 14 [Homo
sapiens], SEQ ID NO: 136
MAPVQLENHQLVPPGGGGGGSGGPPSAPAPPPPGAAVAAAAAAAASPGYRLSTLIEFLLHRAYSELMVLTDLLPRKSDVERKIEIVQFASR
TRQLFVRLLALVKWANNAGKVEKCAMISSFLDQQAILFVDTADRLASLARDALVHARLPSFAIPYAIDVLTTGSYPRLPTCIRDKIIPPDPITK
IEKQATLHQLNQILRHRLVTTDLPPQLANLTVANGRVKFRVEGEFEATLTVMGDDPDVPWRLLKLEILVEDKETGDGRALVHSMQISFIHQ
LVQSRLFADEKPLQDMYNCLHSFCLSLQLEVLHSQTLMLIRERWGDLVQVERYHAGKCLSLSVWNQQVLGRKTGTASVHKVTIKIDEND
VSKPLQIFHDPPLPASDSKLVERAMKIDHLSIEKLLIDSVHARAHQKLQELKAILRGFNANENSSIETALPALVVPILEPCGNSECLHIFVDLHS
GMFQLMLYGLDQATLDDMEKSVNDDMKRIIPWIQQLKFWLGQQRCKQSIKHLPTISSETLQLSNYSTHPIGNLSKNKLFIKLTRLPQYYIVV
EMLEVPNKPTQLSYKYYFMSVNAADREDSPAMALLLQQFKENIQDLVFRTKTGKQTRTNAKRKLSDDPCPVESKKTKRAGEMCAFNKV
LAHFVAMCDTNMPFVGLRLELSNLEIPHQGVQVEGDGFSHAIRLLKIPPCKGITEETQKALDRSLLDCTFRLQGRNNRTWVAELVFANCPL
NGTSTREQGPSRHVYLTYENLLSEPVGGRKVVEMFLNDWNSIARLYECVLEFARSLPDIPAHLNIFSEVRVYNYRKLILCYGTTKGSSISIQ
WNSIHQKFHISLGTVGPNSGCSNCHNTILHQLQEMFNKTPNVVQLLQVLFDTQAPLNAINKLPTVPMLGLTQRTNTAYQCFSILPQSSTHIR
LAFRNMYCIDIYCRSRGVVAIRDGAYSLFDNSKLVEGFYPAPGLKTFLNMFVDSNQDARRRSVNEDDNPPSPIGGDMMDSLISQLQPPPQQ
QPFPKQPGTSGAYPLTSPPTSYHSTVNQSPSMMHTQSPGNLHAASSPSGALRAPSPASFVPTPPPSSHGISIGPGASFASPHGTLDPSSPYTM
VSPSGRAGNWPGSPQVSGPSPAARMPGMSPANPSLHSPVPDASHSPRAGTSSQTMPTNMPPPRKLPQRSWAASIPTILTHSALNILLLPSPTP
GLVPGLAGSYLCSPLERFLGSVIMRRHLQRIIQQETLQLINSNEPGVIMFKTDALKCRVALSPKTNQTLQLKVTPENAGQWKPDELQVLEKF
FETRVAGPPFKANTLIAFTKLLGAPTHILRDCVHIMKLELFPDQATQLKWNVQFCLTIPPSAPPIAPPGTPAVVLKSKMLFFLQLTQKTSVPPQ
EPVSIIVPIIYDMASGTTQQADIPRQQNSSVAAPMMVSNILKRFAEMNPPRQGECTIFAAVRDLMANLTLPPGGRP
>gi|38146094|ref|NP_005472.2| mediator of RNA polymerase II transcription subunit 16 [Homo
sapiens], SEQ ID NO: 137
MCDLRRPAAGGMMDLAYVCEWEKWSKSTHCPSVPLACAWSCRNLIAFTMDLRSDDQDLTRMIHILDTEHPWDLHSIPSEHHEAITCLEW
DQSGSRLLSADADGQIKCWSMADHLANSWESSVGSLVEGDPIVALSWLHNGVKLALHVEKSGASSFGEKFSRVKFSPSLTLFGGKPMEG
WIAVTVSGLVTVSLLKPSGQVLTSTESLCRLRGRVALADIAFTGGGNIVVATADGSSASPVQFYKVCVSVVSEKCRIDTEILPSLFMRCTTDL
NRKDKFPAITHLKFLARDMSEQVLLCASSQTSSIVECWSLRKEGLPVNNIFQQISPVVGDKQPTILKWRILSATNDLDRVSAVALPKLPISLT
NTDLKVASDTQFYPGLGLALAFHDGSVHIVHRLSLQTMAVFYSSAAPRPVDEPAMKRPRTAGPAVHLKAMQLSWTSLALVGIDSHGKLS
VLRLSPSMGHPLEVGLALRHLLFLLEYCMVTGYDWWDILLHVQPSMVQSLVEKLHEEYTRQTAALQQVLSTRILAMKASLCKLSPCTVTR
VCDYHTKLFLIAISSTLKSLLRPHFLNTPDKSPGDRLTEICTKITDVDIDKVMINLKTEEFVLDMNTLQALQQLLQWVGDFVLYLLASLPNQG
SLLRPGHSFLRDGTSLGMLRELMVVIRIWGLLKPSCLPVYTATSDTQDSMSLLFRLLTKLWICCRDEGPASEPDEALVDECCLLPSQLLIPSL
DWLPASDGLVSRLQPKQPLRLQFGRAPTLPGSAATLQLDGLARAPGQPKIDHLRRLHLGACPTEECKACTRCGCVTMLKSPNRTTAVKQW
EQRWIKNCLAVEGRGPDACVTSRASEEAPAFVQLGPQSTHHSPRTPRSLDHLHPEDRP
>gi|116063554|ref|NP_057038.2| putative deoxyribose-phosphate aldolase [Homo sapiens],
SEQ ID NO: 138
MSAHNRGTELDLSWISKIQVNHPAVLRRAEQIQARRTVKKEWQAAWLLKAVTFIDLTTLSGDDTSSNIQRLCYKAKYPIREDLLKALNMH
DKGITTAAVCVYPARVCDAVKALKAAGCNIPVASVAAGFPAGQTHLKTRLEEIRLAVEDGATEIDVVINRSLVLTGQWEALYDEIRQFRKA
CGEAHLKTILATGELGTLTNVYKASMIAMMAGSDFIKTSTGKETVNATFPVAIVMLRAIRDFFWKTGNKIGFKPAGGIRSAKDSLAWLSLV
KEELGDEWLKPELFRIGASTLLSDIERQIYHHVTGRYAAYHDLPMS
>gi|4758668|ref|NP_004854.1| serine palmitoyltransferase 2 [Homo sapiens], SEQ ID NO: 139
MRPEPGGCCCRRTVRANGCVANGEVRNGYVRSSAAAAAAAAAGQIHHVTQNGGLYKRPFNEAFEETPMLVAVLTYVGYGVLTLFGYLR
DFLRYWRIEKCHHATEREEQKDFVSLYQDFENFYTRNLYMRIRDNWNRPICSVPGARVDIMERQSHDYNWSFKYTGNIIKGVINMGSYN
YLGFARNTGSCQEAAAKVLEEYGAGVCSTRQEIGNLDKHEELEELVARFLGVEAAMAYGMGFATNSMNIPALVGKGCLILSDELNHASLV
LGARLSGATIRIFKHNNMQSLEKLLKDAIVYGQPRTRRPWKKILILVEGIYSMEGSIVRLPEVIALKKKYKAYLYLDEAHSIGALGPTGRGVV
EYFGLDPEDVDVMMGTFTKSFGASGGYIGGKKELIDYLRTHSHSAVYATSLSPPVVEQIITSMKCIMGQDGTSLGKECVQQLAENTRYFRR
RLKEMGFIIYGNEDSPVVPLMLYMPAKIGAFGREMLKRNIGVVVVGFPATPIIESRARFCLSAAHTKEILDTALKEIDEVGDLLQLKYSRHRL
VPLLDRPFDETTYEETED
>gi|7705935|ref|NP_057415.1| zinc finger protein 639 [Homo sapiens], SEQ ID NO: 140
MNEYPKKRKRKTLHPSRYSDSSGISRIADGFNGIFSDHCYSVCSMRQPDLKYFDNKDDDSDTETSNDLPKFADGIKARNRNQNYLVPSPVL
RILDHTAFSTEKSADIVICDEECDSPESVNQQTQEESPIEVHTAEDVPIAVEVHAISEDYDIETENNSSESLQDQTDEEPPAKLCKILDKSQAL
NVTAQQKWPLLRANSSGLYKCELCEFNSKYFSDLKQHMILKHKRTDSNVCRVCKESFSTNMLLIEHAKLHEEDPYICKYCDYKTVIFENLS
QHIADTHFSDHLYWCEQCDVQFSSSSELYLHFQEHSCDEQYLCQFCEHETNDPEDLHSHVVNEHACKLIELSDKYNNGEHGQYSLLSKITF
DKCKNFFVCQVCGFRSRLHTNVNRHVAIEHTKIFPHVCDDCGKGFSSMLEYCKHLNSHLSEGIYLCQYCEYSTGQIEDLKIFILDFKHSADL
PHKCSDCLMRFGNERELISHLPVHETT
>gi|304555614|ref|NP_689956.2| BRCA1-associated ATM activator 1 [Homo sapiens], SEQ ID NO: 141
MDPECAQLLPALCAVLVDPRQPVADDTCLEKLLDWFKTVTEGESSVVLLQEHPCLVELLSHVLKVQDLSSGVLSFSLRLAGTFAAQENCF
QYLQQGELLPGLFGEPGPLGRATWAVPTVRSGWIQGLRSLAQHPSALRFLADHGAVDTIFSLQGDSSLFVASAASQLLVHVLALSMRGGA
EGQPCLPGGDWPACAQKIMDHVEESLCSAATPKVTQALNVLTTTFGRCQSPWTEALWVRLSPRVACLLERDPIPAAHSFVDLLLCVARSP
VFSSSDGSLWETVARALSCLGPTHMGPLALGILKLEHCPQALRTQAFQVLLQPLACVLKATVQAPGPPGLLDGTADDATTVDTLLASKSSC
AGLLCRTLAHLEELQPLPQRPSPWPQASLLGATVTVLRLCDGSAAPASSVGGHLCGTLAGCVRVQRAALDFLGTLSQGTGPQELVTQALA
VLLECLESPGSSPTVLKKAFQATLRWLLSSPKTPGCSDLGPLIPQFLRELFPVLQKRLCHPCWEVRDSALEFLTQLSRHWGGQADFRCALLA
SEVPQLALQLLQDPESYVRASAVTAMGQLSSQGLHAPTSPEHAEARQSLFLELLHILSVDSEGFPRRAVMQVFTEWLRDGHADAAQDTEQ
FVATVLQAASRDLDWEVRAQGLELALVFLGQTLGPPRTHCPYAVALPEVAPAQPLTEALRALCHVGLFDFAFCALFDCDRPVAQKSCDLL
LFLRDKIASYSSLREARGSPNTASAEATLPRWRAGEQAQPPGDQEPEAVLAMLRSLDLEGLRSTLAESSDHVEKSPQSLLQDMLATGGFLQ
GDEADCY
>gi|89111122|ref|NP_001034750.1| toll/interleukin-1 receptor domain-containing adapter protein
isoform a [Homo sapiens], SEQ ID NO: 142
MASSTSLPAPGSRPKKPLGKMADWFRQTLLKKPKKRPNSPESTSSDASQPTSQDSPLPPSLSSVTSPSLPPTHASDSGSSRWSKDYDVCVCH
SEEDLVAAQDLVSYLEGSTASLRCFLQLRDATPGGAIVSELCQALSSSHCRVLLITPGFLQDPWCKYQMLQALTEAPGAEGCTIPLLSGLSR
AAYPPELRFMYYVDGRGPDGGFRQVKEAVMRYLQTLS

1-17. (canceled)

18. A method for determining disease progression in a subject after a stroke, comprising:
(a) measuring the level of one or more biomarkers in a first sample obtained from the subject at a first time point;
(b) measuring the level of the one or more biomarkers in a second sample obtained from the subject at a second time point;
(c) comparing the level of the one or more biomarkers at the first time point to the level of the one or more biomarkers at the second time point; and
(d) determining the disease progression between the first and the second time point based on the result of step (c),
wherein the one or more biomarkers at the second time point; and (d) determining the disease progression between the first and the second time point based on the result of step (c), wherein the one or more biomarkers comprise gene products expressed from genes selected from the group consisting of IL-1α, IL-1β, IL-1ra, IL-3, IL-2, IL-4, IL-5, IL-6, IL-7, IL-8, IL-9, IL-10, IL-12 (p40), IL-12(p70), IL-13, IL-15, IL-17, EGF, Eotaxin, FGF-2, FTL-3 ligand, Fractalkine, G-CSF, GM-CSF, GRO, IFN-α2, IFN-γ, IP-10, MCP-1, MCP-3, MCD, MIP-1α, MIP-1β, PDGF-aa, PGDF-aa bb, RANTES, sCD40L, sIL2-rα, TNF-α, TNF-β, VEGF and genes listed in Tables 4, 5, 6 and 8.

19. A method for determining the efficacy of a treatment for stroke in a subject, comprising:
(a) measuring the level of one or more biomarkers in a first sample obtained from the subject at a first time point;
(b) measuring the level of the one or more biomarkers in a second sample obtained from the subject at a second time point, wherein the subject is under treatment at the second time point;
(c) comparing the level of the one or more biomarkers at the first time point to the level of the one or more biomarkers at the second time point; and
(d) determining the efficacy of the treatment based on the result of step (c),
wherein the one or more biomarkers comprise gene products expressed from genes selected from the group consisting of IL-1α, IL-1β, IL-1ra, IL-3, IL-2, IL-4, IL-5, IL-6, IL-7, IL-8, IL-9, IL-10, IL-12 (p40), IL-12(p70), IL-13, IL-15, IL-17, EGF, Eotaxin, FGF-2, FTL-3 ligand, Fractalkine, G-CSF, GM-CSF, GRO, IFN-α2, IFN-γ, IP-10, MCP-1, MCP-3, MCD, MIP-1α, MIP-1β, PDGF-aa, PGDF-aa bb, RANTES, sCD40L, sIL2-rα, TNF-α, TNF-β, VEGF, NMDA receptor, neuronal specific enolase, GFAP, Apo C-III, MMP-9, D-dimer, CRP, brain natriuretic peptide, S100B and genes listed in Tables 4, 5, 6 and 8.

20. A kit for detecting biomarkers for stroke in a biological sample, comprising:
(a) reagents for detecting a panel of biomarkers for stroke, and
(b) a instruction listing the reference range for each of the biomarkers,
wherein the panel of biomarkers comprise two or more biomarkers, and wherein the two or more biomarkers comprise gene products expressed from genes selected from the group consisting of IL-1α, IL-1β, IL-1ra, IL-3, IL-2, IL-4, IL-5, IL-6, IL-7, IL-8, IL-9, IL-10, IL-12 (p40), IL-12(p70), IL-13, IL-15, IL-17, EGF, Eotaxin, FGF-2, FTL-3 ligand, Fractalkine, G-CSF, GM-CSF, GRO, IFN-α2, IFN-γ, IP-10, MCP-1, MCP-3, MCD, MIP-1α, MIP-1β, PDGF-aa, PGDF-aa bb, RANTES, sCD40L, sIL2-rα, TNF-α, TNF-β, VEGF, NMDA receptor, neuronal specific enolase, GFAP, Apo C-III, MMP-9, D-dimer, CRP, brain natriuretic peptide, S100B and genes listed in Tables 4, 5, 6 and 8.

21. The method of claim 1, wherein the one or more biomarkers are polynucleotides.

22. The method of claim 1, wherein the one or more biomarkers are peptides

23. The method of claim 1, wherein step (a) includes measuring a panel of three or more biomarkers in the sample from the subject.

24. The method of claim 23, wherein the three or more biomarkers comprise expression product of at least one gene listed in Table 4, at least one gene listed in Table 5 and at least one gene listed in Table 6.

25. The method of claim 18, wherein step (a) includes measuring a panel of six or more biomarkers in the sample from the subject.

26. The method of claim 25, wherein the six or more biomarkers comprise expression product of at least two genes listed in Table 4, at least two genes listed in Table 5 and at least two genes listed in Table 6.

27. The method of claim 18, wherein step (a) includes measuring a panel of nine or more biomarkers in the sample from the subject.

28. The method of claim 27, wherein the nine or more biomarkers comprise expression product of at least three genes listed in Table 4, at least three genes listed in Table 5 and at least three genes listed in Table 6.

29. The method of claim 18, wherein step (a) includes measuring a panel of twenty or more biomarkers in the sample from the subject.

30. The method of claim 18 further comprising step (c) making a diagnosis based on the result of said comparing step (b).

31. The method of claim 18, wherein the sample is a body fluid sample or a tissue sample.

32. The method of claim 31, wherein the body fluid sample is a blood sample.

33. The method of claim 31, wherein the body fluid sample is a plasma or serum sample.

34. The method of claim 31, wherein the body fluid sample is a cerebrospinal fluid sample.

35. The method of claim 31, wherein the sample is peripheral blood mononuclear cells (PBMCs).
